(19) United States
(12) Patent Application Publication
(10) Pub. No.: US 2026/0060816 A1
(43) Pub. Date: Mar. 5, 2026

(54) SMART SHOULDER TISSUE SPARING APPROACH TECHNIQUES AND RELATED INSTRUMENTATION

(71) Applicant: DePuy Synthes Products, Inc., Raynham, MA (US)

(72) Inventors: Stephen J. Orphanos, Bridgewater, MA (US); Mollie Waters, Boston, MA (US); Raymond Murphy, Manville, RI (US); Robert Ciocca, Woonsocket, RI (US); Jae S. Stelzer, Raynham, MA (US); Paul Favorito, Cincinnati, OH (US); Peter Chalmers, Salt Lake City, UT (US); Anders Ekelund, Bromma (SE)

(21) Appl. No.: 19/071,672

(22) Filed: Mar. 5, 2025

Related U.S. Application Data
(60) Provisional application No. 63/689,631, filed on Aug. 30, 2024.

(51) Int. Cl. A61F 2/46; A61B 17/17

(52) U.S. Cl. CPC A61F 2/4612; A61B 17/1778; A61F 2002/4681; A61F 2002/4687

(57) ABSTRACT

Procedures for performing shoulder arthroplasties are disclosed, such procedures including performing various preparation steps for the humerus and glenoid to receive implants, and proceeding through placement of implants and/or prostheses at the surgical site. The techniques disclosed afford the benefit of being tissue sparing, and thus minimize damage or trauma to tissue at the surgical site, including allowing the subscapularis tendon to remain intact throughout the procedures. Various devices that enable the procedures are disclosed, including but not limited to humeral resection guides, glenoid guides, humeral guides, and handle assemblies, among other tools, components, and the like that can be used with such devices. In at least some embodiments, preparation steps include resecting a humeral head, and reaming and broaching the resulting humeral resection surface to receive an implant, and then a prosthesis, as well as reaming a glenoid so it can also receive an implant.

### CROSS REFERENCE TO RELATED APPLICATION

The present disclosure claims priority to and the benefit of U.S. Provisional Patent Application No. 63/689,631, entitled “Smart Shoulder Tissue Sparing Approach Techniques and Related Instrumentation,” filed Aug. 30, 2024, the disclosure of which is incorporated by reference herein in its entirety.

### FIELD

The present disclosure relates to methods, techniques, and devices used in shoulder repair procedures, such as shoulder arthroplasties, and more particularly relates to methods and associated devices used to prepare a humerus and/or a glenoid in a limited space, such as through the rotator interval, while allowing surrounding tendons such as the subscapularis tendon to remain intact.

### BACKGROUND

The glenohumeral joint (i.e., the “ball-and-socket” joint) is where the humerus bone head (the ball) and the glenoid (the socket) meet. The rotator cuff spans the glenohumeral joint and connects the humerus to the scapula. Muscles of the rotator cuff keep the humerus tightly in the socket, while tendons of the rotator cuff attach the muscle to bone.

During the lifetime of a patient, it may be necessary to perform a shoulder arthroplasty due to, for example, disease or trauma. Various forms of this type of surgery exist, with the overarching purpose being to remove and/or replace damaged or missing portions of the anatomy with prosthetic components. For example, as shown in FIG. 1A, in an anatomic total shoulder arthroplasty, a humeral prosthesis 10 can be used to replace the natural head of a patient's humerus. The humeral prosthesis 10 typically includes an elongated post component 12 that is implanted into an intramedullary canal of the patient's humerus and a hemispherical-shaped prosthetic head component 14 that is secured to the post component 12. Additionally, the natural glenoid surface of the scapula can be resurfaced or otherwise replaced with an anatomic glenoid implant 20. An anatomic glenoid implant 20 typically includes a concave bearing surface 24 upon which the prosthetic head component 14 of the humeral prosthesis 10 articulates. A peg or keel 22 can project from the distal end of the implant 20 and can be secured (e.g., cemented) into the glenoid cavity of the patient's scapula.

While FIG. 1A provides for an anatomic total shoulder arthroplasty, in other instances, the arthroplasty may be a partial shoulder arthroplasty, meaning only a portion of the shoulder anatomy may be replaced with an implant. This may include, for example, only providing for a humeral prosthesis without a glenoid implant, or a glenoid implant without a humeral prosthesis, among other variations appreciated by those skilled in the art. Further, while FIG. 1A provides for an anatomic procedure, reverse procedures are also known procedures for repairing shoulders. They can be helpful, for example, when a patient's natural shoulder has degenerated to a severe degree of joint instability and pain. In a reverse procedure, the mechanics of the shoulder can be changed, reversing the anatomy, or structure, of the healthy shoulder. For example, as shown in FIG. 1B, in a reverse total shoulder arthroplasty, a humeral prosthesis 50 can be used to replace the natural head of the patient's humerus. The humeral prosthesis 50 typically includes an elongated post component 52 that is implanted into an intramedullary canal of the patient's humerus and a concave-shaped prosthetic head component 54, known as a humeral cup, is secured to the post component 52. Additionally, a reverse glenoid implant, e.g., a hemispherical-shaped glenosphere 60, can be secured to the glenoid bone of the patient's scapula. Such a reverse configuration allows the patient's deltoid muscle, which is one of the larger and stronger shoulder muscles, to raise the arm.

During various types of shoulder arthroplasty surgeries, there is soft tissue impeding access to the surgical arthroplasty site. This tissue typically includes a patient's subscapularis tendon. In many traditional surgical approaches, the subscapularis tendon is detached from a humeral attachment point on the humerus to provide better access to the surgical site. In at least some arthroplasty procedures, the humerus is subsequently externally rotated to allow access to the joint space, essentially dislocating the humeral head. This provides a surgeon full visibility to the humeral head, as well as the glenoid after humeral head resection. In an anatomical procedure in which the humeral head is being replaced, once the humeral head is exposed, the convex portion of the bone is resected to a flat plane and prepared to receive a humeral prosthesis. In a reverse procedure, the location where the humeral head is typically located can be prepared to receive a prosthetic head component (e.g., a humeral cup) after the humeral head is externally rotated.

Upon gaining sufficient access to the surgical site and resecting the humeral head, surgeons often employ various tools to create the proper geometry within the resected surface of the humerus (hereinafter referred to as the “humeral resection surface”). This geometry depends, at least in part, on the chosen implant and/or prosthesis and/or any anatomical or disease issues associated with the patient at the surgical site to ensure that the implant and/or prosthesis fits securely with respect to the bone so it can perform properly within the joint space. Generally to create such a geometry, the humeral resection surface is reamed, cut, or otherwise shaped to the desired shape and then the reamed surface is broached or otherwise impacted prior to inserting an implant and/or prosthesis. Existing reaming and broaching tools, among other instruments used in such procedures, are designed for use in procedures in which the subscapularis tendon has been detached from its humeral attachment point to allow a full 360° view of the humeral head anatomy, for instance while operating reaming and broaching tools and using tools to assist in the same. The size, shape, and function of such tools are based on having access and visualization to the joint space that is not inhibited by the subscapularis tendon and/or other tissue that may impede access to the surgical site. For example, to form the proper geometry in the humeral resection surface, a sufficient amount of downward force is placed on the humeral resection surface by the tools. Applying a consistent amount of downward force across the humeral resection surface requires direct access to the humeral resection surface. Following surgery, the subscapularis tendon is reattached. Repair and healing of the tendon are critical to the proper function of the joint and incomplete healing results in complications, pain, and instability.

Alternatively, the procedure can be performed leaving the subscapularis tendon attached-referred to as tissue sparing-such that the surgeon(s) works only within the limited joint space superior and inferior to the tissue borders of the subscapularis. In such instances, because the surgeon is not able to externally rotate and essentially dislocate the humeral head, he or she has limited access to the humeral head to assess the anatomy associated with the procedure, such as assessing a cutting plane and/or bone surfaces, as well as limited access to a deeper anatomy like the surface of the glenoid. To the extent the surgeon uses existing arthroplasty tools that are not specifically designed for a tissue sparing approach, he or she manipulates the subscapularis tendon during tool insertion to allow the tools to access the surgical site. This is because traditional tools are not suitable and/or designed for use in such a tissue sparing procedure. Beyond the visual complications noted above, the narrow joint space makes it difficult for the surgeon to apply sufficient forces in a consistent manner that is necessary for humeral bone preparation. Unless a surgeon dislocates and fully externally rotates the humerus, the coracoid and acromion limits perpendicular access and exposure to a cutting or resecting plane, which is typically how humeral bone preparation is done in known shoulder arthroplasty techniques. Further, traditional arthroplasty tools are not being used during transhumeral procedures at least because, prior to the present disclosure, transhumeral approaches for shoulder arthroplasty were not being performed. At least because existing arthroplasty tools are designed for use when the subscapularis tendon has been detached, they are too bulky and cumbersome to use in a tissue sparing procedure. Existing tools do not have the versatility in movement, size, and function to access a limited joint space and provide consistent and sufficient force to adequately prepare a humeral surface and/or a glenoid surface to receive a prosthesis. The size and/or trajectory of existing tools are insufficient. They are not designed to be low profile so as to be able to work within confined spaces, nor are they modular to allow for assembly/disassembly within the joint space. Further, to the extent existing devices have at least some such capabilities, they are not typically easy to use, meaning existing devices are insufficient to provide suitable usability to surgeons and the like.

Accordingly, there is a need for humeral and glenoid arthroplasty tools and instruments, and related methods, for use in tissue sparing arthroplasty procedures where access is limited while also minimizing damage to surrounding soft tissue, and adjacent neurovascular and boney structures.

### SUMMARY

The present disclosure is generally directed to methods for performing steps of a shoulder arthroplasty in a joint space limited by intact tendons and soft tissue. The disclosed procedures starts from initial steps of accessing and/or assessing the area where the procedure is to be performed, and concludes by completing the repair and associated implantation(s). Various embodiments of surgical tools, including but not limited to humeral resection guides, glenoid guides, and humeral guides, are disclosed. The surgical tools disclosed herein can help resect a humeral head at a precise location and angle with respect to the humerus and then set a path of travel and/or location at which various instruments used to perform the surgical procedures are to be located with respect to the surgical site. The tools can also be used in performing the various portions of the procedure, such as preparing and treating a glenoid using a transhumeral approach (though a non-transhumeral approach is also possible using aspects of the instrument and techniques disclosed herein), resecting, reaming, and/or broaching portions of bone, such as a proximal portion of the humerus. Notably, the handle assemblies and related disclosures herein enable the ability to perform tasks, such as cutting, reaming, and/or broaching, while the subscapularis tendon remains intact with a humeral attachment point for the duration of the surgical procedure.

Various instrumentation and tools disclosed herein, as well as the techniques that can be performed in conjunction with the same, provide unique, adaptable, and/or versatile designs that enable for shoulder procedures to be completed in a tissue sparing manner.

For example, humeral resection guides of the present disclosure can be used to mark and guide the resecting of a humeral head while keeping surrounding tissue (e.g., subscapularis tendon) intact and minimizing any damage to such tissue. The guides can have various degrees of freedom, enabling for virtually any positioning of the guide to be achieved to define a cutting or resecting plane at a desired position and/or accommodate resecting from any approach. These degrees can include inferior movement, superior movement, a vertical movement, a swinging movement, and/or a rotational movement. More particularly, with respect to use in a shoulder procedure, the humeral resection guides of the present disclosure allow a surgeon to adjust and take into consideration the following aspects: (1) positioning vertically on an inferior articular margin; (2) positioning superiorly against a superior articular margin at the attachment of the supraspinatus and greater tuberosity; (3) version alignment with patient anatomy; (4) alignment with the humeral long bone to achieve a specific cut plane angle; and/or (5) adjustment of the resection guide to closely approximate peripheral anatomy, for example for securing and/or pinning to the bone before resection of a boney anatomy. A person skilled in the art will appreciate the glenohumeral joint has six degrees of freedom, axial and rotational movement along three axes. The versatility of the disclosed resection guides account for these degrees of freedom when defining the cutting plane. More specifically, features are present to place the guide at a correct angle of inclination and version angle to mimic patient anatomy. The angle of inclination can be defined as the angle the head of the humerus projects from the longitudinal axis of the humeral shaft, and the version angle can be defined by the angle of rotation of the humeral head in the transverse plane.

By way of further examples, humeral guides and handle assemblies can be used in conjunction with various portions of surgical procedures that enable surrounding tissue (e.g., subscapularis tendon) to be kept intact and minimizing any damage to such tissue. More specifically, the humeral guides can help set a path of travel and/or location at which various instruments used to perform various aspects of the surgical procedures are to be located with respect to the surgical site (e.g., humeral resection surface), while the handle assemblies can be coupled to a guide, like the humeral guide(s) disclosed herein, and used to position various tools for performing various functions of a surgical procedure at the surgical site (e.g., cutting, broaching, etc.). Similar to the humeral resection guides, the humeral guides and handle assemblies, and their related disclosures, enable the ability to perform tasks, such as cutting, broaching, and/or impacting (also referred to as impaction), while the subscapularis tendon remains intact with a humeral attachment point for the duration of the surgical procedure.

By way of still further examples, glenoid guides can be used to prepare a glenoid as part of a surgical procedure, like shoulder arthroplasties or other types of surgical procedures that can be performed using a transhumeral approach. However, while the present disclosure often indicates the primary or main bone preparation steps are performed with a transhumeral approach, that does not mean that each and every step of an entire procedure must be performed transhumerally. Certain portions of one or more procedures may be performed with a non-transhumeral approach, such as within a rotator interval while the subscapularis is intact but performed non-transhumerally. Throughout the present disclosure, references to certain aspects of a procedure involving a transhumeral approach encompasses these variations, and more generally refers to the primary bone preparation steps, particularly with respect to the glenoid, being performed with a transhumeral approach. As provided for herein, the tools to be used in conjunction with treating or otherwise preparing a surface of the glenoid to receive an implant and/or prosthesis can be introduced separate from components that are used to operate the tools, such as drivers or other shafts. For example, a reamer attachment can be introduced to a glenohumeral joint space adjacent to the glenoid being treated through a rotator interval and then a driver configured to operate the reamer attachment can be introduced into the glenohumeral joint space through a transhumeral bone tunnel formed in the humerus. The reamer attachment and driver can be coupled together within the glenohumeral joint space, with the driver then being operated to rotate and translate the reamer attachment so that it reams the surface of the glenoid in preparation for receiving an implant (e.g., a prosthesis). Notably, the present disclosure accounts for multiple types of transhumeral approaches, including approaches when the arm is in adduction and when it is not in adduction. Further, guides, such as humeral guides disclosed herein, can be used to locate and/or otherwise denote a center or approximate center of a surface of the glenoid in conjunction with treating the glenoid surface.

The humeral resection guides, humeral guides, handle assemblies, and glenoid guides, as well as other components used in conjunction with the same, can be universal in nature in that they can be easily used by a surgeon who predominantly uses either hand, left or right, and from any side of the body and/or any side of the guides/frames/assemblies being operated by the surgeon. A variety of other “ease of use” features are provided in the various designs of the instrumentation and tools disclosed herein. These features can enable the use of these components at a surgical site during tissue sparing arthroplasty procedures at least because they provide for alternative approaches to supplying the necessary force(s) to perform functions, such as, by way of non-limiting examples, reaming and/or broaching, to prepare the humeral resection surface to receive an implant. Such features can also make it easy to identify various points of interest, and/or locations on some of the instruments or at the surgical site, during use.

One embodiment of a surgical method includes exposing a glenohumeral joint and glenohumeral joint space, coupling a resection guide to at least one of a humeral head or a humerus of the glenohumeral joint, introducing a cutting instrument to the glenohumeral joint space, and resecting at least a portion of the humeral head located at the glenohumeral joint using the resection guide to guide the cutting instrument and creating a humeral resection surface. The method further includes removing the resection guide and the cutting instrument from the glenohumeral joint space, and coupling a humeral guide to at least one of the humeral resection surface or the humerus such that a proximal portion of the humeral guide is disposed below the humeral resection surface, at a location that is opposed to the humeral resection surface, and a distal portion of the humeral guide is disposed proximate to a rotator interval, approximately aligned with the humeral resection surface. The method still further includes coupling a humeral sizer attachment to the distal end of the humeral guide, identifying an approximate center point of the humeral resection surface with a distal portion of the humeral sizer attachment, forming a transhumeral bone tunnel in the humerus using the humeral guide and the humeral sizer attachment to guide a drill bit through the humerus and the humeral resection surface, and de-coupling the humeral sizer attachment from the distal end of the humeral guide. The method also includes introducing a distal end of a handle assembly to the glenohumeral joint space, the distal end of the handle assembly having coupled thereto a humeral bone preparation instrument, coupling the handle assembly to the distal end of the humeral guide, passing a guide pin through the transhumeral bone tunnel, and coupling the guide pin to the humeral bone preparation instrument. The method still further includes operating the humeral bone preparation instrument to treat the humeral resection surface, de-coupling the guide pin from the humeral bone preparation instrument, and removing the distal end of the handle assembly and the humeral bone preparation instrument from the glenohumeral joint space.

The method can further include performing an inferior release of a subscapularis tendon of the glenohumeral joint. Additionally, or alternatively, the method can include performing a superior release of a subscapularis tendon. The method can include marking one or more anatomical landmarks within the glenohumeral joint space. In some such embodiments, this can include marking a bicipital groove of the humerus. The method can include moving a subscapularis tendon one of inferiorly or superiorly and holding it in place with a displacement wrap.

In at least some embodiments, resecting at least a portion of a humeral head located at the glenohumeral joint can include engaging the humeral head with a superior arm of a resection guide, passing at least one bone pin through a portion of the resection guide and into the humeral head and passing the cutting instrument through a guide slot formed on the resection guide. Engaging the humeral head with a superior arm of a resection guide can include engaging the humeral head at a location that is at least one of at or proximate to a supraspinatus attachment point on the humeral head. The method can include aligning the guide slot to at least one of a bicipital groove of the humerus or a rotator interval. In some embodiments, the method can include aligning a vertical alignment plate of the resection guide with an elongate shaft of the humerus to set a location of the superior arm. The vertical guide rod can be coupled to at least one of the vertical alignment plate or a handle coupled to the vertical alignment plate, with the vertical guide rod extending along the elongate shaft of the humerus in conjunction with aligning the vertical alignment plate of the resection guide with the elongate shaft of the humerus. The method can include moving the vertical alignment plate to change an angle of inclination of a cutting plane defined by the resection guide.

The action of passing at least one bone pin through a portion of the resection guide and into the humeral head can occur such that the at least one bone pin does not pass through soft tissue in the glenohumeral joint space. In at least some embodiments, the method can include mating a handle to the resection guide, and checking angular alignment with a forearm using the handle. This can further include manipulating the handle to adjust a location of the superior arm of the resection guide.

In at least some embodiments, the method can include mating an extender to the resection guide, with the extender being configured to extend a cutting plane defined by the superior arm of the resection guide inferior to the subscapularis tendon. In at least some such embodiments, the method can also include passing at least one inferior bone pin through a portion of the extender and into the humeral head such that the at least one inferior bone pin does not pass through soft tissue in the glenohumeral joint space.

The method can include introducing a reamer to the glenohumeral joint space, reaming a glenoid surface of a glenoid of the glenohumeral joint using the reamer, and removing the reamer from the glenohumeral joint space. In at least some such embodiments, the method can also include introducing a glenoid sizer plate to the glenohumeral joint, using the glenoid sizer plate to at least one of determine a size of the glenoid surface or determine a preferred location on the glenoid surface, and removing the glenoid sizer plate from the glenohumeral joint space. Introducing a glenoid sizer plate to the glenohumeral joint space can include using a sizer handle to pass the glenoid sizer through the rotator interval, the glenoid sizer being positioned substantially flush with the glenoid surface. The method can sometimes include passing a drill bit through the glenoid sizer to form a pilot hole in the glenoid surface. In at least some embodiments, the method can include coupling a glenoid guide to at least one of the humeral resection surface or the humerus such that a distal portion of the glenoid guide is disposed between the humeral resection surface and a surface of a glenoid of the glenohumeral joint, and forming a second transhumeral bone tunnel using the glenoid guide to guide a drill bit through the humerus.

The drill bit can pass through the second transhumeral bone tunnel to then pass through the glenoid sizer to form a pilot hole in the glenoid surface. In at least some such embodiments, the drill bit can be a stepped drill bit with a smaller diameter at a distal tip such that the pilot hole is smaller than the second transhumeral bone tunnel. The action of coupling a glenoid guide to at least one of the humeral resection surface and the humerus can further include passing a glenoid guide drill cannula through a proximal end of the glenoid guide and coupling a distal end of the glenoid guide drill cannula to a lateral surface of the humerus. Coupling a distal end of the drill cannula to a lateral surface of the humerus can set a trajectory for the drill bit forming the second transhumeral bone tunnel that is substantially central and substantially perpendicular to the surface of the glenoid. The method can also include passing the drill bit through the glenoid guide drill cannula and through the humerus to form the second transhumeral bone tunnel. The glenoid guide can be removed from the glenohumeral joint space prior to the actions of introducing a reamer to the glenohumeral joint space and reaming the glenoid surface using the reamer occur. The action of reaming the glenoid surface using the reamer can include passing a driver through the second transhumeral bone tunnel, coupling the reamer to the driver, and operating the driver to ream the surface of the glenoid. The method can further include de-coupling the reamer from the driver and removing at least the reamer from the glenohumeral joint space. The removal can occur through the rotator interval.

In at least some embodiments, the method can include introducing a glenoid implant into the glenohumeral joint space and implanting the glenoid implant into the glenoid surface. The action of introducing a glenoid implant into the glenohumeral joint space can include passing the glenoid implant through the rotator interval and implanting the glenoid implant into the glenoid surface. The glenoid implant can be removably coupled to a glenoid guide such that the glenoid guide is used to pass the glenoid implant through the rotator interval for introduction into the glenohumeral joint space. Further, the glenoid guide can be the same glenoid guide as the earlier-mentioned glenoid guide, or it can be a different glenoid guide. The method can also include detaching the glenoid implant from the glenoid guide, introducing an impactor tool into the glenohumeral space by passing the impactor tool through the rotator interval, and driving the impactor tool into the glenoid implant to further seat the glenoid implant into the glenoid surface. The impactor tool can be coupled to a glenoid guide such that the glenoid guide is used to pass the glenoid implant through the rotator interval for introduction into the glenohumeral joint space. Further, the glenoid guide can be the same glenoid guide as the earlier mentioned glenoid guide(s), or it can be a different glenoid guide.

The action of identifying an approximate center point of the humeral resection surface with a distal portion of the humeral sizer attachment can include positioning the distal portion of the humeral sizer attachment such that is aligned to be substantially parallel with the humeral resection surface and aligning an anatomical landmark of the humerus with an indicator of the humeral sizer attachment. The action of coupling a humeral guide to at least one of the humeral resection surface or the humerus can include passing a drill cannula through a hub of the humeral guide and coupling a distal end of the drill cannula to a lateral cortex of the humerus. The action of forming a transhumeral bone tunnel in the humerus can include passing a drill bit through the drill cannula and through the humerus to form the transhumeral bone tunnel.

In at least some embodiments, the method can include coupling at least one bone pin to the humeral guide and passing the at least one bone pin into the humerus. Coupling at least one bone pin to the humeral guide can include passing the bone pin through a bone pin-receiving fixation feature coupled to the humeral guide and operating the bone pin-receiving fixation feature to secure a position of the bone pin with respect to the bone pin-receiving fixation feature. In at least some such embodiments, the method can include operating the bone pin-receiving fixation feature to adjust at least one of a location of the bone pin with respect to the humerus or an angle of entry of the bone pin with respect to the humerus. Multiple degrees of freedom of the humeral guide can be adjusted to adjust the location (e.g., along a post) and the angle of entry. In at least some embodiments, there are two angles to be adjusted. One such angle allows an orientation adjustment by rotating an upper body of a pin-receiving component, and a second such angle allows the guide to rotate by rotating the whole assembly on a humeral guidepost. The adjustment of these two angles provides the ability to adapt to any type of patient anatomy. The method can also include operating the bone pin-receiving fixation feature to adjust a location of the bone pin-receiving fixation feature with respect to a support rod coupled to the humeral guide and on which the bone pin-receiving fixation feature is disposed.

The method can include coupling at least one support rod to the humeral guide and coupling the at least one bone pin-receiving fixation feature to the at least one support rod. The bone pin-receiving fixation feature can include a bone pin clamp having at least one bone-pin receiving opening for receiving the bone pin, the bone pin clamp being configured to be able to adjust at least one of a location of the bone pin with respect to the humerus or an angle of entry of the bone pin with respect to the humerus. The adjustment of the location and/or the angle of entry can be considered adjustment of an orientation of the bone pin. In at least some such embodiments, the bone pin clamp can have at least one support-rod receiving opening for receiving at least one support rod of the humeral guide. The bone pin clamp can be configured to be able to adjust a location of the clamp with respect to the at least one support rod on which the clamp is disposed. The bone pin clamp can be configured to provide multiple degrees of freedom for at least one of the bone pin and the support rod received therein. This can include, for example at least two of sliding, rotation, and orientation, or each of sliding, rotation, and orientation.

In at least some embodiments, after removing the humeral bone preparation instrument from the glenohumeral joint space, the method can include introducing the distal end of the handle assembly to the glenohumeral joint space, the distal end of the handle assembly having coupled thereto a second humeral bone preparation instrument, coupling the handle assembly to the distal end of the humeral guide, passing a guide pin through the transhumeral bone tunnel, and coupling the guide pin to the second humeral bone preparation instrument. The method can further include operating the second humeral bone preparation instrument to treat the humeral resection surface, de-coupling the guide pin from the second humeral bone preparation instrument, and removing the second humeral bone preparation instrument from the glenohumeral joint space.

The action of coupling the handle assembly to the distal end of the humeral guide, in one or more of the earlier referenced instances can include positioning a receiving opening formed in the handle assembly over the distal end of the humeral guide, passing the distal end of the humeral guide into the receiving opening formed in the handle assembly, and securing the handle assembly to the distal end of the humeral guide using a slidable adapter disposed on the distal end of the humeral guide. The method can also include disposing the slidable adapter on the distal end of the humeral guide. This can further include disposing the slidable adapter on the distal end of the humeral guide such that it occurs after de-coupling the humeral sizer attachment from the distal end of the humeral guide.

The action of operating the humeral bone preparation instrument to treat the humeral resection surface, in one or more of the earlier referenced instances can include continuing to operate the humeral bone preparation instrument, or the respective second humeral bone preparation instrument where applicable, until a bottom portion of the slidable adapter reaches a demarcation line formed on an arm of the humeral guide, which can signal that the humeral bone preparation instrument, or the respective second humeral bone preparation instrument where applicable, disposed on a distal end of the handle assembly is at least one of flush or substantially flush with the humeral resection surface.

The action of operating the humeral bone preparation instrument to treat the humeral resection surface, in one or more of the earlier referenced instances can include using the guide pin to move the humeral bone preparation instrument, or the respective second humeral bone preparation instrument where applicable, towards the humeral resection surface to provide treatment to the humeral resection surface. Operating the humeral bone preparation instrument to treat the humeral resection surface in one or more of the earlier referenced instances can include applying a force to the handle assembly to move the humeral bone preparation instrument, or the respective second humeral bone preparation instrument where applicable, towards the humeral resection surface to provide treatment to the humeral resection surface.

The action of applying a force to the handle assembly can include engaging the handle assembly with an impaction tool and applying a force to the impaction tool to operate the humeral bone preparation instrument to treat the humeral resection surface. The humeral bone preparation instrument can include a reamer and/or a blazer. The method can include coupling a humeral bone preparation instrument to the handle assembly. This can include positioning the humeral bone preparation instrument such that a portion thereof is disposed within a chamber of the distal end of the handle assembly and causing a capture plate of the handle assembly to slide towards the distal terminal end of the handle assembly to engage a portion of the humeral bone preparation instrument disposed within the chamber of the distal end of the handle assembly such that the humeral bone preparation instrument is secured with respect to the handle assembly.

The method can also include introducing a humeral implant to the glenohumeral joint space, implanting the humeral implant into the humeral resection surface, introducing a humeral head prosthesis to the glenohumeral joint space, and coupling the humeral head prosthesis to the humeral implant. For methods in which the glenoid implant is implanted into the glenoid surface, the glenoid implant can be configured to operate like an anatomic glenoid surface. Implanting the humeral implant into the humeral resection surface can include introducing the distal end of the handle assembly to the glenohumeral joint space, with the distal end of the handle assembly having coupled thereto an implant adapter, and operating the implant adapter to implant the humeral implant into the humeral resection surface. Operating the implant adapter to implant the humeral implant into the humeral resection surface can include applying a force to the handle assembly to move the implant adapter towards the implant. The action of applying a force to the handle assembly can include engaging the handle assembly with an impaction tool and applying a force to the impaction tool to operate the implant adapter to implant the humeral implant into the humeral resection surface.

In at least some embodiments, prior to introducing the humeral head prosthesis to the glenohumeral joint space, the method can include de-coupling the humeral guide from the respective at least one of the humeral resection surface or the humerus and removing the humeral guide from a surgical site that includes the glenohumeral joint space at which the method is being performed. Coupling the humeral head prosthesis to the humeral implant can include placing the humeral head prosthesis in contact with the humeral implant and impacting the humeral head prosthesis with an impaction tool to drive the humeral head prosthesis into a secure engagement with the humeral implant.

In instances in which the glenoid implant has been introduced, the glenoid implant can be configured to receive a glenoid prosthesis that is configured to operate like an anatomic humeral head. The method can include further introducing a humeral implant to the glenohumeral joint space, with the humeral implant being configured to provide a surface for receiving a glenoid prosthesis that is configured to operate like an anatomic humeral head, implanting the humeral implant into the humeral resection surface, introducing a glenoid prosthesis that is configured to operate like an anatomic humeral head to the glenohumeral joint space, and coupling the glenoid prosthesis to the glenoid implant. In at least some such embodiments, implanting the humeral implant into the humeral resection surface can include introducing the distal end of the handle assembly to the glenohumeral joint space, the distal end of the handle assembly having coupled thereto an implant adapter and operating the implant adapter to implant the humeral implant into the humeral resection surface. Operating the implant adapter to implant the humeral implant into the humeral resection surface can include applying a force to the handle assembly to move the implant adapter towards the implant. Applying a force to the handle assembly can include engaging the handle assembly with an impaction tool and applying a force to the impaction tool to operate the implant adapter to implant the humeral implant into the humeral resection surface.

Any of the methods provided for above can be performed with a transhumeral approach. For example, the introducing actions performed during the method can be performed such that whatever is being introduced into the glenohumeral joint space is introduced from a location that is lateral to the humeral resection surface. Further, the introducing actions performed during the method can be performed such that the introduction occurs substantially orthogonal to a humeral cut plane. The introducing actions can occur through the rotator interval. Any of the actions provided for herein can be performed while keeping the subscapularis tendon intact. Further, any of the actions provided for herein can be performed without distracting the humeral head from its joint.

Another embodiment of surgical method includes coupling a resection guide to at least one of a humeral head or a humerus of a glenohumeral joint, resecting at least a portion of the humeral head located at the glenohumeral joint using the resection guide to guide a cutting instrument and creating a humeral resection surface; and removing the resection guide from the glenohumeral joint space. The method further includes coupling a humeral guide to at least one of the humeral resection surface or the humerus such that a proximal portion of the humeral guide is disposed below the humeral resection surface, at a location that is opposed to the humeral resection surface, and a distal portion of the humeral guide is disposed proximate to a rotator interval, approximately aligned with the humeral resection surface, forming a transhumeral bone tunnel in the humerus using the humeral guide to guide a drill bit through the humerus and the humeral resection surface, coupling a handle assembly to the distal end of the humeral guide, the handle assembly having a humeral bone preparation instrument coupled to a distal end thereof, and operating the humeral bone preparation instrument by way of a guide pin disposed in the transhumeral bone tunnel to treat the humeral resection surface A subscapularis tendon remains intact throughout the method.

Any of the features mentioned in the preceding paragraphs can be applied to this second embodiment.

### BRIEF DESCRIPTION OF DRAWINGS

This disclosure will be more fully understood from the following detailed description taken in conjunction with the accompanying drawings, in which:

FIG. 1A is a side, partially translucent view of one example of an anatomic shoulder joint reconstruction, including an anatomic glenoid implant of the prior art coupled to a scapula;

FIG. 1B is a side, partially translucent view of one example of reverse shoulder joint reconstruction, including a reverse glenoid implant of the prior art coupled to a scapula;

FIG. 2 is a perspective view of a human glenohumeral shoulder joint, and associated joint space, including a humerus having a humeral head and an elongate shaft;

FIG. 3A is a side perspective view of one embodiment of a humeral resection guide having a guide extender coupled thereto;

FIG. 3B is a front perspective view of the humeral resection guide of FIG. 3A;

FIG. 3C is a side perspective view of the guide extender of FIG. 3A;

FIG. 3D is a side perspective view of another embodiment of a humeral resection guide;

FIG. 3E is a side perspective view of still another embodiment of a humeral resection guide;

FIG. 3F is a side perspective view of another embodiment of a humeral resection guide;

FIG. 3G is a side perspective view of an embodiment of a humeral resection guide for use in a left operative side shoulder procedure;

FIG. 3H is a side perspective view of the outer surface of the humeral resection guide of FIG. 3G;

FIG. 3I is a side perspective view of the inner surface of the humeral resection guide of FIG. 3G;

FIG. 3J is a side perspective view of an embodiment of a humeral resection guide for use in a right operative side shoulder procedure;

FIG. 4 is a side perspective view of the humeral resection guide of FIG. 3A disposed at the human glenohumeral shoulder joint of FIG. 2, the humeral resection guide being coupled to the humerus and having a handle coupled thereto, the subscapularis and the supraspinatus illustrated translucently;

FIG. 5A is a side view of the handle of FIG. 4;

FIG. 5B is a top perspective view of the elongate shaft of the humerus and the humeral resection guide and handle of FIG. 4 with a vertical guide rod coupled to the handle, extending substantially parallel to the elongate shaft of the humerus, and with the subscapularis and the supraspinatus illustrated translucently;

FIG. 6 is a front perspective view of the humeral resection guide, the handle, and the vertical guide rod of FIG. 5B, FIG. 6 also illustrating a second vertical guide rod, in phantom, to represent an alternative user position;

FIG. 7 is a side perspective view of one embodiment of a glenoid guide that includes a rigid arm, a drill cannula, and a drill bit disposed within the drill cannula;

FIG. 8A is a perspective view of the human glenohumeral shoulder joint of FIG. 2 with the humeral head resected, the glenoid guide of FIG. 7 coupled to a resulting humeral resection surface, and the drill bit extending through a transhumeral tunnel formed in the humerus and exiting through the humeral resection surface and into a glenoid surface;

FIG. 8B is a side view of the drill bit of FIG. 8A;

FIG. 9A is a side view of an embodiment of a reamer attachment coupled to a driver within joint space of the human glenohumeral shoulder joint of FIG. 8A;

FIG. 9B is a bottom perspective view of the reamer attachment of FIG. 9A;

FIG. 9C is a side, cross-sectional view of the reamer attachment of FIG. 9B taken along line C-C;

FIG. 9D is a side perspective view of a distal end of the driver of FIG. 9A;

FIG. 9E is a side, fully cross-sectional view of the reamer attachment and the driver of FIG. 9A taken along line E-E;

FIG. 10A is a side perspective view of one embodiment of a humeral guide coupled to the humeral resection surface of FIG. 8A, the humeral guide including a rigid arm, a hub, support rods, and bone pin clamps, the figure also including a drill cannula associated with the hub, a humeral sizer attachment coupled to the rigid arm, bone pins disposed within the bone pin clamps, and a drill bit disposed within the drill cannula and passing through a transhumeral tunnel and the humeral sizer attachment;

FIG. 10B is a side perspective view of the rigid arm, hub, support rods, and bone pin clamps of the humeral guide, as well as the drill cannula, of FIG. 10A;

FIG. 11A is a side view of the rigid arm of FIG. 10B;

FIG. 11B is a side view of one support rod of the support rods of FIG. 10B;

FIG. 11C is a side view of one bone pin clamp of the bone pin clamps of FIG. 10B;

FIG. 11D is side perspective view of the bone pin clamp of FIG. 11C secured to the support rod of FIG. 11B and the rigid arm of FIG. 11A;

FIG. 11E is an exploded perspective view of the bone pin clamp of FIG. 11C;

FIG. 11F is a side view of another embodiment of a bone pin clamp that can be used in conjunction with the humeral guide of FIG. 10B, the bone pin clamp receiving both a support rod and a bone pin;

FIG. 11G is an exploded perspective view of the bone pin clamp of FIG. 11F, also illustrating the support rod and the bone pin of FIG. 11F;

FIG. 12A is a side view of one bone pin of the bone pins of FIG. 10A;

FIG. 12B is a side view of the drill cannula of FIG. 10B;

FIG. 12C is a side view of the drill bit of FIG. 10A;

FIG. 12D is a side view of a humeral drive shaft that can be used in conjunction with the humeral guide of FIG. 10A and/or a reamer attachment;

FIG. 13A is a top perspective view of the humeral guide, drill cannula, and humeral sizer attachment of FIG. 10A;

FIG. 13B is a top view of the humeral sizer attachment of FIG. 13A aligned and centered against the humeral resection surface of FIG. 10A;

FIG. 13C is a perspective view of the humeral sizer attachment and humeral resection surface of FIG. 13B;

FIG. 14A is a top perspective view of the humeral sizer attachment of FIG. 13A;

FIG. 14B is a bottom perspective view of the humeral sizer attachment of FIG. 14A;

FIG. 14C is an exploded side perspective view of another embodiment of a humeral sizer attachment;

FIG. 14D is a bottom view of the humeral sizer attachment of FIG. 14C;

FIG. 14E is a side perspective view of the humeral sizer attachment of FIG. 14C with an embodiment of a removeable sizer plate secured thereto;

FIG. 14F is a bottom perspective view of the sizer plate of FIG. 14E;

FIG. 14G is a top perspective view of the sizer plate of FIG. 14F as it is removed from the humeral sizer attachment of FIG. 14C and from the joint space;

FIG. 14H is a top perspective view of another sizer plate being coupled to the humeral sizer attachment of FIG. 14C;

FIG. 15 is a side perspective view of the humeral guide of FIG. 10B coupled to the humeral resection surface of FIG. 8A, the figure also including a handle assembly coupled to the rigid arm of the humeral guide via an adapter, the handle assembly having a reamer attachment coupled thereto, with the reamer attachment illustrated translucently, and the drill bit of FIG. 12C being disposed in the drill cannula;

FIG. 16 is a perspective view of the adapter of FIG. 15;

FIG. 17A is a side perspective view of the handle assembly of FIG. 15;

FIG. 17B is a bottom perspective view of a distal end of an attachment portion of the handle assembly of FIG. 17A;

FIG. 17C is an exploded side perspective view of the handle assembly of FIG. 17A;

FIG. 18A is a side perspective view of the reamer attachment of FIG. 15;

FIG. 18B is an exploded side perspective view of the reamer attachment of FIG. 18A;

FIG. 18C is a side view of the reamer attachment of FIG. 18A coupled to the handle assembly of FIG. 16B with the drive shaft of FIG. 12D associated therewith, and with the reamer attachment illustrated translucently;

FIG. 19A is a side perspective view of another embodiment of a reamer attachment;

FIG. 19B is an exploded side perspective view of the reamer attachment of FIG. 19A;

FIG. 19C is a partially translucent side perspective view of a portion of the reamer attachment of FIG. 19A, with a portion thereof illustrated as hidden from view, the figure including a guide pin engaging with a capture plate of the reamer attachment;

FIG. 19D is a bottom view of the capture plate and guide pin of FIG. 19C;

FIG. 20A is an exploded side perspective view of an embodiment of a humeral blazer attachment;

FIG. 20B is a bottom perspective view of the humeral blazer attachment of FIG. 20A;

FIG. 20C is a top perspective view of the humeral blazer attachment of FIG. 20A;

FIG. 21A is a side perspective, cross-sectional view of another embodiment of a humeral blazer attachment, the humeral blazer attachment being coupled to a guide pin;

FIG. 21B is a partially translucent top perspective view of the humeral blazer attachment of FIG. 21A having a number of components thereof illustrated as being translucent;

FIG. 22A is a side perspective view of a skin incision exposing the human glenohumeral shoulder joint, and associated joint space, of FIG. 2 and associated anatomy;

FIG. 22B is a side perspective view of the humeral head of the human glenohumeral shoulder joint of FIG. 22A exposed and marked prior to resection;

FIG. 23A is a reproduction of FIG. 6, illustrating the front perspective view of the humeral resection guide, the handle, and the vertical guide rod of FIG. 5B, and thus also illustrating the second vertical guide rod, in phantom, to represent an alternative user position;

FIG. 23B is a reproduction of FIG. 4, illustrating the side perspective view of the humeral resection guide of FIG. 23A disposed at the human glenohumeral shoulder joint of FIG. 2, the humeral resection guide being coupled to the humerus and having the handle of FIG. 23A coupled thereto, the subscapularis and the supraspinatus illustrated translucently;

FIG. 24 is a side perspective view of the humeral resection guide disposed at the human glenohumeral joint space of FIG. 23B, the humeral resection guide being coupled to the humerus and having the handle coupled thereto, and the handle having the vertical guide rod of FIG. 23A coupled thereto;

FIG. 25A is a side perspective view of the humeral resection guide of FIG. 24 having the guide extender of FIG. 3C coupled thereto, the handle and vertical guide rod of FIG. 24, and a superior bone pin passing through the humeral resection guide to couple the humeral resection guide to the humerus;

FIG. 25B is a detailed perspective view of the guide extender of FIG. 25A having an inferior bone pin passing therethrough to couple the guide extender, and thus the humeral resection guide, to the humerus, with the subscapularis illustrated translucently;

FIG. 25C is a side perspective view of the humeral resection guide, handle, vertical guide rod, and superior bone pin of FIG. 25A, and a second bone pin, inferior to the superior bone pin, passing through the humeral resection guide to further couple the humeral resection guide to the humerus, and the guide extender of FIG. 25B now detached from the humeral resection guide;

FIG. 26A is a side perspective view of the humeral resection guide and associated components and anatomy of FIG. 25C having the guide extender of FIG. 3C coupled thereto with retractors and a cutting tool introduced into the human glenohumeral joint space;

FIG. 26B is detailed view of the cutting tool of FIG. 26A entering the humerus and guided by the humeral resection guide of FIG. 26A;

FIG. 27A is a perspective view of the human glenohumeral shoulder joint after completion of the resecting in FIG. 26, including a glenoid surface, a humerus in an adduction position, and the resulting humeral resection surface, the figure also illustrating a sizer handle positioning a glenoid sizer proximate to the glenoid surface and the drill bit of FIG. 8B being passed through a transhumeral opening formed in the humerus to engage with the glenoid sizer and glenoid surface;

FIG. 27B is a detailed perspective view of the human glenohumeral shoulder joint and related anatomies, the sizer handle, the glenoid sizer, and the drill bit of FIG. 27A, however this time the drill bit is not passed through a transhumeral opening, but instead enters the joint space, passes through a channel in the sizer handle, and through an opening in the glenoid sizer;

FIG. 28A is a perspective side view of the glenoid guide of FIG. 7 coupled to the humeral resection surface of the human glenohumeral shoulder joint of FIG. 27B with a distal end of the glenoid guide disposed in the joint space and the drill bit of FIG. 27B being engaged with the glenoid guide and passed through the transhumeral opening of FIG. 27A;

FIG. 28B is a detailed perspective side view of the distal end of the glenoid guide of FIG. 28A with the drill bit passing through a distal opening of the glenoid guide and into the glenoid surface, with the humerus and the glenoid illustrated translucently;

FIG. 29A is a detailed perspective view of the distal end of the glenoid guide of FIG. 28A with the drill bit passing through a distal opening of the glenoid guide and into the glenoid surface and being used in conjunction with the glenoid sizer of FIG. 27B, the glenoid sizer being disposed above the drill bit;

FIG. 29B is a detailed perspective view of the distal end of the glenoid guide of FIG. 29A with the glenoid sizer being centered on the glenoid and having the grill bit disposed therethrough;

FIG. 30A is side perspective view of the reamer attachment of FIG. 9A placed within the joint space of FIG. 28A and the driver of FIG. 9D entering the joint space towards the reamer attachment;

FIG. 30B is a side perspective view of the driver of FIG. 30A coupled to the reamer attachment of FIG. 30A in the joint space;

FIG. 30C is a side perspective view of the reamer attachment of FIG. 30B driven by the driver and entering the glenoid surface;

FIG. 30D is a side perspective view of the reamer attachment of FIG. 30C entering the glenoid surface of the human glenohumeral should joint, still coupled to the driver;

FIG. 30E is a side perspective view of the reamer attachment of FIG. 30D being removed from the driver;

FIG. 31A is a side perspective view of an alternative embodiment of a glenoid guide, though similar to the glenoid guide of FIG. 7, having an embodiment of an implant coupling effector coupled to a distal end thereof, the implant coupling effector being coupled to a glenoid implant;

FIG. 31B is a perspective view of the human glenohumeral shoulder joint of FIG. 30D with the glenoid guide of FIG. 31A positioning the implant coupling effector and the glenoid implant component into the glenoid surface;

FIG. 32A is a side perspective view of the glenoid guide of FIG. 31A having an embodiment of an impactor coupled to the distal end thereof;

FIG. 32B is a perspective view of the human glenohumeral shoulder joint of FIG. 31B with the glenoid guide of FIG. 32A positioning the impactor against the glenoid implant;

FIG. 33 is a reproduction of FIG. 13A, illustrating the top perspective view of the humeral guide with the humeral sizer attachment coupled to the rigid arm of the humeral guide of FIG. 10A;

FIG. 34A is a reproduction of FIG. 13B, illustrating the top view of the humeral sizer attachment of FIG. 33 aligned and centered against the humeral resection surface of FIG. 32B;

FIG. 34B is a reproduction of FIG. 13C, illustrating the perspective view of the humeral sizer attachment and humeral resection surface of FIG. 34A;

FIG. 35A is a side perspective view of the humeral guide of FIG. 33 coupled to the humeral resection surface of FIG. 34B, by way of the humeral sizer attachment, and the drill cannula coupled to the humerus, with the drill bit of FIG. 12C disposed within the drill cannula;

FIG. 35B is a side perspective view of the humeral guide, the humeral sizer attachment, and the humeral resection surface of FIG. 35A, the humeral guide having a plurality of the bone-engaging pins of FIG. 12A associated therewith;

FIG. 35C is a side perspective view of the humeral guide of FIG. 35B having the drill bit passed through the drill cannula and through the humeral resection surface, creating a transhumeral tunnel in the humerus;

FIG. 35D is a side perspective view of the humeral guide of FIG. 35C having the drill bit removed from the drill cannula;

FIG. 36A is a side perspective view of the humeral sizer attachment of FIG. 35D being removed from the humeral guide of FIG. 35D at the surgical site;

FIG. 36B is a side perspective view of the adaptor of FIG. 16 coupled to the arm of the humeral guide of FIG. 36A;

FIG. 37A is a reproduction of FIG. 18A, illustrating the side perspective view of the reamer attachment of FIG. 15;

FIG. 37B is a side perspective view of the reamer attachment of FIG. 37A prior to being coupled to the handle assembly of FIG. 17A;

FIG. 37C is a side perspective view of the reamer attachment of FIG. 37B coupled to the handle assembly of FIG. 37B;

FIG. 38A is a side perspective view of the handle assembly and reamer attachment of FIG. 37C coupled to a portion of the humeral guide of FIG. 36B, the reamer attachment being positioned proximate to the humeral resection surface;

FIG. 38B is a side perspective view of the drive shaft of FIG. 12D passing into the reamer attachment and the handle assembly of FIG. 38A, with the reamer of the reamer attachment illustrated translucently;

FIG. 38C is a side perspective view of the humeral guide of FIG. 38A coupled to the humerus having the humeral resection surface, the reamer attachment attached to the handle assembly of FIG. 38B, with the handle assembly coupled to the humeral guide and engaged with the drill bit and being moved to ream the humeral resection surface, and with the reamer attachment illustrated translucently;

FIG. 38D is a side perspective view of the reamer attachment attached to the handle assembly of FIG. 38C substantially flush with the humeral resection surface;

FIG. 38E is a side perspective view of the reamer attachment attached to the handle assembly of FIG. 38D being disengaged from the humeral resection surface;

FIG. 38F is a side perspective view of the handle assembly with the reamer attachment of FIG. 38E being disengaged from the humeral guide;

FIG. 38G is a side perspective view of the humeral resection surface reamed with the reamer attachment of FIG. 38F, with the humeral guide still coupled thereto and the handle assembly, and thus the reamer attachment, no longer being coupled to the humeral guide;

FIG. 38H is a side perspective view of the reamer attachment of FIG. 38F being disengaged from the handle assembly of FIG. 38F;

FIG. 39 is a side perspective view of the humeral blazer attachment of FIG. 20C coupled to the attachment portion of the handle assembly of FIG. 38H;

FIG. 40A is a side perspective view of the humeral guide of FIG. 38G, the humeral blazer attachment and handle assembly combination of FIG. 39 being coupled to the humeral guide and positioned proximate to the reamed humeral resection surface for subsequent broaching;

FIG. 40B is a side perspective view of an impaction tool having an end effector configured to provide an impaction force to the humeral blazer attachment and handle assembly of FIG. 40A;

FIG. 40C is a top perspective view of the impaction tool and end effector of FIG. 40B;

FIG. 40D is a side perspective view of the impaction tool of FIG. 40C coupled to the handle assembly having the blazer attachment of FIG. 40A;

FIG. 40E is a side perspective view of the impaction tool and handle assembly of FIG. 40D, illustrating additional aspects of the humeral guide and surgical site in conjunction with the impaction tool being used to provide an impaction force to the humeral blazer attachment;

FIG. 40F is a detailed side perspective view of the humeral blazer attachment coupled to the handle assembly of FIG. 40E being substantially flush with a surface of the reamed humeral resection surface;

FIG. 40G is a side perspective view of the humeral blazer attachment coupled to the handle assembly of FIG. 40F being disengaged from the broached and reamed humeral resection surface;

FIG. 40H is a side perspective view of the handle assembly of FIG. 40G being disengaged from the humeral guide;

FIG. 41A is a perspective view of the broached and reamed humeral resection surface with a humeral driver shaft located proximal thereto;

FIG. 41B is a top perspective view of an insertion tool grasping an implant to be implanted at the broached and reamed humeral resection surface of FIG. 41A;

FIG. 41C is a side perspective view of the implant of FIG. 41B being seated with respect to the broached and reamed humeral resection surface of FIG. 41A with the drill bit of FIG. 38C being disposed proximal thereto in lieu of the humeral driver shaft of FIG. 41A;

FIG. 41D is a side perspective view of the implant of FIG. 41C being seated with respect to the broached and reamed humeral resection surface of FIG. 41A, with the humeral driver shaft of FIG. 41A located proximal thereto;

FIG. 42A is a perspective view of an implant adapter;

FIG. 42B is a side perspective view of the implant adapter of FIG. 42A coupled to the attachment portion of the handle assembly of FIG. 39;

FIG. 42C is a side perspective view of the implant adapter coupled to the handle assembly of FIG. 42B engaging the implant of FIG. 41C;

FIG. 42D is a side perspective view of the implant, implant adapter, and handle assembly of FIG. 42C coupled to the humeral guide of FIG. 40H;

FIG. 42E is a side perspective view of the impaction tool of FIG. 40B coupled to the handle assembly having the implant adapter of FIG. 42D;

FIG. 42F is a side perspective view of the implant adapter coupled to the handle assembly of FIG. 42E being disengaged from the broached and reamed humeral resection surface;

FIG. 42G is a side perspective view of the implant adapter of FIG. 42F being disengaged from the handle assembly of FIG. 42F;

FIG. 42H is a side perspective view of a distal end of the impaction tool of FIG. 40B having the implant adapter of FIG. 42A coupled thereto;

FIG. 42I is a side perspective view of the impaction tool and implant adapter of FIG. 42H being used with the broached and reamed humeral resection surface of FIG. 42F;

FIG. 43A is a side perspective view of the implant of FIG. 41C disposed in the broached and reamed humeral resection surface;

FIG. 43B is a side perspective view of the humeral guide of FIG. 33 after the implant of FIG. 41C has been implanted in the broached and reamed humeral resection surface;

FIG. 44A is a side perspective view of one embodiment of an insertion tool for grasping at least one of a humeral head trial or a humeral head prosthesis;

FIG. 44B is a side perspective view of the insertion tool of FIG. 44A positioning a humeral head trial at the implant disposed in the broached and reamed humeral resection surface of FIG. 43A;

FIG. 44C is a front perspective exploded view of one embodiment of a humeral head prosthesis;

FIG. 44D is a side view of another embodiment of an insertion tool for grasping at least one of a humeral head trial or a humeral head prosthesis, the tool grasping the humeral head prosthesis of FIG. 44C;

FIG. 44E is a side perspective view of the humeral head prosthesis of FIG. 44C being disposed above the broached and reamed humeral resection surface and implant of FIG. 43A;

FIG. 44F is a side perspective view of the impaction tool of FIG. 40B having an implant-engagement end effector in place of the end effector of FIG. 40B, the implant-engagement end effector being coupled to the humeral head prosthesis of FIG. 44E to provide an impaction force to the humeral head prosthesis; and

FIG. 44G is a detailed side perspective view of the impaction tool and humeral head prosthesis of FIG. 44F.

### DETAILED DESCRIPTION

Certain embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting embodiments and that the scope of the present disclosure is defined solely by the claims. The features illustrated or described in connection with one embodiment may be combined with the features of other embodiments. Such modifications and variations are intended to be included within the scope of the present disclosure. Accordingly, aspects and features of every embodiment may not be described with respect to each embodiment, but those aspects and features are applicable to the various embodiments unless statements or understandings are to the contrary. Further, to the extent portions of a human anatomy are illustrated, but other portions of the anatomy in that same region are not explicitly illustrated, a person skilled in the art will appreciate the location of the omitted anatomies. In at least some instances the omitted anatomies are not included to improve visualization for the provided explanations and illustrations. A person skilled in the art will still understand how the devices and methods provided for herein can interact with such omitted anatomies without requiring specific illustration of the same.

While in some embodiments movement of one component and/or portion of the body is described with respect to another, a person skilled in the art will recognize that other movements are possible. Additionally, a number of terms may be used throughout the disclosure interchangeably but will be understood by a person skilled in the art. By way of non-limiting example, the terms subscapularis, subscapularis tissue, subscapularis tendon, subscapularis muscle, and other variations of the same, may be used interchangeably with one another, and to the extent some such terms do not appear, they are encompassed by use of the others. By way of further non-limiting examples, the terms “prosthesis” and “implant” may be used interchangeably with one another, the terms “cut” and “resect” (and other forms thereof, e.g., cutting and resecting) may be used interchangeably with one another, and the terms “broach” and “blaze” (and other forms thereof, e.g., broaching and blazing) may be used interchangeably with one another.

To the extent the present disclosure describes “coupling,” “mating,” or uses other similar terms as it relates to having an instrument or tool contact part of a patient's anatomy, such as bone, the term includes engagement or contact between the instrument or tool and the part of the patient's anatomy, and does not necessarily require any securing or attaching relationship between the two unless otherwise indicated or understood by a person skilled in the art to inherently create a secured/attached relationship and/or for such securement/attachment to be required for proper performance. Further, to the extent that linear or circular dimensions are used in the description of the disclosed devices, components, systems, and methods, such dimensions are not intended to limit the types of shapes or sizes of such devices, components, and systems, etc. A person skilled in the art will recognize that an equivalent to such linear and circular dimensions can be easily determined for any geometric shape (e.g., references to widths and diameters being easily adaptable for circular and linear dimensions, respectively, by a person skilled in the art). To that end, to the extent the term “circumference” is used, a person skilled in the art will appreciate a “perimeter” or “edge” is an equally acceptable term to use to the extent an equivalent of what is being described is not circular. Likewise, to the extent the present disclosure discusses identifying a center or center point, or other similar location (e.g., substantially center), and/or disposing and/or passing an instrument through and/or at a center or center point, or other similar location (e.g., substantially center), a person skilled in the art will appreciate in at least some instances the “center” or “center point” may be substantially center with respect to the referenced surface and/or another location that is not a center can be used, such location being selected by a surgeon based, at least in part, on an anatomy of a patient, the configuration of the instruments and/or tools being used, and/or surgeon preferences, among other factors.

Sizes and shapes of the components of the humeral resection guides, humeral guides, glenoid guides, handle assemblies, reamer attachments, blazer attachments, implants, and related components, instruments, etc. can depend, at least in part, on the sizes and shapes of the other components with which the guides and related components are being used, the anatomy of the subject being operated on, and the type of procedure being performed. Still further, to the extent features, sides, or steps are described as being “first” or “second,” such numerical ordering is generally arbitrary, and thus such numbering can be interchangeable. Similarly, the order in which actions are presented in claims is by no means limiting. For example, a claim that recites an action of removing a glenoid guide from a surgical site, with that action being listed after an action of reaming a glenoid does not mean that it is required that reaming occurs and then the glenoid guide is removed. In at least some embodiments, the glenoid guide can be removed from the surgical site before the glenoid is reamed, even in instances where they ordered differently in the claim. Only if the claim explicitly requires a particular order is that order applicable. By way of further example, a recitation of passing a guide pin through a second transhumeral bone tunnel, although listed after a recitation of introducing a handle assembly to the surgical site, may occur before such introduction, for example because the guide pin was used to form the second transhumeral bone tunnel and/or was used with a humeral sizer attachment.

While terms like “proximal” and “distal” as used herein, they are primarily used as a point of reference for describing two portions or ends of an instrument, tool, component, device, system, location in a body, etc. Accordingly, no meaning should be attributed to a specific location with respect to “proximal” or “distal” beyond distinguishing one side from another unless explicitly indicated. For example, what is referred to herein as a proximal portion or end may be considered distal in operation, and thus, likewise, what is referred to herein as a distal portion or end may be considered proximal in operation.

In the present disclosure, like-numbered components of various embodiments generally have similar features when those components are of a similar nature and/or serve a similar purpose, unless otherwise noted or otherwise understood by a person skilled in the art. To the extent terms like “approximately,” “about,” and “substantially” are used herein, a person skilled in the art will appreciate the scope those words convey in the context of their usage. During a surgical procedure, obtaining a certain degree of placement, a certain distance, and/or a certain alignment, among other positioning and the like may be difficult, and thus use of terms like “approximately,” “about,” and “substantially” is intended to address this difficulty. A person skilled in the art will understand what constitutes how close a particular dimension or placement should be to still fall within the spirit of the quantification and description provided for herein. Even in instances where such terminology is not used, and a dimension or placement just includes the number or placement term (e.g., “parallel” is used instead of “substantially parallel”), a person skilled in the art will appreciate that, unless explicitly indicated otherwise, terms like “approximately,” “about,” and “substantially” are applicable to those dimensions and placements as well. The foregoing notwithstanding, a person skilled in the art will appreciate that terms like “approximately,” “about,” and “substantially” at least encompass dimensions that are ±10%, 10°, etc. of the provided amount, or encompass dimensions that are ±5%, 5°, etc. of the provided amount, unless indicated otherwise or otherwise known to those skilled in the art. The present disclosure appreciates that a person skilled in the art, in view of the present disclosure, understands suitable placements for various features of the disclosed systems, devices, instrumentation, and/or implants, and related components of any of the same, and thus to the extent a particular placement or location is described, unless it is explicitly indicated that placement or location is required, a person skilled in the art will appreciate other placements or locations that are possible without impacting the overall procedure(s).

The present disclosure is directed to an entirety of a process for performing a shoulder arthroplasty in a tissue sparing manner. This includes preparing and resecting a humeral head, reaming and/or broaching (broaching can also be referred to as blazing herein) the resected surface of the humerus (referred to herein as the “humeral resection surface”) to enable it to receive an implant and/or prosthesis (e.g., a stemless implant and/or a humeral head), preparing and reaming a glenoid to enable it to serve as a receiving surface of the implant and/or prosthesis, and/or to receive its own implant and/or prosthesis, and inserting the implant(s) and/or the prosthesis(es) to the surgical site, among other provided actions herein. The procedure allows for proper preparation of the surgical site, and insertion of the implant(s) (also referred to as prosthetics in some instances) to the surgical site, in a tissue sparing manner, leaving the major rotate cuff muscles and tendons, such as the subscapularis tendon, attached to their respective bone(s). The procedures can be performed, at least in part, through a narrow rotator interval where actions such as resecting, reaming, broaching, and implanting could not previously be performed without having to detach one or more tendons and/or muscles from bone. In addition to providing such procedures, the disclosure provides various instrumentation, devices, tools, instruments, and related attachments and the like that enable these tissue sparing procedures to be performed.

### Relevant Anatomy for Shoulder Procedures

The present disclosure relates to methods, and their related systems and devices, for preparing a humeral head and/or a glenoid surface to receive a prosthesis (also referred to as a prosthetic implant(s), among other terms), and more particularly methods that include preparing a humeral head and a glenoid surface for receiving one or more prosthesis in conjunction with a shoulder arthroplasty procedure performed in a tissue sparing manner. The actions performed in conjunction with the various disclosed methods include preparing, resecting, reaming, and/or broaching one or both of a surface of the humerus and a surface of the glenoid. Systems and devices used in conjunction with the same are also described herein. Such systems and devices include, for example, embodiments of a surgical guide, described as a humeral resection guide, and related components, which can be used at least to resect a portion of a humeral head prior to replacing the resected portion with a prosthetic. By way of further non-limiting example, such systems and devices include a glenoid guide, and related components, which can be used at least for preparing a glenoid surface to receive a prosthesis, such prosthesis being configured to receive the prosthetic associated with the humerus. By way of still further non-limiting example, such systems and devices include a surgical guide, described as a humeral guide, and related components, which can be used at least for preparing a humeral resection surface to receive a prosthesis. Two such bone preparation devices that can be used with such humeral guides include devices for reaming bone and devices for broaching bone.

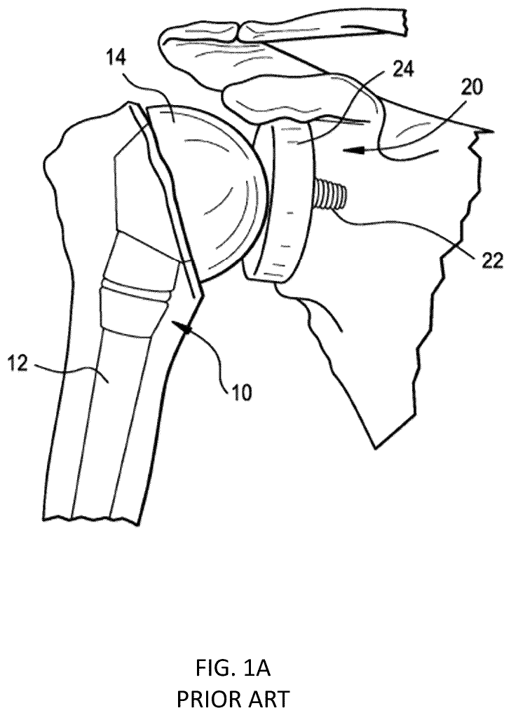

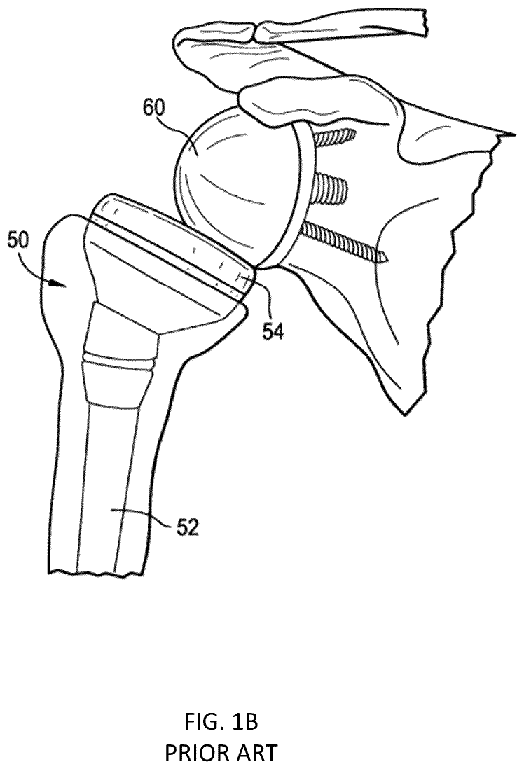

The humeral prosthesis and/or implant can be, for example, a humeral head prosthesis, like the prosthesis 10 of FIG. 1A that includes a convex humeral head 14, which is intended to mimic an anatomically correct humeral head in an anatomic shoulder arthroplasty procedure. The prosthetic convex humeral head 14 is then received by a concave receiver prosthesis, like the prosthesis 24 of FIG. 1A. Alternatively, the humeral implant or prosthesis can be, by way of further example, a concave receiver humeral prosthesis, like the prosthesis 50 of FIG. 1B that includes a concave receiver surface 54, which is intended to mimic a glenoid surface formed on the humerus to receive a prosthetic head component associated with the glenoid, like the convex prosthetic head 60 of FIG. 1B, as used in a reverse shoulder arthroplasty procedure. Although the prosthesis 10 of FIG. 1A includes a stem, as provided for herein, in other instances the equivalent implant or prosthesis to the prosthesis 10 can be stemless.

Whether installing an implant(s) and/or prosthesis(es) for an anatomic or reverse shoulder procedure, an accurate cut of the bone to prepare it to receive the implant(s) and/or prosthesis(es) is important. The humeral resection guides of the present disclosure enable accurate cuts. Further, the guides include various features that allow the guides to be used in smaller spaces, such as procedures performed using tissue sparing techniques that do not detach the subscapularis tendon from its natural attachment points. In other words, the subscapularis remains intact during the procedure. The foregoing notwithstanding, the instrumentation and techniques provided for herein can be used for training and/or with more traditional procedures in which the subscapularis is detached. More generally, the disclosed humeral resection guides are attached to a humerus, such as at a humeral head and/or to bone proximate to the humeral head, and set a desired path for cutting. The desired path is designed to create a surface onto which the implant(s) and/or prosthesis(es) can be secured. The humeral resection guide defines a cutting or resecting plane across which a cutting tool (e.g., a sagittal saw) is designed to run through, cutting away the humeral head and leaving behind an attachment surface (referred to herein as a “humeral resection surface”) to which the implant(s) and/or prosthesis(es) is coupled.

The present disclosure provides for tissue sparing procedures to be performed in which the subscapularis tendon remains intact throughout the procedure. Maintaining attachment of the subscapularis tendon means there is more limited space to perform procedures, and the devices, tools, and systems disclosed herein allow for the same types of procedures to be performed (e.g., shoulder arthroplasty) while causing less harm and damage to tissue and the surrounding anatomy. In some embodiments, such as when a tight joint is involved, a portion of the subscapularis tendon may be cut or sacrificed to increase access to the joint. This may entail, for example, cutting a top of the subscapularis tendon a few millimeters, with it being able to be sutured back after having gained sufficient access to perform the procedure(s). By way of further example, about 10% or less of the subscapularis may be sacrificed while about 90% or more of the subscapularis can remain intact. Instances where a portion of the subscapularis tendon, and/or other tendons, are cut or sacrificed can still be considered tissue sparing with respect to the present disclosures, and are still considered to involve the subscapularis being intact. In other words, “intact” does not have to mean fully intact, it can include anything less than fully intact and/or can encompass the subscapularis tendon being “substantially intact,” where “substantially intact” can include any instance when 20% or less of the subscapularis is scarified, or when 15% or less of the subscapularis is scarified, or when 10% or less of the subscapularis is scarified.

In the surgical procedures provided for herein, the humeral resection, as well as the surface of the glenoid, must be prepared properly to receive the respective one or more implant(s) and/or prosthesis(es) so such implant(s) and/or prosthesis(es) can be associated with the respective humerus or glenoid securely. Specifically, the humeral resection surface and the glenoid are typically reamed, and, at least more typically in the context of the humeral resection surface, broached, to a geometry corresponding to the geometry of the chosen implant(s) and/or prosthesis(es). The humeral and glenoid guides of the present disclosure enable a surgeon to perform these preparation steps, as well as other steps related to shoulder repair procedures, such as steps like implanting the implant(s) and/or prosthesis(es). Further, the guides include various features that enable a surgeon to perform these repairs in smaller spaces, such as procedures performed using tissue sparing techniques that do not detach the subscapularis tendon from its natural attachment points. In other words, the subscapularis remains intact during the procedure. The foregoing notwithstanding, the instrumentation and techniques provided for herein can be used for training and/or with more traditional procedures in which the subscapularis is detached.

Generally, the disclosed humeral guide(s) are attached to a humerus with a resected humeral head to define the angle at which a bone tunnel is to be drilled into the humerus, sometimes referred to herein as the trajectory of the bone tunnel. Alternatively, or additionally, the humeral guide(s) can be mated or coupled to the humeral resection surface that results from resecting the humeral head. The bone tunnel can be drilled from the lateral surface of the humerus, e.g., a lateral cortex, exiting center, or substantially center, and orthogonal, or substantially orthogonal, to the humeral resection surface. Once the bone tunnel is drilled, the guide can maintain its position on the humerus and further assist in guiding actions such as reaming and/or broaching the humeral surface. As described in greater detail below, the force required for reaming and/or broaching can be provided in at least some instances through the transhumeral bone tunnel by connecting appropriate attachments to the guide and using a pulling force through the bone tunnel in the lateral direction.

Generally, the disclosed glenoid guide(s) are also fixed to a humerus to guide drilling of a bone tunnel from a lateral surface of the humerus 1012, e.g., the lateral cortex 1023, exiting substantially central and substantially perpendicular to the humeral resection surface, referred to herein as a transhumeral bone tunnel. Preparation steps such as reaming a glenoid surface can be accomplished through the transhumeral bone tunnel. Locked engagements between a guide pin passed through the bone tunnel and the attachments placed in the joint space can provide the required force for such surface preparation steps. The same guide can be utilized to further guide preparation of the glenoid surface similar to the humeral resection surface.

The surgical techniques provided for herein generally have a transhumeral approach, meaning the approach involves coming from a location that is lateral to the humerus. More specifically, in at least some instances, as provided for herein, the transhumeral approach can occur from a location that allows for a surgical access hole that is orthogonal, or substantially orthogonal, to a humeral cut plane defined or otherwise created by appropriate instrumentation (e.g., a humeral resection guide, as referenced below). Based on that created cut plane, the transhumeral hole can begin at an anterolateral position that can be defined by guide components provided for herein to ensure planar orthogonal access.

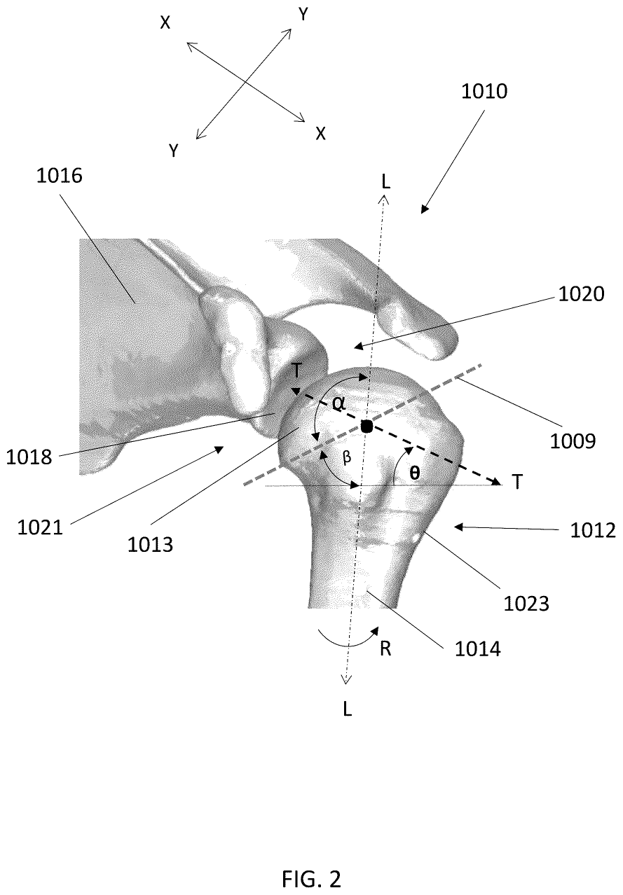

A patient's glenohumeral joint 1010, which is part of a shoulder region of a patient, is illustrated in FIG. 2. The glenohumeral joint 1010 is also referred to herein as the joint space 1010 or shoulder joint 1010, among other names. The natural shoulder joint 1010 includes a humerus 1012 and a scapula 1016. The humerus 1012 includes a generally convex head 1013, referred to herein as the humeral head, an elongate shaft 1014, and a lateral cortex 1023, also referred to as a lateral surface in some instances. As shown the humeral head 1013 has a generally circular cross-sectional area, although in other instances it can be considered elliptical or other shapes. The natural angle α of inclination of the humeral head 1013 relative to the shaft 1014 in most humans is approximately in the range of about 125° to about 145°, and in some instances can be about 135°, the angle α being defined by a longitudinal axis L of the shaft 1014 and an articular margin 1009 where the humeral head 1013 meets the elongate shaft 1014 of the humerus 1012. The angle β is approximately in the range of about 35° to about 55° and forms a linear pair with the natural angle of inclination α, which defines the longitudinal axis L of the shaft 1014. The humeral head 1013 can also have an angle of retroversion θ, which is defined as the angle of rotation in the transverse plane or the Z plane as illustrated. The natural retroversion angle θ in most humans is approximately in the range of about 0° to about 30°. The scapula 1016 includes a concave surface or glenoid 1018. During movement of the shoulder joint 1010, the humeral head 1013 articulates within the glenoid 1018 of the scapula 1016. If the natural shoulder joint 1010 suffers a traumatic injury or degenerative changes, a surgeon may replace either or both the naturally convex humeral head 1013 and the glenoid 1018 with prosthetic components, using either an anatomic or reverse procedure, as detailed above.

Although not illustrated, a person skilled in the art will understand that a subscapularis tendon extends from the scapula 1016 to the humerus 1012, and that in a tissue sparing procedure in which the subscapularis tendon is not detached from the humerus (i.e., the subscapularis tendon remains intact), the subscapularis tendon can be manipulated to improve visibility to a surgical site, such as by using displacement wrap that is disposed around at least a portion of the subscapularis tendon and is tensioned in a manner that causes the tendon to be moved inferiorly or superiorly. Non-limiting examples of such a wrap are disclosed in U.S. Patent Application Publication No. 2024/0108433, entitled “Devices and Methods for Minimizing Damage to Soft Tissue during a Surgical Procedure,” the content of which is incorporated by reference herein in its entirety.

Additional tools, such as a double bent Hohmann retractor(s), posterior cuff retractor(s) (e.g., twisted Hohmann retractor(s)), anterior subscapularis retractor(s) (e.g., right angle Hohmann retractor(s)), inferior subscapularis retractor(s) (e.g., subscap Hohmanns retractor(s)), and/or other types of retractor(s) (e.g., double bent Hohmann retractor(s)), including retractors designed for use from a right side and/or a left side of a patient, can be used to further manipulate surrounding soft tissues. Even when a displacement wrap(s) and/or other tools are utilized, however, the amount of space created is typically insufficient for traditional bone preparation instruments and/or tools to be used to perform various bone preparation actions. In such instances, typically a rotator interval 1020, which can be defined between the superior border of the subscapularis and the anterior border of the supraspinatus, provides a first, superior entry point for accessing the humeral head 1013 and/or the humeral resection surface 1015, and an inferior border 1021 of the subscapularis, which can be defined as the lower inferior border of the subscapularis at the lever of the anterior circumflex vessels (i.e., the “Three Sisters”), provides a second, inferior entry point for accessing the humeral head 1013 and/or the humeral resection surface 1015.

In addition to the disclosed procedures being able to be performed while keeping the subscapularis intact and/or without resecting the subscapularis, and the present devices and systems allowing for the same, the present disclosure also allows for the disclosed procedures to be performed without having to externally rotate the humerus to allow access to the joint space. To the contrary, the present procedures, devices, and systems enable the humerus to not be distracted from its joint during the surgical techniques provided for herein.

During surgery to replace the humeral head 1013 in an anatomic arthroplasty procedure, or remove the humeral head 1013 so that a humeral prosthesis that mimics a receiving surface of the glenoid in a reverse arthroplasty procedure, an initial, or at least early, step in the procedure is to resect the humeral head 1013. This is typically done in a manner that leaves a flat planar surface onto which the prosthesis is eventually secured for use as an implant. The resection or cut is generally made at the articular margin 1009. Thus, a cutting or resecting plane (also referred to as a cut plane, among other terms) for the procedure is typically aligned with the articular margin 1009 such that illustration of the articular margin 1009 can double as an illustration of the cutting plane. As shown, the articular margin 1009 is substantially aligned with the natural angle α of inclination such that the cutting plane will be at the natural angle α of inclination, allowing the anatomy to be properly mimicked. A person skilled in the art will appreciate that to the extent the implant(s) used alters what would otherwise be the natural angle α of inclination, the resection or cut, and thus the resulting humeral resection surface and cutting plane, can be made at a different angle than what is illustrated as the articular margin 1009. This different angle along which the cutting plane is formed can be one that is planned to account for impact caused by the implant(s) such that, when the implant(s) are secured to the resulting humeral resection surface, the resulting angle formed mimics the natural angle α of inclination.

Although the present disclosure is often described herein as being applicable to tissue sparing procedures in which the subscapularis tendon remains intact, a person skilled in the art will appreciate that the devices, systems, and techniques described herein can also be used in conjunction with procedures in which the subscapularis tendon is detached from the humerus.

Traditional tools to prepare the humeral resection surface for receiving a prosthesis rely on adequate visibility and access to the joint space provided by removing the subscapularis tendon and externally rotating the humerus 1012 so the humeral resection surface 1015 faces out the of the glenoid 1018. With the adequate space, the surgeon can use downward force against the humeral resection surface to ream and broach (sometimes referred to as blaze) the surface, thereby creating a geometry within the humeral resection surface 1015 corresponding to the chosen prosthesis. For tissue sparing procedures, as well as other types of procedures performed in more limited space and/or with more limited displacement of tissue and the like, devices like the humeral guides of the nature provided for herein are necessary.

Likewise, traditional tools to prepare the glenoid surface for receiving a prosthesis also rely on adequate visibility and access to the joint space provided by removing the subscapularis tendon and externally rotating the humerus 1012. As a result, and further aided by the humeral head being resected, a surgeon can have full visibility to the glenoid surface that is being treated to receive an implant and/or prosthesis. With the adequate space, the surgeon can have improved visibility and can impart the necessary forces needed to ream or otherwise create a geometry on the surface of the glenoid 1018 that corresponds to the chosen implant and/or prosthesis. For tissue sparing procedures, as well as other types of procedures performed in more limited space and/or with more limited displacement of tissue and the like, devices like a guide like those provided for herein and those incorporated by reference herein, are helpful.

### Humeral Resection Guide(S)

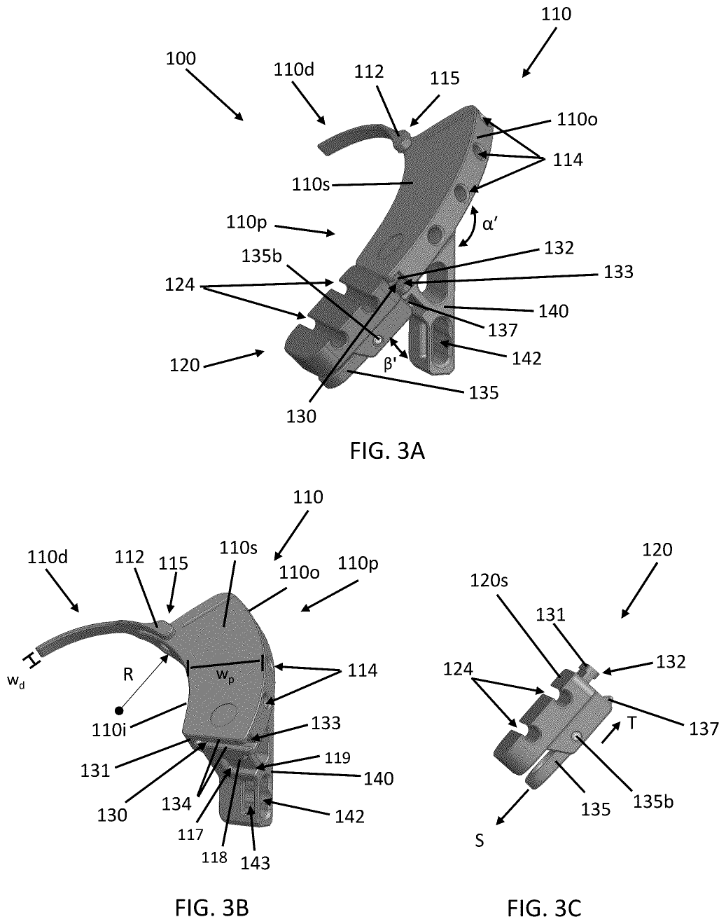

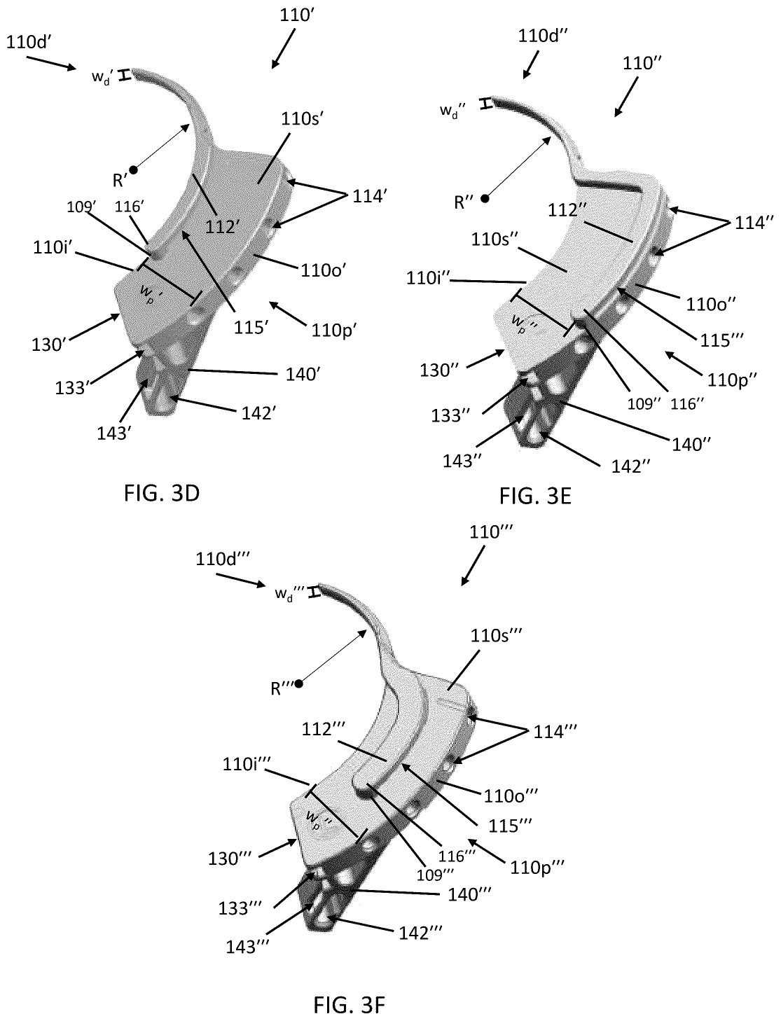

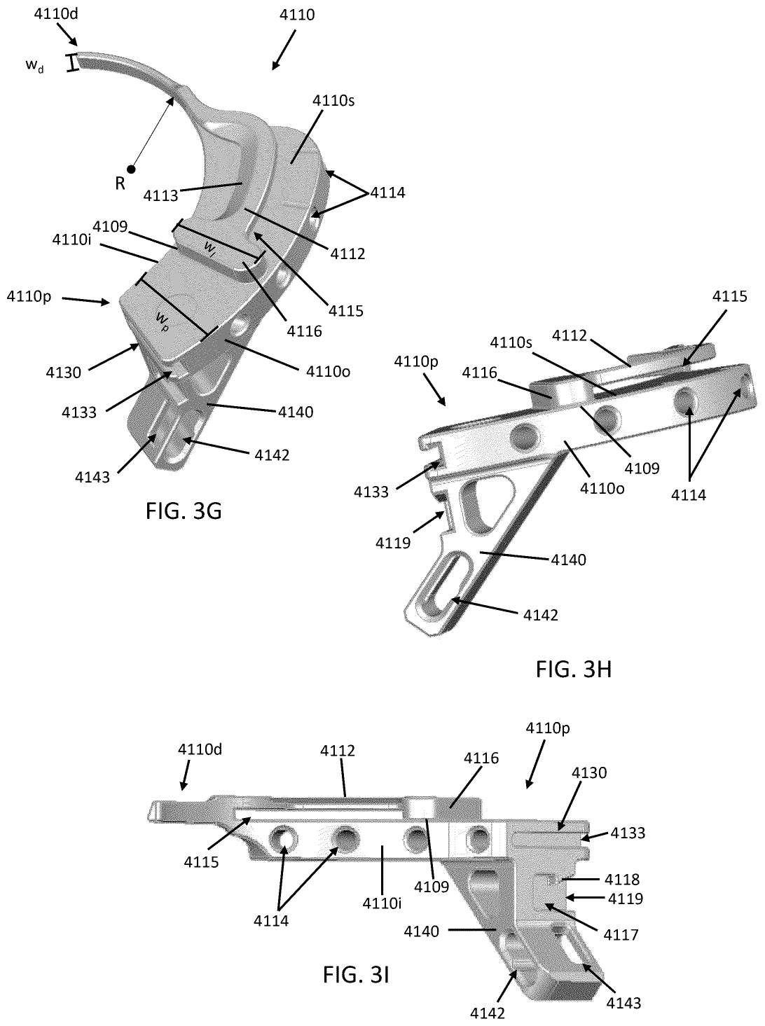

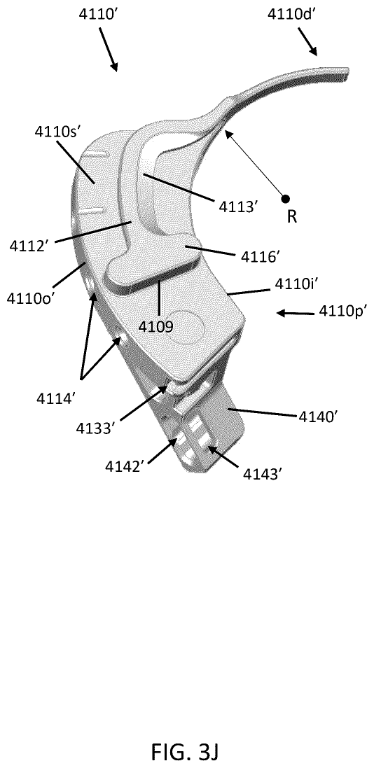

FIGS. 3A-3C and 4-6 illustrate an embodiment of a humeral resection guide 100, also referred to as a humeral cut guide, a resection guide, or a cut guide. Generally, the guide 100 is designed in a manner that it can securely grasp the humeral head, set a cutting or resecting plane, and help guide a cutting tool along the cutting plane to resect the humeral head and form a planar surface on the proximal portion of the humerus (referred to herein as the “humeral resection surface”) onto which an implant and/or prosthesis can be coupled. These actions of the guide 100 can be performed in the narrow glenohumeral joint space while the subscapularis tendon is still attached to the humerus. The illustrated embodiment, as with at least some other embodiments provided for herein, is for use in a left shoulder procedure, the “left” typically referring to the operative side of the patient. A person skilled in the art, in view of the present disclosures, will understand how a resection guide for a right shoulder procedure would be configured in view of the present disclosures.

The resection guide 100 can include a superior radial arm 110 and an optional resection guide extender 120, the superior radial arm 110 being the portion of the guide 100 designed to enter a joint space and engage a humeral head, and/or anatomy adjacent or proximate to the humeral head (e.g., other portions of the humerus), to set a desired cutting or resecting plane or angle. The superior radial arm 110 can be configured (e.g., sized, shaped, and have particular features illustrated and/or described herein) to enter the narrow joint space known as the rotator interval, which is superior to the subscapularis and inferior to the supraspinatus. Further, the guide extender 120, also referred to as an inferior extender or inferior arm, can be configured (e.g., sized, shaped, and have particular features illustrated and/or described herein) to extend the resecting or cutting plane defined by the superior radial arm 110 inferior to the subscapularis tendon to achieve a “patient specific anatomic” resection. The arm 110 and the guide extender 120 in the illustrated embodiment are two distinct and separate components. The guide extender 120 can be optionally coupled to a proximal portion 110p of the superior arm 110. More particularly, a slot 130 formed in the proximal end of the superior radial arm 110p can receive protrusion 132 formed on the guide extender 120. The slot 130 and protrusion 132 are merely one example of a coupling mechanism that can be used to mate the guide extender 120 to the superior radial arm 110, and other components known to those skilled in the art can be used to removably mate the superior radial arm 110 and extender 120.

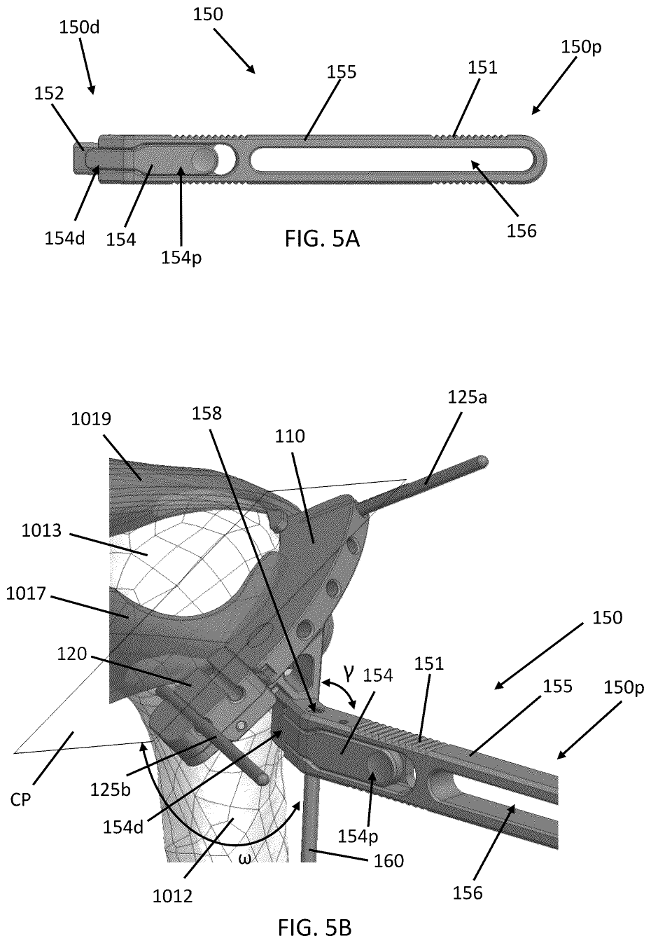

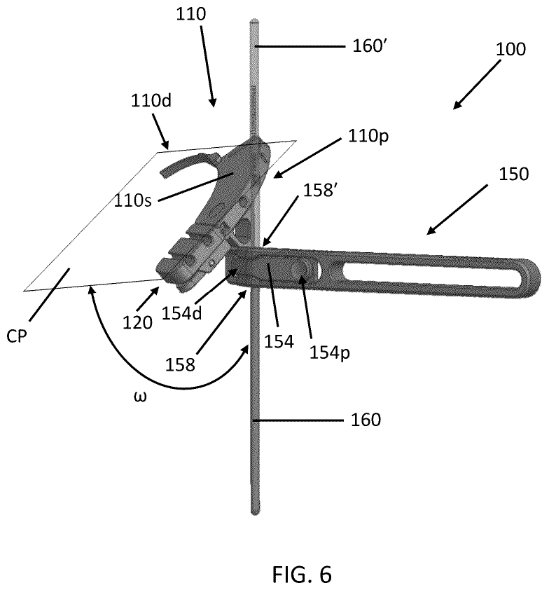

The resection guide 100 can also include a vertical alignment plate 140, which can be integrally formed with or coupled to the superior radial arm 110, for example by a weldment or a press fit, between the two components. As shown, the vertical alignment plate 140 extends distally from the superior radial arm 110. A length of the vertical alignment plate 140 forms an angle α′ with the superior radial arm 110, and more particularly a top or superior surface 110s of the superior radial arm 110. By aligning the vertical alignment plate 140 with the longitudinal axis L of the elongate shaft 1014 of the humerus 1012, and having the angle α′ match the natural angle α of inclination, a resecting plane defined by the X-Y plane of the superior radial arm 110 can be aligned with the cutting plane illustrated by the articular margin 1009 in FIG. 2. Accordingly, a cutting or resecting plane defined by the resection guide 100 (see cutting plane CP in FIGS. 5B and 6) can likewise have an angle α′ approximately in the range of about 125° to about 145°, for example 135°, a cutting or resecting plane angle ω (see FIGS. 5B and 6) being defined by the angle α′ formed by the vertical alignment plate 140 and the superior radial arm 110 as shown. Accordingly, the angle α′ defined by the length of the alignment plate 140 and the superior surface 110s of the superior radial arm 110 defines the resecting plane angle ω, and in the illustrated embodiment, the angle α′ is congruent with the resecting plane angle ω. A person skilled in the art will appreciate that in other configurations it may be possible for the angle α′ to define the resecting plane angle ω without the angles being congruent.

A counterpart angle β′, as shown, can likewise have similar values as the counterpart angle β described above. In the illustrated embodiment, the angle β is not adjustable, although in other embodiments, including modification of the illustrated embodiment, it can be. By aligning the vertical alignment plate 140 with the long humeral bone, that sets the desired the location of the superior radial arm 110 and guide extender 120. That is, rotating or otherwise moving the vertical alignment plate 140 can effectively change the angle of inclination of the resecting plane with respect to the humerus because it changes the positioning of the superior radial arm 110 and a coupled inferior extender 120 with respect to the humeral head. In some instances, the vertical alignment plate 140 can be considered its own component that can be selectively attached and detached to the guide 100, while allowing angular rotation to optimize the cut angle α′ in at least some instances, while in other instances it can be considered as part of the guide 100 and/or as part of the superior radial arm 110. Any known technique for coupling two components can be utilized to couple the vertical alignment plate 140 with the superior radial arm 110.

As shown, an opening 142 can be formed through an outer, planar surface of the vertical alignment plate 140 and extend to an opposing planar surface of the vertical alignment plate 140. The opening 142 can be used, for example, to receive a version handle 150, also referred to as a handle, for use in checking and/or adjusting a version alignment of the resection guide 100, and thus the opening can be considered a handle-receiving opening. An embodiment of the handle 150 that can be coupled to the alignment plate is discussed herein with respect to FIG. 5A. The version handle 150 can assist with providing both version alignment and humeral shaft alignment. Version alignment takes into account retroversion and/or anteversion, as understood by those skilled in the art in view of the present disclosures. More particularly, the version alignment capabilities of the present disclosure allows a surgeon to rotate the guide 100 and check angular alignment with a foreman of the patient using the handle 150 coupled to the alignment plate 140. The surgeon can either match exact patient anatomy as desired, or use this version angular alignment check to set the version to a desired value based on other patient factors, such as existing range of motion with the contralateral arm. The illustrated version handle 150 can be considered a 30° version handle because it can be used to set an approximately 30° of retroversion angle. This angle allows a surgeon to visually approximate and set the desired retroversion angle, which can sometimes be approximately in the range of about 20° to about 40°, with the illustrated embodiment providing for a 30° angle. A person skilled in the art will appreciate other features besides an opening 142 can be used to removably couple the handle 150 (or other configuration(s) of a handle) or other tool to the alignment plate 140.

FIGS. 3B and 3C illustrate the superior radial arm 110 and the guide extender 120 of the humeral resection guide. The superior radial arm 110, shown in FIG. 3B, includes a proximal portion 110p and an elongated distal portion 110d, sometimes referred to as a distal member. As noted above, in use, what is referred to herein as the proximal portion 110p may be considered distal in operation, and thus, likewise, what is referred to herein as the distal portion 110d may be considered proximal in operation. The proximal portion 110p is configured for receiving one or more bone pins, and, in at least some instances, for coupling to or otherwise mating with the guide extender 120. As shown, a connection feature 130 is formed as part of the proximal portion 110p to aid in the mating. The connection feature 130 in the illustrated embodiment is a slot 130 that extends from a closed terminal end 131 proximal to an inner surface 110i of the arm to an opposed opening 133 formed in an outer surface 1100 of the superior radial arm 110. The slot 130 includes two opposed ledges 134 that define sidewalls of the slot 130 between the closed terminal end 131 and the opening 133. The ledges 134 and the slot 130 are able to receive the protrusion 132 of the guide extender 120 through the opening 133.

Also formed in the proximal portion 110p can be one or more bone or guide pin receiving holes or openings 114. The openings 114 can be formed in the proximal portion 110p extending from the outer surface 1100 of the proximal portion 110p to an inner surface 110i of the proximal portion 110p for the purpose of receiving one or more bone pins, drill bits, or other similar structures that can engage the humeral head, and/or bone surrounding the humeral head, to help hold the arm 110, and thus the guide 100, at a location or position with respect to the humeral head. The location of the openings 114 can be non-parallel, and further can be such that pin placement occurs above or below the subscapularis tendon (within a bicipital groove location), thus preventing any pin(s) disposed therein from passing through the tendon. More particularly, one or more of the openings 114 can be disposed in a manner such that a drill bit or bone pin that passes therethrough can be below the resecting plane defined by the arm 110 to prevent interference with the cutting surface or cutting tool (e.g., cutting tool 102, discussed below) that is used to cut and/or resect the humeral head. As shown in FIG. 3B, the plurality of openings 114 are disposed along the curvature of the proximal portion 110p at various angular positions, meaning the openings 114 are non-parallel. Any number of openings 114, including one or more than two, can be used to secure the arm 110 to the humeral head. The foregoing notwithstanding, it can be beneficial for the openings 114 to align with the resecting plane such that longitudinal axes extending through a length of the openings 114, and a length of pins disposed therein, can be parallel or substantially parallel to, the resecting plane.

The distal portion 110d of the superior radial arm 110 can be configured to engage the humeral head, and/or bone proximate to the humeral head, to help set a location of the resection guide 100 with respect to the humeral head in conjunction with defining the resecting plane. More particularly, the distal portion 110d can have a generally arcuate shape with an inner, contoured surface 110i′ configured to help grip or otherwise engage bone. The distal 110d and proximal portions 110p together can have a general radius of curvature R designed to fit a natural (or considered normal amongst a designated population for that particular arm 110) curvature around the humeral head.

As shown, this same radius of curvature R can also be formed with proximal portion 110p of the arm 110, while in other embodiments a different radius of curvature can exist for the distal portion 110p and the proximal portion 110p. In some embodiments, the distal portion 110d can include one or more gripping protrusions or teeth disposed on the inner surface 110i, which can help to better grasp and hold the surface of the humeral head and/or bone proximate to the humeral head. Gripping protrusions or teeth can likewise be placed along any portion of the inner surface 110i of the superior radial arm 110.

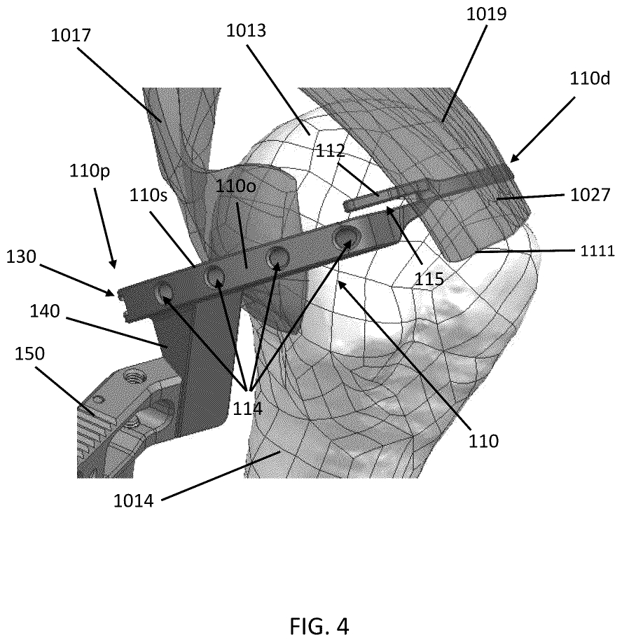

The superior surface 110s of the superior arm 110 can be substantially flat, thus allowing a cutting tool to pass smoothly along the cutting or resecting plane/surface defined by the resection guide 100. The substantially flat planar surface defined across the superior surface 110s of the superior arm 110 also helps reduce possible trauma to soft tissue as the arm 110 is inserted to a surgical site. As shown, a width w_{d} of the distal portion 110d of the arm 110 is typically substantially smaller than a width wₚ of the proximal portion 110p of the arm 110 at least because it is the distal portion 110d that is primarily inserted into the surgical site and is the portion that has to extend furthest into the body, thus likely having to navigate through the most tissue. As shown in FIGS. 3A-3B, and as also shown at least in FIG. 4, a portion (as shown, a proximal portion) of the distal portion 110d can extend over the superior surface 110s of the proximal portion 110p to form a ledge 112 defining a guide slot 115, also referred to as a mini mail slot, between the ledge 112 and superior surface 110s. The guide slot 115 is configured to guide a cutting blade used to resect the humeral head. More particularly, the guide slot 115 can receive a blade or cutting instrument while the ledge 112 maintains the cutting instrument parallel to the superior surface 110s or intended cutting surface. The guide slot guides the cutting instrument along the resecting plane while cutting the humeral head, and keeps the cutting instrument parallel, or substantially parallel, to the resecting plane.

The guide extender 120, shown in FIGS. 3A and 3C, includes a proximal end 120p, and distal end 120d. The proximal end 120p is configured for coupling to or otherwise mating with the superior radial arm 110 as shown in FIG. 3A. The extender 120 can be used to extend the cutting or resecting plane inferiorly to assist in achieving a “patient specific anatomic” resection. A person skilled in the art appreciates what an anatomic resection is, and thus a further explanation of the same is unnecessary. A connection feature 132 is formed as part of the proximal portion 120p to aid in the mating, the connection feature 132 including a protrusion with a ridge 131. The protrusion 132 and ridge 131 are configured to pass through the opening 133 and into the slot 130 formed in the proximal end of superior radial arm 110p such that the ledges 134 of the slot 130 capture the ridge 131 within the slot 130. Further, a ball plunger or lever 135 can be provided to assist in selectively locking and unlocking the extender 120 from the superior arm. For example, in the illustrated embodiment, the lever 135 can be biased, for example by a spring bias, in a direction T, which is towards the superior radial arm 110 in FIG. 3A. As a result, in use with the superior radial arm 110 and the vertical alignment plate 140, the extender 120 is biased into a locked position in which a tip 137 of the lever 135 can engage a surface of the alignment plate 140. In some embodiments, as shown in FIG. 3B, a ramped ledge 118 can be disposed within a recess 117 on the surface of the alignment plate 140 to help capture and secure the tip 137 of the lever 135 in the locked position. In use, the lever 135 can be depressed or otherwise moved in a direction S, opposite to the direction T, by applying a force in the direction S, causing the tip 137 to move away from the vertical alignment plate 140 such that the extender 120 is in an unlocked position with respect to the vertical alignment plate 140, and thus the superior radial arm 110, and the connection feature 132 can slide with respect to the slot 130. In at least some embodiments, the button 135b can be depressed to assist in unlocking the lever 135. When the extender 120 is mounted at a desired location with respect to the vertical alignment plate 140, and thus the superior radial arm 110, the force applied in the direction S can be released, thus causing the lever 135 to be biased in the direction T and the tip 137 to engage the vertical alignment plate 140. When the tip 137 engages the vertical alignment plate 140, an audible click or the like can be heard to notify the user that the extender 120 is properly mated to the vertical alignment plate 140, and thus the superior arm. As shown, a button 135b can also be included as part of the lever 135, the button being configured to assist in selectively locking and unlocking movement of the lever 135. For example, pressing the button 135b inwards, towards the extender 120, can help unlock the lever 135 to allow movement thereof, and releasing the button 135b can cause the button 135b to be biased away from the extender to return the lever 135 to a locked configuration.

One or more bone or guide pin receiving grooves or openings 124, which can be referred to as inferior grooves or openings among other names, can be formed in the superior surface 120s of the extender 120. The openings 124 can be for the purpose of receiving one or more bone pins, drill bits, or other similar structures that can engage the humeral head, and/or bone surrounding the humeral head, to help hold the extender 120, and thus the guide 100, at a location or position with respect to the humeral head. The location of the grooves 124 can be such that pin placement occurs above or below the subscapularis tendon, thus preventing any pin(s) disposed therein from passing through the tendon. In some embodiments, depending on patient anatomy, the pin(s) may be passed through the tendon or soft tissue to achieve a desired pin(s) angle for adequate fixation of the extender 120. As shown the two grooves 124 are parallel, or substantially parallel, to each other, and are also disposed approximately along or parallel to the X axis (although other configurations, including fewer slots, e.g., one slot, are possible). Further, any number of grooves 124, including one or more than two, can be used, and when multiple grooves 124 are used, they do not have to be parallel, or substantially parallel, to each other. The foregoing notwithstanding, it can be beneficial for the grooves 124 to align with the resecting plane such that longitudinal axes extending through a length of the grooves 124, and a length of pins disposed therein, can be aligned or substantially aligned with, or parallel or substantially parallel to, the resecting plane.

Similar to the superior radial arm 110, a superior surface 120s of the guide extender 120 can be substantially flat, thus allowing a cutting tool to pass smoothly along a cutting or resecting plane/surface defined by the resection guide 100. A width of the extender 120 can the same or substantially similar to the width wₚ of the proximal portion 110p of the superior radial arm 110. In the illustrated embodiment when the inferior extender 120 is coupled to the superior radial arm 110, the surface 120s of the extender can align with the surface of the superior arm to create a substantially flat, continuous planar surface.

The shapes and sizes of the superior radial arm 110 and guide extender 120 can vary on a variety of factors, including but not limited to the anatomy of the patient, the size and shape of the components with which the arm 110 and extender 120, and the guide 100 more generally, are being used, the type of procedure being performed, and/or the preferences of the surgeon, among other factors. For example, the extender 120 can be optionally coupled to the superior radial arm 110 to extend the resecting plane in the inferior direction. This extension can be beneficial, for example, if a “patient specific anatomic” resection angle approximately in the range of about 135° to about 155° is desired. Further, different sized arms and extenders, and other components of a guide(s) and/or used in conjunction with the guides of the present disclosure, can be provided together as a kit. While most of the components disclosed herein for each of the humeral resection guide 100, the glenoid guide 200, the humeral guide 300, the handle assembly 400, and all components used in conjunction with each of those, can be introduced through the rotator interval 1020, in at least some embodiments the extender 120 can be introduced to the surgical site inferior to the subscapularis 1017.

FIGS. 3D-3F illustrate three alternative embodiments of superior radial arms 110′, 110″, and 110″ that can be used as, or as part of, a resection guide like the guide 100. These embodiments of the radial arms 110′, 110″, and 110″ are similar to the radial arm 110, and thus many of the labeled features do not require additional explanation, nor does each reference numeral illustrated need to be named explicitly herein as those skilled in the art, in view of the present disclosures, can map the reference numerals in view of the descriptions herein related to the superior radial arm 110 and associated components. For example, features like distal portions 110d′, 110d″, and 110d″, proximal portions 110p′, 110p″, and 110p″, and associated components thereof, can be similar unless otherwise discussed herein or visibly different in the illustrated embodiments. The same holds true for vertical plates 140′, 140″, and 140′″, and associated components.

A primary difference between the superior radial arms 110′, 110″, and 110′″ and the superior radial arm 110 is the configuration of ledges 112′, 112″, and 112″, and thus the resulting configuration of guide slots 115′, 115″, 115′″ as compared to the ledge 112 and the guide slot 115. In each of the embodiments of FIGS. 3D-3F, the ledges 112′, 112″, and 112″ are more elongate, and thus a size—at least length and volume—of the guide slots 115′, 115″, and 115′″ is larger as compared to the ledge 112 and the guide slot 115. This additional length and volume can provide for more ability to reach portions of the bone to be resected while still protecting surrounding tissue. Further, the guide ledges 112′, 112″, and 112′″ each extend to a proximal portion 116′, 116″, 116″ that is fixed to a superior surface 110s′, 110s″, 110s″ at an attachment point 109′, 109″, 109″, thereby enclosing the slots 115′, 115″, and 115″. As shown in FIG. 3D, the ledge 112′ extends from the distal portion 110d′ of the arm, along a superior surface 110s′, proximal to the inner surface 110i′ of the arm 110′, the extending being radial, substantially radial, or somewhat radial in at least some instances, and thus the resulting guide slot 115′ is disposed proximate to where a bone anatomy will be located when in use. In the illustrated embodiment, the ledge 112′ extends over halfway along a length of the superior surface 110s′ to an attachment point 109′ where the proximal end 116′ of the ledge 112′ is fixed to the superior surface 110s′. The fixed proximal end 116′ encloses the slot 115′ along proximal portion 110p′ of the arm 110′. As shown in FIG. 3E, the ledge 112″ extends along the superior surface 110s″, proximal to the outer surface 1100″ of the arm 110″, the extending being radial, substantially radial, or somewhat radial in at least some instances, and thus the resulting guide slot 115″ is disposed proximate to a point of entry for a cutting tool with respect to the superior arm 110″, and thus the resection guide. In the illustrated embodiment, the ledge 112″ extends over halfway along a length of the outer surface 1100″ that is part of the superior surface 110s″. As shown in FIG. 3F, the ledge 112″ extends more centrally over the superior surface 110s″ as compared to the ledges 112′ and 112″. As a result, the guide slot 115″ is more centrally located, making it closer to a location where a cutting tool is received as compared to the guide slot 115′, and closer to a location where a bone anatomy to be resected will be located when in use as compared to the guide slot 115″. Similar to the ledges 112′ and 112″, the ledge 112′″ extends to an attachment point 109″ over halfway along a length of the superior surface 110s′″ over which it is disposed.

FIGS. 3G-3I illustrate yet another embodiment of a superior radial arm 4110 that can be used as, or as part of, a resection guide like the guide 100. This illustrated embodiment is designed for use in left shoulder procedures. This embodiment of the radial arm 4110 has many similarities to the radial arm 110, as well as radial arms 110′, 110″, and 110″, and thus many of the labeled features do not require additional explanation, nor does each reference numeral illustrated need to be named explicitly herein as those skilled in the art, in view of the present disclosures, can map the reference numerals in view of the descriptions herein related to the superior radial arm 110 and associated components. For example, arm 4110 includes an arcuate distal portion 4110d, a proximal portion 4110p, and a vertical alignment plate 4140 extending therefrom. The proximal portion 4110p includes one or more bone pin receiving openings 4114 extending from an outer surface 41100 of the arm to an inner surface 4110i surface arm, and is configured to receive one or more bone pins to secure the arm 4110 to bone. The proximal portion 4110p and vertical alignment plate 4140 further include various coupling features to facilitate the coupling of additional components to the arm 4110. Distinguishing features of this embodiment include the configurations of a ledge 4112 and a guide slot 4115.

The ledge 4112 can be elongate and positioned substantially central along a superior surface 4110s of the arm 4110, similar to the embodiment of FIG. 3F. Additionally, the illustrated ledge 4112 can include a wide proximal end 4116 fixed to the superior surface 4110s of the arm at an attachment point 4109. A width wₗ of the proximal end 4116 can be similar to a width wₚ of the proximal end 4110p of the arm 4110. As shown, the width wₗ is slightly less than the width wₚ, though in other embodiments they can be substantially equal. As shown in FIG. 3G, the wide proximal end 4116 can be approximately perpendicular to a tangent of an inner surface 4110i of the arm 4110. The extra width at the proximal end 4116 of the ledge 4112 can act as a guide for a blade or other cutting tool passed through a slot 4115. For example, an edge of the cutting tool can be aligned along the proximal end 4116 within the slot 4115 and the proximal end 4116 can guide the cutting tool into a humeral head at a trajectory approximately perpendicular to a tangent of the outer surface of the humeral head. This guidance can be beneficial for making the initial plunge cut into the humeral head at the correct location and trajectory. Further the fixed proximal end 4116 of the ledge 4112 can enclose the slot 4115 at distal and proximal ends, which can help prevent a cutting blade from entering locations where it can pass through or otherwise damage surrounding soft tissue.

In the illustrated embodiment, the ledge 4112 further includes a tapered inner edge 4113. As the superior arm 4110 is navigated through soft tissue and towards the humeral head, the tapered edge 4113 can prevent the ledge 4112 from catching, pulling, or otherwise damaging the surrounding soft tissue.

As shown in FIG. 3I, the proximal portion 4110p can include connection feature 4130, which is similar to coupling feature 130, and formed as part of the proximal portion 4110p of the arm, to aid mating the arm 4110 to, for example, a guide extender. The coupling feature 4130 in the illustrated embodiment is a slot 4130 configured to receive a corresponding coupling feature of another component, such as the protrusion 132 of the guide extender 120, through an open end or opening 4133. Further, a recess 4117 can be formed on the vertical alignment plate 4140 below the slot 4130 to help in securing the coupling between the superior arm 4110 and a guide extender. The recess 4117 can include an open end or opening 4119 configured to receive a locking feature of a guide extender therethrough. For example, the recess 4117 can receive a tip of a lever, such as lever 135 of the guide extender 120, to selectively lock the guide extender 120 to the arm 4110. The tip 137 of the lever 135 can be passed into the recess 4117 through the opening 4119 while the protrusion 132 is passed into the slot 4130 through opening 4133. In some embodiments, the recess 4117 can include a ramped ledge 4118. The tip 137 of the lever 135 can slide along the ramped ledge 4118 and into the recess 4117 from the opening 4119. After the tip 137 slides past the ramped ledge 4118, the ramped ledge 4118 can contact and capture the tip 137 within the recess 4117 to lock the mating of the guide extender 120 to the arm 4110. The guide extender 120 can be unlocked from the arm 4110 by depressing the lever 135 to cause the tip 137 to move out of contact with the ramped ledge 4118, thereby allowing the tip 137 to slide out of the recess 4117, through the opening 4119. The vertical alignment plate 4140 can further include openings 4142, 4143 to facilitate coupling of a handle (e.g., a handle 150), as described with respect to openings 142, 143 and a handle 150 in FIG. 5A below.

FIG. 3J illustrates an embodiment of a superior radial arm 4110′ designed for use in right shoulder procedures, and thus is essentially a mirror image of the superior radial arm 4110. Accordingly, the various features described with respect to the arm 4110 of FIGS. 3G, 3I, and 3J are also provided for in the arm 4110′ of FIG. 3H. A further description of the same is not needed as it would be duplicative, and nor is it necessary to label and/or describe each such feature.

FIG. 4 illustrates the resection guide 100 inserted into a joint space and surrounding a humeral head 1013. As shown, the distal portion 110d can be joined with the proximal portion 110p such that the distal portion 110d extends at a height above the superior surface 110s of the proximal portion 110p or the intended resecting plane. More particularly, the distal portion 110d of the superior arm 110 wraps around, or can be described as being proximate to, a substantial portion of the humeral head 1013.

FIG. 5A illustrates an embodiment of a handle 150, also referred to as a version handle, which can be coupled to the alignment plate 140 of the superior radial arm 110. The handle 150 can be used in checking and adjusting the version alignment, and/or can be used to manipulate the resection guide 100, and thus can sometimes be referred to as a manipulation tool. The handle 150 in the illustrated embodiment includes a distal end or portion 150d configured to engage with the opening 142 formed in the vertical alignment plate 140 and a proximal portion 150p or body. The proximal portion 150p and distal portion 150d can be joined at an angle γ matching the natural angle of retroversion, which can be approximately in the range of about 0° to about 30°. In the illustrated embodiment, the angle γ is 30°, and as such the handle can be considered a 30° version handle.

The distal end 150d includes block 152 sized and shaped to pass into the opening 142 and a latch 154 configured to enter a side opening 143 and contact a portion of the block 152 within the opening 142 to secure the handle 150 to the vertical alignment plate 140. The latch 154 can include a distal securing portion 154d and a proximal handle portion 154p. In the illustrated embodiment, the latch 154 is coupled to the handle 150 with a pin and slot such that the latch 154 can pivot with respect to the handle 150. In at least some embodiments, a spring and/or other biasing mechanism(s) can be disposed within a latch coupling to bias the latch 154 in a closed or secured position illustrated in FIG. 5B. In the secured position, the proximal handle portion 154p of the latch 154 can be disposed within an opening 156 formed in the body 150p of the handle and the distal securing portion 154d can contact the block 152. In some embodiments, the proximal handle portion 154p can protrude slightly from the body opening 156. To secure the handle 150 to the resection guide 100, the proximal handle portion 154p of the latch can be pressed in a direction that is into or through the opening 156 to pivot the latch to an open position. When the latch 154 is pivoted to the open position, the distal securing portion 154d can move in a direction away from the block 152 such that the block can be inserted into the opening 142. When the block 152 is seated within the opening 142, the proximal handle portion 154p can be released and a biasing mechanism can pivot the latch 154 back to the secured position. As shown, a depression can be formed in the proximal handle portion 154p, which can be configured, for example, to be complimentary to a fingertip of a user. A tactile click or other audible side can be made when the latch 154 is pivoted into the secured, locked position so the user knows that the handle 150 is secured and stationary with respect to the superior radial arm 110. The distal securing portion 154d of the latch can be shaped and sized to enter the side opening 143 until it contacts the block 152 within the opening 142 thereby securing the version handle 150 to the vertical alignment plate 140. A person with skill in the art will appreciate that various other biased pivoting coupling components can be used to couple the latch 154 to the handle 150 in lieu of the pin and slot coupling illustrated in FIG. 5B. Further, a person skilled in the art will appreciate that the latch 154 is one form of a locking mechanism that can be used in conjunction with the version handle 150 to secure a location of the version handle 150 with respect to the vertical plate 140, and that other mechanisms and techniques known to those skilled in the art can be used to lock a location of the version handle 150 with respect to the vertical plate 140.

The handle 150 can be operated to adjust the position of the superior radial arm 110 relative to the humeral head (e.g., location along the X or Y axis, angle of inclination, retroversion, and/or anteversion). More generally, a person skilled in the art, in view of the present disclosures, will appreciate that the handle 150 can be easily and quickly inserted into and removed from the vertical alignment plate 140, providing for a quick and easy way to manipulate the humeral resection guide 100 while adjusting it to set the desired resecting plane, among other features of the guide 100. In fact, the version handle is designed to be a quick-release tool for efficient manipulation of the guide 100 with only one hand during use.

The version handle can further include a threaded slot or opening 158 configured to receive a vertical guide rod 160, also referred to as a silhouette resection guide pin or a vertical extension rod, among other names, as shown in FIGS. 5B and 6. In at least some embodiments, the vertical guide rod 160 can be optionally coupled to or otherwise associated with the humeral resection guide 100. This can occur prior to securing the guide 100 to the humerus 1012, although it is possible such coupling or otherwise association can occur after securing the guide 100 to the humerus 1012. In the illustrated embodiment, the vertical guide rod 160 is coupled to the handle 150. More specifically, a threaded terminal end of the guide rod 160 is disposed within the threaded slot 158 of the handle 150 such that the guide rod 160 extends distally, along the humerus 1012 as shown in FIG. 5B. The vertical guide rod 160 can assist in providing visualization of the orientation of various features of the resection guide 100 when the guide 100 is disposed at the surgical site, as well as providing a way to manipulate aspects of the guide 100 from a location remote of the surgical site, including outside of a patient's body. For example, the guide rod 160 coupled to the handle 150 can be aligned along the same axis as the vertical alignment plate 140 such that the guide rod 160 extends the length of the plate 140 to help make it easier to align the same with, for example, the elongate shaft 1014 of the humerus 1012. The guide rod 160 can also be used to help manipulate vertical alignment plate 140 and superior radial arm 110 from a location a distance away from the surgical site, including outside of the body. In alternative embodiments, the guide rod 160 can extend in the opposite direction, as illustrated by a phantom guide rod 160′ extending proximally in FIG. 6, and still provide alignment guidance. In such alternative embodiments, the guide rod 160′ can be threaded into a threaded slot 158′ formed opposed to the slot 158, or alternatively, the slot 158 can extend through the body of the handle 150. The ability for a guide rod to be disposed in either direction allows the version handle 150, and the associated guide rod 160, 160′, to be used in an inferior position and/or a superior position. Further, while the slots 158 are described as being threaded, in other embodiments, they are not and/or other mating features can be used to couple a guide rod to the version handle 150 and/or the vertical alignment plate 140.

FIG. 5B also illustrates a pin 125a passed through one of the receiving openings 114 formed in the guide 100 to help secure the guide 100 to the humerus 1012. Thus, as shown, the pins 125a and 125b are used to help secure the guide 100 to the humerus 1012.

Different sized arms for use with different patient and patient anatomies, and other components of a guide(s) and/or used in conjunction with the guides of the present disclosure, can be provided together as a kit. This humeral resection guide kit can include, for example, any combination of superior arms 110, guide extenders 120, vertical alignment plates 140, handles 150, and/or vertical guide rods 160, 160′ of various sizes, among other components and features provided for herein. A person skilled in the art will appreciate that such kits are not limited to only the embodiments disclosed and explicitly illustrated herein, but rather, includes various configuration and iterations accounted for in the text and/or otherwise understood to achieve similar purposes as provided for herein. The various components can be sized and/or shaped for different patient anatomies (e.g., adult, child, patient having certain bone formations due to various ailments or diseases, etc.). Further, a humeral resection guide kit, and/or components thereof, can be more generally be part of a shoulder arthroplasty surgery kit, or surgical kit more generally.

Additional aspects and embodiments of humeral resection guides, and components used in conjunction therewith, are described in U.S. patent application Ser. No. 18/823,212, which is incorporated by reference herein in its entirety.

### Glenoid Guide(s)

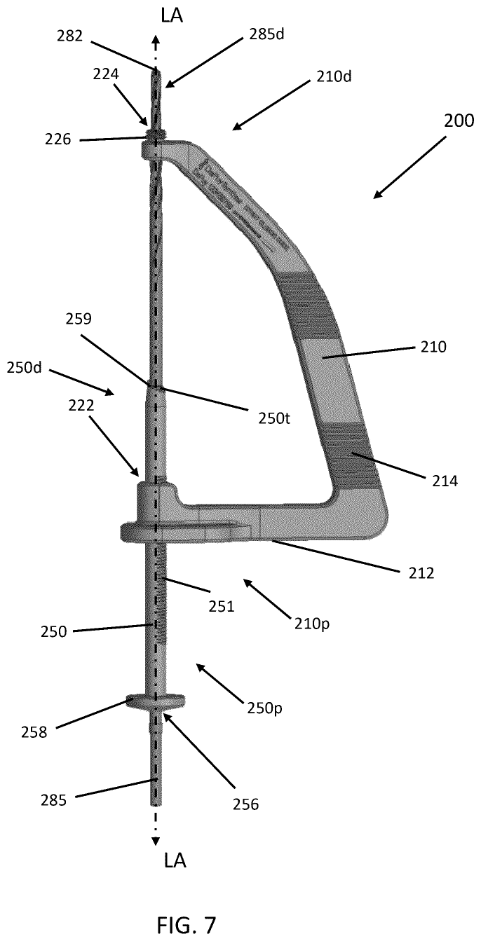

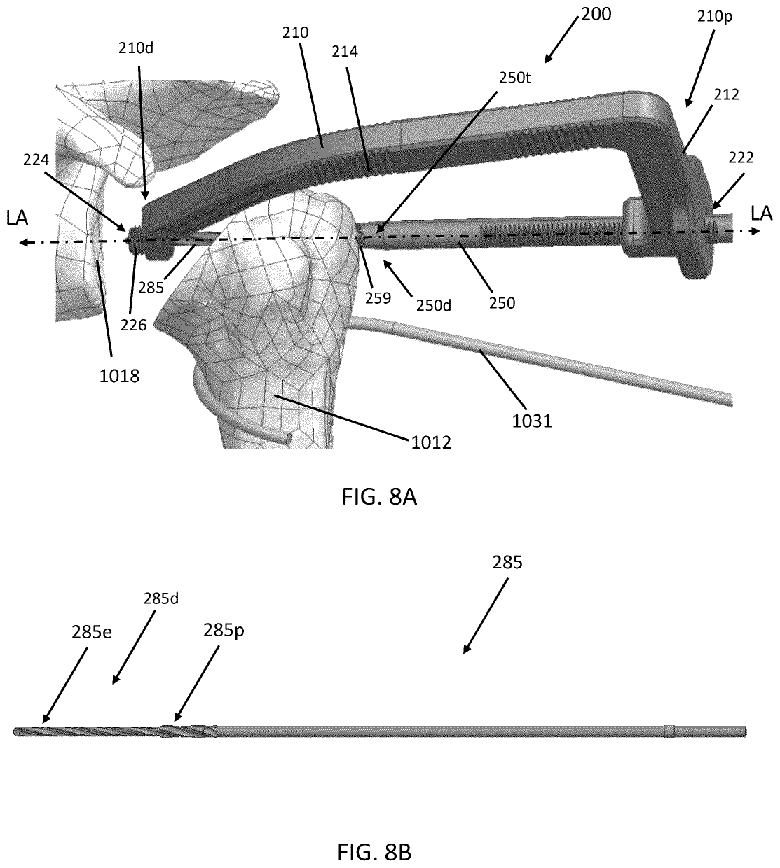

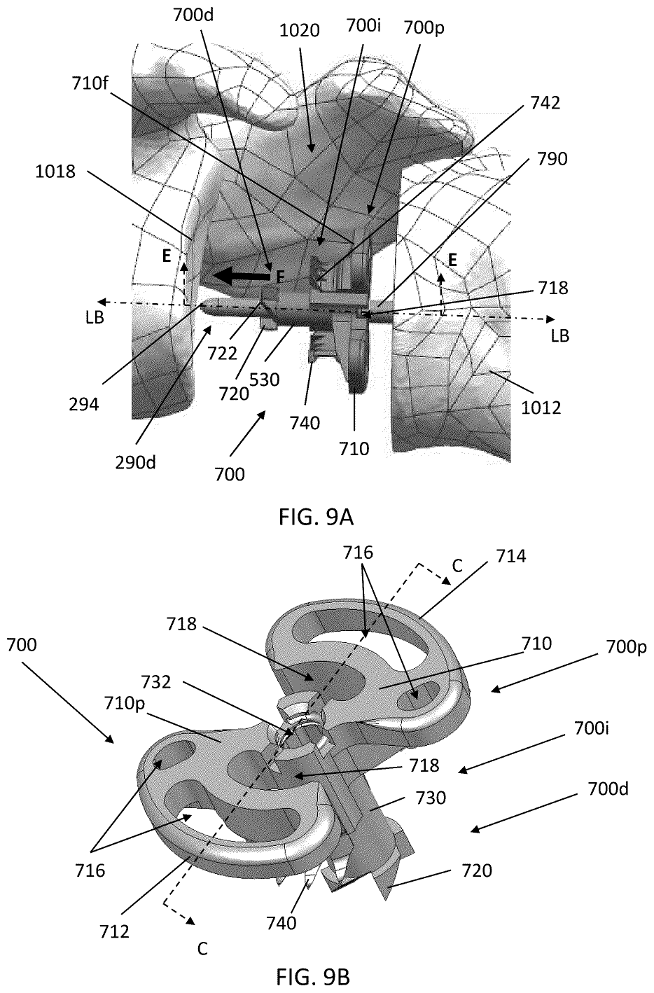

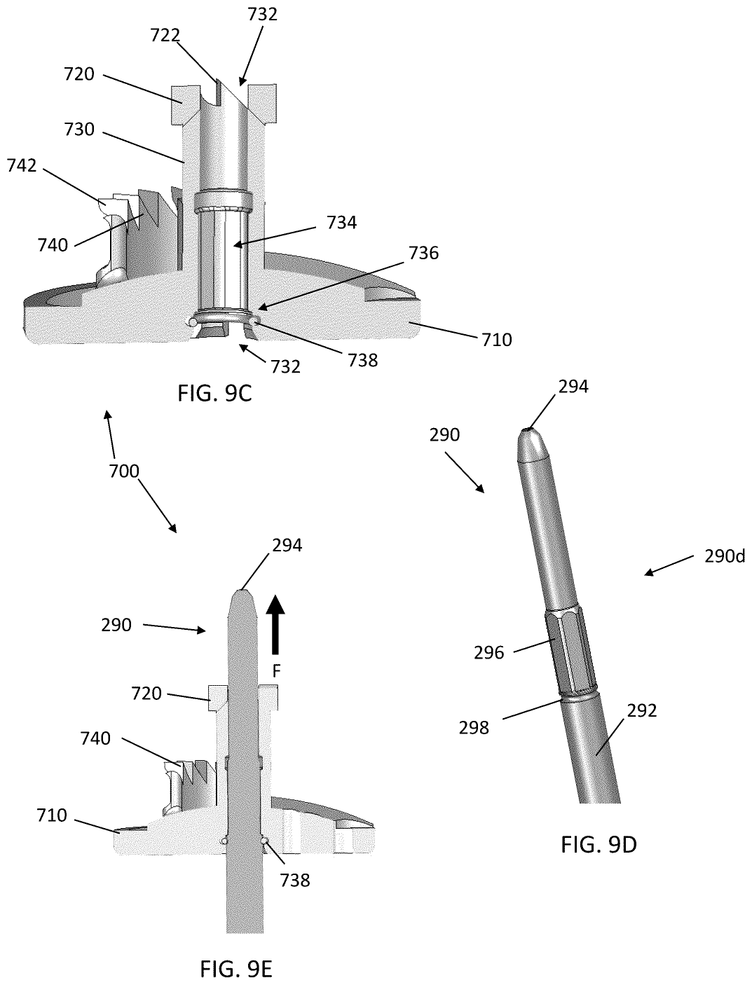

FIGS. 7 and 8A illustrate an embodiment of a glenoid guide 200, also referred to as a glenoid guide frame or an offset glenoid guide in view of an arm thereof having an offset configuration, and FIGS. 9A-9E illustrate components, such as a reamer attachment 700 and driver or drive shaft 290, that can be used in conjunction therewith. Glenoid guides such as the guide 200 can include a rigid, offset arm 210 with a distal portion 210d and a proximal portion 210p. The rigid arm 110 can be sized and shaped to allow for proper centering and alignment between, for example, a cannulated bullet or drill cannula 250 coupled to or otherwise associated with proximal portion 210p of the arm and a center of the glenoid surface 1018, as shown in FIG. 8A. The size and/or shape of the arm 210 can depend, at least in part, on the size and anatomy of the patient (e.g., child vs. adult, male vs. female, etc.) and/or the preferences of the surgeon. In the illustrated embodiment, the length of the rigid arm 210 includes curved or arcuate shape as it extends from the proximal portion 210p of the arm 210 to the distal portion 210d of the arm 210, terminal ends thereof defining the length of the arm 210. In a variety of embodiments, including in the one shown, the rigid arm 210 can be sized and shaped to allow for the glenoid guide to be able to pass into the shoulder joint 1010 in a minimally invasive manner. Further, in a variety of embodiments, including in the one shown, the ridged arm 210 can be sized and shaped to allow for grasping with one hand during a surgical procedure, which in turn can provide for universal adaptation such that it can be grasped in an equally convenient and easy-to-use manner by a user's right hand or left hand without having to change grips and/or positions during the surgical procedure. Accordingly, the glenoid guide 200 can be considered a universal glenoid guide.

In the illustrated embodiment, a first, proximal opening 222 and a second, distal opening 224 are formed in respective proximal and distal ends of the proximal and distal portions 210p, 210d of the arm 210. The proximal opening 222 can be considered a cannula-receiving opening as it can first receive a distal end of a drill cannula (e.g., the drill cannula 250) as the drill cannula is inserted into and through the guide 200, as described in greater detail below, while the distal opening 224 can be considered an alignment opening as it can be proximate to a location at which the distal end of the distal portion 210d of the arm 210 is placed so the guide 200 is properly positioned with respect to the glenoid surface 1018. The glenoid guide 200 can be sized and shaped such that the distal opening 224 and cannula-receiving opening 222 align along a same longitudinal axis LA. As such, the distal and proximal openings 224 and 222 can be considered to be colinear. The proximal end of the arm 210 can include a flat surface 212 that is substantially perpendicular to the longitudinal axis LA. The flat surface 212 can be configured to engage with a user's hand or a tool such as a hammer or mallet, for example to provide a force in a direction towards the surgical site (i.e., towards the glenoid surface 1018). The applied force can be passed through the arm 210 and to the distal end of the guide 210, and thus to any other component associated therewith, such as an end effector or implant coupled to the distal end.

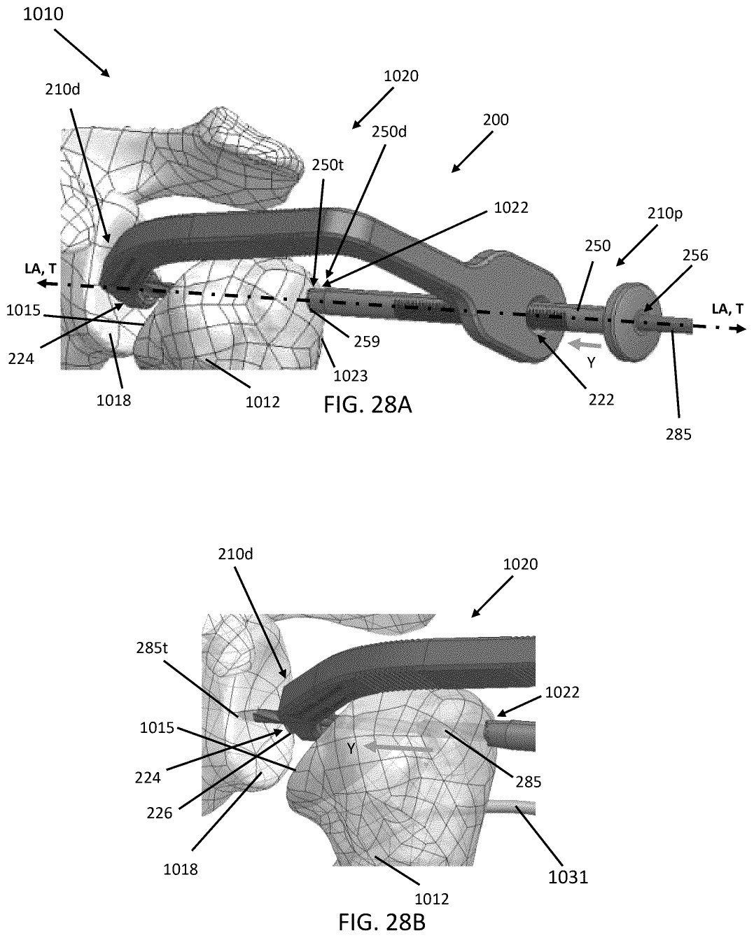

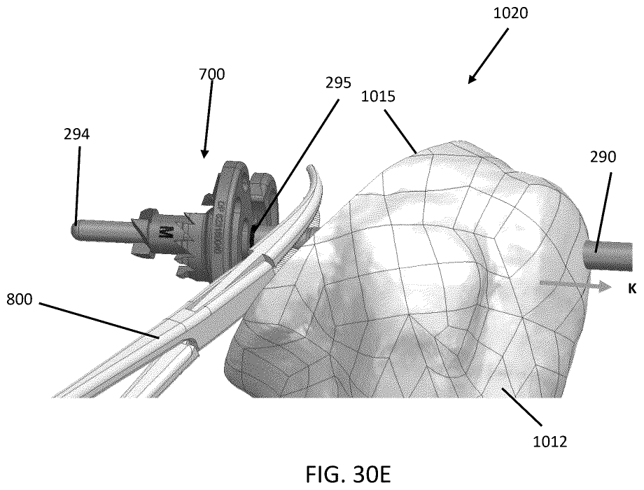

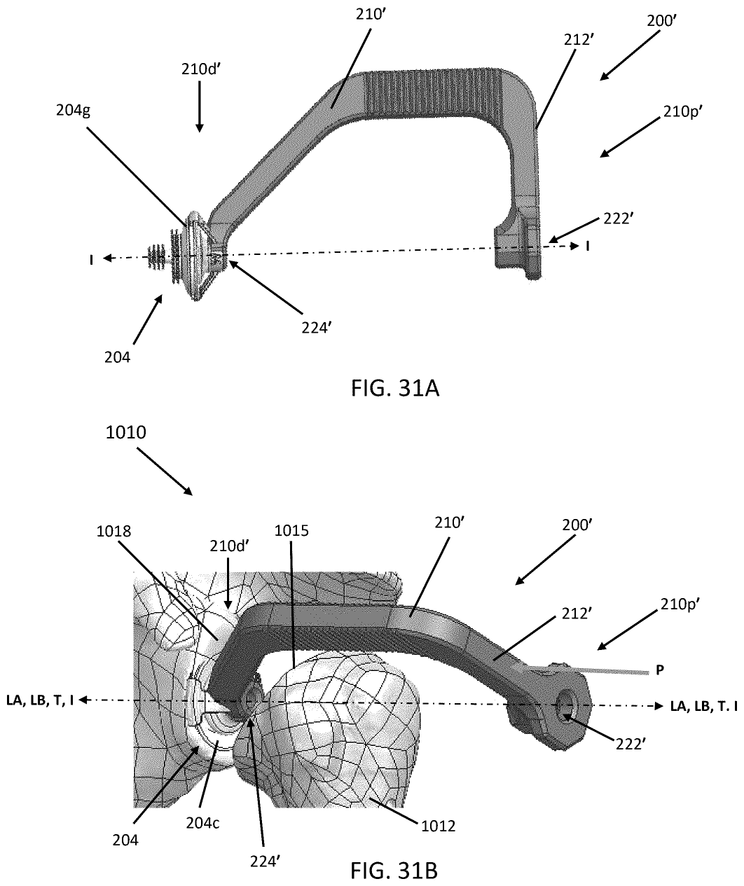

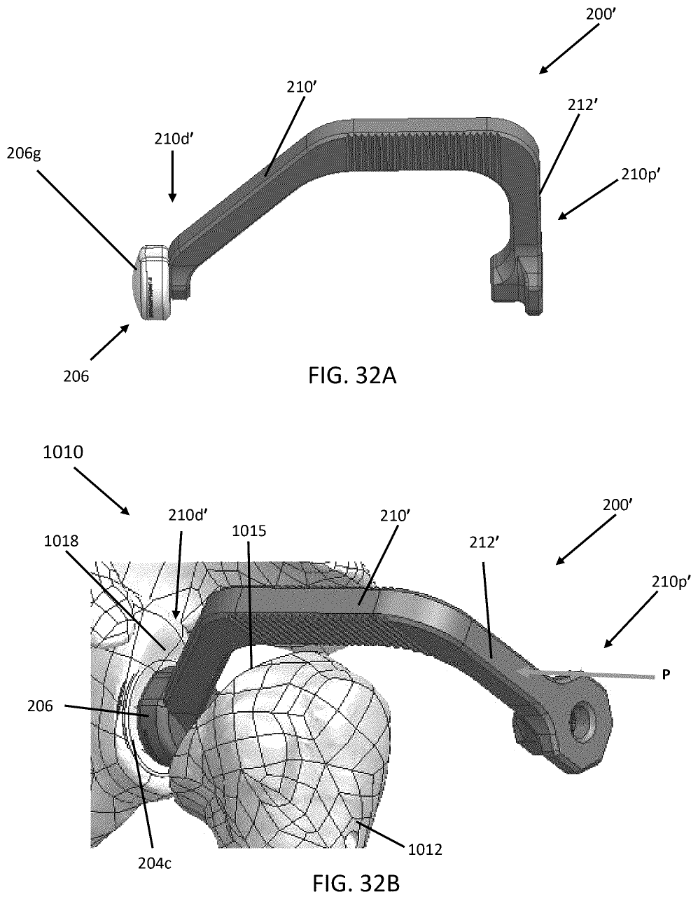

The distal end of the distal portion 210d can include a post 226 that extends from the distal end along the longitudinal axis LA. The distal opening 224 can extend through the post 226 such that the distal end of the arm 210 includes the post 226 with the post 226 being integrally formed with a body of the arm 210, although in other embodiments the post 226 can be a component that is removable and replaceable with respect to body of the arm 210. Further, the opening 224 and post 226 can be configured to receive and mate with an end effector, such as an impactor (see, e.g., FIG. 31A), or implant (see, e.g., FIG. 32A), and/or can help stabilize the glenoid guide 200 with respect to the glenoid surface 1018 (see, e.g., FIG. 28A). In the illustrated embodiment, the post 226 is threaded, although other mating features for purposes of mating with an end effector or implant (e.g., snap-fit, male-female engagement mechanisms, etc.) can be used. As shown, an outer surface of the arm 210 can include gripping features 214 formed thereon. In the illustrated embodiment, the gripping features 214 are ridges, though other gripping features can be used in lieu of, or in addition to, the ridges. The post 226 can be used for implant placement and impaction as desired.

An intermediate portion of the arm 210 that extends from the proximal portion 210p to the distal portion 210d can be offset with respect to the longitudinal axis LA. In particular, as illustrated in use, the longitudinal axis LA can extend through a humerus when coupled to it while the offset arm can extend around the humerus.

The rigid arm 210 can operate in conjunction with the drill cannula 250 to define the location and trajectory at which a bone tunnel is to be drilled into the humerus 1012, and at which the glenoid 1018 will be contacted in conjunction with the various procedures described herein. In the present embodiment, the drill cannula 250 is an elongate, substantially cylindrical or tubular shaft having a base or handle 258 located at a proximal end of a proximal portion 250p of the drill cannula 250, a distal end or tip 250t of a distal portion 250d of the drill cannula 250 configured to engage with bone, and an intermediate portion or length extending therebetween. As shown, ratcheting teeth 251 can be disposed on an outer surface of the intermediate portion of the drill cannula 250. The drill cannula 250 further includes an opening 256 extending through an entirety of a length of the drill cannula 250. The opening 256 can be sized and shaped to allow a drilling component, such as a drive shaft or drill bit 285, also referred to as a driver, to pass through the drill cannula 250 and into the bone in which a bone tunnel is to be formed. The terms drill bit and drill may be used interchangeably herein. As shown, the distal tip 250t can be tapered, for instance by varying a thickness of a wall of the drill cannula 250, to make it easier to push the drill cannula 250 through soft tissue and against the surface of the humerus. In some embodiments, bone engaging features, such as teeth 259, can be formed at the distal tip 250t. The teeth 259 can help stabilize the location of the distal tip 250t of the drill cannula 250 with respect to the bone. The stabilization can provide more accurate trajectory for the bone tunnel. The cannula-receiving openings 222, 224, along with the drill cannula 250, sets the location and trajectory of a bone tunnel or bore to be drilled through the humerus 1012 from the lateral cortex 1023 to the humeral resection surface 1015 and further to the glenoid surface 1018, the tunnel center being substantially perpendicular to the glenoid surface 1018. As shown, the drill cannula 250 passes through and is selectively held within the proximal opening 222 by the guide 200.

Once the glenoid guide 200 is placed proximal the humerus with the distal opening 224 of the arm 210 marking the center of the glenoid surface 1018, the distal portion 250d of the drill cannula 250 can be passed through the cannula-receiving openings 222, 224 and pressed against the lateral cortex 1023 of the humerus 1012 to mark proximal end of the intended bone tunnel and define the entry location for the drilling component. The wall of the arm 210 forming the cannula-receiving opening 222 can be configured to allow for a sliding friction bit between the drill cannula 250 and the guide 200. The ratcheting teeth 251 formed on the outer surface of the intermediate portion of the drill cannula 250 correspond to a one-way ratchet mechanism on the inner surface of the cannula-receiving opening 222 to maintain the position of the drill cannula 250. The ratcheting teeth 251 allow the drill cannula 250 to pass distally through the cannula-receiving opening 222 but prevents the drill cannula 250 from moving proximally thereby maintaining force on the surface of the bone at the distal tip 250t of the drill cannula 250. The drill cannula 250 passes through the cannula-receiving opening 222 until the distal tip 250t meets the lateral surface of the humerus 1012, as shown the lateral cortex 1023.

A drill bit, shown as drill bit or drill 285, can be passed through the drill cannula 250 to create the transhumeral bone tunnel following the trajectory set by the glenoid guide 200. A proximal end can include a connection, which can include a modified trinkle connection, that can be connected to a power source and used to rotate and/or advance a distal end 285d of the drill bit 285, including at a distal tip 285t. Once the bone tunnel has been created, guide pins or drivers with various attachment features at their distal tip can be passed through the drill cannula 250 and attached to various modular attachments inserted into the rotator interval. Embodiments of such guide pins or drivers and their attachment features are described below and/or are incorporated by reference herein and/or are otherwise known to those skilled in the art.

As shown in FIG. 8A, the glenoid guide 200 is employed during tissue sparing arthroplasty surgery after the humeral head has been resected. The distal portion 210d of the arm 210 can be inserted superior to the subscapularis and aligned facing the center of the glenoid surface 1018. The proximal end 210p of the arm can be placed proximal to the lateral cortex 1023 of the humerus 1012 to guide the drill cannula 250 until the distal end of the drill cannula 250 meets the lateral cortex 1023 of the humerus 1012, setting the location and trajectory for a transhumeral bone tunnel. More particularly, the glenoid guide 200 ensures that a trajectory for a guide pin or drill passed through the drill cannula 250 substantially central and substantially perpendicular to a surface of the glenoid 1018. A bone hook 1031 is illustrated in FIG. 8A, and as described below with respect to glenoid preparation methods, can be used for lateral humeral manipulation to increase visualization within the joint.

The drill bit 285 can contact and enter the glenoid surface 1018 to form a pilot hole in the glenoid. In the illustrated embodiment, as shown in FIG. 8B, the drill bit 285 can have a plurality of diameters and be referred to as a stepped drill bit or a stepped glenoid drill bit. The configuration can be such that a distal-most end 285e of the distal portion 285d can have a smaller diameter, such as a diameter of about 3.2 millimeters, and a more proximal portion 285p of the distal portion 285d can have a larger diameter, such as a diameter of about 4.0 millimeters. The smaller diameter can be used to form a pilot hole in a glenoid surface, while the larger diameter can be used to form a transhumeral bone tunnel formed in the humerus 1012, or expand a size of such a tunnel initially formed by the smaller diameter. A length of the smaller diameter typically, although not exclusively, can be such that the larger diameter is not involved in forming the pilot hole. In other embodiments, a drill having a single diameter, or even a drill having three or more diameters, can be used to form a pilot hole in the glenoid and/or a transhumeral tunnel formed in the humerus 1012. Still further, in some embodiments, multiple drill bits, the multiple drill bits being of different diameter(s), can also be used in lieu of a single stepped drill bit.

Additional aspects and embodiments of glenoid guides, and components used in conjunction therewith, are described in U.S. patent application Ser. No. 18/823,275, which is incorporated by reference herein in its entirety.

### Glenoid Reamer(s)

After the center point of the surface of the glenoid 1018 is defined, one or more instruments can be introduced to the joint space to treat or otherwise prepare the glenoid 1018 to receive a prosthesis. One such instrument is a glenoid reamer, which can be used to carve out a geometry in the surface of the glenoid 1018 that corresponds with the geometry of the chosen prosthesis. Reaming during a tissue sparing shoulder arthroplasty can be accomplished by providing force to a reamer tool onto the glenoid surface through the transhumeral bone tunnel.

FIGS. 9A-9E illustrates one embodiment of a reamer attachment or reamer 700, and/or components with which the reamer attachment 700 can be used, configured to carve a geometry in a surface of the glenoid 1018. Reaming a bone requires a sufficient amount of pressure applied to the bone surface by the reamer to carve the proper geometry. In traditional methods that were performed using known tools, the narrow rotator interval maintained by the surrounding soft tissue did not provide adequate space to ream the bone surface using a force applied down onto the glenoid surface 1018. Accordingly, the present disclosure provides the required force to operate the reamer by way of a force provided to the reamer attachment 700 by way of a driver 290, also referred to as a bullet tip glenoid drive shaft or bullet tip driver, passed through the drill cannula 250 and transhumeral bone tunnel. FIG. 9A illustrates the reamer attachment 700 disposed on the driver 290 and disposed in the joint space along the axis LA.

As shown in FIG. 9B, the present embodiment of the reamer attachment 700 includes a base 710 formed at a proximal end 700p of a body of the reamer attachment 700, pilot blades or cutting surfaces 720 formed at a distal end 700d of the body of the reamer attachment 700, and a central tunnel 730 having reaming blades or cutting surfaces 740 disposed therearound along an intermediate portion 700i of the body of the reamer attachment 700. The base 710 can extend between a proximal-most surface 710p (FIGS. 9A and 9B) and a reamer face 710f (FIG. 9A), which is opposed to the proximal-most surface 710p, the proximal-most surface 710p also being the proximal-most surface of the reamer attachment 700. In the illustrated embodiment, the base 710 has a partial circle shape, more particularly having opposed wings 712, 714 disposed symmetrically around a central opening 732 that extends through the body, including through the central tunnel 730, of the reamer attachment 700. Other shapes of the base are possible, including embodiments that have additional wings and embodiments that do not include wings. A plurality of relief holes or openings 716 can be formed in the base 710, extending from the proximal-most surface 710p to the reamer face 710f, the relief holes 716 providing paths for cut tissue, fluids, debris, and other materials to pass through during operation of the reamer attachment 700. As shown, there are four relief openings 716, at least some of which have different sizes and shapes, although other configurations, shapes, and number of relief openings can be provided. Still further, opposed channels 718 can be formed in the base 710 and can extend into the intermediate portion 700i of the reamer attachment 700 as part of the central tunnel 730. The channels 718 can provide, for example, relief for bone and/or cartilage materials as the reamer attachment 700 plunges deeper into bone in use. The flat profile of the base 710 allows the reamer attachment 700 to enter the joint space through the narrow rotator interval 1020 above the subscapularis.

The central tunnel 730 can extend distally from the base 710 and towards the distal portion 700d. The central opening 732 can extend through the tunnel 730, allowing instruments like the driver 290 to pass therethrough. Geometries complementary to the instruments to be disposed through the tunnel 730, and thus through the central opening 732, can be formed therein. For example, a portion of an inner surface of the central opening 732 can have a hex shape, complementary to a hex shape formed in an instrument that passes therethrough.

The reaming blades 740 can be disposed around the central tunnel 730, radially outward from the central opening 732, extending from each of the wings 712, 714. The blades 740 can be sized and shaped to form any number of configurations in the surface of the glenoid 1018, including but not limited to form a concave, smooth surface when rotational force is applied to the reamer 700 and the blades 740 contact the bone. As shown, teeth 742 provided at distal ends of the blades 740 can extend radially outwards at a distal tip thereof, although other teeth configurations are possible.

The pilot blades 720 formed at the distal end 700d, at a terminal end of the central tunnel 730, can also be disposed around the central tunnel 730, radially outward from the central opening 732. The blades 720 can be sized and shaped to form an initial, smaller diameter hole or other shape in the surface of the glenoid 1018, to help prepare the surface to receive the reaming blades 740. As shown, teeth 722 formed on the blades 720 are substantially flat, not extending radially outwards at a distal tip thereof like the teeth 742, although other teeth configurations are possible. After the blades 720 form a pilot hole, the blades 740 engage the surface of the glenoid 1018 to enlarge the previously created pilot hole when rotational force is provided to the reamer attachment 700. Particularly, in at least some embodiments, when a rotational force is provided to the reamer attachment 700, the blades 720 on a distal end of the central tunnel 730 and the blades 740 create ring and post recesses within the surface of the glenoid 1018 to receive an INHANCE™ implant (from Johnson & Johnson of New Brunswick, NJ), although one skilled in the art will recognize that various blade configurations can be placed on the reamer attachment to correspond to varying implant geometries.

The central tunnel 730 and its associated central opening 732 can be configured to accept and secure the driver 290 within as shown in FIGS. 9A and 9E. More specifically, as illustrated in FIG. 9C, the central tunnel 730 extends from the base 710 to the pilot blades 720 with the central opening 732 extending therethrough. A portion 734 of an inner wall of the central tunnel 730 that defines the central opening 732 can be hex-shaped. Further, a snap ring groove 736 can be formed within the opening 732, the groove 736 being configured to receive a snap ring 738 (e.g., a metal snap ring) that extends around the circumference of the central opening 732. In at least some embodiments, the ring 738 can be manufactured into the groove 736 such that it cannot be, or at least not easily be, removed by a user. The ring 738 can provide feedback to a user and/or can resist disassembly, as described in greater detail below.

FIG. 9D illustrates one embodiment of a portion of an instrument with which the reamer attachment 700 can be used. The instrument is a driver 290, and the illustrated portion is a distal end 290d of the driver 290. A person skilled in the art will appreciate the various configurations a proximal end and an intermediate portion of an instrument like the driver 290 can be, and it is thus not necessary to include illustrations or descriptions of the same. As shown, a portion of an elongate shaft 292 of the driver 290 can terminate at a distal end 290d at a rounded distal tip 294, also referred to a bullet-shaped tip or a bullet tip. Further, proximal of the round distal tip 294 can include a hex-shaped portion 296 of the shaft 292. The hex-shaped portion 296 can be complementary to the hex-shaped portion 734 of the central opening 732 formed in the central tunnel 730 of the reamer attachment 700 such that when the hex-shaped portion 296 sit substantially fully with the hex-shaped portion 734, a location of the driver 290 is substantially fixed and held in place with respect to the reamer attachment 700. Although hex-shaped portions 734, 296 are illustrated, a person skilled in the art will appreciate other shapes and configurations that can be used in lieu of hex shapes, and thus these portions can more generally be referred to as keyed portions. When the complementary keyed portions 296, 734 are engaged, the driver 290 can rotate and translate (advance and/or retract) the reamer attachment 700. Additionally, or alternatively, complementary slots and keyways can be formed in the driver 290 and reamer attachment 700 to assist in mating the two components together. An indentation or circular groove 298 can be formed directly proximal of the hex-shaped portion 296. The ring 738 can interact with the groove 298 to provide audible and/or tactile feedback that notifies a user that inserting and seating of the driver 290 into the central opening 732 is complete. The ring 738 and groove 298 can also work together to limit and/or stop further distal movement of the driver 290 in the direction F. Further, the snap ring 738 can provide resistance to disassembly and can keep components assembled to perform reaming. When done with the driver 290, the user can pull back, and the snap ring 738 can expand due, at least in part, to features on an elongate shaft 292 of the driver 290. The shaft 292 can be released and the snap ring 738 can remain in the groove 736.

The reamer attachment 700 can come in different sizes, with a reamer attachment being selected based, at least in part, on the anatomy, age, and other demographics of the patient, along with surgeon preference, among other factors. For example, there may be an extra small, small, medium, large, and extra large sizes, with the larger sizes having larger dimensions for features like the base 710, the pilot blades 720, and the reaming blades 740, among other features.

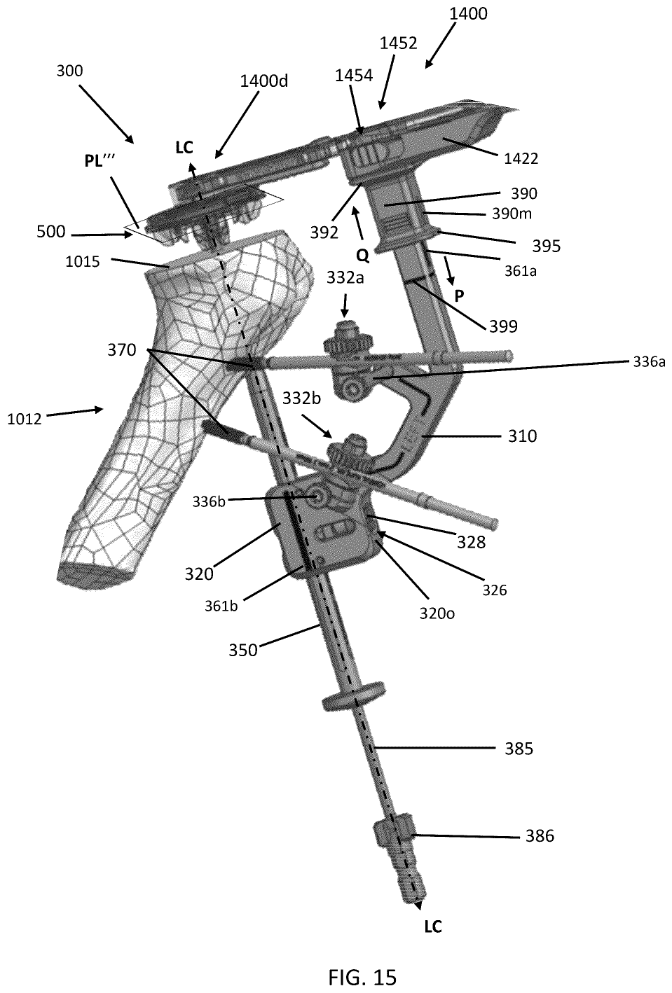

Returning to FIG. 9A, the reamer attachment 700 can be placed into joint space through the rotator interval 1020. The base 710 of the attachment can be aligned substantially planar to the humeral resection surface 1015. The driver 790 can then be pushed in the direction F such that the distal end 790d of the driver enters the central opening 732 and passes through the central tunnel 430, engaging coupling features between the driver 790 and reamer attachment 700, such as the aforementioned hex-shaped portion 734 and/or the groove 736. The driver 290 can then be further advanced in the direction F with the attachment 700 coupled until the distal tip 294 of the driver 290 enters the previously drilled pilot hole in the center of the surface of the glenoid 1018. The secure connection between the driver 290 and the reamer attachment 700 forces the blades 720, 740 of the reamer attachment 700 into the surface of the glenoid 1018 in response to a rotational force being applied to a proximal end of the driver 290 (not shown, but akin to a hub 386 illustrated in FIG. 15) and therefore the attachment 700. The guidance provided by the driver 290 passed through the transhumeral bone tunnel ensures the plane of the reamer 700 aligns substantially parallel to the plane of the surface of the glenoid 1018 and central to the glenoid 1018 for accurate reaming.

Although the reamer attachment 700 is illustrated, a person skilled in the art will appreciate other actions that can be performed in a similar manner using a different attachment than the reamer attachment. This can include other types of cutting or resecting actions, as well as other ways by which a glenoid surface can be treated for purposes of receiving an implant.

Different sized glenoid guides and reamers, and components thereof, for use with different patient and patient anatomies, and other components of a glenoid guide(s)/reamer(s), and/or used in conjunction with the guide(s)/reamer(s) of the present disclosure, can be provided together as a kit. This glenoid guide kit can include, for example, any combination of arms 210, posts 226, drill cannulas 250, drill bits 285, drivers 290, and/or reamers 700 of various sizes, among other components and features provided for herein. Any other components disclosed or otherwise provided for herein can also be part of such a kit(s). A person skilled in the art will appreciate that such kits are not limited to only the embodiments disclosed and explicitly illustrated herein, but rather, includes various configuration and iterations accounted for in the text and/or otherwise understood to achieve similar purposes as provided for herein. The various components can be sized and/or shaped for different patient anatomies (e.g., adult, child, patient having certain bone formations due to various ailments or diseases, etc.). Further, a glenoid guide kit, and/or components thereof, can be more generally be part of a shoulder arthroplasty surgery kit, or surgical kit more generally.

### Humeral Guide(s)

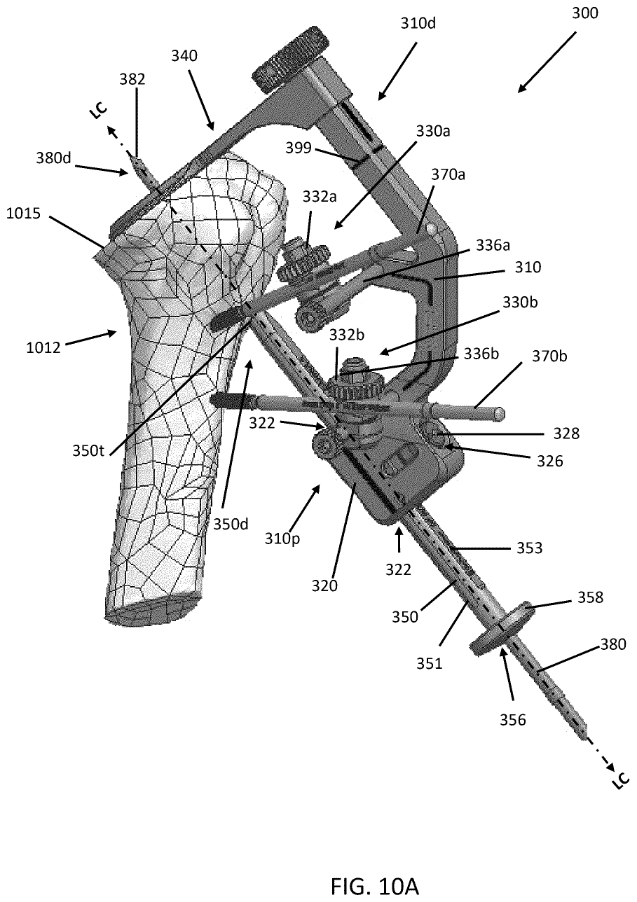

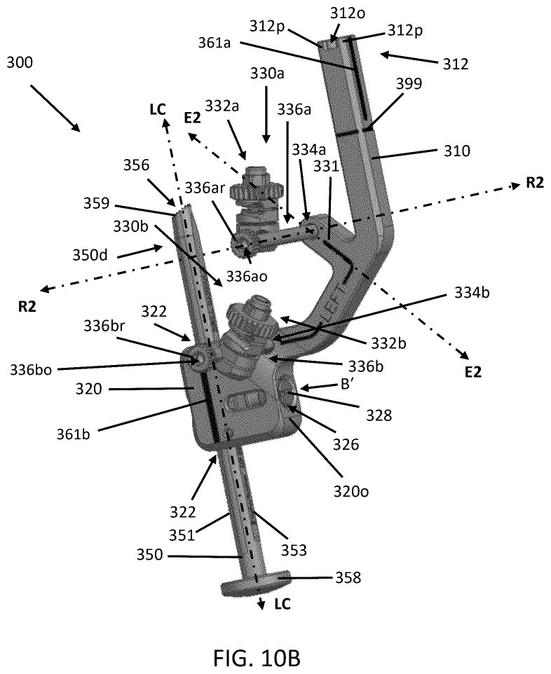

FIGS. 10A-10B illustrate an embodiment of a surgical device, referred to herein as a humeral guide, a guide, a frame, a surgical guide, a surgical guide frame, or a humeral guide frame 300. The humeral guide 300 can be used in various surgical procedures and/or for various purposes, but in at least some instances it can be used to maintain positional guidance during various steps in a tissue sparing shoulder arthroplasty procedure in which the subscapularis remains attached to the scapula and proximal humerus.

### Rigid Arm(s)

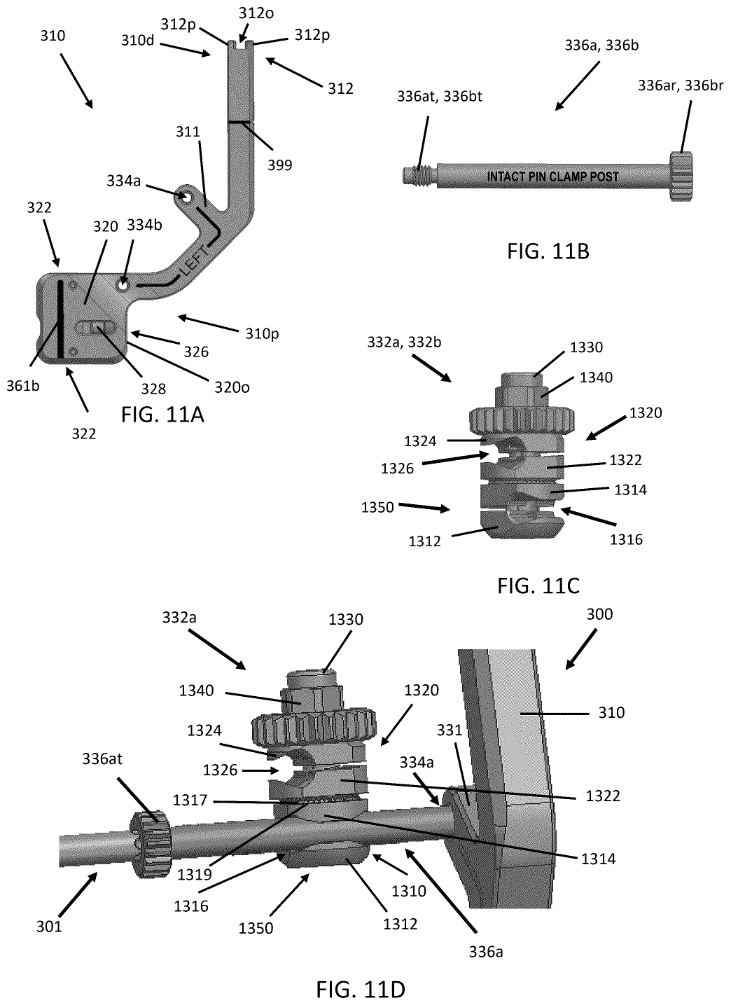

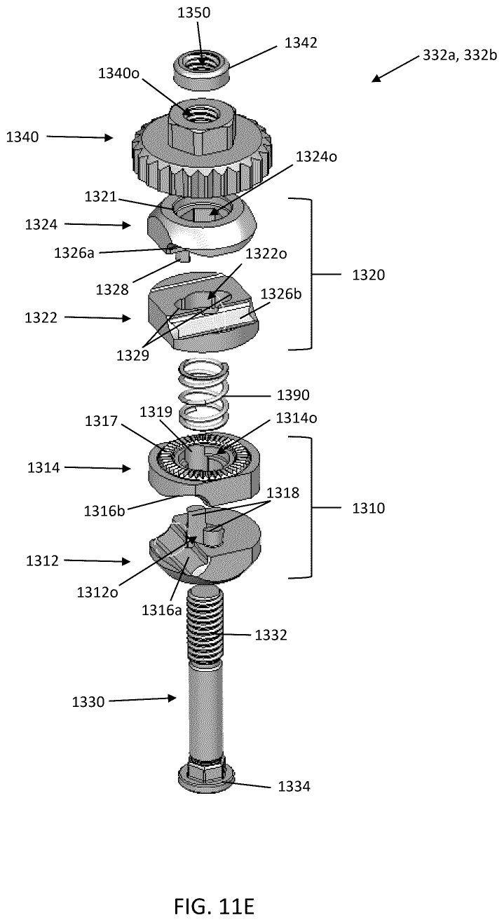

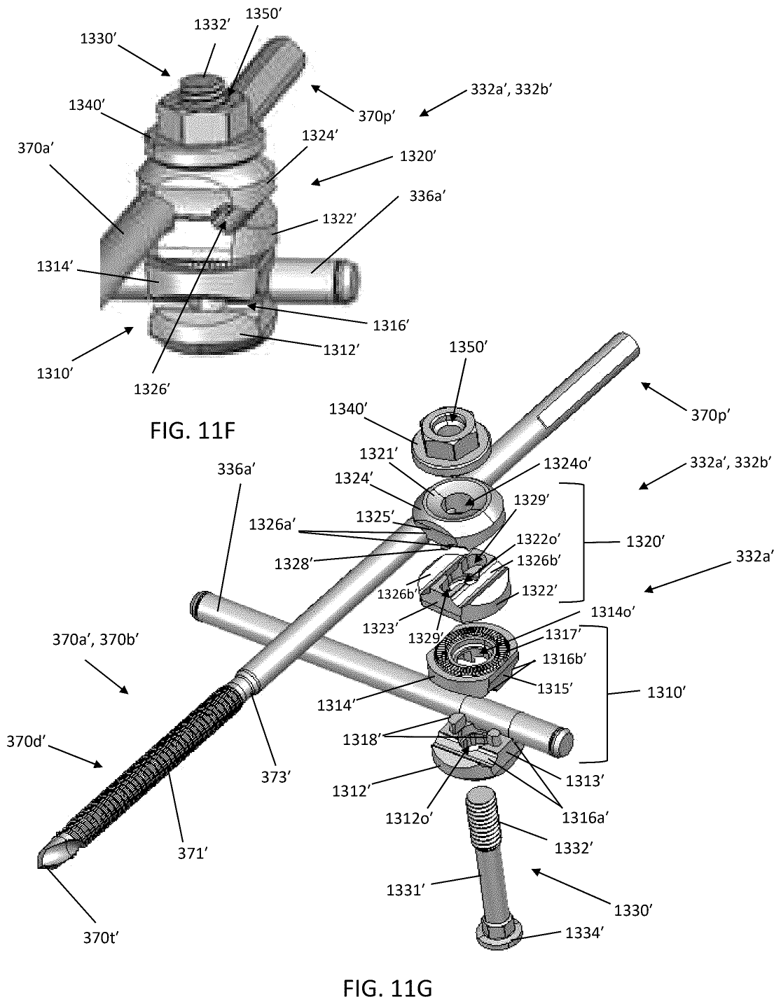

The humeral guide 300 can include a rigid arm 310, as shown separate from the guide 300 in FIG. 11A. The rigid arm 310 can include a proximal portion 310p that is configured to receive a drill cannula 350, also referred to as a cannula, a bullet, or a cannulated bullet, as shown in FIGS. 10A-10B, and a distal portion 310d that is configured to receive one or more modular attachments that can be used in conjunction with the guide. These attachments include, for example, a humeral sizer attachment 340, as illustrated in FIG. 10A, and a handle assembly 1400, as illustrated at least in FIG. 15. In at least some instances, the rigid arm 310 may be referred to as a humeral guide, with the guide (e.g., 300) being the rigid arm 310 and other components of the guide as disclosed herein being components that can be used with the guide/arm 310.

The proximal portion 310p of the arm 310 can include a carriage or hub 320. The hub 320 can be integrally formed as part of the arm 310, or it can be a separate component that attaches to the arm 310. In some embodiments, the hub 320 can just be the proximal portion 310p of the arm 310 such that it is not considered a component separate and apart from the arm 310 with the proximal portion 310p being configured to receive the drill cannula 350. A person skilled in the art will appreciate a variety of mating features that can be provided for on the proximal end of the proximal portion 310p of the arm 310 for receiving and selectively coupling and decoupling the hub 320 from the arm 310 in such instances. In some embodiments, the proximal end of the proximal portion 310p of the arm 310 can be coupled to the hub 320 by press fit or weldment.

The hub 320 can include various features, such as a cannula-receiving opening 322, to assist in interacting with other portions of the guide 300 and/or components used in conjunction with the guide 300. The cannula-receiving opening 322 can extend through an entire length of a body of the hub 320. The opening 322 can be generally cylindrical in shape and can be sized for receiving the drill cannula 350 therethrough. In other embodiments, such the opening 322 can be formed in the proximal portion 310p of the arm with the proximal portion 310p having a similar size and configuration as the rest of the arm rather than the illustrated hub 320. When disposed within the opening 322, the drill cannula 350 can translate along a longitudinal axis LC that extends through the cannula-receiving opening 322, the longitudinal axis LC defining a path of travel for the drill cannula 350.

As best illustrated in FIG. 10B, locking components can be included within the hub 320 and configured to lock a drill cannula, like the drill cannula 350, within the opening 322 at a position against the lateral cortex 1023 of the humerus 1012. In the illustrated embodiment, the internal locking components include a spring-loaded release button 328 disposed in an opening 326 formed in the hub 320. The opening 326 can extend from an outer surface 3200 of the hub to the cannula-receiving opening 322. The spring-loaded release button 328 can be biased towards the cannula-receiving opening 322 such that a distal end thereof extends into the cannula-receiving opening 322 to be able to engage a surface of the drill cannula 350 passing therethrough. The distal end of the button 328 can include, for example, a tooth, which can engage with ratchet teeth 351 of the drill cannula 350. In one non-limiting embodiment, the button 328 can be configured to allow the bias to be acted against by pressing the button 328 in a direction B′, into the hub 320, in turn causing the distal end of the button 328 to move away from the cannula-receiving opening 322 and disengage from the drill cannula 350 disposed in the cannula-receiving opening 322. Releasing the force in the direction B′ can cause the button 328 to return to its biased state, which can include reengaging with the drill cannula 350 if it is disposed in the cannula-receiving opening 322. A person skilled in the art will appreciate a variety of other configurations and set-ups that can be used within the hub 320 to selectively engage and disengage the drill cannula 350 when it is disposed in the cannula-receiving opening 322, including but not limited to a threaded screw and nut and/or a one-way ratchet mechanism.

The distal end of the distal portion 310d of the arm 310 can include an attachment feature 312 configured to receive a modular attachment like the humeral sizer attachment 340. As shown in FIGS. 10B and 11A, the attachment feature 312 can include, for example, opposed U-shaped posts 312p that define an opening 3120 disposed therebetween that can be configured to engage with a complementary mating feature of various modular attachments. A person skilled in the art will appreciate a variety of other mating features that can be provided for on the distal end 310d of the arm 310 for receiving and selectively coupling and decoupling modular attachments from the arm 310.

### Fixation Features for Assisting to Mate Humeral Guide(s) to Humerus

One or more features to receive fixation features, as described herein fixation features 330a, 330b that include bone pin clamps 332a, 332b and support rods 336a, 336b, can be included as part of the arm 310. In the illustrated embodiment, an extension or flange 331, also referred to as a support or a guide support, extends from an intermediate portion of the arm 310 and includes an opening 334a therein for receiving the support rod 336a, which as shown is a superior support rod, and thus the flange 331 and its associated opening 334a supports the superior fixation feature 330a. A second feature includes an opening 334b formed in the proximal portion 310p of the arm 310, the opening 334b receiving the support rod 336b, which as shown is an inferior support rod, and thus the proximal portion 310p of the arm 310 and its associated opening 334b support the inferior fixation feature 330b. The openings 334a, 334b can be internally threaded throughout, thus allowing the support rods 336a, 336b to be threadably connected to either side. A person skilled in the art will appreciate other embodiments may include multiple extensions or flanges like the flange 331 or no extensions or flanges, with openings 334a, 334b being disposed along a length of the arm 310. Likewise, a person skilled in the art will appreciate other ways by which the support rods 336a, 336b can be coupled to the arm 310, and that fewer or more than two such features to receive fixation features can also be provided. In alternative embodiments, the rods 336a, 336b can pass through the openings 334a, 334b such that the rods 336a, 336b extend on both sides of the respective openings 334a, 334b.

While in the illustrated embodiment the extension 331 is integrally formed with the arm 310 and thus rigidly attached, in other embodiments one or more of the extensions 331 can be coupled to the arm 310 and/or the hub 320 using any known technique for coupling one component to another (e.g., fasteners, male-female attachments, adhesives, welding, etc.). Accordingly, in alternative embodiments, additional flexibility can be provided by allowing the extensions or flanges 331 to not be rigidly attached to the arm 310 and/or hub 320.

### Support Rod(s)

As noted, one component of the fixation features 330a, 330b can be support rods 336a, 336b, respectively. FIG. 11B illustrates the support rods 336a, 336b, also referred to as pin clamp posts, more clearly. While in the illustrated embodiment the support rods 336a, 336b are identical, in other embodiments, they can be different. As shown, the support rods 336a, 336b, include threaded ends 336at, 336bt, respectively, for being selectively coupled and de-coupled from the arm 310 by way of the openings 334a, 334b. Ends 336ar, 336br opposed to the threaded ends 336at, 336bt can include features to assist in rotating the rods 336a, 336b into openings like the openings 334a, 334b. As shown, the ends 336ar, 336br include grips to allow rotation by hand, and further, an opening 336ao, 336bo can be formed on respective heads of the rods 336a, 336b for receiving a tool to thread the rods 336a, 336b into the respective openings 334a, 334b. FIG. 11D illustrates an example of a distal end of a tool 301, like an INHANCE™ T20 driver, performing this action.

As illustrated in FIG. 10B, the rods 336a, 336b can extend substantially perpendicular or transverse to a tangential surface of the arm 310 and/or hub 320 from a location at which the openings 334a, 334b are formed such that a substantially right angle is formed by a longitudinal axis extending through an entirety of the rod 336a, 336b and a longitudinal axis that substantially bisects the flange 331 and respective portion of the arm 310. By way of illustration, for the flange 331, the substantially perpendicular or transverse relationship with the rod 336a can be between a longitudinal axis R2 extending through the entirety of the rod 336a and a longitudinal axis E2 that substantially bisects the extension 331.

The rods 336a, 336b provide structure upon which one or more bone pin clamps 332a, 332b can be mounted. Because the flange 331 and/or other mating locations, e.g., the opening 334b, can be placed at different locations with respect to the arm 310 and/or the hub 320, the rods 336a, 336b themselves, and thus the clamps 332a, 332b, can also be placed at different locations with respect to the arm 310 and/or the hub 320, and thus the guide 300 and the surgical site more generally. In the illustrated embodiment, two support rods 336a, 336b are fixed to the guide 300, although any number of rods can be fixed at any portion along the length of the rigid arm 310 and/or hub 320.

### Bone Pin Clamp(s)

Bone pin clamps 332a, 332b are configured to receive and position bone pins 370a, 370b (see FIG. 10A), respectively, at desired locations with respect to the humerus, and thus the surgical site.

FIGS. 11C and 11E illustrate one embodiment of the clamp 332a, 332b. Like the support rods 336a, 336b, in the illustrated embodiment, the clamps 332a, 332b are identical, and thus the components can be described together, although in other embodiments, the clamps used in the same embodiment do not have to be identical. Reference is made to a single clamp 332a below, but the description is applicable to both clamps 332a, 332b.

As shown, the bone pin clamp 332a can include a rod-engaging portion 1310, a pin-engaging portion 1320, a threaded screw 1330, and a locking nut 1340. A central opening 1350 can extend therethrough, for example from the rod-engaging portion proximally up through the locking nut 1340 that receives the screw 1330. The rod-engaging portion 1310, also referred to as a guide-coupling portion, and the pin-engaging portion 1320 create multiple degrees of freedom for each clamp 332a because of the way in which the clamp 332 a engages bone pins and the way in which the clamp 332a can be moved relative to the respective support rod (e.g., the support rod 336a). The clamp 332a can be configured to be able to be directed to a preferred position and orientation on the humeral diaphyseal bone.

As described in further detail below, the pin-engaging portion 1320 of the bone pin clamp 332a can include a first body portion or plate 1324 and a second body portion or plate 1322 that define an opening 1326 configured to receive a bone pin therein. The opening 1326, for example, can be configured to receive a bone pin (e.g., the pin 370a) and/or a 4.0 millimeter humeral guide pin, such a guide pin being able to provide tactile feedback in at least some instances as it is inserted into a bone. The bone-engaging portion 1320 can be used to selectively unlock and lock a location of a bone pin disposed in the opening 1326 used to secure the guide 300 with respect to the surgical site, thus allowing a location of entry of each bone pin 370a, 370b with respect to the bone and an angle of entry of each bone pin 370a, 370b with respect to the bone to be easily adjusted.

As also described in further detail below, the rod-engaging portion 1310 of the bone pin clamp 332a can include a first body portion or plate 1314 and a second body portion plate 1312 that define an opening 1316 configured to receive a support rod therein. The rod-engaging portion 1320 can be used to selectively unlock and lock a location of the clamp 332a with respect to a support rod (e.g., the support rod 336a) disposed in the opening 1316, and thus with respect to the guide 300 and the surgical site. This allows a surgeon to selectively position the clamps, e.g., the clamp 332a, with respect to the guide 300 and the surgical site, providing for further adjustment capabilities as to the location and angle of entry of each bone pin 370a, 370b with respect to the bone.

A top surface of the plate 1314 of the rod-engaging portion 1310 can be configured to engage a bottom surface of the plate 1322 of the pin-engaging portion 1320. Each includes a ring of radial teeth 1317, 1319, respectively for the plates 1314, 1322, extending circumferentially around their opposed surfaces. The radial teeth 1317, 1319 for the two surfaces are complementary with each other such that when the rod-engaging portion 1310 and the pin-engaging portion 1320 are coupled, the radial teeth engage and grip each other, allowing rotation of one with respect to the other in a ratchet-like manner when the clamp 332a is in an unlocked configuration and being locked with respect to each other when the clamp 332a is in a locked configuration.

The selectively locking and unlocking of the rod-engaging and pin-engaging portions 1310, 1320 can be effected by the threaded screw 1330 and the locking nut 1340, also referred to as a clamp nut, configured to receive the screw 1330. Movement of at least one of the screw 1330 and the locking nut 1340 with respect to the other can cause the clamp 332a to be placed in unlocked and locked positions to set locations of at least one of the clamp 332a with respect to the support rod 336a and/or the pins 370a, 370b with respect to the guide 300. As shown, the portions 1310, 1320 are disposed between the head 1334 of the screw 1330 and the locking nut 1340. A post of the screw 1330 shown in FIG. 11E extending distally from the head 1334 can extend into the rod-engaging and pin-engaging portions 1310, 1320, through the central opening 1350, such that rotating the screw 1330 in a locking manner can cause respective plates 1314, 1312 and 1324, 1322 that define the respective openings or channels 1316, 1326 formed in the respective rod-engaging and pin-engaging portions 1310, 1320 to collapse around the components disposed in the respective channels 1316, 1326 (i.e., the support rod 336a and a bone pin, such as the bone pin 370a, 370b) to lock the location of the components disposed in the openings 1316, 1326 with respect to the arm 310 of the guide 300. The threaded screw 1330 can be welded in place with respect to the locking nut 1340 to prevent it from being screwed completely off the clamp 332a.

As shown in FIG. 11E, each portion 1310, 1320 can include a bottom plate or securing plate, as shown bottom plates 1312, 1322, and a top plate or coupling plate, as shown top plates 1314, 1324. Each plate 1312, 1314, 1322, 1324 includes a central opening 13120, 13140, 13220, 13240, respectively, the openings aligning with each other to help constitute the central opening 1350 of the bone pin clamp 332a. The plates 1312, 1314, 1322, 1324 are each substantially circular in nature meaning a base and a perimeter of each is substantially circular. Further, surfaces of the plates 1312, 1314, 1322, 1324 that are adjacent to each other (e.g., a top surface of a first plate that is adjacent to a bottom surface of a second plate disposed directly above the first plate) are generally complementary of each other, thus allowing the plates 1312, 1314, 1322, 1324 to mate together within the context of forming the clamp 332a. The term bottom surface as used herein can constitute a downward-facing surface as shown, and the term top surface as used herein can constitute an upward-facing surface as shown, unless otherwise indicated or understood by a person skilled in the art.

While each plate 1312, 1314, 1322, 1324 can have many different configurations, in the illustrated embodiment, the securing plate 1312 is similarly configured as the coupling plate 1324 and the coupling plate 1314 is similarly configured as the securing plate 1322. Accordingly, to the extent some features are not visible, a person skilled in the art will understand where those features can be located in view of the mirror images provided by the corresponding plate and/or as otherwise understood by a person skilled in the art in view of the present disclosures.

The securing plate 1312 has a bottom surface that can be configured to be engaged by the head 1334 of the screw 1330. Although this bottom surface of the plate 1312 is not visible, because of the provided mirror image configuration, a recessed region 1321 provided for in a top surface of the coupling plate 1324 provides for the same configuration. As shown, the recessed region 1321 can be tapered, which for purposes of the securing plate 1312 allows the screw head 1334 to progressively slide into the securing plate 1312 and tighten against it, thus helping to place the bone pin clamp 332a in the locked configuration.

A top surface of the securing plate 1312 can be configured to engage and secure the support rod (e.g., the support rod 336a) from a bottom direction. In the illustrated embodiment, one or more grooves 1316a are formed in the top surface of the securing plate 1312 for this purpose. In the illustrated embodiment, a groove 1316a is on one side of the central opening 13120. More grooves can be provided in other embodiments, for example, a second groove can be disposed on the opposing side of the central opening 13120, as shown for central opening 13120′ in FIG. 11G, in which a plurality of locations at which a support rod 336a′ can be disposed with respect to a bone pin clamp 332a′ are shown. With reference back to FIG. 11E, the groove 1316a can generally be shaped and sized to allow the support rod 336a, or other rods, to be seated therein and selectively locked within the groove 1316a as provided for herein. In the illustrated embodiment, the groove 1316a has a plurality of surfaces creating a hex shaped channel 1316 which can benefit in preventing rotation of a hex shaped support rod received therein. Other complementary geometries between the bone pin and groove can be utilized.

The top surface of the securing plate 1312 can also including one or more mating features to help couple the securing plate 1312 to the coupling plate 1314. In the present embodiment, the mating feature includes two protrusions 1318 extending upwardly from the top surface of the securing plate 1312, towards the coupling plate 1314, on opposed sides of the central opening 13120. As shown, the protrusions 1318 can be disposed on an axis parallel to the groove 1316a. The protrusions 1318 can be configured to sit within complementary mating features formed in the bottom surface of the coupling plate 1314, such as recesses or openings 1319.

In addition to including recesses to receive the protrusions 1318, the bottom surface of the coupling plate 1314 can have formed therein a groove 1316b that is complementary to the groove 1316a to engage and secure the support rod 336a from a top direction. Again relying on the mirrored image configuration, the groove 1316b formed in the bottom surface of the coupling plate 1314 can be equivalent of groove 1326b formed in the top surface of the securing plate 1322. The groove 1316a of the securing plate 1312 and the groove 1316b of the coupling plate 1314 can be sized and otherwise adapted to seat a portion of the length of the support rod 336a therein, the grooves 1316a, 1316b forming a channel 1316 therebetween as shown in FIG. 11C. When the plates 1312, 1314 are aligned to form the rod-engaging portion 1310, the grooves 1316a, 1316b align to secure the support rod 336a within them. Other complementary surfaces can be formed in the plates 1312, 1314 to assist further in the two plates being securely fit with respect to each other.

A top surface of the coupling plate 1314 can be configured to engage a bottom surface of the securing plate 1322 of the pin-engaging portion 1320. In the illustrated embodiment, because of the mirrored image configuration, these two surfaces are actually the same. Each includes a ring of radial teeth 1317 extending circumferentially around their opposed surfaces. The radial teeth for the two surfaces are complementary with each other such that when the rod-engaging portion 1310 and the pin-engaging portion 1320 are coupled, the radial teeth engage and grip each other, allowing rotation of one with respect to the other in a ratchet-like manner when in the clamp 332a is in the unlocked configuration and being locked with respect to each other when the clamp 332a is in the locked configuration.

A top surface of the securing plate 1322 can be configured to engage and secure the bone pin 370 from a bottom direction. As shown, this engagement is achieved by way of the groove 1326b disposed on opposed sides of the central opening 13220. As also shown, the recesses 1329 are formed in the top surface of the securing plate 1322, those recesses 1329 being configured to receive protrusions 1328 (only one is visible) extending downwards from a bottom surface of the coupling plate 1324. A bottom surface of the coupling plate 1324 can be configured to engage and secure the bone pin (e.g., the bone pin 370) from a top direction by way of groove 1326a, which is akin to the grooves 1316a illustrated for the securing plate 1312. Likewise, the bottom surface of the coupling plate 1324 can include protrusions 1328 akin to the protrusions 1318, the protrusions 1328 being configured to engage with the recesses 1329. The recessed portion 1321 formed in the top surface of the coupling plate 1324 can be configured to receive the locking nut 1340, allowing the locking nut 1340 to seat therein as shown in FIG. 11C.

The bone pin clamp 332a can be moved between locked and unlocked configurations by moving at least one of the screw 1330 and the locking nut 1340 with respect to the other, for example by rotating one with respect to the other. More particularly, in the illustrated embodiment, the clamp 332a controls locked and unlocked configurations for both the support rod (and other support rods provided for herein) and locked and unlocked configurations for the bone pin (and other bone pins provided for herein).

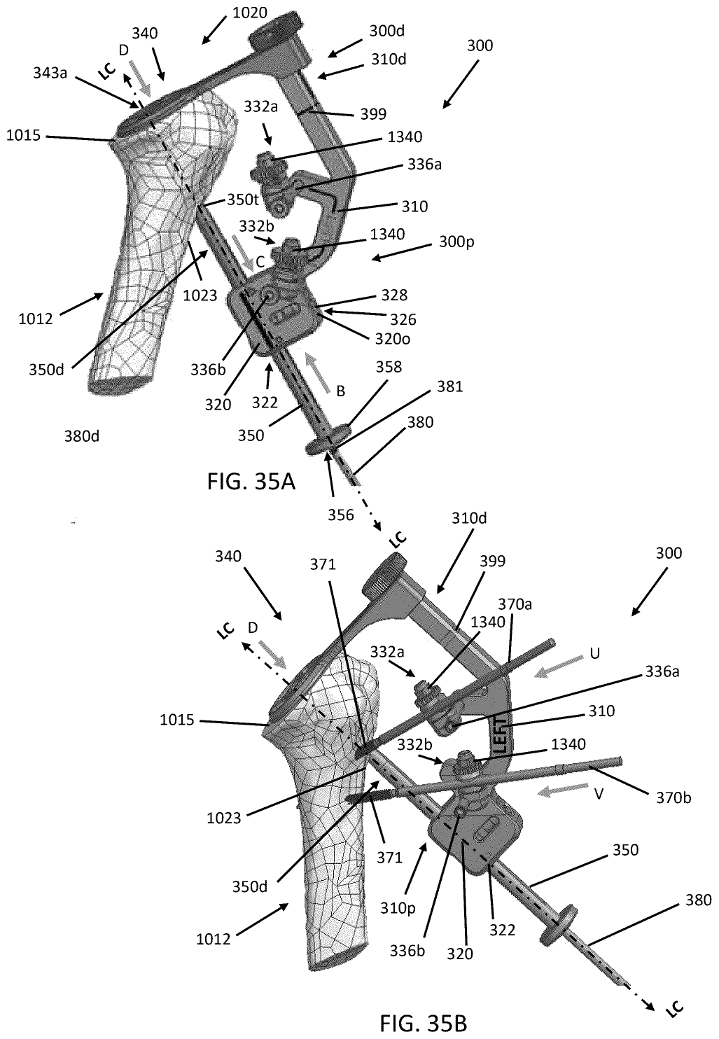

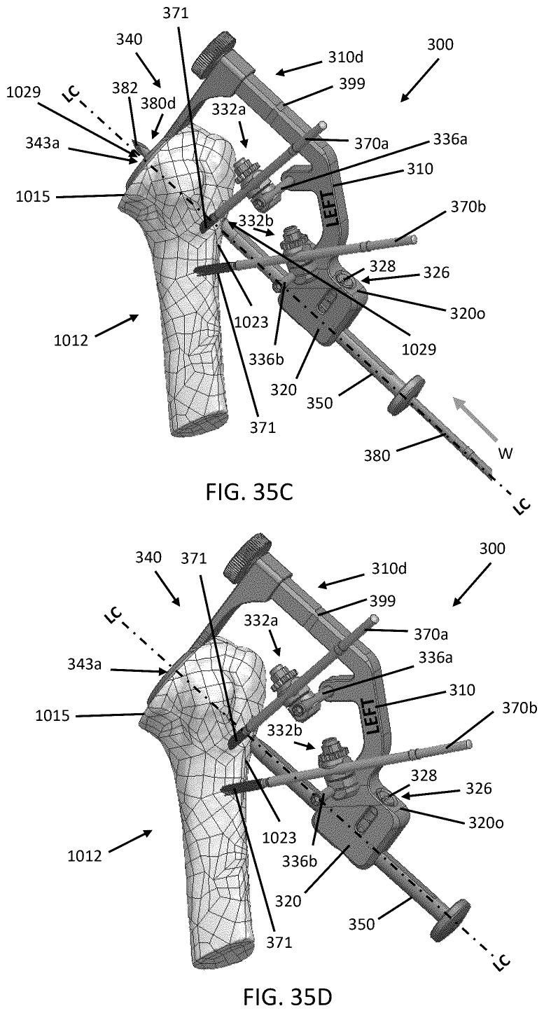

FIG. 11D illustrates the bone pin clamp 332a having the support rod 336a disposed therein with the support rod 336 coupled to the flange 331 of the arm 310. In an unlocked configuration, a support rod (e.g., the support rod 336a) has axial and rotational freedom of movement within the channel 1316 formed by the grooves 1316a, 1316b. Likewise, in an unlocked configuration, a bone pin (e.g., the bone pin 370a), which as shown elsewhere can be disposed in the channel 1326 (see, e.g., FIGS. 35B-35D), has axial and rotational freedom of movement with the channel 1326 formed by the grooves 1326a, 1326b. As the screw 1330 and/or the locking nut 1340 are rotated with respect to each other, the screw head 1334 and locking nut 1340 move closer together compressing the rod-engaging portion 1310 and the bone pin-engaging portion 1320, which moves the bone pin clamp into a locked configuration. In the illustrated embodiment, the distal end 1332 of the screw 1330 is threaded and received within a threaded central opening 13400 of the locking nut 1340. The locking nut 1340 can include a stop washer 1342 that can be integrally formed with the locking nut 1340 or provided separate to the locking nut 1340 as shown in FIG. 11E to extend the threaded opening 13400 and further secure the distal end 1332 of the screw 1330. In the illustrated embodiment, the stop washer 1342 is disposed at the distal end 1332 of the screw 1330, which can then be welded in a specific location to ensure functionality, while also preventing a user from unintentionally disassembling the bone pin clamp 332a, 332b by loosening the locking nut 1340 too much.

The bone pin clamp 332a can also include a spring 1390 disposed around a portion of the screw 1330 between the head 1334 and distal end 1332. The spring 1390 can bias the screw proximally out of the central opening 1350 in the unlocking configuration when the distal end 1332 is unthreaded from the central opening 13400 of the locking nut 1340. In the locked configuration, the threaded engagement between the distal end of the screw 1332 and the central opening 13400 overcomes the biasing force of the spring 1390 thereby securing the screw within the central opening 1350 of the clamp 332a. In the locked configuration, at least one of the support rod or the bone pin is constricted from moving axially and rotationally. In some instances, both the support rod and the bone pin can be so constricted, although it is not necessary that both are constricted simultaneously. It is possible for one to be constricted and in a locked configuration while the other remains free to move axially and/or rotationally in an unlocked configuration. Placing the support rod in the locked configuration secures the position of the clamp 332a along the support rod, while placing the bone pin in the locked configuration secures the position of bone pin within the clamp 332a. Locking the bone pin can also secure the bone pin at a desired location or position at the surgical site. A wrench or other instrument can be used to secure bone pin relative to the clamp 332a.

Subsequent loosening of the screw 1330 and/or the locking nut 1340 can cause one or both of the support rod or the bone pin, or the bone pin clamp 332a more generally, to return to the unlocked configuration. In at least some instances, the clamp 332a can secure the bone pin to the support rod such that a length of the pin is substantially transverse to a length of the rod while the rotational position of the pin engaging portion 1320 relative to the rod engaging portion 1310 can be adjusted before tightening the clamp 332a to set the bone pin 370a at any angle relative to the support rod. This rotational position can be adjusted, for example, by rotating the pin-engaging portion 1320 relative to the rod-engaging portion 1310 by way of the teeth 1317 disposed on the top surface of the coupling plate 1314 and the complementary teeth disposed on the bottom surface of the securing plate 1322. Additionally, the clamp 332a can slide along the support rod when in the unlocked configuration to adjust the position of the clamp 332a and thereby an entry location of the bone pin into bone.

FIGS. 11F-11G provides for an alternative embodiment of a bone pin clamp 332a′. The bone pin clamp 332a′ is similar to that of the bone pin clamp 332a, with a primary difference being that the configuration of the rod-engaging and pin-engaging portions 1310′, 1320′ are such that a support rod 336a′ and the pin 370a can be disposed on opposite sides therein. Nevertheless, a person skilled in the art, in view of the present disclosures, will understand similarities and differences between the two bone pin clamp embodiments 332a, 332a′, and how different features of one embodiment can be incorporated into the other embodiment. Further, the illustration in FIGS. 11F-11G help illustrate placement of the bone pin 370a and the support rod 336a with respect to the clamp 332a′. Similar placement can be used for the bone pin clamps 332a, 332b.

The bone pin clamps 332a, 332b, 332a′ allow for flexibility when mounting the guide 300 to the humerus 1012. The locking nut 1340 can be loosened to allow side loading of the clamp 332a to the guide 300 and/or of the pins 370a, 370b to the clamp 332a. Once the nut 1340 is tightened, side loading and pin removal options are reduced and/or eliminated. The clamps 332a, 332b, 332a′ can be mounted to the support rods 336a, 336b using either opening provided for herein, and although illustrated with the locking nut 1340 being disposed closer to the distal end 310d of the arm 310 than an opposed terminal end at which the bottom plate 1312 is disposed, in other embodiments the clamp 332a, 332b, 332a′ can be rotated 180 degrees such that is what is illustrated as a top of the clamp 332a, 332b, 332a′ is the bottom of the clamp 332a, 332b, 332a′ and vice versa.

The bone pin clamps 332a, 332b, 332a′ are designed to provide multiple degrees of freedom for one or both of a bone pin (e.g., bone pin 370a, 370b, 370a′) and/or a support rod (e.g., support rod 336a, 336b, 336a′) received within the openings 1326, 1316. The multiple degrees of freedom, as provided for herein, include any combination of sliding, rotation, and orientation.

It can be advisable for a surgeon to plan placement of the bone pin clamps 332a, 332b, 332a′, pins 370a, 370b, and other components prior to initiating drilling. Thus, a surgeon can place the pins 370a, 370b in the bone pin clamps 332a, 332b, 332a′ and layout placement of the pins 370a, 370b with respect to the bone 1012 prior to drilling. Factors to consider in planning include but are not limited to surgeon preference, patient anatomy, and the location of soft tissue and neurovascular structures. It is typically best if the pins 370a, 370b are spaced apart by at least 5 mm, that they have bi-cortical fixation, and that they be located below the entry point of the drill cannula 350 to ensure clearance to humeral bone preparation instrumentation and implants. It can also be advisable to prevent the pins 370a, 370b from intersecting the longitudinal axis L extending through the drill cannula 350 to ensure that there is no conflict with transhumeral drilling instrumentation.

For a procedure involving the left shoulder, placement of the pins 370a, 370b and bone pin clamps 332a, 332b on a left side of the guide 300 may provide better anatomical fixation, while for a procedure involving the right shoulder, placement of the pins 370a, 370b and clamps 332a, 332b on a right side of the guide 300 may provide for better anatomical fixation. The guide 300 and related components is flexible to allow pin placement on either side simultaneously as desired for best securement.

A person skilled in the art will appreciate other ways by which the bone pins 370a, 370b can be coupled to the guide 300. For example, in another embodiment of a humeral guide described in U.S. patent application Ser. No. 18/823,258, a guide and related components are provided that aid in guiding and positioning bone pins at the surgical site. Even with respect to the bone pin clamps 332a, 332b, 332a′ a person skilled in the art will appreciate that the bone pin clamps 332a, 332b, 332a′ are just examples of many embodiments that can be used to assist in providing the ability to manipulate a location of entry of the bone pin into the humerus and/or an angle of entry of the bone pin into the humerus. The present disclosure contemplates other clamps or other bone pin-coupling mechanisms that allow for multiple degrees of freedom to make such adjustments to the bone pin with respect to the humerus, including those having similar capabilities without as many components or parts.

### Additional Aspects of the Rigid Arm(s)

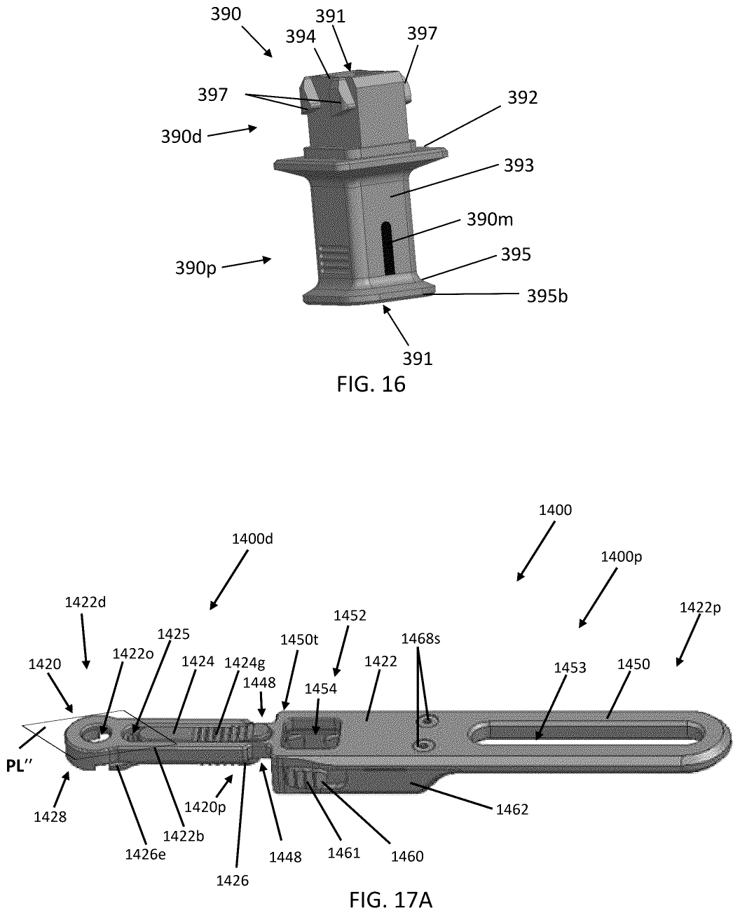

A variety of labels can be provided for on the arm 310 to provide case of use for a user(s). For example, as shown on the distal portion 310d of the arm 310, a demarcation line 399, as shown a laser line, can be formed in or on the arm 310. As described below, the demarcation line 399 can be used in conjunction with other components (e.g., an adapter as illustrated in FIG. 16) to help know when a tool being used at the surgical site has entered into the humerus to a desired depth. In at least some instances, the demarcation line 399 can be machined into the arm 310 such that the line 399 can act as a mechanical stop. As also shown, a label of “LEFT” can be displayed on one side of the arm 310, and, not visible, a label of “RIGHT” can be displayed on the opposed side of the arm 310. These labels match the operative side of the patient. Still further, one or more alignment lines 361a, 361b can be formed on various portions of the arm 310. In the illustrated embodiment, the alignment line 361a is formed on the distal portion 310d to assist in aligning a component, like the adapter (see FIG. 16) on the arm 310, and the alignment line 361b is formed on the hub 320 to assist in aligning the drill cannula 350.

The rigid arm 310 can be sized and shaped to allow for proper centering and alignment between, for example, the drill cannula 350 and one of the various modular attachments that can be coupled to the distal portion 310d. The size and/or shape of the arm 310 can depend, at least in part, on the size and anatomy of the patient (e.g., child vs. adult, male vs. female, etc.) and/or the preferences of the surgeon. In the illustrated embodiment, the length of the rigid arm 310 is generally curved or angular in shape as it extends from a proximal end of the proximal portion 310p of the arm 310 to a distal end of a distal portion 310d of the arm 310, the proximal and distal ends being terminal ends of the arm 310 that define the length of the arm 310. The rigid arm 310 can be sized and shaped to allow for grasping with one hand during a surgical procedure, and can provide for universal adaptation such that it can be grasped in an equally convenient and easy-to-use manner by a user's right hand or left hand without having to change grips and/or positions during the surgical procedure. The guide 300 can provide further universal adaptation such that it can be placed on either the left or right side of a patient's anatomy. For example, the same guide 300 can be utilized for an arthroplasty procedure on a right shoulder or a left shoulder. Accordingly, the humeral guide 300 can be considered a universal humeral guide. More particularly, the support rods 336a, 336b used in conjunction with the supporting bone pin clamps 332a, 332b can be disposed on one side of the guide 300 or the other depending on whether the procedure being performed is on the right of left shoulder. In the illustrated embodiment, the rods 336a, 336b are disposed on one side of the arm 310 for use in a left shoulder procedure. In a right shoulder procedure, the rods 336a, 336b can be disposed on the opposite side of the arm 310.

Additional aspects and embodiments of humeral guides, and components used in conjunction therewith, are described in U.S. patent application Ser. No. 18/823,258, referenced above, and which is incorporated by reference herein in its entirety.

### Bone Pin(s)

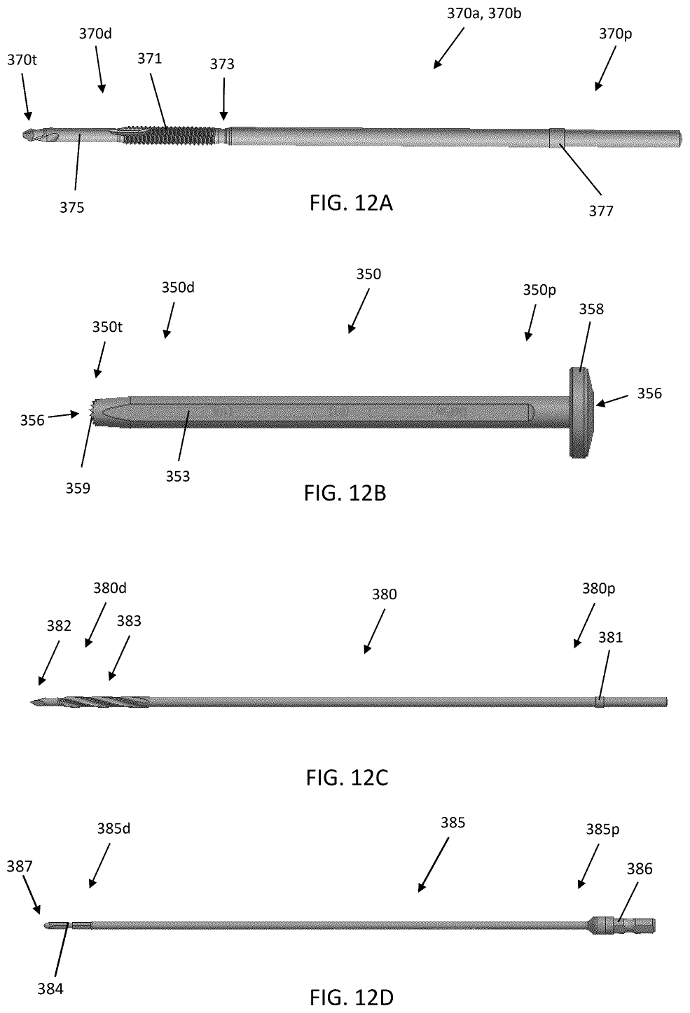

Bone pins 370a, 370b that can be used in conjunction with the bone pin clamps 332a, 332b, and with the humeral guide 300 more generally, are illustrated in FIG. 12A. The pins 370a, 370b are identical in the present disclosure, though a person skilled in the art will appreciate different pins can be used in the same embodiment. Further, in some embodiments, the pins can be used as guide pins or the like used in other contexts, such as helping to use tools attached to a handle assembly (e.g., a handle assembly 1400).

As shown in FIG. 12A, the bone pins 370a, 370b can include a proximal portion 370p, a distal portion 370d, and an intermediate length extending therebetween. The proximal portion 370p can include an indicator 377, as shown a raised radially raised portion, though in other embodiments it can be a laser marking or other indicator known to those skilled in the art, designed to provide a visual indication about the location of the bone pin 370a with respect to another component. This is akin to an indicator 381 formed on a drill bit 380, as described below with respect to FIGS. 12C and 35A.

The distal portion 370d can include a bone-engaging or distal tip 370t, a relief section 375, threads 371, and an indentation 373 formed proximal of the threads 371. The relief section 375 can provide a tactile feel to the user after entering an initial cortical bone, as the bone pin 370a can quickly advance without pressure to a far cortical wall. In some procedures, advancing the bone pin 370a approximately an additional about 3 millimeters to about 5 millimeters can embed the drill bit 380 in a far cortex while simultaneously securing the threaded section 371 to resist cantilever forces and provide sufficient stability. The threads 371 can allow for measured insertion of the pin 370a, 370b into the bone by rotating the same, while the indentation 373 can provide relief for manufacturing the threads 371 of the bone pin 370a, 370b. A non-limiting embodiment of the bone pin 370a, 370b is a 4.00 mm threaded Schanz screw, available from DePuy Synthes (Raynham, MA). In alternative embodiments, the bone pins may be non-threaded, which, for at least some surgeons, can provide desired tactile feel and/or comfort. The configuration of the bone pins 370a, 370b can be identical or substantially identical as used herein, with the pin 370a being a superior bone pin and the pin 370b being an inferior bone pin as illustrated in various embodiments. Accordingly, reference to configurations and the like of one pin can be equally applicable to the other.

### Drill Cannula(s)

As illustrated in FIGS. 10A, 10B, and 12B, the drill cannula 350, also referred to as a cannula, a bullet, or a cannulated bullet, can be an elongate, substantially cylindrical or tubular shaft having a base or handle 358 located at a proximal end 350p, a distal end 350d having a distal tip 350t, and an intermediate portion or length extending therebetween. A planar contact surface 353 can be disposed on an outer surface of the intermediate portion of the drill cannula 350 to provide a surface for the locking nut to contact and lock the position of the drill cannula 350. In some embodiments, ratcheting teeth 351 (see at least FIG. 10B) can be disposed on an outer surface of the intermediate portion of the drill cannula 350 or along the planar contact surface 353, which as described above, can be engaged by a tooth associated with the distal end of the button 328. The drill cannula 350 further includes an opening 356 extending through an entirety of a length of the drill cannula 350. The opening 356 can be sized and shaped to allow a drilling component, such as a drill bit or guide pin 380, to pass through the drill cannula 350 and into the bone in which a bone tunnel is to be formed such that a distal end 380d of the drill exits center and perpendicular to the humeral resection surface 1015 as shown in FIG. 35C. The distal end 350d of the drill cannula 350 can be tapered, for instance by varying a thickness of a wall of the drill cannula 350, to make it easier to push the drill cannula 350 through soft tissue and against the surface of the humerus. In some embodiments, bone engaging features, such as teeth 359, can be formed at the distal tip 350t. The teeth 359 can help stabilize the location of the distal end 350t of the bullet with respect to the bone. Further a distal most end of the distal portion 350d of the drill cannula 350 can be angled to match the angle of the humerus 1012. The stabilization provided by features such as the teeth 359 and angled distal end 350d can set a more accurate trajectory for the bone tunnel. The cannula-receiving opening 322, along with the drill cannula 350, sets the location and trajectory of a bone tunnel or bore to be drilled through the humerus from the lateral cortex 1023 to the humeral resection surface 1015, the tunnel center, or substantially center, and perpendicular, or substantially perpendicular, to the humeral resection surface 1015. The terms tunnel and bore will be used interchangeably throughout the specification as it pertains to forming a hole in a bone.

### Drill Bit(s)

FIG. 12C illustrates one non-limiting embodiment of a drill bit that can be used in conjunction with the present disclosures. As shown, the drill bit 380 can include a proximal portion 380p, a distal portion 380d, and an intermediate length extending therebetween. The proximal portion 380p can include an indicator 381, as shown a raised radially raised portion, though in other embodiments it can be a laser marking or other indicator known to those skilled in the art, designed to provide a visual indication about the location of the drill bit 380 with respect to another component. For example, in the illustrated embodiment, and as further illustrated by FIG. 35A, the indicator 381 is designed to inform a user that the drill bit 380 has traveled to a location that provides desired docking of the guide 300. More particularly, when the drill bit 380 is advanced approximately 10 millimeters into the humerus 1012, the indicator 381 is disposed at the base 358, thus providing visual indication that the drill bit is at a desired docking location. The distal portion 380d includes a distal tip 382, and then a portion 383 having a wider diameter. The distal tip 382 allows for better formation of a pilot hole, with the wider diameter portion 383, in turn, being able to drill a larger diameter bore.

FIG. 12D illustrates a non-limiting embodiment of a drive shaft 385 that can be used in conjunction with the present disclosures. The drive shaft 385 can include a proximal portion 385p that can include hub 386 that can be used to couple the drive shaft 385 to a power source. In the illustrated embodiment, the hub 386 is a modified trinkle connection. A distal end 385d of the drive shaft 385 can include a rounded distal tip 387. The drive shaft 385 can further include a groove 384 formed proximal of the distal tip 387 of the distal end 385d, around an outer surface or perimeter of the shaft 385. The distal end of the drive shaft can couple to various attachments, like a reamer attachment (see FIG. 15) at the groove 384. In use, the power source coupled to the proximal end 385p provides a rotation force to the shaft 385 and attachment or component coupled thereto. In some embodiments, features of the guide pin or drill bit 380 and the drive shaft 385 can be combined into one component for use throughout the procedure. For example, a groove similar to groove 384 of the drive shaft 385 can be included on the drill bit 380 to facilitate coupling of the drill bit 380 to an attachment.

### Humeral Sizer Attachment(s)

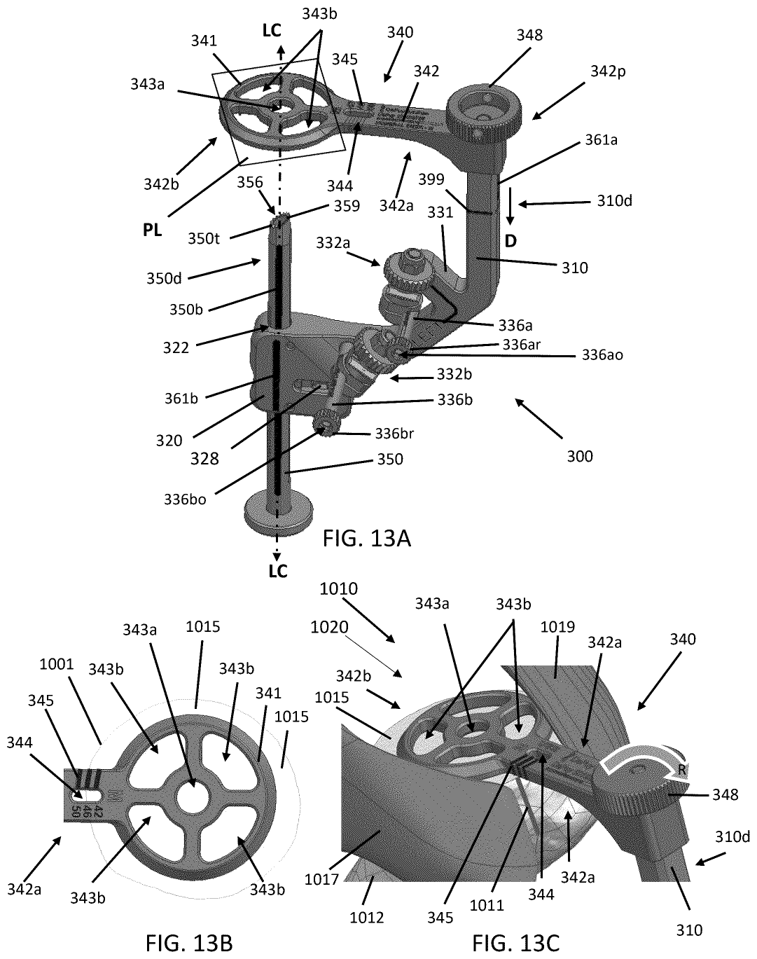

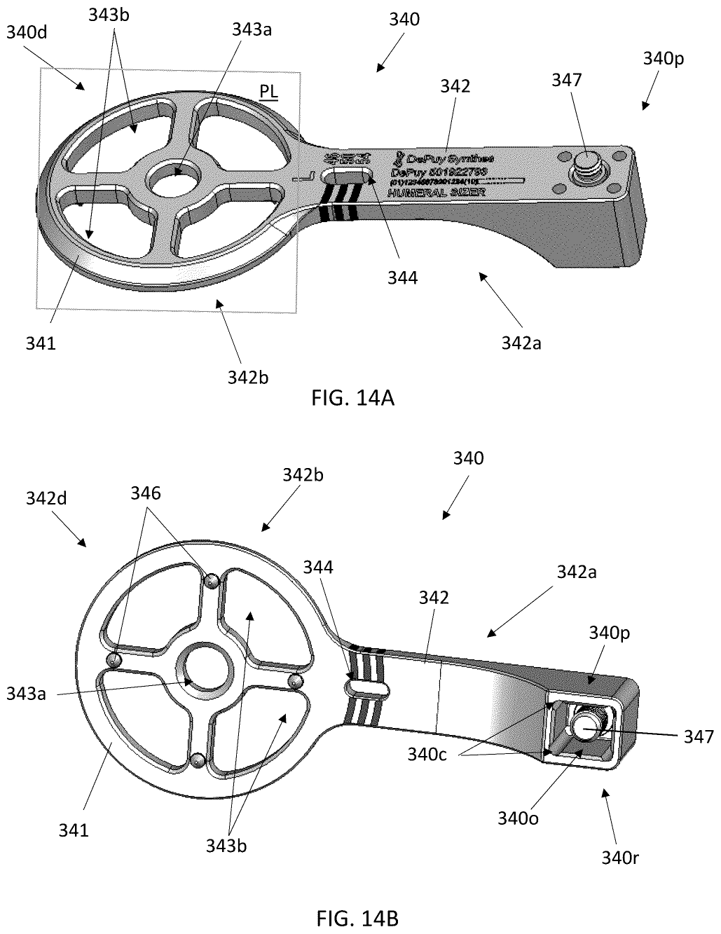

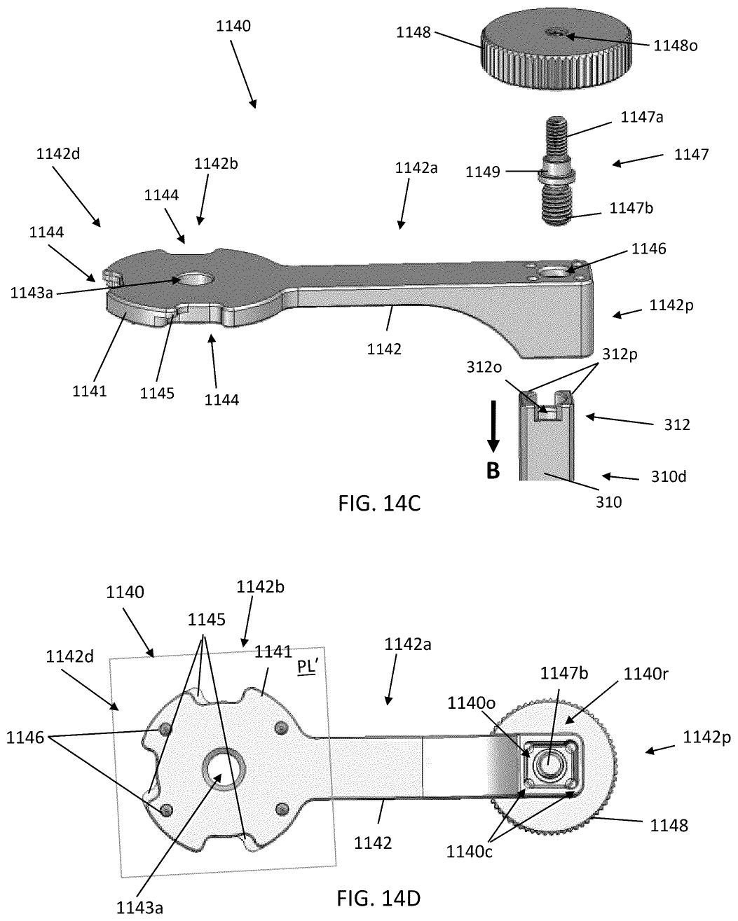

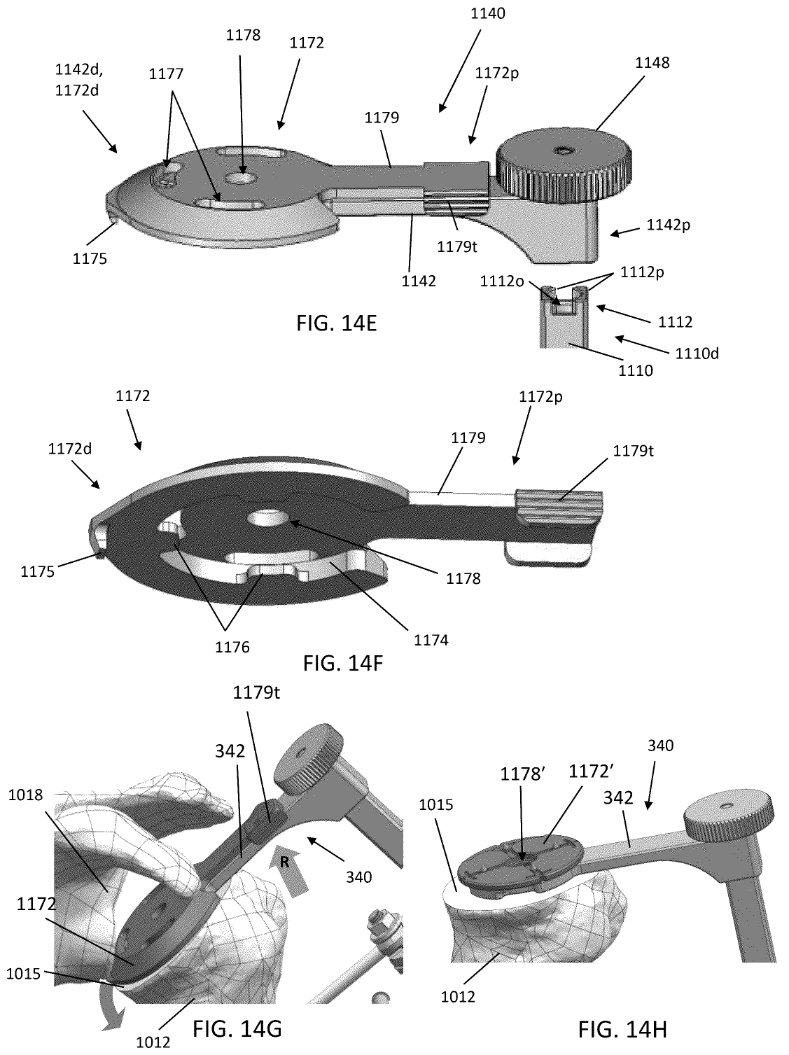

As noted, various modular attachments can be coupled or otherwise associated with the guide 300 at the distal end 310d of the rigid arm 310. In FIGS. 10A and 13A, such a modular attachment is provided in the form of a sizer attachment or a humeral sizer attachment 340. The attachment 340, which is illustrated in FIGS. 14A-14B, can include a substantially flat, rigid base 342 having an arm portion 342a and an alignment and sizing portion 342b, also referred to as a plate portion. As shown, the arm portion 342a can extend from a proximal-most end 342p, the location at which the attachment 340 can be coupled to the rigid arm 310, and terminates where a circular shape that primarily constitutes the plate portion 342b begins. In the illustrated embodiment, the plate portion 342b comprises a circular body 341 formed at a distal end 342d of the base 342, the circular body 341 having a plurality of openings 343a, 343b formed therein. As shown, there are four outer openings 343b, each similarly sized and shaped and disposed radially around a central opening 343a. The rigid base 342 has a length from a proximal-most end illustrated at a proximal end 342p to a central opening 343a such that when the humeral sizer attachment 340 is mated with the rigid arm 310, the central opening 343a is on the same axis LC as the cannula-receiving opening 322 of the hub 320. Further, a plane PL defined by the plate portion 342b of the attachment 340 can be orthogonal or substantially orthogonal to the longitudinal axis LC. As shown, the plane PL extends through a primary surface of the sizing portion 342b of the attachment 340, such as an entirety of the top surface.

The central opening 343a can be centrally located with respect to the circular body 341 and can be configured to receive a drill bit or guide pin, such as the drill bit 380 (not shown in FIG. 13A, but see FIG. 10A), passed through the drill cannula 350. As will become clearer by way of the present disclosure, the drill bit or guide pin 380 can more generally be referred to as a tool-operating shaft or drive shaft as various guide pins provided for herein can also be used to operate one or more tools associated with the guide 300, for example by way of a handle assembly (e.g., a handle assembly 1400), which is also disclosed further herein. The central opening 343a can be used, for example, to provide alignment of other portions of the guide 300, or components with which the guide 300 is used such as the drill cannula 350 and drill bit 380. More particularly, the central opening 343a can be used, for example, to locate and/or mark an approximate center point of the humeral resection surface 1015, as shown in FIGS. 13B and 13C.

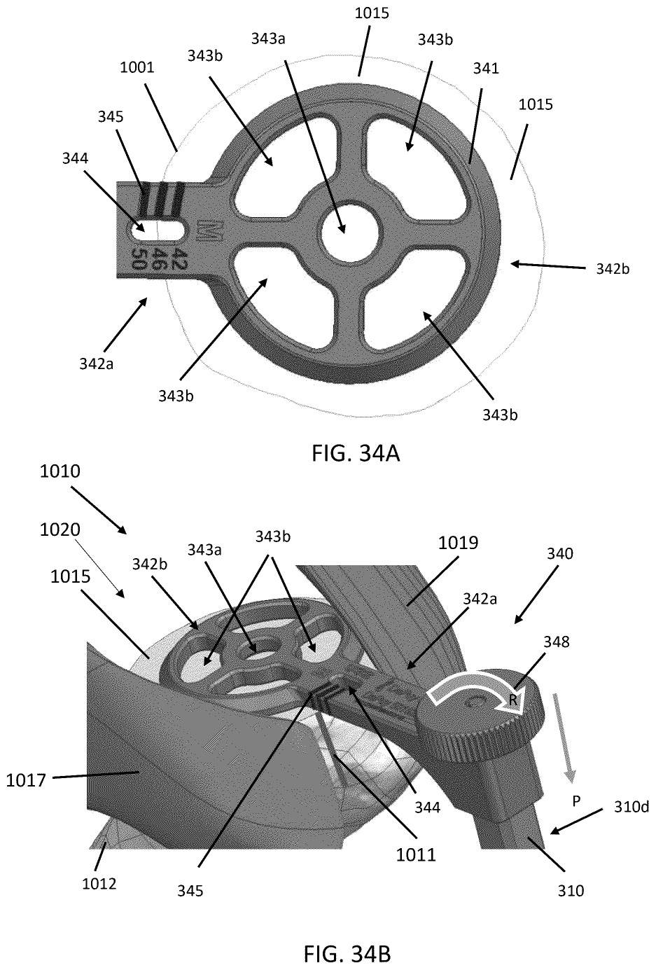

A window 344 can be formed in the distal end of the arm portion, proximal to where the arm portion 342a joins with the plate portion 342b. A user can align an anatomical landmark, such as the bicipital groove, within the window 344. The arm portion can further include markings relating to various measurements of a humeral resection surface or an appropriately sized implant. As described in greater detail below with respect to FIGS. 34A-34B, referencing the humeral resection surface 1015 like as shown in FIG. 13B allows a user to pick a nominal sizer and reference a marking or indicator 345 on the arm portion 342a. Accordingly, when the sizer attachment 340 is inserted into the joint space as shown in FIG. 13C, the user can laterally line-up a line 1011 formed on the bicipital groove within the window 344, and push the sizer attachment 340 into the joint until that reference marking 345 is reached, thus setting a medial lateral positioning on the humeral resection surface 1015. Holding that position, the user can manually center, for example by palpation or visual reference, the anterior to posterior centering using the bone and the plate portion 342b. The provided locators centralize the sizer attachment 340 to what a user may want and can, if desired, ignore most inferior position where they may nibble bone off inferiorly in the end. This locating process can set the definitive implant to be located properly within rotator cuff muscles.

Features for mating the attachment 340 to the attachment feature 312 of the arm 310 are provided in the proximal portion 340p. As shown best in FIG. 14B, a receiving feature 340r can be located at a proximal end 340p of the humeral sizer attachment 340. The receiving feature 340r can include a central opening 3400 and a plurality of channels 340c that are complementary to the U-shaped posts 312p of the attachment feature 312 of the arm 310. Accordingly, the humeral sizer attachment 340 can slide onto the distal end 310d of the arm 310 by advancing the attachment 340 in the direction D, as shown in FIG. 13A, causing the posts 312p of the arm 310 to slide within the channels 340c. The humeral sizer attachment 340 can be locked into place with respect to the arm 310 by way of a knob 348, also shown in FIG. 13A, that engages a threaded post 347 to selectively lock and unlock the attachment 340 from the arm 310. As also shown in FIG. 14B, one or more engagement teeth protrusions 346 can be formed on a bottom or humeral resection surface facing surface of the attachment 340. As shown, there are four teeth 346, each radially and equidistantly disposed around a perimeter of the circular body 341. The teeth 346 can be configured to engage bone to help set the location of the humeral sizer attachment 340 with respect to the bone. Any number and configuration of teeth can be used without departing from the spirit of the present disclosure.

The sizer attachment 340 can be used to both define a center, or a central location, of the humeral resection surface 1015, or other desired location to receive an implant, as well as size the humeral resection surface 1015 for determination of the size of the implant and/or prosthesis to be used at the surgical site. Sizer attachments 340 of various lengths and having variously sized plate portions 342b of various diameters (e.g., extra small, small, medium, large, extra large) can be provided with the guide 300 to accommodate varying patient humeral resection surface diameters and anatomies. The illustrated embodiment of the attachment 340 has a large diameter, as designated by the “L” shown in FIG. 14A (the equivalent attachment 340 in FIGS. 13A-13D has a medium diameter, as designated by the “M” shown in those figures). The sizes of the sizer attachments 340 can be based, for example, on a size of an implant (e.g., a stemless implant) and/or a reamer or reamer attachment that will be used to ream the humeral resection surface 1015.

In at least some instances, an appropriately sized sizer attachment 340 can be chosen such that an outer edge of the plate portion 342b lies within the cortex border of the humeral resection surface 1015. Often it can be helpful for a perimeter of the circular body 341 to lie immediately within the cortex border and not contact any portion of the cortex perimeter. As shown in FIG. 13B, markings or indicators 345 on the arm portion 342a of the sizer attachment 340 can allow the sizer attachment 340 to center and measure the humeral resection surface 1015 with a circumference larger than the circular body 341, as also described in greater detail with respect to FIGS. 34A-34B. The markings 345 can be laser lines or other types of markings known to those skilled in the art. The circular body 341 can be substantially flat in shape and designed to enter a rotator interval 1020 above the subscapularis.

Once in the joint space, the plate portion 342b can be aligned parallel, or substantially parallel, and against the humeral resection surface 1015 such that the opening 343a, 343b mark the center point, or the approximate center point, of the humeral resection surface 1015. The alignment of the center opening 343a with the center point of the humeral resection surface 1015 can place the drill cannula 350 at the correct location on the lateral cortex 1023 of the humerus 1012 such that when a drill bit or guide pin 380 is passed through the drill cannula 350 and humerus 1012, the distal tip 382 disposed at a distal end 380d of the drill bit 380 exits center and perpendicular, or substantially perpendicular, also referred to as orthogonal or substantially orthogonal, to the humeral resection surface 1015. Further, the distal tip 382 can be used to puncture tissue and/or drill in bone as desired. The humeral sizer attachment 340 further operates with the distal tip 350t of the drill cannula 350 to create a clamping force on the humeral resection surface 1015, which can maintain the position of the guide on the humerus until bone pins 370a, 370b are passed through the fixation feature(s) 330a, 330b, as shown through the bone pin clamps 332a, 332b, to secure the guide 300 to the bone.

A person skilled in the art will appreciate sizer plates, such as INHANCE™ humeral head trials (from Johnson & Johnson of New Brunswick, NJ), can also be used in conjunction with, or in lieu of, the humeral sizer attachment 340 and/or other similarly designed humeral sizer attachments.

Another embodiment of a sizer attachment 1140 is illustrated in FIGS. 14C-14E, with FIGS. 14E-14H illustrating removeable sizer plates 1172 that can be used with the sizer attachment 1140. A person skilled in the art will appreciate how sizer plates like the plates 1172 can be used with other sizer attachments, such as the humeral sizer attachment 1140. In the illustrated embodiment, the sizer attachment 1140 is configured to have sizer plates 1172 twisted onto the attachment 1140 such that the plates 1172 can be selectively coupled to and removed from the attachment 1140.

The attachment 1140 can include a substantially flat, rigid base 1142 having an arm portion 1142a and an alignment and sizing portion 1142b. As shown, the arm portion 1142a can extend from a proximal-most end 1142p, the location at which the attachment 1140 can be coupled to the rigid arm 310, to the end of the elongate section of the arm portion 1142a, which in the illustrated embodiment terminates where a circular shape that primarily constitutes the alignment and sizing portion 1142b begins. In the illustrated embodiment, the alignment and sizing portion 1142 comprises a circular body 1141 formed at a distal end 1142d of the base 1142, the circular body 1141 having a central opening 1143a formed therein. The rigid base 1142 has a length from a proximal-most end illustrated at a proximal end 1142p to a central opening 1143a such that when the sizer attachment 1140 is mated with the rigid arm 1110, the central opening 1143a is on the same axis as the cannula-receiving opening 322 of the hub 320. Further, a plane PL′ defined by the alignment and sizing portion 1142b of the attachment 1140, which can be akin to the plane PL, can be substantially orthogonal to the longitudinal axis LC. As shown, the plane PL′ extends through a primary surface of the sizing portion 1142b of the attachment 1140, such as an entirety of the top surface.

In the illustrated embodiment, the circular body 1141 includes a plurality of cutouts 1144 formed in a perimeter of the circular body 1141, the cutouts 1144 being configured to receive various sizer plates. In the present embodiment, the cutouts 1144 are formed along a perimeter of the circular body 1141 and configured to receive and secure a sizer plate 1172, also referred to as a humeral resection surface sizer or a humeral resection surface sizer plate. As shown in FIGS. 14C-14D, three substantially equally sized and shaped cut outs can be formed radially around a perimeter of the circular body 1141, although any number of cut outs in any shape, configuration, and/or layout can be formed along the perimeter. A portion of the cutout 1144 extending through a lower surface of the circular portion (the surface facing towards the hub 320 in use) can be larger in width than the portion of the cutout 1144 extending through an upper surface of the circular body 1141 that is opposed to the lower surface. As shown in FIGS. 14C-14D, this results in tabs 1145 formed on the circular body 1141 within the cutout 1144 that can be used to help secure sizer plates to the attachment 1140.

Disposed on the lower surface of the circular body 1141 can be one or more engagement teeth or protrusions 1146, illustrated in FIG. 14D. As shown, there are four teeth 1146 disposed symmetrically around the central opening 1143a, although in other embodiments they may not be symmetrically disposed or they can be radially and equidistantly disposed around a perimeter of the circular body 1141. The teeth 1146 can be configured to engage bone to help set the location of the sizer attachment 1140 with respect to the bone. Any number and configuration of teeth can be used without departing from the spirit of the present disclosure.

The central opening 1143a can be centrally located with respect to the circular body 1141 and can be configured to receive a drill, drill bit, drive shaft, or guide pin (e.g., drill bit 380) passed through the drill cannula 350. The central opening 1143a can be used, for example, to provide alignment of other portions of the guide 300, or components with which the guide 300 is used, such as the drill cannula 350 and the drill bit 380. More particularly, the central opening 1143a can be used, for example, to locate and/or mark a center point of the surface of the glenoid and/or the glenoid surface, and/or the humeral resection surface, more generally. In conjunction with the same, in at least some instances, prior to and/or in conjunction with using the bone pins 370a, 370b to secure a location of the guide 300 with respect to the humerus 1012, the attachment 1140 and the drill cannula 350 can be operated together to create a clamping force on the humerus 1012 to hold the position of the guide 300. The drill bit 380 can be passed through the drill cannula 350, into the humerus 1012, and exiting the humeral resection surface and the central opening 1143a of the attachment 1141, creating a transhumeral bone tunnel.

The sizer attachment 1140 can be mated to the distal end 310d of the rigid arm 310 via the attachment feature 312, for instance by moving the sizer attachment 1140 in a direction B towards the distal end 310 of the rigid arm 310. FIG. 14D illustrates a receiving feature 1140r located at a proximal end of the sizer attachment 1140. As shown, the receiving feature 1140r can include a central opening 11400 and a plurality of channels 1140c that are complementary to the U-shaped posts 312p of the attachment feature 312 of the arm 310. Accordingly, the sizer attachment 1140 can slide onto the distal end 310d of the arm 310 by advancing the attachment 1140 in a direction F, causing the posts 312p of the arm 310 to slide within the channels 1140c. The humeral sizer attachment 1140 can be locked into place with respect to the arm 1110 by way of a knob 1148 (FIG. 14C) that passes into and through an opening 1146 formed in the arm portion 1142a of the rigid base. The knob 1148 can include a threaded piece 1147 having a threaded top portion 1147a configured to threadingly engage a central opening 11480 of the knob 1148 and a threaded bottom portion 1147b configured to pass through the opening 1146 and engage the opening 3120 formed in the arm 310. A seating extension 1149 can be disposed between the threaded top portion 1147a and the threaded bottom portion 1147b, the seating extension 1149 being configured to set within the opening 1146. Rotation of the knob 1148 can be operable to securely couple the sizer attachment 1140 to the arm 310 and to decouple the sizer attachment 1140 from the arm 310 by way of the interaction between the threaded bottom portion 1147b and the attachment feature 312.

In use, the sizer attachment 1140 can sit just inside the cortex border of the humeral resection surface 1015. The sizer attachment 1140 can be used to both define a center, or a central location, of the humeral resection surface 1015, or other desired location to receive an implant, as well as size the humeral resection surface 1015 for determination of the size of the implant and/or prosthesis to be used at the surgical site. It can also be used in conjunction with sizing aspects of implanting a prosthesis in the glenoid 1018.

A plurality of various sized head trial components or sizer plates 1172 can be used in conjunction with the sizer attachment 1140, as illustrated in FIGS. 14E-14F. The sizer plates 1172 can be used to determine a diameter of the humeral resection surface 1015 and to assist in locating a center, or approximate center, of the humeral resection surface 1015. The sizer plate 1172, illustrated separate from the sizer attachment 1140 in FIG. 14F, can be manufactured in various diameters to accommodate varying patient humeral resection surface diameters. In at least some instances, an appropriately sized sizer plate 1172 can be chosen such that an outer edge of the sizer plate 1172 lies within the cortex border of the humeral resection surface 1015. Often it can be helpful for a perimeter of the sizer plate 1172 to lie immediately within the cortex border and not contact any portion of the cortex perimeter. Both the circular body 1141 and the sizer plate 1172 can be substantially flat in shape and designed to enter a rotator interval 1020 above the subscapularis. The sizer plate 1172 can be considered to be a twist-on sizer because it is configured to be twisted onto and off the sizer attachment 1140. The plate 1172 can be coupled to the sizer attachment 1140 prior to or after the sizer attachment 1140 is coupled to the distal end 310d of the arm 310.

As shown in FIG. 14F, a circular recess 1174 can be formed in a lower surface of the sizer plate 1172, the lower surface being the surface that faces the attachment 1140. The recess 1174 can be sized to seat on the attachment 1140 such that the circular body 1141 of the rigid base 1142 sits within it. A plurality of tabs 1176 can extend from a sidewall of the recess 1174 towards a central opening 1178 formed in the plate 1172. The tabs 1176 can be sized such that they can pass through the cutouts 1144 formed in the perimeter of the circular body 1141 and then the plate 1172 can be twisted to allow the tabs 1176 to engage with the tabs 1145 formed on the circular body 1141. These twist-on connection features allow various plates to be easily secured and removed from the base 1142 to determine the size of the humeral resection surface 1015. In the present embodiment, three tabs 1176 are disposed radially around the recess 1174 to correspond with the three cutouts 1144 and three tabs 1145 formed as part of the attachment 1140. Further, three openings 1177 can be formed in the sizer plate 1172, such openings 1177 being aligned with the tabs 1176. This configuration allows the openings 1177 to be used to help see that the tabs 1176 have engaged the tabs 1145 to help secure a location of the sizer plate 1172 with respect to the attachment 1140.

In the illustrated embodiment, the sizer plate 1172 also comprises an arm or extension 1179 as part of a proximal portion 1172p thereof. As shown, the arm 1179 can be sized to sit substantially within a width of the attachment body 1142. Further, at a proximal end 1179p of the arm 1179, opposed engagement tabs 1179t can be provided to provide a further way by which the sizer plate 1172 can be secured or otherwise positioned with respect to the attachment 1140.

A bone-engaging tang or protrusion 1175 can be disposed on a distal end 1172d of the sizer plate 1172. The tang 1175 can be configured to grasp an edge of the humeral resection surface 1015 when centering the attachment 1140 having the sizer plate 1172 coupled thereto on the humeral resection surface 1015, such as by locating the cortical rim. The central opening 1178 of the sizer plate 1172 can be used for alignment with the central opening 1143a of the attachment 1140 and the cannula-receiving opening 1122 in the hub 320. A person skilled in the art will understand how such sizer plates can be used to size a humeral resection surface. A non-limiting embodiment of sizer plates that can be used are INHANCE™ humeral head trials from DePuy Synthes.

Once in the joint space, the sizer attachment 1140 can be aligned parallel and against the humeral resection surface 1015 such that the openings 1143a, 1178 mark the center point of the humeral resection surface 1015. The alignment of the center openings 1143a, 1178 with the center point of the humeral resection surface 1015 can place the drill cannula 350 at the correct location on the lateral cortex 1023 of the humerus 1012 such that when a drill or guide pin, like the drill bit 380, is passed through the drill cannula 350 and humerus 1012, the distal tip 382 exits center and perpendicular, also referred to as orthogonal, to the humeral resection surface 1015. Further, the distal tip 382 can be used to puncture tissue and/or drill in bone as desired. The sizer attachment 1140 further operates with the distal tip 350d of the drill cannula 1150 to create a clamping force on the humeral resection surface 1015, which can maintain the position of the humeral guide on the humerus 1012 at least until the bone pins 370a, 370b are passed through the bone pin clamps 332a, 332b to secure the humeral guide 300 to the bone.

After the sizer plate 1172 has been used, and/or it needs to be replaced by a differently sized and/or configured plate, it can be detached from the humeral sizer attachment 1140. As illustrated in FIG. 14G, this can be accomplished, for example, by detaching the engagement tabs 1179t from the base 1142, and then removing the sizer plate 1172 from the joint space. More particularly, the engagement tabs 1179t can be pushed and the sizer plate 1172 disconnected from the attachment 1140, for example by moving the portion that include the tabs 1179t in the direction R and then rotating the sizer plate 1172 to detach it from the attachment 1140. Additional sizer plates 1172 can be attached and detached until centering of the guide 300 is accomplished and the proper size is determined for an instrument to be used with the humeral resection surface 1015, implant, prosthesis, and/or other equipment to be used and/or implanted at the surgical site. The guide 300 can remain at the set location for a remainder of the surgical procedure to assist in guiding other instruments for accurate glenoid and/or humeral bone preparation. The attachment 1140 can likewise remain attached to the guide 300 for at least some portion of procedures that are subsequently performed.

In some embodiments, the centering and sizing can be further confirmed by introducing another sizer component, as shown an INHANCE humeral sizer or sizer plate 1172′ available from DePuy Synthes, as shown in FIG. 14H. The INHANCE humeral sizer 1172′ can include a central boss (not visible) that extends distally in the illustrated embodiment of FIG. 14H, towards the attachment 1140. A central opening 1178′ can extend through the sizer 1172′, including through the central boss. The boss can be inserted into the opening 1143a of the attachment 1140 such that the opening 1143a and the central opening 1178′ are aligned. As shown, the sizer 1172′ can sit immediately inside the cortical rim and at no point contacts the cortical rim. During a procedure, a user can evaluate this positioning of the sizer 1172′ with respect to the humeral resection surface 1015. After the positioning of the guide 300 has been confirmed, a user can ensure that all connection points are fully secured, including but not limited to the bone pins 370a, 370b, the rods 336a, 336b, the bone pin clamps 332a, 332b, the drill cannula 350, and the attachment 1140 (or the attachment 340 in other embodiments).

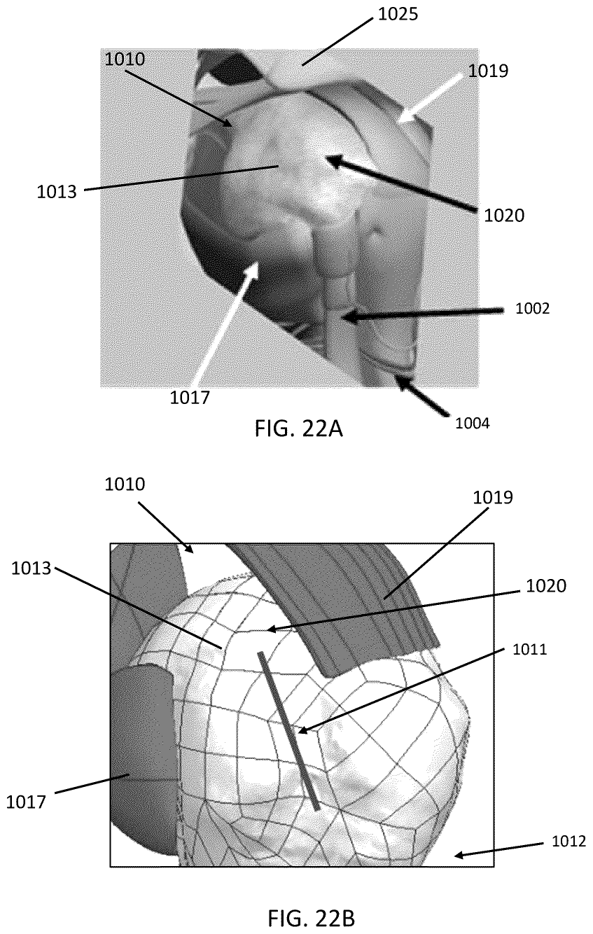

Although not included as part of the procedure described with respect to FIGS. 22A-44G, a person skilled in the art, in view of the present disclosures, would be able to use one or more sizer plates, like the sizer plates 1172, 1172′, and INHANCE™ humeral head trials, and associated components in conjunction with sizing a humeral resection surface 1015.

### Handle Assembly(ies)

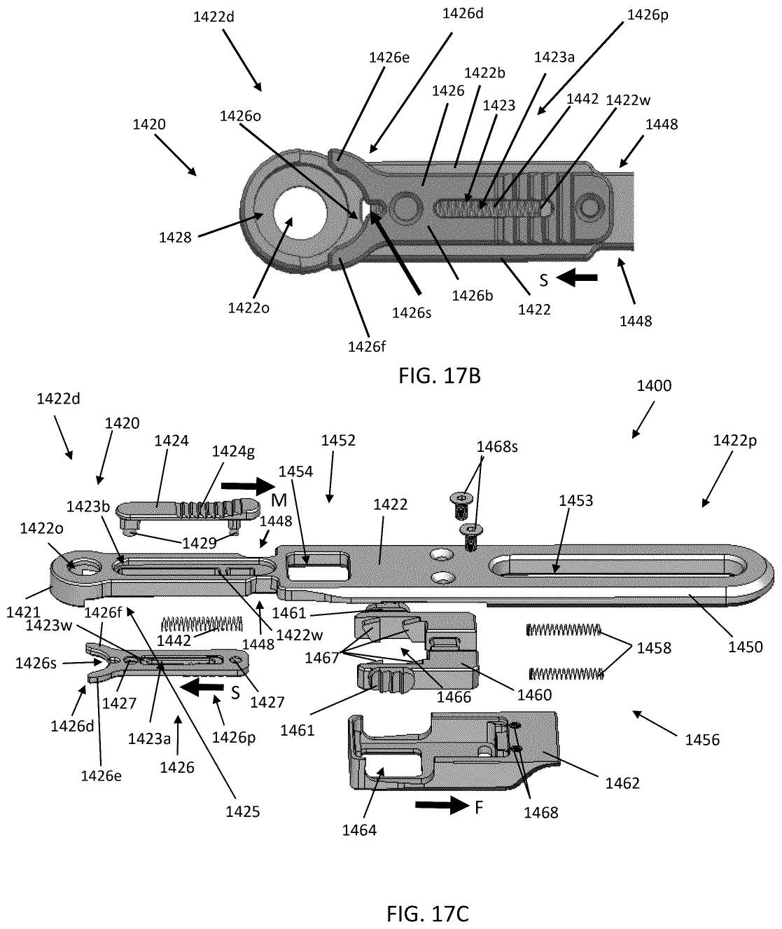

After the humeral guide 300 is positioned on the humerus and the transhumeral bone tunnel is drilled through the drill cannula 350, a number of modular attachments can be coupled to the distal end 310d of the rigid arm 310. To accomplish this, a handle assembly 1400 can be employed. FIG. 15 illustrates the handle assembly 1400 mated to the guide 300, while FIGS. 17A-17C illustrate more detailed views of the handle assembly 1400. FIG. 16 provides for an adapter 390 that can be used to couple a handle assembly (e.g., the assembly 1400) to the distal end 310d of the rigid arm 310.

As shown, an adapter 390 can be disposed on the distal end 310d of the arm 310 of the humeral guide 300. The adapter 390 helps provide a feature to which the handle assembly 1400 can be mated, and also provides stability for use of the handle assembly 1400 with respect to the guide 300, thus helping to maintain desired alignments like the alignment of the drill bit 380 or drive shaft 385 with respect to the humeral resection surface 1015. As shown, the drive shaft 385 can be associated with a modified trinkle connection 386, which can be connected to a power source and used to rotate and/or advance the drive shaft 385 during any portion of a procedure.

With reference to FIG. 16, the adapter 390 can be generally rectangular in shape, with a flared portion 392 at a distal end 390d of the adapter, the flared portion 392 forming a wider base than a more proximal portion 390p of a body 393 of the adapter 390, thus providing for additional support of a handle assembly (e.g., the handle assembly 1400) with respect to a humeral guide (e.g., the guide 300). A handle portion 395, also located as part of the proximal portion 390p, can provide a feature that allows a user to easily grasp and move the adapter 390 away from a handle assembly (e.g., the handle assembly 1400). Further, an illustrated bottom 395b of the handle portion 395 can be used for purposes of alignment, as described below. The adapter 390 can generally be spring-loaded and biased towards the handle assembly 1400, to allow the handle assembly 1400 to be selectively disengaged from the arm 310. When released, the adapter 390 can generally advance distally, towards a terminal end of the distal end 310d of the arm 310.

An opening 391 can be formed through an entirety of the body 393, thus allowing the distal end 310d of the arm 310 to pass therethrough, allowing the adapter 390 to be mounted on the same for use with the same. The distal end 390d of the adapter 390 can also include a mount 394 that extends distally from the flared portion 392, the mount 394 being configured to receive the handle assembly 1400. As shown, a guide-receiving opening 1454 formed in a receiving portion 1452 of the handle assembly 1400 can be sized and shaped to be fitted onto the mount 394, with the mount 394 likewise being complimentary in configuration to receive the receiving portion 1452, and more particularly the guide-receiving opening 1454, of the handle assembly 1400. The mount 394 can be complementary in shape to a portion of the handle assembly that is designed to receive it when mounting the handle assembly (e.g., the handle assembly 1400) to the guide (e.g., the guide 300). In the illustrated embodiment, four engaging protrusions 397 are formed as part of the mount 394, the engaging protrusions 397 being complementary to engaging protrusions 1467 formed on a slider 1460 of the handle assembly (see FIG. 17C).

The adapter 390 can be mated to the distal end 310d of the arm 310 in a variety of ways, but in the illustrated embodiment, the adapter 390 can be selectively and slidably mounted to the arm 310 to allow the adapter to be quickly and easily selectively moved between a locked position or configuration in which the adapter 390 can receive the handle assembly 1400 and an unlocked position or configuration in which the adapter 390 can be moved proximally along the arm 310 in the direction P, away from a location where the handle assembly 1400 mounts, to assist in allowing the handle assembly, or another instrument, to be later engaged by the adapter 390 for use with the guide 300, for example by advancing the adapter 390 in a direction Q, which is opposite to the direction P. A gripping feature, like the illustrated ribbed portions formed on the flared portion 392, can provide for easy grasping by a user to pull the adapter 390 proximally and slide it along the arm 310 in the direction P.

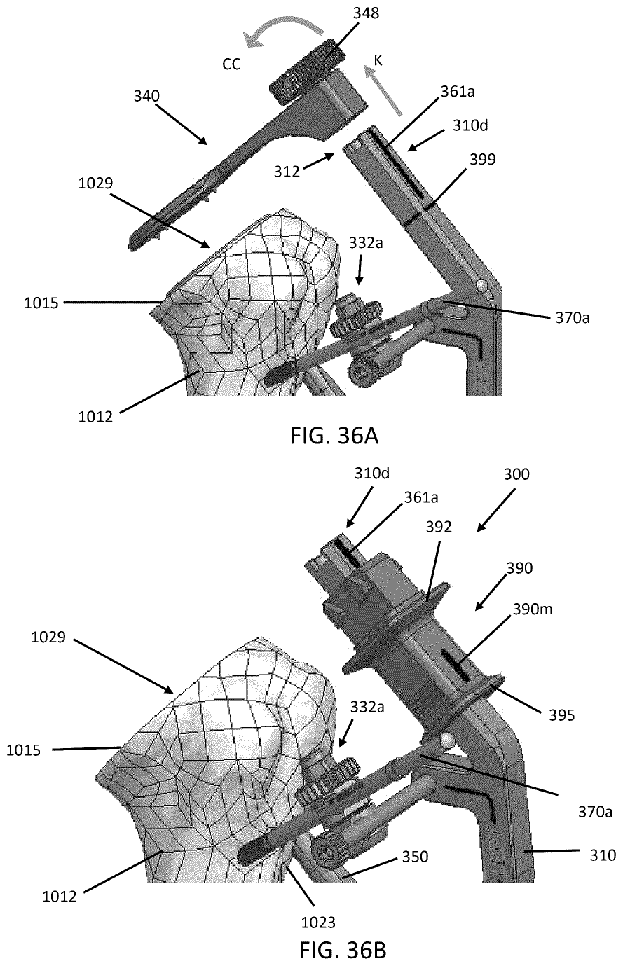

As shown in FIG. 16, the adapter 390 can also have formed thereon an alignment line 390m formed on the body 393. Similar to other portions of the guide 300, and related components, that include various alignment and/or demarcation lines, the line 390m can be used to assist and/or verify proper alignment between the adapter 390 and the arm 310. This alignment between the line 390m of the adapter 390 and the line 361a of the arm 310 is illustrated at least in FIGS. 15 and 36B.

Before describing how the adapter 390 engages with the handle assembly 1400, it can be helpful to discuss some features of the handle assembly 1400. FIGS. 17A-17C illustrate the handle assembly 1400.

The handle assembly 1400 includes an elongate arm 1422 having a proximal portion 1422p and a distal portion 1422d. The proximal portion 1422p includes the handle or gripping portion 1450 as well as the receiving portion 1452, which is the portion configured to mate to a distal end (e.g., the distal end 310d) of an arm (e.g., the arm 310) of a universal guide (e.g., the guide 300). The distal portion 1422d includes features configured to work with an attachment portion 1420 to selectively capture and release attachments or tools. The distal portion 1422d of the arm 1422 defines a plane PL″. The plane PL″ can extend through a body 1422b of the distal portion 1422d of the arm 1422d, though the plane PL″ defined by the body 1422b can just as easily extend through another similar surface of the attachment portion 1420.

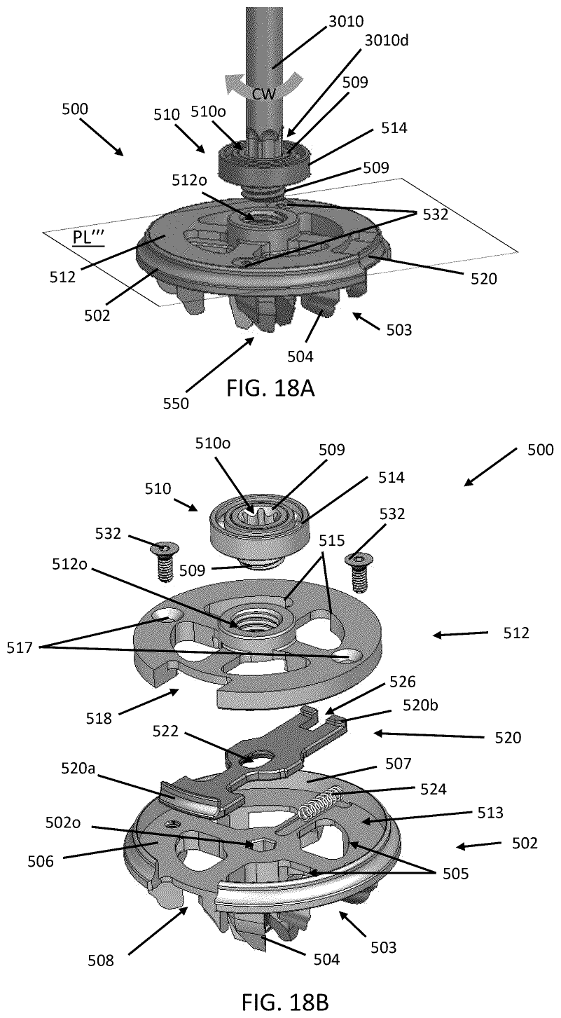

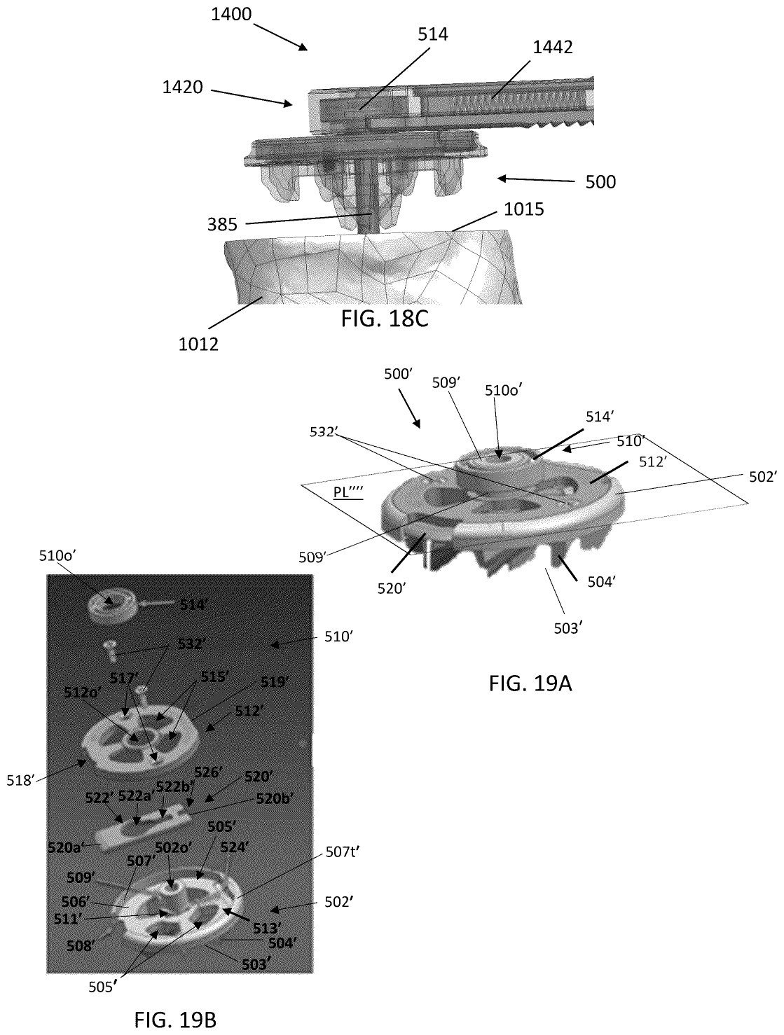

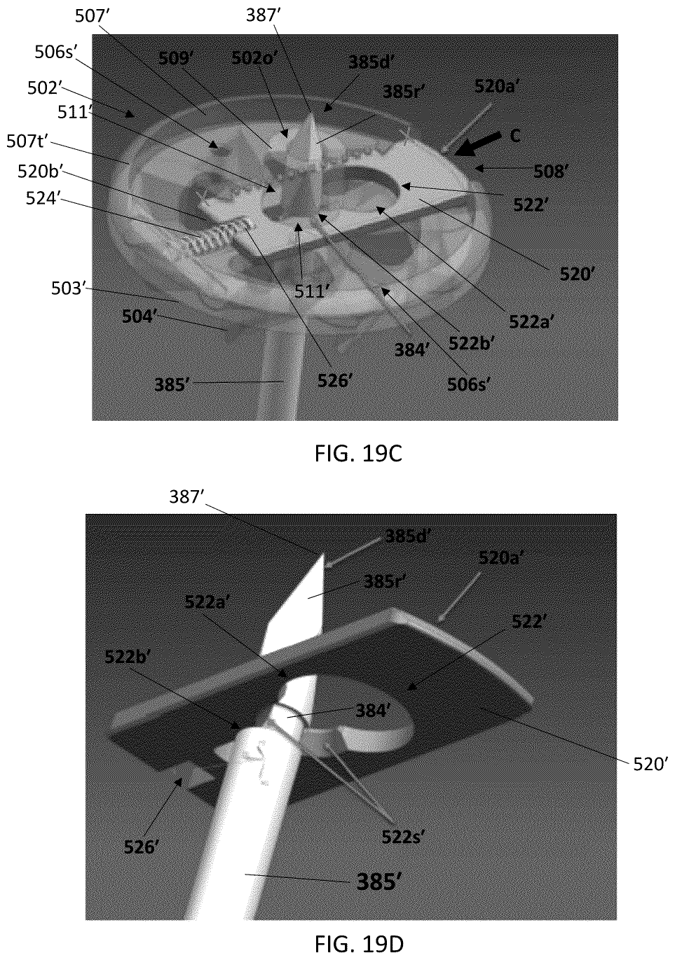

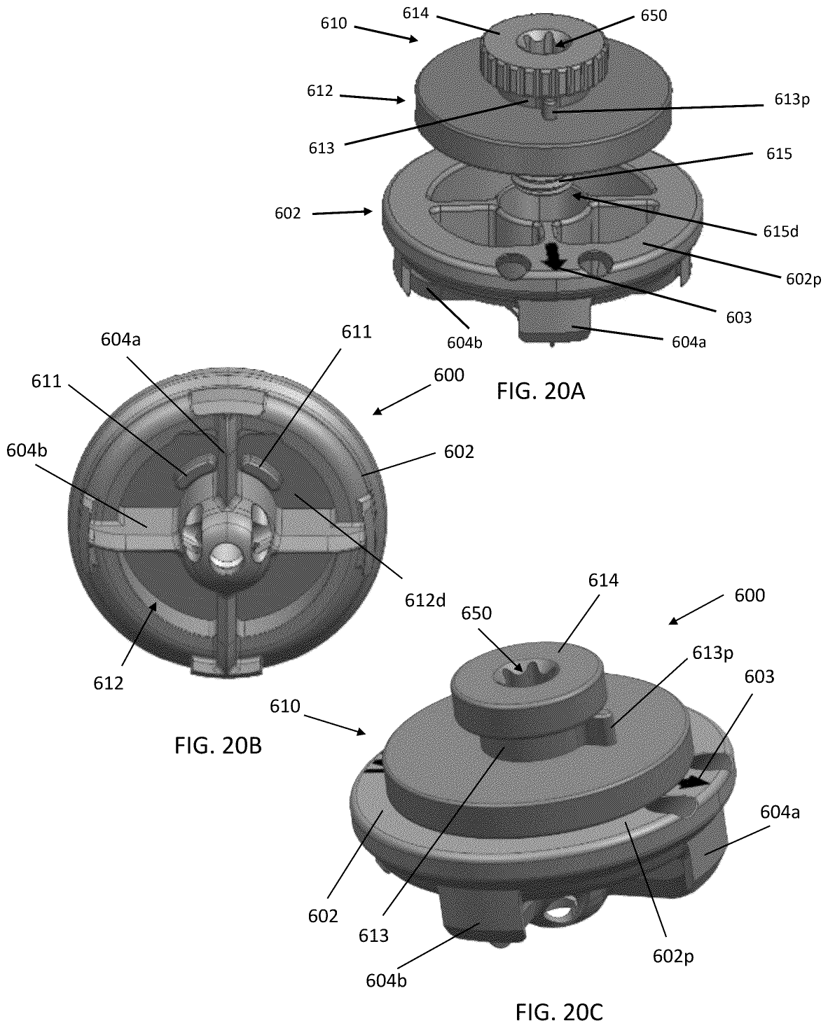

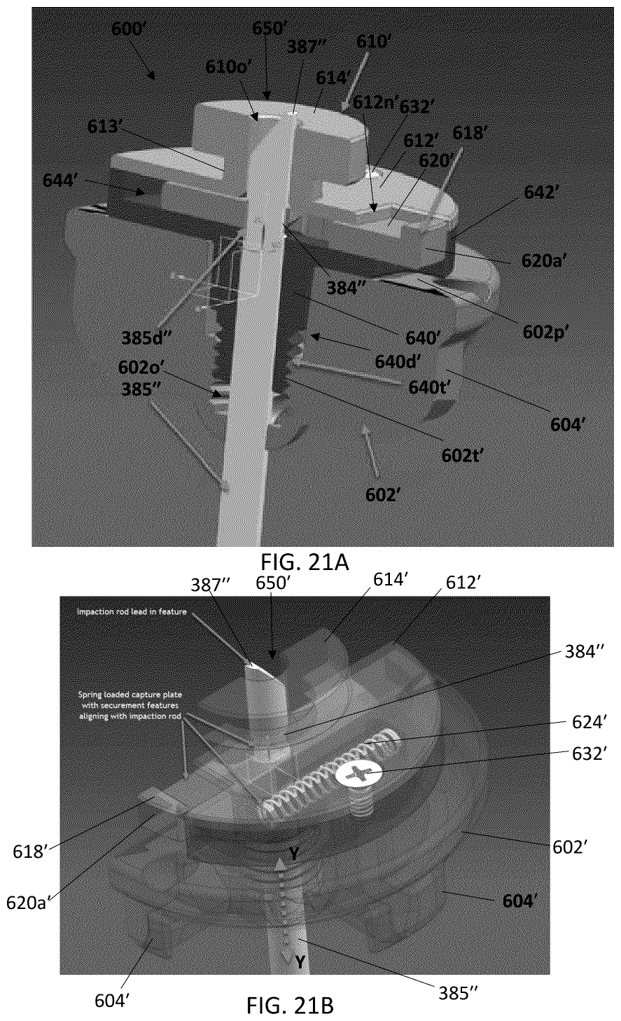

The handle assembly 1400 can include the receiving portion 1452 that is configured to mate to the distal end 310d of the arm 310 of the guide 300. In the illustrated embodiment, the receiving portion 1452 includes an outer housing or cover, and the guide-receiving opening 1454 defined by a portion of the outer housing and sized and shaped to allow at least a portion of the mount 394 to be passed into it for mating the mount 394 with the receiving portion 1452. When the mount 394 is disposed within the guide-receiving opening 1454, the handle assembly 1400 can be mated to the mount 394 by way of a selectively lockable guide attachment mechanism 1456. In the illustrated embodiment the selectively lockable guide attachment mechanism 1456 includes a pair of springs 1458, a slider 1460, and a spring-receiving receptacle 1462. The springs 1458 can be disposed within the receptacle 1462 and biased distally, i.e., away from the gripping portion 1450 and towards a location where tools, such as a reamer attachment 500, 500′ (see, e.g., FIGS. 18A-18C and 19A-19D) or a blazer attachment 600, 600′ (see, e.g., FIGS. 20A-20C and 21A-21B, and sometimes referred to as a blazing attachment or broaching attachment), are selectively coupled to the handle assembly 1400. The bias can place the guide attachment mechanism 1456 into an engagement position or configuration such that the springs 1458 are biased to engage the mount 394 when the mount 394 is disposed in the guide-receiving opening 1454. The attachments like the reamer attachments 500, 500′ and the blazer attachments 600, 600′ can more generally be referred to as tool attachments, with the term tool attachment being encompassing of any tool that can be operated in conjunction with the present disclosures. Other terms that may be used to reference all tool attachments include humeral bone preparation (or prep) instruments or tools or bone-treating tools.

The receiving portion 1452 of the handle assembly 1400 can also include a slider 1460 and a spring-receiving receptacle 1462. As discussed in part above, these slider 1460 and spring-receiving receptacle 1462, and their associated components, work with the proximal portion 1422p of the arm 1422 and an adapter (e.g., adapter 390) to selectively couple and decouple the handle assembly 1400 from a guide (e.g., the guide 300). As shown, the slider 1460 and spring-receiving receptacle 1462 are complimentary to each other, forming chamber 1469 in which springs 1458, as shown two springs, can be disposed. The slider 1460 can be designed to slide relative to the receptacle 1462, in a direction F and in a direction opposite to the direction F. The springs 1458 provide a biasing force in the opposite direction of the illustrated direction F to cause the slider 1460 to be biased towards a distal portion 1422d of the arm 1422. The slider 1460 can include opposed gripping blocks 1461, each block having grooves formed therein allowing the slider 1460 to be grasped and slid in the direction F, causing the slider 1460 to act against the biasing force of the springs 1458 and slide the slider 1460 relative to the spring-receiving receptacle 1462. The gripping blocks 1461 of the slider 1460 can move together to move the two springs 1458 simultaneously. It is possible that the gripping blocks 1461 of the slider 1460 can be operated independently to individually move the springs 1458. The slider 1460 and the spring-receiving receptacle 1462 include openings 1466, 1464, respectively, formed therein for receiving a distal portion (e.g. the distal portion 390d) of an adapter (e.g., the adapter 390). The spring-receiving-receptacle 1462 also includes a plurality of holes 1468 for receiving screws 1468s that can be used to attach the spring-receiving receptacle 1462 to the proximal portion 1422p of the arm 1422.

The slider 1460 can include features for engaging the distal portion of an adapter to assist in securing the handle assembly 1400 to a guide (e.g., the guide 300). More particularly, as shown, four engaging protrusions 1467—two each on opposed inner-facing surfaces of the slider 1460—are configured to engage with complimentary engaging protrusions (see protrusions 397 from FIG. 16) formed on a distal portion of an adapter (see adapter 390 from FIG. 16). In use, the slider 1460 can slid in the direction F to allow the mount 394 to be passed into and through the opening 1464, the opening 1466, and the opening 1454. Once the mount 394 is properly situated, the slider 1460 can be released, biased back in the direction opposite to the direction F. The engaging protrusions 1467 can engage with the engaging protrusions 397 to help secure the handle assembly 1400 to the adapter 390, and thus a guide (e.g., the guide 300). A person skilled in the art, in view of the present disclosures, will understand other configurations and features that can be used for a similar purpose as the selectively lockable guide attachment mechanism 1456 to allow for selective mounting of the handle assembly 1400 to the guide 300. Further, it is features of the receiving portion 1452, such as the receiving opening 1454 and related components and the slider 1460, as well as other components of the handle assembly 1400 provided for herein that are easily accessible from either side of the handle assembly 1400, that help make the handle assembly 1400 universal such that it can be operated from either side of the body with either hand.

The handle 1450, also referred to as a gripping portion, can have various contours and features to make gripping by either a right-hand or a left-hand, from either side of the assembly 1400, easy and comfortable, and without having to change grips and/or positions during the surgical procedure. In the illustrated embodiment, the proximal portion 1422p includes an elongate slot 1453 formed therein. The slot 1453 can be used, for example, to have a gripping feature coupled to it. The gripping portion 1450 can include a comfortable gripping material (e.g., rubber) that can be attached to a guide (not shown) that helps form the gripping portion 1450 of the handle assembly 1400. A person skilled in the art will understand how a comfortable gripping material (e.g., rubber) or other gripping features can be mated to the arm 1422 by way of the slot 1453. Further, a person skilled in the art will appreciate other manners in which the slot 1453 can be used, not necessarily involving the use of a gripping feature. That is, the arm 1422 as shown can be the handle held by a surgeon in conjunction with performing a surgical procedure. The receiving portion 1452 also includes a receiving opening 1454 formed in the proximal portion 1422p of the arm 1422. As described herein, this receiving opening 1454 can be configured to receive a distal end of the adapter 390.

A distal portion 1400d of the handle assembly 1400 can include an attachment portion 1420 configured to selectively receive various attachments or tools that can be coupled to the handle assembly 1400 for use in a surgical procedure. In at least some embodiments, the attachment portion 1420 can be selectively mated to the gripping portion 1450 such that differently configured gripping portions and differently configured attachment portions can be utilized in combination. A person skilled in the art will appreciate how various removable and replaceable coupling configurations can be provided for to enable such mixing and matching of gripping and attachment portions. As illustrated, the attachment feature includes, among other components, a recess 1425 formed in the distal end 1422d of the arm, a capture plate 1426, and a latch 1424. The capture plate 1426 and latch 1424 are on opposed sides of the arm 1420 and are configured to move together to capture a mount of an attachment within the recess 1425.

While various attachments or tools can be utilized with the attachment portion 1420, in the embodiment illustrated in FIG. 15, the attachment is a reamer attachment 500 that is coupled to the handle assembly 1400. A person skilled in the art will appreciate other attachments and/or tools that are disclosed herein (e.g., any of the other reamer or blazer attachments 500′, 600, 600′) and/or known to those skilled in the art can be also be used as attachments or tools in conjunction with the humeral guides (e.g., the guide 300) and handle assemblies (e.g., the handle assembly 1400) of the present disclosures.

A wall 1421 can extend down from the outer edge of the distal portion 1442d of the arm 1422 defining a substantially circular recess 1425 configured to receive an attachment having a mount (e.g., a mount 510 as illustrated and described at least with respect to FIGS. 18A-18B). The recess 1425 can be sized and shaped to be larger than the mount such that the mount can freely move in and out of the recess 1425 when the capture plate 1426 is moved proximally with respect to the recess 1425. In the illustrated embodiment the recess has a central opening 14220 formed therein that can be aligned with the cannula-receiving opening 322 of the guide 300, and thus the drill cannula 350 disposed therein and the longitudinal axis LC, similar to the opening 343a of the humeral sizer attachment 340. This can create a singular path through which the drill bit 380 can pass, the travel path being colinear with the longitudinal axis LC. Further, the plane PL″ defined by the handle assembly 1400 can be orthogonal, or substantially orthogonal, to the longitudinal axis LC.

The capture plate 1426 includes a proximal portion 1426p that can be generally elongate and a distal portion 1426d that can be more circular. The circular distal portion 1426d can have a curvature similar to the curvature of the recess 1425 formed in the distal end 1422d of the arm. The curvature can be compatible against the curvature of a mount of an attachment (e.g., a mount 510 as illustrated and described at least with respect to FIGS. 18A-18B) such that the distal end 1442d can contact and secure the mount against the wall 1421 that defines recess 1425. The attachment portion 1420 can further include a spring 1442 configured to bias the capture plate 1426 in a distal direction S, causing the distal portion 1426d of the capture plate to close off a portion of a recess 1425.

The capture plate 1426 is disposed proximate to the distal portion 1422d of the arm 1422 and is configured to move (e.g., slide) relative to the same to selectively engage and disengage a tool or attachment (e.g., the reamer attachment 500). More particularly, the capture plate 1426 is disposed below and planar to the distal portion 1422 and configured to slide in the direction S such that a distal portion 1426d of the capture plate 1426 closes off a portion of the recess 1425 formed in the distal portion 1422d of the arm 1422. In the illustrated embodiment the capture plate 1426 includes a slot 1423a formed in its body 1426b for receiving a spring 1442 such that a distal portion of the spring 1422 engages a wall 1423w. The slot 1423a can extend a similar length as slot 1423b of the arm 1422 and align with slot 1423b such that the distal portion of the spring 1442 engages a distal wall 1423w of the slot 1423a and the proximal portion of the spring 1442 engages a proximal wall 1422w of the slot 1423b to bias the capture plate 1426 in a distal direction S to close off a portion of the recess 1425.

A distal portion 1426d of the capture plate 1426 can include opposed arms 1426c, 1426f configured for engagement with a tool or tool attachment. An alignment slot 1426s can be formed between the two arms 1426e, 1426f for purpose of receiving a complimentary alignment feature formed in a tool or tool attachment (e.g., the protrusion 613 of the blazer attachment 600, as shown at least at FIGS. 20A and 20C).

The capture plate 1426 can be moved by way of a latch 1424 coupled to the capture plate 1426 to open the recess 1425. As shown, the latch 1424 is coupled to the capture plate 1426 through a slot 1423b formed in the distal portion 1422d of the arm 1422. In the illustrated embodiment, the latch 1424 includes two posts 1429 extending from a bottom surface of the latch 1424, through the slot 1423b, and into complementary openings 1427 formed in the capture plate 1426. In other embodiments, the latch 1424 can be mated to the capture plate 1426 by weldment or other means known in the art such that they travel together to capture and release tools from the attachment portion 1420. A portion of the latch 1424 can extend above the arm 1422 and can include gripping features, shown as grooves 1424g that can help a user grasp the latch 1424 to assist in moving the same. In some instances, the latch 1424 and the capture plate 1426 can be considered a unitary capture component, and thus in at least some instances, reference to a latch can include both the latch and the capture plate, and likewise, reference to a capture plate can include both the capture plate and the latch. As designed, movement of one causes the other to move, thus allowing a user to use the latch 1424 to act against a bias experienced by the capture plate 1426.

In use, a user can grasp the grooves 1424g and pull in a direction M to counter the biasing force in the direction S provided by the spring 1442. This causes the capture plate 1426 to slide proximally, sliding relative to the distal portion 1422d of the arm 1422. As a result, the more distal portion 1426d of the opening capture plate 1426 moves proximally, creating a larger radius of the recess 1425 to receive an attachment or tool. The roller bearing (or other equivalent mount) can be disposed within the recess 1425. Once the roller bearing is at the desired location with respect to the capture plate 1426 and arm 1422, the force in the direction M can be removed, allowing the capture plate 1426 to slide distally in the direction S created by the biasing force of the spring 1442. This, in turn, causes the distal portion 1426d of the capture plate 1426 to move towards a terminal end of the distal portion 1422d of the arm 1422 thereby reducing the radius of the recess 1425 and trapping the roller bearing (or other equivalent mount) between the distal portion 1426d of the capture plate 1426, including the arms 1426e, 1426f, and the wall 1421 of the recess 1425, securing the attachment to the handle assembly 1400 for subsequent operation of the same. The same action for loading the attachment can be performed to unload the attachment, with pulling the direction M allowing the attachment to be disengaged by the distal portion 1426d of the capture plate 1426 such that the attachment can be moved out of the recess 1425. Optionally, another attachment or tool can be coupled to the handle assembly 1400 for further surgical actions to be performed.

### Attachments for Handle Assembly(ies)

Modular attachments like reamer attachment 500 and blazer attachment 600 can have a variety of configurations, non-limiting versions of which are disclosed herein as reamer attachment 500′ and blazer attachment 600′. Reference to one version of such an attachment is also applicable to the other version(s) unless explicitly stated otherwise or otherwise appreciable by those skilled in the art in view of the descriptions and figures provided herein. In the illustrated embodiments, the attachments 500, 600 include a tool 502, 602 and a mount 510, 610. The tool 502, 602 can include features that are configured to perform a particular function a person skilled in the art will appreciate is the typical function for such a tool, and in some instances can be referred to as a bone-engaging instrument, bone preparation instrument, or humeral bone preparation instrument, among other names. Accordingly, for the reamer attachment 500, the tool 502 is a reamer having a plurality of reaming teeth 504 for reaming bone. Likewise, for the blazer attachment 600, the tool 602 is a blazer (sometimes referred to as a blazing tool or a broaching tool) having a plurality of blazing fins or arms 604 (sometimes referred to as broaching fins or broaching arms) for broaching bone.

FIGS. 18A and 18B illustrate an embodiment of a reamer attachment, the reamer attachment 500, shown in FIG. 15. The tool 502 is a reamer configured to carve a geometry into the humeral resection surface 1015 to prepare it for receiving a prosthesis. Reaming a bone requires a sufficient amount of pressure applied to the bone surface by the reamer 502 to carve the proper geometry. In traditional methods that were performed using known tools, the narrow rotator interval maintained by the surrounding soft tissue did not provide adequate space to ream the bone surface using a force applied down onto the bone surface. Accordingly, the present disclosure provides the required force to operate the tools by way of a retraction force provided to the reamer attachment 500 by way of the drive shaft 385 passed through the drill cannula 350 and transhumeral bone tunnel.

The reamer attachment 500 includes the tool 502, a capture plate or quick-release latch or button 520, and a mount 510 that includes a roller bearing 514, the mount 510 also being referred to as a reamer bearing, adapter, or a reamer adapter. The mount 510 can be separate from the tool 502 as the mount 510 can be intended for a single use while the tool 502 can be cleaned and reused in subsequent procedures. The reamer attachment 500 can be generally circular in shape with a substantially flat profile, though other shapes and configurations are possible. Similar to the humeral sizer attachment 340, the reamer attachment 500 can be manufactured in various diameters to fit against varying humeral resection surface diameters. The flat profile allows the reamer attachment 500 to enter the joint space through the narrow rotator interval above the subscapularis. The reamer attachment 500 further includes a central opening 550 that is configured to accept and secure the drive shaft 385 within it. The central opening 550 can be defined by openings formed in the various components of the reamer attachment 500, such openings and components being described in greater detail below with reference to FIG. 18B. The tool 502 can define the plane PL″, akin to the planes PL and PL′.

The tool 502 can include a cutting surface 503 with blades or teeth 504 formed thereon, and a receiving surface 506 that opposed to the cutting surface 503 such that it is an opposite side of the tool 502. A plurality of relief holes or openings 505 can be formed between the receiving surface 506 and the cutting surface 503, the relief holes 505 providing paths for cut tissue, fluids, debris, and other materials to pass through during operation of the reamer attachment 500. As shown, there are four relief holes 505, spaced radially around a central opening 5020, although other configurations, shapes, and number of relief holes can be provided. A lip 507 can be formed on an outer edge or perimeter of the tool 502, with a plane that extends across a top terminal end 507t of the lip 507 and the receiving surface 506 defining a chamber 513 in which components of the reamer attachment 500 can be disposed. An access port 508 can be formed in the lip 507 to provide access to the chamber 513. The central opening 5020 extends from the receiving surface 506 to the cutting surface 503.

The capture plate or release button 520 includes an opening 522. The opening 522 can have a first portion 522a that is larger to allow the drive shaft 385 to pass through it and a second portion 522b narrower than the first portion 522a such that the second portion can engage and capture a drive shaft 385. An internal surface 522s of the opening 522 can be ramped. A terminal edge 520a of the capture plate 520 can align with the access port 508 and a spring 524 can be located in the chamber 513 to bias the capture plate 520 towards the access port 508 when the capture plate 520 is in a captured or locked position or configuration, a position that is described in greater detail with respect to FIG. 18C (see also, description related to FIGS. 19C and 19D for similar locking capabilities). At least the terminal edge 520a can be polished to aid in being able to more easily locate it at the surgical site. The capture position can be the default or resting position or configuration for the capture plate 520. The spring 524 can sit within a notch 526 formed on an opposed terminal edge 520b of the capture plate 520. The spring 524 presses against the lip 507 and biases the capture plate 520 towards the access port 508.

The distal disc or plate 512 includes a central opening 5120. The central opening 5120 can be large enough to allow the shaft 509 of the roller bearing mount 510 to pass therethrough. In the illustrated embodiment, the shaft 509 is threaded, with the threads being complementary to threads formed in an opening 5020 of the tool 502. A plurality of relief holes or openings 515 can be formed through the disc 512, the relief holes 515 being configured in a manner similar to the relief holes 505 formed in the tool 502, thus providing paths for cut tissue, fluids, debris, and other materials to pass through during operation of the reamer attachment 500. Screw-receiving openings 517 can also be formed through the disc 512. As shown, the screw-receiving openings 517 can be configured to receive screw 532, which can be used to mate the disc 512 to the tool 502. Complementary screw-receiving openings 506s can be formed in the receiving surface 506 of the tool 502. Further, an access port 518 can be formed at an edge of the disc 512, the access port 518 being configured to be aligned with the access port 508 of the tool 502, thereby allowing the terminal edge 520a of the capture plate 520 to be contacted to move it from its biased capture position to an unlocked position or configuration. As shown, an edge of the disc 512 can be complementary in shape to a radially-inward facing surface of the lip 507 of the tool 502 to allow the disc 512 to be press fit or otherwise coupled together.

The proximal disc 514 of the mount 510 is a roller bearing. As shown, the roller bearing 514 has an opening 5100 formed therein, the opening 5100 being sized to allow the roller bearing to be disposed around the roller bearing mount 509, as shown in FIG. 18A (see also, FIG. 19A for a similar configuration). The opening 5100 can also be configured to receive a shaft 3010, as described in further detail below. The shaft can be, for example, a T30 driver. In the illustrated embodiment, both the opening 5100 and a distal end 3010d of the shaft 3010 are six-sided, creating a hex-key engagement, although other configurations, including but not limited to other key configurations, can be used. With respect to the handle assembly 1400, the roller bearing 514 can be configured to mate with the distal portion 1426d of the capture plate 1426, including the arms 1426e, 1426f, and being locked in the chamber 1428. Further, the exposed portion of the roller bearing mount 509 can help provide a location at which the reamer attachment 500 can be mated to the distal portion 1426d of the capture plate 1426 when the capture plate 1426 is in its biased, locked position with no force being applied to the latch 1424 to counteract the bias force applied to the capture plate 1426. By using the roller bearing 514, the associated tool 502, can spin freely while it is held by the handle assembly 1400. The roller bearing 514 provides smooth rotary and/or planar motion with no frictional seizing. A person skilled in the art, in view of the present disclosures, will understand how the roller bearing 514 can be modified for engagement with the handle assembly 1400, for instance by including a protrusion as part of the roller bearing, 514, or another component (see, e.g., a protrusion 613p associated with an intermediate disc 613 in FIG. 20A of the blazer attachment 600), that can engage with the walls of the arms 1426e, 1426f that define the alignment slot 1426s.

The mount 510 can be a separate component from the tool 502 allowing one component to be disposed of while the other can be reused. For example, in some instances, the mount 510 can be disposable, allowing the tool 502 to be reused.

In use, the spring 524 can push the capture plate 520 towards an access port 508 formed in the tool 502, to cause the capture plate 520 to engage the drive shaft 385 disposed within the reamer attachment 500. Further, as described in greater detail elsewhere herein, the mount 510, and thus the reamer attachment 500, is held in place by the attachment portion 1420, which is biased into this locked configuration by the spring 1442, as shown in FIG. 18C. Once the capture plate 520 secures the drive shaft 385 with respect to the tool 502, the drive shaft 385 can be used to operate the tool 502. Pushing on a terminal edge 520a of the capture plate 520 can allow the capture plate 520 to release the drive shaft 385 from the reamer attachment 500.

FIGS. 19A-19D illustrate another embodiment of a reamer attachment 500′. The reamer attachment 500′ is similar to other reamer attachments provided for herein, such as the reamer attachment 500, and thus a full description of the same is not necessary, nor is the identification of each illustrated feature of the attachment 500′. As shown, from a proximal portion of the exploded view to a distal portion of the exploded view, the reamer attachment 500′ can include a mount 510′ that includes a roller bearing 514′ and a distal disc or plate 512′, a capture plate 520′, and a tool 502′, as shown a reamer. Screws 532′ can be used to secure the mount 510′, by way of the distal disc 512′, to the tool 502′, and the capture plate 520′ can be biased in manners provided for elsewhere herein by a spring 524′ that engages the tool 502′ and the capture plate 520′. A plane PL″″ can be defined by the tool 502′, akin to the planes PL, PL′, and PL′″. The plane PL″″ can extend through a primary surface of the tool 502′, though the plane defined by the tool 502′ can just as easily extend through another similar surface of the tool the tool 502′.

A mount 509′ can extend upward from the center of the tool 502′ with the central opening 5020′ of the tool 502′ extending therethrough, from the roller bearing mount 509′ to a distal-most end of the cutting surface 503′. The roller bearing mount 509′ can be configured to receive the roller bearing 514′ around it. For example, the roller bearing 514′ can be pressed on and welded to the roller bearing mount 509′ in a manner that spaces the roller bearing 514′ a distance apart from the distal disc 512′. As shown, this space can form a channel 511′ between a distal end of the roller bearing mount 509′, defined as the end of the roller bearing mount 509′ that is closest to the cutting surface 503 and exposed by the space between the roller bearing 514′ and the distal disc 512′, and the receiving surface 506′. The channel 511′ can be configured to receive the capture plate 520′ to enable the capture plate 520′ to engage a guide pin when it passes through the roller bearing mount 509′ via the central opening 5020′ of the tool 502′.

A flat surface 519′ can be formed on the edge of the disc 512′ that is opposed to the access port 518′, the flat surface 519′ being complementary to a flat portion of the radially-inward facing surface of the lip 507′.

The capture plate 520′ illustrated in FIG. 19B includes an opening 522′ having a plurality of diameters, the opening being sized to pass over the roller bearing mount 509′ such that the capture plate 520′ sits on the receiving surface 506′, within the channel 511′. A first portion 522a′ of the opening 522′ can be larger to allow the drive shaft 385 to pass through it, while the second portion 522b′ can be smaller such that it is configured to engage and capture the drive shaft 385, as shown in FIGS. 19C and 19D. An internal surface 522s′ of the opening 522′ that defines a transition from the first portion 522a′ to the second portion 522b′ can be ramped as shown best in FIG. 19D. A terminal edge 520a′ of the capture plate 520′ can align with the access port 508′ and a spring 524′ can be located in the chamber 513′ to bias the capture plate 520′ towards the access port 508′ when the capture plate 520′ is in a captured or locked position or configuration, a position that is described in greater detail with respect to FIG. 19D. The capture position can be the default or resting position or configuration for the capture plate 520′. The spring 524′ can sit within a notch 526′ formed on an opposed terminal edge 520b′ of the capture plate 520′. The spring presses against the lip 507′ and biases the capture plate 520′ towards the access port 508.

FIGS. 19C and 19D illustrate the reamer attachment 500′ engaging a drive shaft 385′. It is the engagement between the drive shaft 385′ and the reamer attachment 500′ that can set the position at which the reamer 502′ will be used to ream bone. The drive shaft 385′ is similar to the drive shaft 385, and includes a distal end 385d′ configured to operate tools associated with the modular attachments coupled to a handle assembly, like the reamer attachment 500′ when it is attached to the handle assembly 1400, and a pointed distal tip 387′ disposed at the distal end 385d′. In the illustrated embodiment, the drive shaft 385′ includes a groove 384′ formed proximal of the distal tip 387′ of the distal end 385d′, around an outer surface or perimeter of the shaft 385′. As discussed above, the first portion 522a′ of the opening 522′ has a diameter larger than a diameter of the roller bearing mount 509′ to allow the plate 520′ to pass over the roller bearing mount 509′, for example during assembly, and/or when the capture plate is in an unlocked position or configuration that allows the drive shaft 385′ to move longitudinally through the roller bearing mount 509′ and the opening 522′. Meanwhile, the second portion 522b′ of the opening 522′ has a diameter that is larger than a diameter of the drive shaft 385′ at the groove 384′ but smaller than a diameter of a shaft of the drive shaft 385′ immediately above and below the groove 384′, i.e., the diameter of the drive shaft 385′ for a majority of its length.

As noted above, the capture plate 520′ can be biased in a direction towards the access port 508′ by the spring 524′, causing the default or resting position or configuration of the capture plate 520′ to be the captured or locked position. When the capture plate 520′ is in the default or resting position, the second portion 522b′ of the opening can be substantially aligned with the opening 5020′ of the tool 502′. As a result, the capture plate 520′ can prevent or block certain sized instruments, such as the drive shaft 385′, from passing through the central opening 502o′ of the tool 502′, and thus through the central opening 550′ of the reamer attachment 500′ because a size of the diameter of the second portion 522b′ of the opening 522′ is too small to allow for movement therethrough. The capture plate 520′ can be moved to an unlocked or releasing position or configuration by causing the capture plate 520′ to slide or otherwise move in a direction towards the portion of the lip 507′ against which the spring 524′ is disposed to counteract the biasing force of the spring 524′. This movement can be along the X-X axis illustrated. As this movement occurs, the first portion 522a′ of the opening 522′ can move towards the location at which the second portion 522b′ was disposed in the resting position. As the first portion 522a′ is aligned, or at least becomes aligned sufficiently to provide a diameter large enough to no longer restrict movement of instruments through the central opening 5020′ of the tool 502′, the capture plate 520′ can be considered to be in the unlocked or releasing position or configuration. The capture plate 520′ can be subsequently returned to the resting position to secure an instrument, like the drive shaft 385′, at a desired location, the capture plate 520′ trapping the drive shaft 385′ at that location.

The capture plate 520 can be moved from the resting position to the releasing position in a variety of ways. For example, in at least some embodiments, when the distal end 385d′ of the drive shaft 385′ passes through the central opening 5020 of the tool 502, the larger diameter of the drive shaft 385′ can press against the inner surface 522s that define a portion of the opening 522, forcing the capture plate 520 to travel along the X-X axis towards the portion of the lip 507 against which the spring 524 is disposed. This can place the capture plate 520 in the releasing position. The distal end 385d′ of the guide pin 385′ can be shaped into the pointed tip 387′ with ramped surfaces 385r′ extending from a main body of the shaft 385′ and to the pointed tip 387′. In alternative embodiments, the pointed tip 387′ can be replaced by a bullet or parabolic-shaped tip, similar to the drive shaft 385 shown in FIG. 12D, which can provide a blunt surface to minimize damage to surrounding tissue and the like during use while still allowing for the same functionality of the drive shaft 385′. The ramped surfaces 385r′ can assist with counteracting the bias of the spring 524, moving the capture plate 520 to the releasing position by contacting and sliding against the ramped inner surface 522s that define at least a portion of the opening 522. This, in turn, can cause the capture plate 520 to slide to the releasing position. When the capture plate 520 is in the releasing position, the drive shaft 385′ can continue to be advanced, for example until the groove 384′ is disposed within the opening 522. When that occurs, the biasing force of the spring 524 may no longer be counteracted, and thus the spring 524 can push the capture plate 520 back towards its resting position. The capture plate 520, however, will not make it all the way back to its original resting position. This is at least because the capture plate 520 can engage the drive shaft 385′, and more specifically the groove 384′, to lock the location of the drive shaft 385′ with respect to the reamer attachment 500, and with respect to the handle assembly and the humeral guide with which the reamer attachment 500 are being used. Engagement of the groove 384′ by the capture plate 520 can provide audible and/or tactile feedback to the user that the drive shaft 385′ is locked at a location with respect to capture plate 520, a handle assembly, and/or a humeral guide.

Once the drive shaft 385′ is locked with respect to the capture plate 520, the guide pin 385′ can be used to operate the tool 502 that is part of the attachment 500. For example, a surgeon can use a drill to power the drive shaft 385′ and rotate the reamer attachment 500 at the necessary speed will pull the drive shaft 385′ back through a bone tunnel and drill cannula 350 in which they are disposed, forcing the reaming teeth 504 against the humeral resection surface 1015 to carve or otherwise cut the correct geometry into the humeral resection surface 1015.

After the reaming is completed, the drive shaft 385′ can be released from the reamer attachment 500 by applying a force in the direction C to the terminal edge 520a of the capture plate 520 that is accessible by way of the access port 508. Application of this force can counteract the biasing force of the spring 524, allowing for movement of the drive shaft 385′ along the longitudinal axis LC. For example, the drive shaft 385′ can be passed out of the reamer attachment 500 and out of a drill cannula in which it can have been disposed. In alternative embodiments, in addition to applying a force in the direction C to the terminal edge 520a of the capture plate 520 to release the drive shaft 385′, application of such a force in the direction C can also be used to selectively move and position the drive shaft 385′ with respect to the opening 5020 of the tool 500 to engage and lock the shaft 385′ with respect to the tool 502 for operation of the same.

Each individual preparation step can be accomplished in this general manner using the required attachments. For example, after reaming is completed, the reamer attachment 500 can be replaced with a blazer attachment 600 as shown in FIGS. 20A-20C, or a blazer attachment 600′ as shown in FIGS. 21A-21B, and similar steps can be performed for operation of the same. Alternative terms for a blazer attachment include a blazing attachment or a broaching attachment. A person skilled in the art will appreciate the blazer attachments 600, 600′ can be used to impact the reamed humeral resection surface 1015 in further preparation for an implant and/or prosthesis.

Turning to the blazer attachment 600 illustrated in FIGS. 20A-20C and as noted above, the blazer attachment 600 includes the blazing tool 602, also referred to as a blazer, a blaze, or a broaching tool, and a mount 610, also referred to as an adapter, a blazer adapter, a blazing adapter, or a broaching adapter. The blazer 602 includes the plurality of blazing or broaching arms or fins 604a, 604b for blazing or broaching bone. The mount 610 can have a similar purpose as the mount 610, and it can include one or more features to allow orientation of the blazing fin 604a on the humeral resection surface 1015. As shown, the mount 610 can include a larger, distal disc 612 configured to mate with the tool 602, a smaller, proximal disc 614 configured to mate with, for example, the distal portion 1422d of the arm 1422, sitting in the attachment portion 1420, and an even smaller, intermediate disc 613 disposed between the distal and proximal discs 612, 614. In at least some embodiments, the proximal disc 614 can be a roller bearing. Disposed on a distal-facing surface 612d of the distal disc 612 can be a plurality of alignment bosses 611, as shown two. As shown in FIG. 20B, the blazing fin 604A can be positioned between the two bosses 611 such that the bosses 611 maintain a location of the blazing fin 604a with respect to the mount 610. As a result, the blazing fin 604a can remain aligned with the rotator interval, thus allowing for easier implant insertion. A protrusion 613p, also referred to as a mating protrusion and/or an alignment protrusion, can be formed on the intermediate disc 613, which can be used for mating and/or alignment purposes related to a handle assembly (e.g., the handle assembly 1400), as described in further detail below. As shown, the protrusion 613p can be aligned with an alignment arrow 603 formed on a proximal-facing surface 602p of the blazer 602. Still further, a threaded shaft or bolt 615 can be part of the mount 610, extending distally below the distal-facing surface 612d of the distal disc 612. The threaded bolt 615 can be part of the distal disc 612, or it can be part of one of the intermediate or proximal discs 613, 614 and can extend distally through the distal disc 612. The threaded bolt 615 allows for threaded engagement being the mount 610 and the blazer 602. More particularly, a tool such as a T30 driver, can be used to rotate the mount 610 with respect to the blazer 602, thus securing a locked engagement between the two.

The blazer attachment 600 can have a central opening 650 formed therein to accept and secure a drive shaft, like the drive shaft 384, within it. The central opening 650 can be defined by openings formed in the various components of the blazer attachment 600. More particularly, each of the tool 602, the distal disc 612, the intermediate disc 613, and the proximal disc 614 can have central openings formed therein such that the central opening 650 extends through an entire depth of the blazer attachment 600. In the illustrated embodiment, a top portion of the central opening 650, the part visible in FIG. 20A, includes a hex-shaped configuration for engagement with a hex-shaped shaft, like the T30 driver, for use in coupling the mount 610 to the blazer 602. Similar to the mount 510, the mount 610 can be separate from the tool 602 as the mount 610 can be intended for a single use while the tool 602 can be cleaned and reused in subsequent procedures.

The blazer attachment 600, as well as the blazer attachment 600′ of FIGS. 21A-21B, can operate similar to the reamer attachment 500. For example, it can be operated by connecting the attachment 600 to a guide pin, like the drive shaft 385″ and/or others provided for herein or otherwise known to those skilled in the art, and impacting the humeral resection by retracting the drive shaft 385″ through the transhumeral bone bore in the lateral direction. Broaching the humeral resection surface can be done free-handed by coupling the blazer attachment 600 to the handle assembly 1400 or the blazer attachment 600 can be coupled to the handle assembly 1400 and used in conjunction with the guide 300 to ensure that broaching is completed on axis perpendicular to the plane of the humeral resection surface.

Turning to the blazer attachment 600′ illustrated in FIGS. 21A-21B, and similar to the modular attachments 500, 500′, 600, the blazer attachment 600′ can include a tool 602′ and a mount 610. Further, as shown in FIG. 21A, a mount adapter 640 can be used to assist in mating the mount 610′ to the tool 602′. A component like the mount adapter 640′ can be used in instances where a tool and mount are not configured in a manner that allows them to mate together directly, such an adapter being configured in a manner that allows it to couple to both the tool and the mount, in turn helping to allow the tool and mount to mate to each other.

As shown, the tool 602′ can include blazing arms or fins 604′ configured to broach bone. A bottom surface of the blazing fins 604′ can be shaped with or otherwise be operated to form in the bone the same geometry as the prosthesis to be implanted. A central opening 6020′ can extend through the tool 602′, from a distal-most surface of the tool 602′, which in at least some instances can be defined by a distal-most surface of the fins 604′, to a proximal-most surface 602p′. The central opening 6020′ can be configured to receive a guide pin, like the drive shaft 385″, therethrough. Further, as shown, the central opening 6020′ can include one or more features configured to engage with the mount adapter 640′. More particularly, the central opening 6020′ includes a threaded portion 602t′ that can be configured to couple to complementary threads 640t′ formed on a distal end 640d′ of the mount adapter 640′. Still further, as shown, the central opening 6020′ includes a plurality of diameters, the diameters of the central opening 6020′ being complementary to diameters of the portion of the mount adapter 640 that are disposed in the central opening 6020′ of the tool 602′.

The mount adapter 640′ can be configured to assist in mating the tool 602′ with the mount 610′. As shown, the distal end 640d′ of the mount adapter 640′ serves as a male mating feature, with the distal end 640d′ comprising a plurality of diameters and the threaded portion 602t′ that is configured to engage a threaded portion 602t′ of the central opening 6020′ of the tool 602′. A proximal end 640p′ of the mount adapter 640′ can be configured to receive the mount 610. As shown, the proximal end 640p′ includes a lip 642′ that defines a receiving portion or chamber 644′. The receiving portion 644′ can have a capture plate 620′ disposed therein, as well as a spring 624′ configured to bias the capture plate 620′ in a resting, locked position or configuration. An access port 646′, akin to the access port 508 of the tool 502, can be formed in the lip 642′, provided access to a terminal edge 620a′ of the capture plate 620′. A proximal-most surface of the lip 642′ can be adapted, e.g., it can be substantially flat, to receive a distal disc 612′ of the mount 610′. The mount 610′ can be coupled to the mount adapter 640′ by way of one or more screws 632′.

The capture plate 620′ can slide within the chamber 644′ and operate in a manner akin to the capture plate 520, and thus can move between a resting, locked or capture position or configuration and an unlocked or releasing position or configuration. In the locked or capture position, the capture plate 620′ can be configured grasp a groove 384″ formed in a distal end 385d″ of the drive shaft 385″. As shown, the capture plate 620′ includes a terminal edge 620a′, which can help allow the capture plate 620′ to serve as a quick-release latch. In the illustrated embodiment the terminal edge 620a′ is part of a tab 618 that extends above a primary surface of the capture plate 620′, the tab 618′ providing an easy feature to be engaged for purposes of providing a quick-release.

The mount 610′, which can sometimes be referred to as an adapter, blazer adapter, blazing adapter, or broaching adapter, can further include a plurality of plates or discs. As shown the mount 610′ includes a larger, distal disc or plate 612′, a smaller, proximal disc 614′, which in at least some instances can be a roller bearing, and an even smaller, intermediate disc 613′ disposed between the distal and proximal discs 612′, 614′, which in at least some embodiments, can also be part of the roller bearing. As shown, a notch 612n′ can be formed in the distal disc 612′ to provide a path along which the tab 618′ can slide when operating the capture plate 620′ against a bias provided by the spring 624′. The discs 612′ and 614′ can be akin, for example, to the discs 512 and 514, and thus additional explanation of the same is unnecessary.

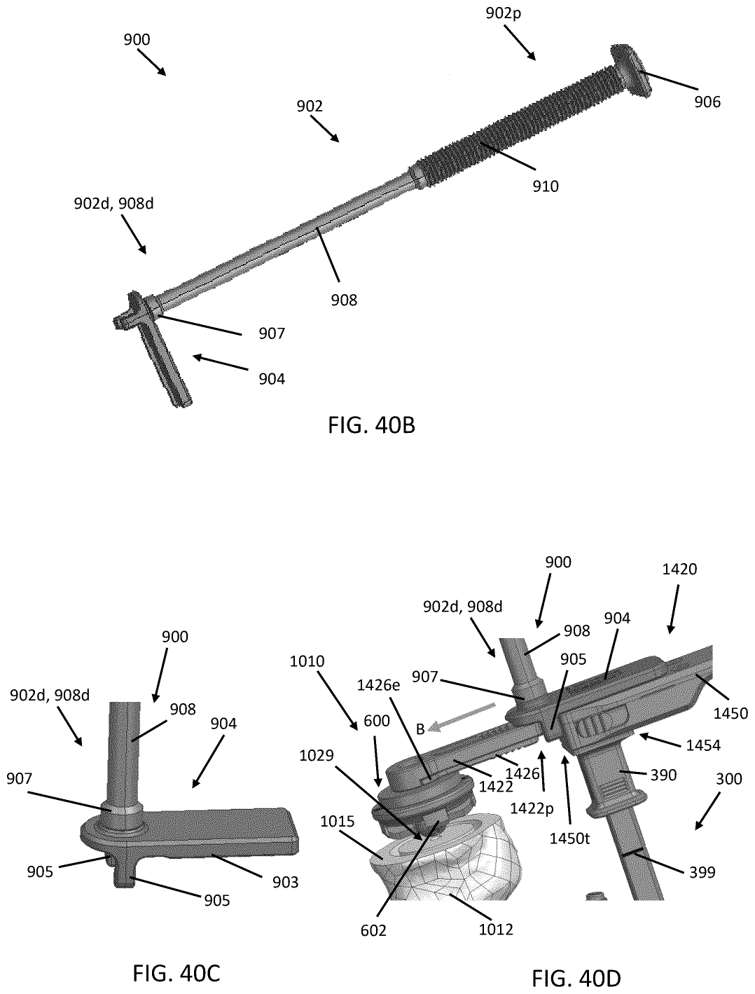

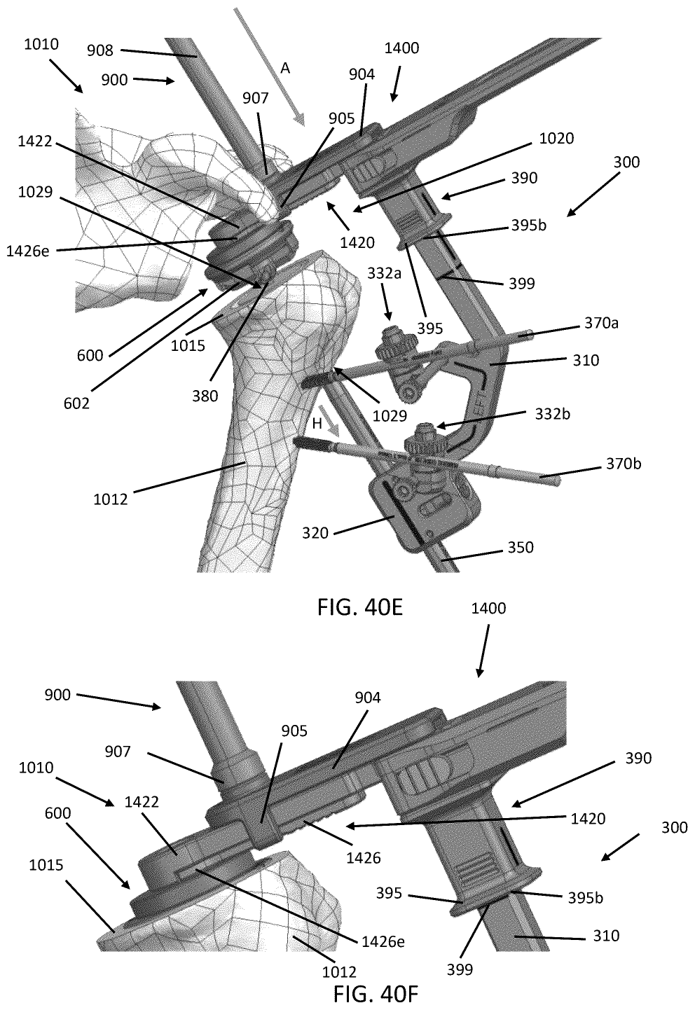

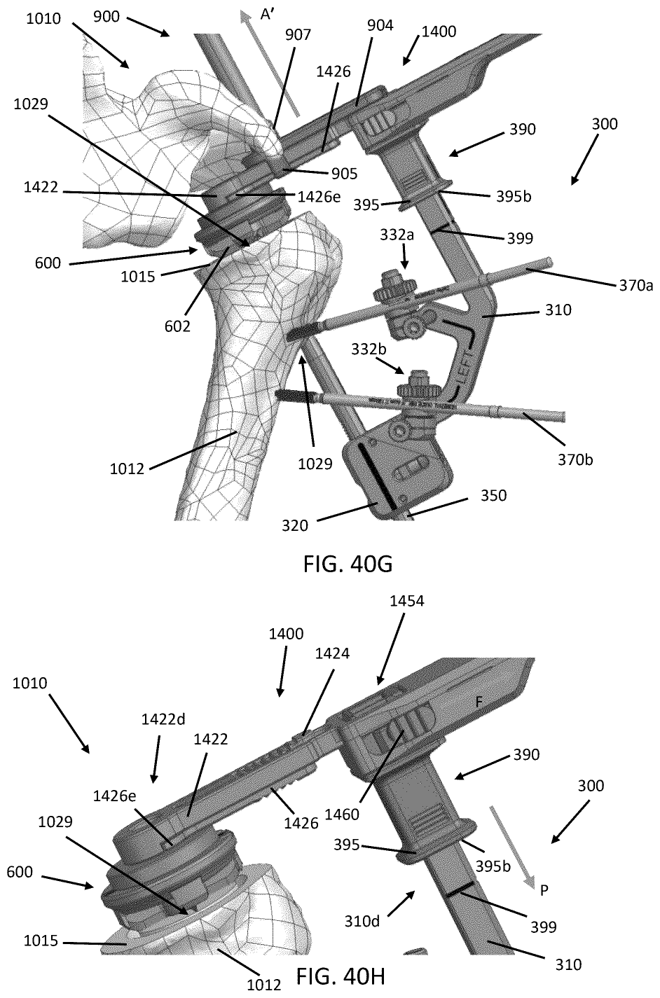

As shown in FIGS. 21A-21B, a distal tip 387″ of the drive shaft 385″ can have a surface formed to operate as a lead-in such that the drive shaft 385″ can help move the capture plate 620′ from the resting, locked position to the unlocked or releasing position. Similar to other embodiments, the drive shaft 385″ can move along the Y-Y axis illustrated, which extends through the central opening 650′. In at least some instances, an amount of force needs to be applied to blazer attachment 600′ to allow the tool 602′ to provide the desired broaching exceeds an amount of force that can be applied by the drive shaft 385″ to the blazer attachment 600′. In at least some such embodiments, an impaction tool can be provided to assist in providing the necessary force to the blazer attachment 600′ for the desired amount of broaching to occur. An example of such an impaction tool, impaction tool 900, is illustrated and described at least with respect to FIGS. 40B-40G below.

In use, modular attachments like the reamer attachments 500, 500′ and the blazer attachments 600, 600′ can be used after the humeral sizer attachment 340 has been used to position the humeral guide 300 at the desired location and the path through which the a drive shaft, like the drive shafts 385, 385′, and 385″, is to travel to operate such attachments is set. The humeral sizer attachment 340 can be detached from the humeral guide 300 while the guide 300 remains securely fixed to the humerus via bone pins 370a, 370b. If not already attached, the adapter 390 can be coupled to the arm 310 of the humeral guide 300 and the handle assembly 1400 can be coupled to the humeral guide 300, via the adapter 390. In other embodiments, the adapter 390 may not be necessary and other techniques for coupling the handle assembly 1400 to the humeral guide 300 can be employed. Modular attachments can be selectively coupled to the distal end 1400d of the handle assembly 1400, such as the reamer attachments 500, 500′ and the blazer attachments 600, 600′. The drive shaft 385, 385′, or 385″ can then be passed into the central opening, e.g., the central opening 550 of the reamer attachment 500, and used to operate the tool, e.g., the reamer tool 502.

In other embodiments, the modular attachments can be all inclusive such that they include both the tool, i.e., a portion configured to perform a function, and the mount for engaging with the handle assembly 1400. In at least some instances, the handle assembly 1400 positions the attachments, such as the reamer attachments 500, 500′ and the blazer attachments 600, 600′, at a desired location that allows the distal end 385d of the drive shaft 385 to engage and operate the attachments. Further, it is contemplated that in at least some instances, attachments or tools that are mounted to the distal end 1400d of the handle assembly 1400 can additionally or alternatively be operated by features associated with a handle assembly like the handle assembly 1400.

Modular attachments like the reamer attachments 500, 500′ and the blazer attachments 600, 600′ can be more generally referred to as humeral bone preparation instruments or attachments (sometimes the word tools can be used as well), also referred to as humerus treatment instruments or attachments (or tools). A person skilled in the art will appreciate other types of tools and attachments that can be used in a similar manner as the reamer attachments and blazer attachments provided for herein to treat the humeral resection surface 1015. References to humeral bone preparation instruments or attachments herein include, but are not limited to, reamer attachments and blazer attachments. Similar to the humeral sizer attachment 340, the reamer attachments 500, 500′ and the blazer attachments 600, 600′ can come in different sizes, with a reamer and/or a blazer attachment being selected based, at least in part, on the anatomy, age, and other demographics of the patient, along with surgeon preference, among other factors.

Different sized and/or configured humeral guides, and components used with or part of such guides, which can include but are not limited to support rods, bone pin clamps, bone pins, adapters, humeral sizer attachments, plates for use with humeral sizer attachments, handle assemblies, and/or humeral bone preparation instruments or attachments, and components thereof, can be provided together as a kit. This humeral guide and related components kit can include, for example, any combination of arms 310, hubs 320, support rods 336a, 336b, 336a′, bone pin clamps 332a, 332b, 332a′, 332b′, bone pins 370a, 370b, adapters 390, humeral sizer attachments 340, 1140, plates 1172, 1172′, INHANCE™ humeral head trials, drill cannulas 350, drill bits 380, 380′, drive shafts 385, 385′, 385″, handle assembly 1400, and/or humeral bone preparation instruments or attachments 500, 500′, 600, 600′, as well as any other such items derivable from the present disclosures. Alternatively, or additionally, some of these components can be broken up into smaller kits, such as a handle assembly kit, which can include various components of handle assemblies and humeral bone preparation instruments or attachments, such as handle assembly 1400 and related components, and/or humeral bone preparation instruments or attachments 500, 500′, 600, 600′, and their related components. A person skilled in the art will appreciate that such kits are not limited to only the embodiments disclosed and explicitly illustrated herein, but rather, includes various configuration and iterations accounted for in the text and/or otherwise understood to achieve similar purposes as provided for herein. The various components can be sized and/or shaped for different patient anatomies (e.g., adult, child, patient having certain bone formations due to various ailments or diseases, etc.). Further, a humeral guide kit, and/or components thereof, can be more generally be part of a shoulder arthroplasty surgery kit, or surgical kit more generally.

### Surgical Procedure(s)

### Accessing the Surgical Site

Traditional tools to prepare to perform a procedure such as a shoulder arthroplasty procedure rely on adequate visibility and access to the joint space provided by removing the subscapularis tendon and externally rotating the humerus 1012 so the humeral resection surface 1015 faces out the of the glenoid 1018. With the adequate space, the surgeon can resect the humeral head at the appropriate angle of inclination and retroversion, use downward force against the humeral resection surface to ream and broach the surface, thereby creating a geometry within the humeral resection surface 1015 corresponding to the chosen implant and/or prosthesis. Additionally, the space provided by removing the subscapularis tendon allows the surgeon to use downward force against the glenoid surface to ream the surface, thereby creating a geometry within the glenoid surface 1018 corresponding to the chosen implant and/or prosthesis.

The present disclosure, however, is directed to non-traditional techniques, and thus requires non-traditional tools. More particularly, the present disclosure allows for tissue sparing procedures to be performed, in which the subscapularis tendon remains intact throughout the procedure. Maintaining attachment of the subscapularis tendon means there is more limited space to perform procedures, and the devices, tools, and systems disclosed herein allow for the same types of procedures to be performed (e.g., shoulder arthroplasty) while causing less harm and damage to tissue and the surrounding anatomy. In some embodiments, such as when a tight joint is involved, a portion of the subscapularis tendon may be cut or sacrificed to increase access to the joint. As discussed above, the sacrificing of a portion of the subscapularis tendon can still be considered leaving the subscapularis tendon intact.

A non-limiting example of a method for tissue sparing shoulder arthroplasty utilizing the devices disclosed is illustrated in FIGS. 22A-44G and described below. One with skill in the art will recognize that the disclosed steps can be performed in a different order than the order presented herein and/or that variations of the disclosed methods, systems, devices, and tools are possible, in view of the present disclosures. Also incorporated by reference herein are previous aspects of the disclosed techniques, and related systems, devices, and tools disclosed in the following provisional patent applications, each of which is incorporated by reference herein in its entirety: U.S. Provisional Patent Application No. 63/579,942, entitled “Humeral Cut Guides, and Related Methods, for Use in Tissue Sparing Shoulder Arthroplasties,” U.S. Provisional Patent Application No. 63/579,952, entitled “Humeral Surgical Guides, Instruments, and Techniques for Use in Tissue Sparing Shoulder Arthroplasties,” and U.S. Provisional Patent Application No. 63/579,947, entitled “Transhumeral Glenoid Techniques and Instrumentation for Use in Tissue Sparing Shoulder Arthroplasties,” each of which was filed on Aug. 31, 2023.

To allow full access to the joint 1010, subscapularis sparing exposure can be performed, which can include, as described herein, both an inferior release and a superior release. FIGS. 22A and 22B help illustrate relevant portions of the surgical site, i.e., the glenohumeral joint space 1010, including the subscapularis 1017, the supraspinatus 1019, the coracoid 1025, the humerus 1012, the humeral head 1013, and the rotator interval 1020, as well as anatomies proximate to the same, such as vessels 1004 and a biceps sheath or latissimus dorsi tendon 1002.

To start, the patient can be positioned in a beach chair position to allow mobility of the entire arm, ensuring the humerus 1012 can be brought into full adduction, extension, and external rotation. This may include positioning a torso of the patient upright at approximately a 45° angle. The surgical site can be exposed using techniques known to those skilled in the art. For example, to access a glenohumeral joint such as the joint illustrated in FIG. 22A, an incision can be made in the skin. The skin incision can be a standard deltopectoral incision. The biceps sheath can be opened, in turn allowing the surgeon to follow the biceps into the rotator interval. At that location, a biceps tenodesis can be performed.

An inferior release of the subscapularis tendon 1017 can then be performed. In conjunction with the same, if desired, the adducted and flexed arm can be externally rotated to provide visualization of the subscapularis 1017. The inferior border of the subscapularis 1017 is defined by a plurality of vessels known as the circumflex vessels or the three sisters 1004 and the biceps sheath or latissimus dorsi tendon 1002. The vessels 1004 can be coagulated and/or suture ligated, for example from a location below the subscapularis 1017.

A surgeon can locate and open the space where the lower border is the subscapularis 1017 and the upper border is the latissimus dorsi tendon 1002. The subscapularis 1017 and capsule can be separated medially, for example, with surgical scissors. One or more retractors, such as an inferior subscapularis retractors(s), Senn retractor(s), and/or Lagenback retractor, can be used to gently lift the subscapularis 1017 to visualize the capsule underneath it. The capsule can, in turn, be opened following the curvature of the calcar and the capsule can be released off the surgical neck of the humeral head 1013. Capsular release from inferior to posterior can then be completed using techniques known to those skilled in the art, for example working from anterior to posterior by progressively externally rotating and flexing the arm. One or more retractors, such as a Darrach retractor(s) and/or a Hohmann retractor(s) can be placed to retract and protect the axillary nerve during capsular releases.

A superior release of the subscapularis tendon 1017 can also be performed. The rotator interval 1020 between the upper rolled border of the subscapularis 1017, the anterior leading edge of the supraspinatus 1019, and the lateral edge of the coracoid 1025 can be located. The rotator interval 1020, which can include tissue approximately in a triangle shape, can be opened, for example, by following the rolled superior edge of the subscapularis. In turn, the rotator interval 1020 can be excised between the superior edge of the subscapularis 1017 and the anterior edge of the supraspinatus 1019. The glenohumeral ligaments posterior to the subscapularis 1017, such as the middle glenohumeral ligament behind the superior border of the subscapularis 1017, can be located. The medial glenohumeral ligaments can, in turn, be exposed, for example with a Hohmann retractor(s), on the glenoid neck and they can be released and/or excised.

Once both the inferior and superior releases are complete, and/or as each is completed, the humeral resection surface and associated osteophytes can be identified and the osteophytes removed. In conjunction with the same, the axillary nerve can be protected, for example, with one or more retractors, such as a Narrow Darrach retractor(s), a Curved Hohmann retractor(s), and/or other Darrach, Hohmann, and/or other types of retractors. Visualization and/or access to posterior inferior osteophytes can also be increased by externally rotating the humerus in the adducted and flexed position. Existing inferior osteophytes should be visible and can be removed, for example with a curved osteotome.

After subscapularis sparing exposure is complete and the joint 1010 is exposed, anatomical landmarks, such as the humeral resection surface and bicipital groove, can be marked, for example with a marking pen or using other marking techniques known to those skilled in the art.

Various anatomical landmarks can be used to guide placement of devices throughout the procedure. For example, the bicipital groove of the humerus 1012 can be marked with a line 1011, for example using a surgical marking pen or electrocautery to draw the line on articular cartilage at the bicipital groove. The line 1011 can be used during the procedure(s) to align a resection guide, such as the guide 100, to the humeral head, among other instruments. For example, the bicipital groove can be further used as an entry point for a cutting tool during resection. As described above with respect to FIG. 2, the humeral resection surface 1015 can be used to identify a desired cutting plane along which the humeral head 1013 will be cut so that a prosthetic can be attached to the elongate shaft 1014 of the humerus 1012 within the glenohumeral joint 1010. The approximate preferred location for the cut to be performed can be marked, for example by having a surgeon use a surgical marking pen to draw a line on a surface of a subscapularis tendon or tissue (not shown, but known where it is by a person skilled in the art) and textile (e.g., displacement wrap, such as the displacement wraps disclosed in U.S. Patent Application Publication No. 2024/0108433, referenced and incorporated by referenced above; also, not shown), from an inferior location to a superior location. The line can be akin to the humeral resection surface 1015 illustrated in FIG. 2, and can be used to visualize the humeral resection surface 1015 for purposes of estimating the desired inclination angle (e.g., the angle β, which can be commensurate with the natural angle α of inclination.

### Resecting a Humeral Head

Traditional resection guides used to create a cutting or resecting plane as part of a shoulder arthroplasty procedure rely on the adequate visibility of the humeral resection surface and access to the joint space provided by removing the subscapularis tendon and externally rotating the convex humeral head out of the glenoid surface to perform this cut. Thus, for tissue sparing procedures, and other types of procedures performed in more limited space and/or with more limited displacement of tissue and the like, humeral resection guides of the nature provided for herein are necessary.

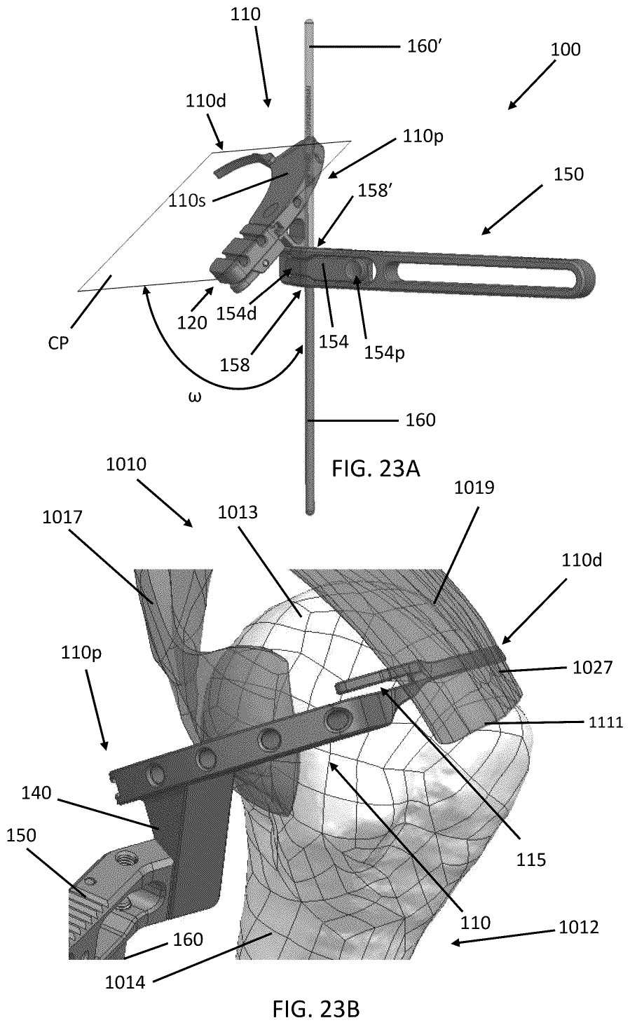

As described herein, and as shown in FIG. 23A, the humeral resection guide 100 can be a single piece guide including a superior radial arm 110 and a vertical alignment plate 140. Features like the guide extender 120 and handle 150 can be considered separate components that can be used in conjunction with the arm 110, or alternatively, one or more of these components can be considered portions of the guide 100, thus making the guide 100 having more than one piece. In some other embodiments, the vertical alignment plate 140 can also be a separate component from the superior radial arm 110 and/or other combinations of these various components can be included or not included as part of the humeral resection guide 100—integrally formed, removable and replaceably attached, etc.

Because the superior arm can be separately disposed, the arm 110 can be introduced into the patient's glenohumeral joint 1010 separately and additional components can be coupled to the arm, or alternatively, the additional components can be coupled to the superior radial arm 110 prior to insertion into the glenohumeral joint 1010. In the illustrated embodiment of FIG. 23B, the handle 150 is coupled to the superior radial arm 110 as described above and a vertical guide rod 160 is threaded into the thread slot 144 of the handle 150 prior to insertion. The handle 150 and vertical guide rod 160 can assist in positioning the guide 100 in the joint space by extending out of the body or surgical site and providing space for the surgeon to grip and manipulate the guide 100 from a distance outside of the surgical site.

As shown in FIG. 23B, the superior radial arm 110, including the distal member or portion 110d thereof, respectively, can be inserted into the joint space through the rotator interval 1020 and towards a posterior portion of the humeral articular margin while aligning against a supraspinatus insertion or attachment point 1111. This placement can set a height of the resection guide 100 at the surgical site. In the joint space, the superior radial arm 110 can be positioned between a supraspinatus 1019 and a humeral head attachment point 1111 to define the articular margin 1009 (see FIG. 2). The arm 110 can sit against an upper surface of the humeral head 1013, for instance at the articular margin 1009 or other desired resection location. During insertion, a surgeon can palpate along the superior radial arm 110 to ensure the arm 110 is tight against the supraspinatus insertion at a greater tuberosity 1027 of the humeral head 1013, as well as positioned to follow the articular margin 1009 posteriorly. This creates a tight attachment of the guide 100 against the supraspinatus attachment point. The blade slot 115 of the superior radial arm 110 can be aligned to an anatomical landmark, such as the bicipital groove of the humerus 1012 or the rotator interval, to accurately define an entry point for a cutting tool. The rotator interval alignment can be used, for example, for an anatomical procedure or area. This positioning can also help ensure any soft tissue is retracted away from a cutting blade that passes through the blade slot 115. The superior radial arm 110 can be considered proximate to the humeral head when it is passed through the rotator interval 1020 and within approximately three centimeters of the humeral head 1013. For most anatomies, when properly positioned, approximately 1 millimeter of bone can remain at the insertion after the resection. Any posterior cuff and/or deltoid retractor(s) can be removed, and the arm can be slightly abducted to help ensure optimal placement of the superior radial arm 110.

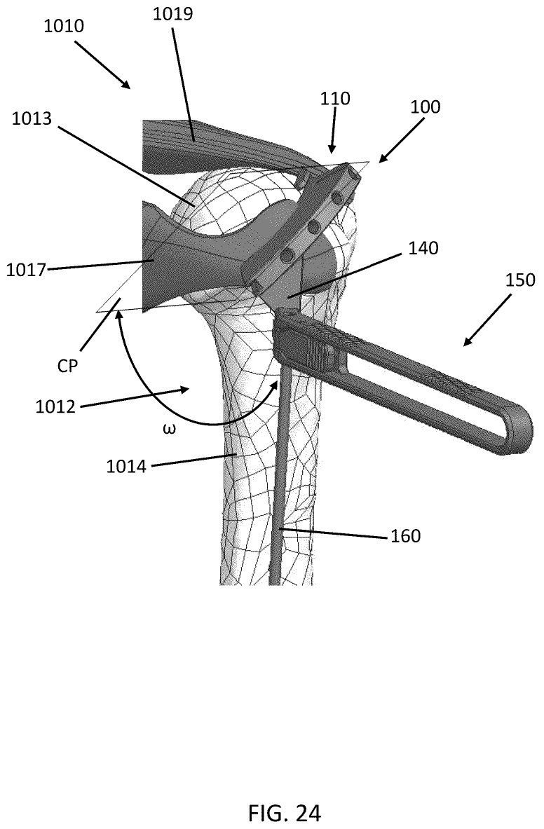

In place at the surgical site, the resection guide 100 can define the cutting plane CP. More particularly, in at least some embodiments, the resection guide can approximate a 135° angle (or, more generally, approximately in a range of about 125° to about 145° as indicated earlier), as shown the cutting plane angle ω, that serves as the humeral resection angle. The approximate 135° angle can be defined, for example, when the vertical guide rod 160 is aligned with the shaft 1014 of the humerus 1012, as shown in FIG. 24. Before, during, and/or after introduction of the arm 110 to the glenohumeral joint 1010, a displacement wrap(s), retractor(s), and/or other component(s) useful in manipulating a location of a subscapularis tendon can be used to move the subscapularis tendon out of the way to improve visualization.

As illustrated in FIG. 24, the version handle 150 can be coupled to the vertical alignment plate 140 associated with the superior radial arm 110. The latch 154 can be operated to secure the version handle 150 to the alignment plate 140. The vertical guide rod 160 can be threaded into the thread slot 144 of the handle 150 and extend distally along the humerus 1012 to help better align the vertical alignment plate 140, and thus the guide 100, with the elongate shaft 1014 of the humerus 1012. As shown, the vertical guide rod 160 can be aligned, i.e., parallel or substantially parallel to, the elongate shaft 1014 of the humerus 1012. The rod 160 can typically be maintained in this parallel or substantially parallel configuration with respect to the shaft 1014 at least until one or more bone pins, such as the bone pins 125a, 125b, 125c, are used to couple the guide 100 to the humeral head 1013 and/or the humerus 1012. The rod 160 can be secured in this parallel or substantially parallel manner before and/or after it is secured to the version handle 150. More particularly, in conjunction with positioning the guide 100 at the anatomic neck, the vertical guide rod 160 can be used to align the flexion/extension angle of the humeral head, i.e., aligning the angles α′ and β′ to form a linear pair long the longitudinal axis L (see, e.g., FIGS. 2 and 3A). When the vertical guide rod 160 is aligned with the elongate shaft 1014 of the humerus 1012, the humeral resection angle, i.e., the cutting plane angle ω, can be approximately 135° (or, more generally, approximately in a range of about 125° to about 145° as indicated earlier). Further, when attached, the handle 150 can be set at about approximately 30° of retroversion and can be aligned with the forearm. Alternatively, the handle 150 can be manually set to an alternate version. Movement of guide 100 to achieve the desired alignment can be accomplished, for example, by manipulating the vertical guide rod 160 and/or handle 150 to adjust the angle of inclination or retroversion of the superior radial arm 110 with respect to the humerus 1012.

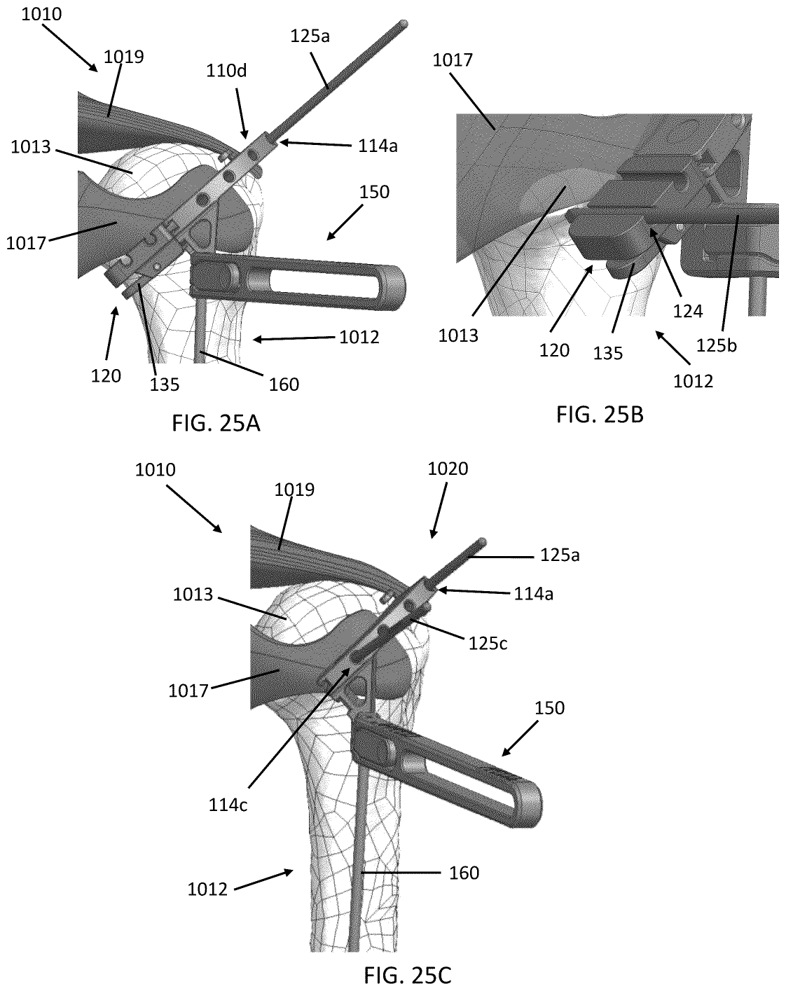

After a position of the guide 100 with respect to the humeral head 1013 has been set, one or more pins can be introduced to maintain the guide 100 at the desired location with respect to the humeral head 1013 to couple the guide 100 to the humeral head 1013, and thus the humerus 1012. As shown in FIGS. 25A and 25C, a pin 125a can be placed bi-cortically into the humeral head 1013, in the bicipital groove area, without pinning through soft tissue. More particularly, one pin 125a, which can be considered a superior pin based on its illustrated placement, can be positioned in an opening or hole 114a formed in the superior radial arm 110 and into the humeral head 1013 at a location between the supraspinatus 1019 and subscapularis 1017 tendons such that the pin 125a does not violate the supraspinatus 1019. The pin 125a can be inserted into any one of the openings 114 of the superior radial arm 110 to avoid passing the pin 125a through the supraspinatus 1019 and subscapularis 1017 tendons or other soft tissue surrounding the joint area.

The guide extender 120 can be optionally coupled to the proximal end of the superior radial arm 110 to extend the resecting plane beyond the inferior border 1021 of the subscapularis tendon as shown in FIG. 25A. The use of the guide extender 120 can provide a more “patient specific anatomic” humeral resection, which may not be an approximately 135° resection angle. The resection angle that can result from using the extender 120 is approximately in the range of about 135° to about 155°. When using the guide extender 120, the vertical guide rod 160 can still be used to reference bone alignment, but it may be ignored for purposes of defining the inclination angle.

To introduce the extender 120 to the surgical site, a retractor(s), such as an inferior subscapularis retractor(s), a small Hohmann retractor(s), and/or a Senn retractor(s), can be used to gently lift the subscapularis to visualize and palpate the inferior articular margin using a pin 125b, as shown in FIG. 25B. The resection guide 100 can be adjusted as necessary until a desired “patient specific anatomic” inclination is achieved, i.e., the angle ranges provided in the preceding paragraph.

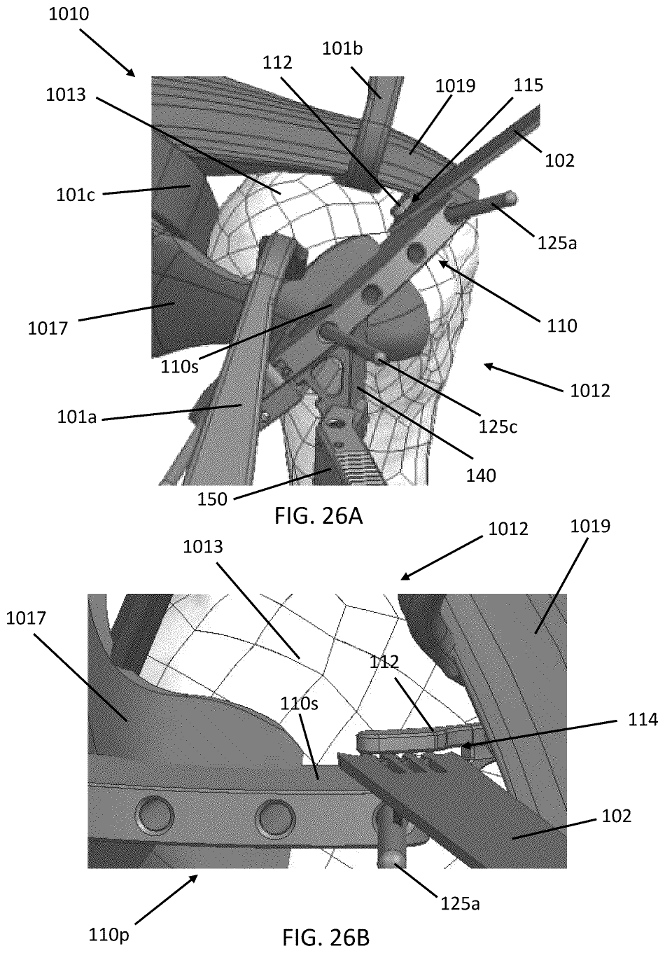

A top surface of the extender 120 represents the intended humeral resection plane, i.e., the equivalent of the cutting plane CP. Each of the grooves 124 formed in the extender 120 can be positioned tangent to the resection plane. When positioning the guide 100, a top surface of the pin can be referenced, which in turn becomes the bottom of the extended cutting tool that performs the resection (e.g., the tool 102 as shown in FIG. 26B).

If the guide extender 120 is used, another guide pin, for example the pin 125b, which can be considered an inferior pin based on its illustrated placement, can be positioned in the grooves 124 formed in the inferior extender 120. The pin 125b can inserted into the humerus at a location below the subscapularis 1017. Use of the guide extender 120 helps prevent passing a pin through the subscapularis 1017, while also achieving a “patient specific anatomic” resection. The illustrated positioning of the pins 125a, 125b helps ensure that the pins do not pass through or otherwise violate supraspinatus 1019 tissue and/or subscapularis 1017 tissue. In embodiments where the guide extender 120 is not used to achieve a patient specific anatomic resection, such as the embodiment illustrated in FIG. 25C, an inferior pin 125c can be passed through an opening or hole 114c of the superior radial arm 110 at a location inferior to the superior pin 125a to further secure the guide 100. In such instances, the extender 120 can be used to provide a visual confirmation inferiorly. In some cases, the inferior pin can pass through the subscapularis tendon 1017 and into the humeral head 1013. A person skilled in the art will appreciate any combination of the openings or grooves 114, 124 can be used to have pins disposed therein for positioning the guide 100 with respect to the humeral head 1013 and/or the humerus 1012. The position of the guide 100 can typically be maintained while placing the pins (e.g., the pins 125a, 125b, 125c). While a variety of pin configurations can be used, in at least some embodiments, one pin, as shown the pin 125a, can often be disposed at a most superior position within the rotator interval to ensure that none of the pins violate the supraspinatus tissue 1019, and a second pin, as shown the pin 125c, can be in an inferior location positioned below or through the subscapularis 1017. As shown in FIG. 25C, the pin 125a is in the most superior position and the pin 125c is at the inferior location, disposed through the subscapularis 1017. The 135° angle can sometimes be preferred when there is a high potential for poor bone quality as it can allow for easy conversion to a shoulder system anatomic surgical technique.

FIGS. 26A and 26B illustrate the cutting or resection action performed to complete the procedure. A cutting tool 102 for use in performing the resection can be a saw, such as a narrow rigid saw (e.g., a sagittal saw). In some embodiments, the saw can have a width of approximately 13 millimeters. One or more retractors, such as anterior subscapularis retractor(s) and/or posterior cuff retractor(s) (e.g., angled or double bent Hohmann retractors) 101a, 101b, or other soft tissue retractors known in the art, can be used to move surrounding soft tissue to help protect the subscapularis tendon 1017 and the supraspinatus tendon 1019 during the humeral cut and/or to provide additional visualization. One or more retractor(s) 101c, such as a Darrach retractor(s) or a curved Hohmann retractor(s), can be positioned over the humeral head 1013 and against the glenoid 1018 for protection during resection.

As noted above, a blade slot 115 formed over the superior arm surface 110s can be used to align the cutting tool 102 planar with the superior arm surface 110s while also ensuring an entry position for the tool 102 is between soft tissue humeral attachment points, specifically at the bicipital groove as denoted by the line 1011. A first blade plunge of the tool 102 can follow the blade slot 115 and into the humeral head 1013. The tool 102 can then be retracted, pivoted, and a second plunge can occur, again keeping the blade flat on the superior planar surface 110s and within the blade slot 115. This cutting technique can continue to be repeated until the humeral head has been resected and/or cut as desired, all the while being able to avoid damaging the subscapularis tendon 1017 and the supraspinatus tendon 1019 because of the configuration of the device and surgical procedures performed in view of the same. For example as discussed above with respect to FIG. 4, the distal portion 110d of the superior arm can act as a shield to protect the far side anatomical structures from the saw blade, particularly if care is taken to ensure the cutting tool 102 follows the cutting plane defined by the blade slot 115 and superior surface 110s. When performing the plunging cut actions and reaching completion medial and posteromedial, care should be taken to avoid injury to the axillary nerve. In some patients, minimal release of the upper border of the subscapularis 1017 can provide additional clearance for the cutting tool 102.

After a series of plunge cuts are performed to resect the humeral head 1013 as desired, thus completing the humeral osteotomy, the pins, as shown the pins 125a, 125c, can be removed, as can the guide 100. Removal of at least some of the components, such as the guide 100, can occur through the rotator interval 1020. In at least some instances, the final portion of the humeral cut can be performed with an osteotome or other suitable instrument(s). If appropriate and/or desired, the guide 100 can be repositioned for one or more additional cuts to be performed. After the pins 125a, 125c and guide 100 have been removed, the resection plane can be palpated, and a rongeur, osteotome, and/or other instrument(s) can be used to remove any residual bone above the resection plane and/or residual osteophytes.

After the humeral head 1013 has been resected, the bone quality can be evaluated, for example by applying thumb pressure to the resulting humeral resection surface. If a thumb can be depressed into the humerus without much resistance, the bone may not be sufficient to support a stemless implant with the approach provided for herein and a stemmed implant may provide better fixation. A person skilled in the art, in view of the present disclosures, will understand how to use a stemmed implant in conjunction with disclosed procedures.

### Preparing a Glenoid Surface

Following resection of the humeral head, one or both of the glenoid surface 1018 and humeral resection surface 1015 can be prepared to receive an implant. Depending on patient anatomy, glenoid preparation can be achieved following the INHANCE™ surgical technique (from Johnson & Johnson of New Brunswick, NJ), as disclosed at https://www.jnjmedtech.com/en-US/pdf/inhancetm-shoulder-system-anatomic-surgical-technique, the content of which is incorporated by reference herein in its entirety, using inferior and posterior humeral head retraction. However, to the extent a patient anatomy does not allow for guide pin placement and glenoid reaming along a desired glenoid central axis, and/or a transhumeral approach is more preferred for reasons described herein, a guide like the glenoid guide 200 can be used to achieve a transhumeral approach.

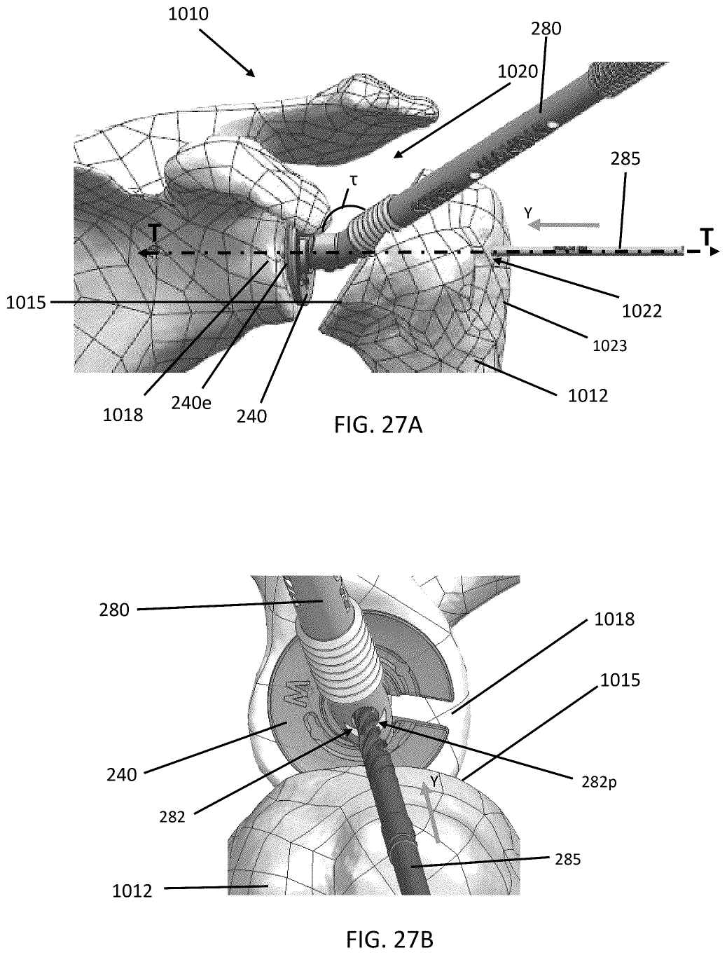

A non-limiting example of a surgical procedure for preparing a glenoid surface to receive an implant and/or a prosthesis using a glenoid guide 200 of the present disclosure is illustrated in FIGS. 27A-32B. Prior to introducing a glenoid guide or other glenoid preparation tools to the surgical site, the glenoid can be exposed using techniques known to those skilled in the art and the labrum can be removed or otherwise moved out of the way for access to the surgical site. For example, one or more glenoid retractors can be used for these purposes, as is known to those skilled in the art and described. This may or may not involve using the retractor(s) that were used in conjunction with resecting the humeral head, and to the extent the retractor(s) are used for both, they may or may not be manipulated or otherwise moved to provide desired exposure to the glenoid area of the joint space. In some instances, retractor(s) and related components from the DePuy Global Enable Retractor set (from Johnson & Johnson of New Brunswick, NJ) can be used. These can include an anterior glenoid neck retractor(s) and/or a forked posterior retractor(s). A standard blunt or sharp-tipped Hohmann retractor(s) can be used superiorly. Additionally, use of a bone hook 1031 (see FIGS. 8A and 28B) can be used for lateral humeral manipulation, for instance to increase visualization within the joint.

With the glenoid retractor(s) in place, various instruments can be used to determine the correct glenoid implant size and/or location, and tools such as the glenoid guide 200 can be positioned for use at a surgical site. In the illustrated embodiment, the surgical site is the human glenohumeral shoulder joint 1010 at which the glenoid 1018 and the humeral resection surface 1015 of the humerus 1012 are located, as also illustrated in FIGS. 2 and 22A-22B.

Starting first with determining an appropriate size and location of a glenoid implant, a sizer plate or disc, such as a sizer plate, disc, or sizer 240, which can be one of a plurality of differently sized and otherwise configured sizer plates, can be used for these purposes. The sizer 240 can be introduced into the glenohumeral joint, for example through the rotator interval 1020, to determine the appropriate size of instrumentation and/or prostheses to use at the surgical site and/or to locate the center point or center, or an approximate center point or center, of the glenoid surface 1018. FIGS. 27A and 27B illustrate two manners in which such a sizer 240 can be introduced to the glenoid surface 1018—one in which a transhumeral bone tunnel 1022 is formed in the humerus 1012 (FIG. 27A), and one in which such a tunnel is not (FIG. 27B). The selected sizer 240 can be based on information about the patient, experience of those performing the procedure, determinations made with a humeral sizer attachment 340, and/or sizers 240 having different sizes that can be introduced into the joint space until the proper size is determined. Non-limiting examples of sizers 240 include INHANCE™ glenoid sizers (from Johnson & Johnson of New Brunswick, NJ). The sizer plate 240 can be attached to a tool, such as a sizer handle 280 as shown in each of FIGS. 27A and 27B, to introduce the plate 240 into the limited joint space through the rotator interval 1020 while keeping surrounding soft tissue intact. As shown, the convex external surface 240e can be aligned against the surface of the glenoid 1018.

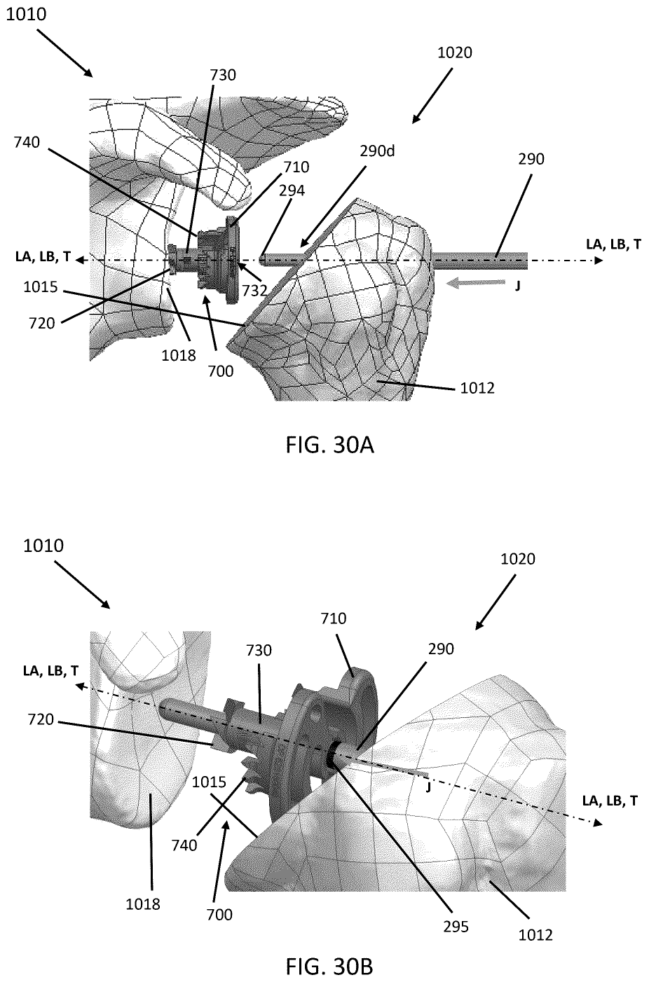

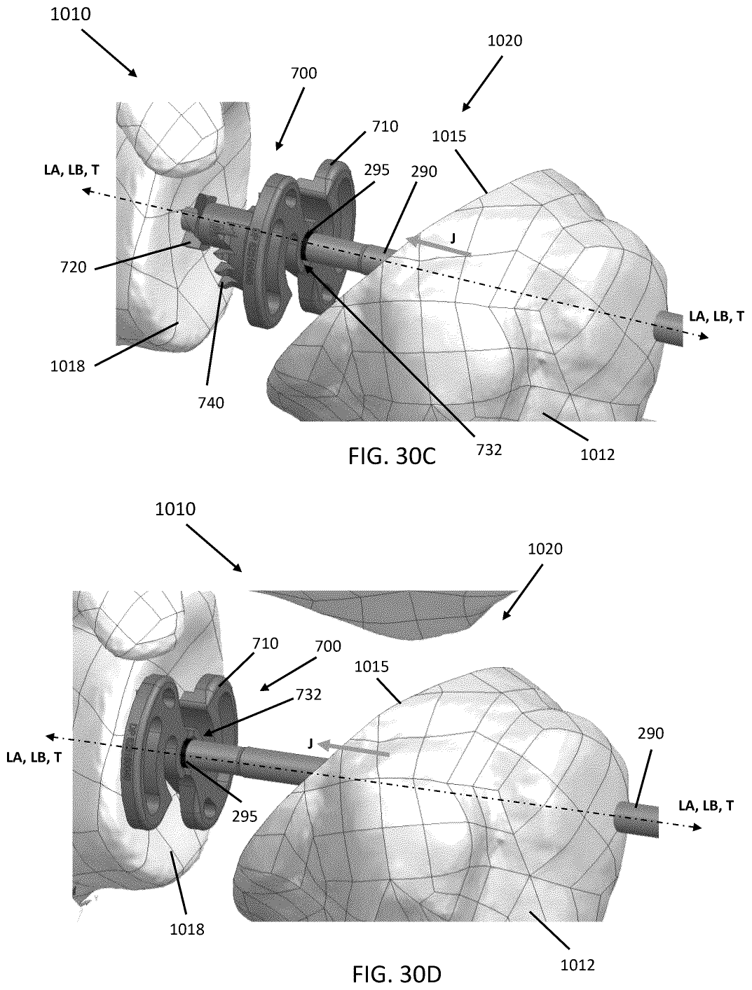

As shown in FIG. 27A, in some embodiments, access to a surface of the glenoid 1018 during tissue sparing arthroplasty can be obtained, in part, by drilling a transhumeral bone tunnel 1022 from the lateral cortex 1023 of the humerus 1012, through the humerus 1012, and to the humeral resection surface 1015 such that an axis T of the transhumeral bone tunnel 1022 extends substantially perpendicular and substantially center to the face or surface of the glenoid 1018. As described, for example, with respect to at least FIGS. 30A-30E, any force required to prepare the surface of the glenoid 1018 to receive an implant and/or prosthesis can be provided pushing a driver (e.g., the driver 290) engaged with an attachment (e.g., the reamer attachment 700) placed against the surface of the glenoid 1018 through the transhumeral bone tunnel, thereby forcing the attachment against the glenoid surface 1018. Tools such as the glenoid guide 200 or a sizer handle 280 can be used to set the trajectory and guide the drilling of the transhumeral bone tunnel.

The sizer handle 280 includes a distal end 280d configured to couple to a sizer plate such as the plate 240 and a proximal or handle portion (not shown, but known and understood by a person skilled in the art). The distal end 280d can be joined to the proximal handle portion at a fixed angle τ approximately in the range of about 0° to about 60° to allow the sizer plate 240 to sit flush, or substantially flush, with the glenoid surface 1018 while the handle portion 280p extends out of the rotator interval 1020 and further out of the joint space 1010. In the illustrated embodiment, the angle τ is about 45°. A person skilled in the art, in view of the present disclosures, will appreciate that a surgeon can flip the handle 280 over and use the long axis of the handle, as it too can be cannulated, which can provide “face-on” access that would be approximately 0°. The illustrated sizer handle 280 further includes a channel 282 extending through the distal portion 280d, as best seen in FIG. 27B. A proximal opening 282p of the channel 282 provides access to the surgical site, i.e., the glenoid surface 1018 as shown, and a distal opening (not visible) of the channel 282 is configured to align with a central opening of the sizer plate 240 when the sizer plate 240 is coupled to the sizer handle 280. This alignment allows for a glenoid drill, such as the drill bit or drill 285, passed therethrough to access the glenoid surface 1018 to form at least a pilot hole therein.

The transhumeral hole or tunnel 1022 can be formed using any techniques known to those skilled in the art for forming a hole or tunnel in bone. For example, the sizer handle 280 and sizer plate 240 can be introduced through the rotator interval 1020, for instance with the subscapularis tendon still intact, such introduction being performed using techniques disclosed herein or otherwise known to those skilled in the art. With the sizer plate 240 centered, or approximately centered, against the glenoid surface, the center point, or approximate center point, of the glenoid surface can be defined by the central opening of the sizer plate 240 and the channel 282 of the sizer handle 280. To create a transhumeral tunnel 1022, also referred to as a transhumeral bone tunnel, the drill bit 285 can be advanced through the humerus 1012 along an axis T, through the channel 282, and through the central opening of the sizer plate 240 until the distal end of drill enters the glenoid surface 1018, creating a pilot hole in the glenoid surface 1018. Because the drill bit 285 has a stepped configuration, as best shown in FIG. 8B, the smaller diameter can be used to initially form the transhumeral tunnel 1022 and the pilot hole, and the larger diameter can be used to increase a diameter of the transhumeral tunnel but not enter the glenoid so the pilot hole remains the smaller diameter. The pilot hole serves as a central starting hole as it can be substantially centered with respect to the glenoid surface 1018.

Alternatively, as shown in FIG. 27B, if patient anatomy allows and/or is manipulated in known manners, the drill bit 285 can be passed through the rotator interval 1020 inserted into the channel 282 to form a starter hole and/or a pilot hole in the glenoid surface 1018 at the approximate centerpoint of the glenoid defined by the central opening of the sizer plate 240, without having to pass through a transhumeral tunnel, like the tunnel 1022, formed in the humerus 1012. For example, a starter hole can be formed, with the hold being approximately in the range of about 3 millimeters to about 5 millimeters deep. This initial starting hole may not be in the optimal trajectory, but it can be used to approximately centrally dock and guide the drill bit 285 when creating the pilot hole. Because the illustrated drill bit 285 is a stepped configuration, the resulting pilot hole can have multiple diameters, though in other embodiments, a single diameter drill bit may be used such that the resulting pilot hole has a single diameter.

The sizing and creation of the central starting and pilot holes can be used to guide the transhumeral glenoid preparation workflow that follows. In some embodiments, the starter hole is formed as illustrated in FIG. 27B and then a pilot hole is formed, for example as described with respect to FIGS. 28A-28B below, with the starter hole being able to be used to centrally dock the glenoid guide 200 and guide the drill bit 285 when creating the pilot hole.

Following the creation of a starter and/or pilot hole in the glenoid surface 1018, a glenoid guide, such as the guide 200, can be introduced to assist in preparing the glenoid to receive an implant(s) and/or to further define the position and trajectory of the transhumeral tunnel 1022 and its entry into the glenoid surface 1018. The glenoid guide 200 can be introduced into the joint space 1010 through the rotator interval 1020. For this transhumeral glenoid preparation, an external, articulated arm positioner, such as arm positioners like a Smith & Nephew Spider arm or limb positioner, can be used, for example by attaching it to an operating room bed. Such a positioner can be used, for example, to approximately align a desired entry point on the humerus 1012 with the glenoid face or surface 1018. A surgical assistant can also be used in lieu of, or in conjunction with, the positioner.

The arm of the patient can be placed in a neutral position with the humeral resection surface 1015 facing the glenoid fossa. The foregoing notwithstanding, a person skilled in the art will appreciate that optimal positioning can depend, at least in part, on patient anatomy, the humeral resection, and/or surgeon preference, and thus other positioning may be more appropriate. It can be helpful to plan and identify the optimal trajectory for glenoid drill insertion. This may include, for example, targeting a zone that is approximately in the range of about 1 centimeter to about 2 centimeters below the plane of the humeral resection surface 1015 for a lateral entry point of the teeth 259 of the drill cannula 250. The aforementioned arm positioner and/or a surgical assistant can be used to help maintain the humerus in the desired position.

While the present disclosure discusses determining a center, or an approximate center, of the glenoid surface 1018, such as by using glenoid sizer plates (e.g., the sizer plates 240), glenoid sizer plates more generally can be used to determine a preferred location on the glenoid surface 1018, which may or may not be a center or approximate center. A preferred location may be, for example, more inferiorly positioned. A preferred location can depend, at least in part, on an anatomy of a patient, the configuration of the instruments and/or tools being used, and/or surgeon preferences, among other factors.

FIG. 28A illustrates the glenoid guide 200 coupled to the humeral resection surface 1015, and thus the humerus 1012, with a distal portion 200d of the glenoid guide 200 being disposed between the humeral resection surface 1015 and a surface of the glenoid 1018. In use, a proximal portion 200p of the glenoid guide 200 can be disposed outside of a patient's body. More particularly, as shown, the generally arcuate, or offset, arm 210 of the guide can be placed such that the distal portion 210d of the arm 210 is disposed within the rotator interval 1020 between the humeral resection surface 1015 and the glenoid surface 1018 and the proximal end 210p is disposed proximal of the humeral resection surface 1015, outside of the joint space 1010 and/or outside of the patient's body. As shown, the post 226 having the distal opening 224 formed therein can contact or otherwise abut the glenoid surface 1018 at an approximate centerpoint. The drill cannula 250 can be inserted into the opening 222 at the proximal end 210p and be advanced towards the humeral resection surface 1015 in a direction Y until the distal tip 250t of the distal portion 250d of the drill cannula 250, and thus the teeth 259, contacts the lateral cortex 1023 of the humerus 1012, for example at the aforementioned targeted zone that is approximately in the range of about 1 centimeter to about 2 centimeters below the plane of the humeral resection surface 1015. For example, the teeth 259 can be at least 10 millimeters below the resection surface. In instances in which the transhumeral tunnel 1022 have not yet been created, such as FIG. 27B, or instances in which the created transhumeral tunnel 1022 needs to have a larger diameter, the drill 285 can be advanced in the direction Y to form the desired transhumeral tunnel 1022.

As shown, the cannula-receiving opening 222 at the proximal end 210p, the drill cannula 250, and the opening at the distal end 210d are aligned along the axis LA, which is the same axis as the longitudinal axis T extending through the transhumeral tunnel 1022. Therefore, aligning the distal opening 224 with the pilot hole formed in the glenoid surface 1018 will align the opening 256 of the drill cannula 250 and the cannula-receiving opening 222 along the axis T of the transhumeral tunnel 1022.

The drill bit 285 can be passed through the drill cannula 250 and into the humerus 1012, starting from the lateral cortex 1023 until it exits the humeral resection surface 1015. A tip 285t of the drill 285 can be visualized extending beyond the post 226, prior to entering into the glenoid 1018. The bone hook 1031 (see, e.g., FIG. 28B) can help to increase visualization within the joint. This includes while the tip 285t has not yet entered the glenoid 1018. The guide 200 can be slid back, towards the humeral resection surface 1015, to better visualize the drill tip 285t. As the drill bit 285 is advanced out of the humeral resection surface 1015, it can follow the trajectory into the distal opening 224 and into the starter and/or pilot hole at the center of the glenoid surface 1018, as shown in FIG. 28B. Using the pilot hole formed in the glenoid surface 1018, the drill bit 285 can be docked therein. The glenoid guide 200, in turn, can be slid towards the glenoid surface 1018, positioned on the glenoid face, in a desired version and inclination, with the post 226 in contact with the glenoid surface 1018. A position of the humerus 1012 can be adjusted as desired to achieve a desired axis T.

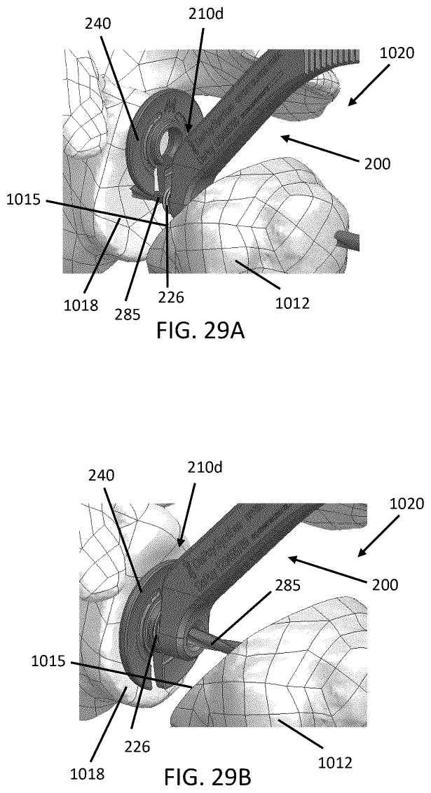

FIGS. 29A and 29B illustrate an alternative embodiment in which the sizer 240 is used to provide additional guidance and can provide enhanced tactile feedback. As shown in FIG. 29A, the sizer 240 is disposed at the surgical site. With the drill tip 285t (not visible) of the drill 285 docked in the glenoid 1018, the guide 200 can be retracted and a tool, such as various snaps tools described herein or otherwise known to those skilled in the art, can be used to slide the sizer 240 over the shaft of the drill 285, as shown in FIG. 29B. A push towards the glenoid 1018 can mate the sizer 240 to the drill 285 and can also center the guide 200, and in particular the post 226 with the sizer 240. With medial pressure, the guide 200 trajectory can be adjusted to optimize drill axis while the sizer 240 provides additional tactile feedback. The humerus position can also be adjusted as necessary to achieve optimal trajectory.

Once that desired alignment is achieved, the drill bit 285 can be advanced further in the direction Y until bicortical penetration is achieved. For example, the drill bit 285 can be advanced down the glenoid fault at least about 25 millimeters. It can be confirmed that the drill bit 285 exits the scapula as desired. Subsequently, the drill bit 285 can be removed from glenoid surface 1018 and the guide 200, thus leaving the surgical site, and the glenoid guide 200 also can be removed from the surgical site to provide additional space for further glenoid preparation steps. These components can be existed through the rotator interval 1020, for example. Optionally, after the glenoid guide 200 has been evacuated from the surgical site, the drill bit 285, or another elongate structure, can be inserted through the tunnel 1022 in the direction Y and into the face of the glenoid 1018 for clear visualization to confirm placement. In some embodiments, the drill bit 285 or another component can be inserted through the drill cannula 250, and the transhumeral tunnel 1022, to serve as a guidewire during the procedure.

### Bullet Driver(s) Reaming

FIGS. 30A-30E illustrate the use of the glenoid reamer attachment 700 and the driver 290 of FIGS. 9A-9E. A size of the reamer attachment 700 can be selected based on the size of the glenoid sizer 240, or other sizes disclosed herein or otherwise known to those skilled in the art. More particularly, with the glenoid sizer 240 being determined to be the appropriate size for the glenoid of the patient, which in turn informs the appropriate size of the prosthesis or implant that will engage or otherwise be used with the glenoid 1018, the glenoid sizer 240 also informs the selection of the size of the reamer attachment 700 as the selected reamer attachment 700 will influence the size, shape, speed, and/or other parameters associated with modifying the surface of the glenoid 1018 in the desired manner for the surgical procedure.

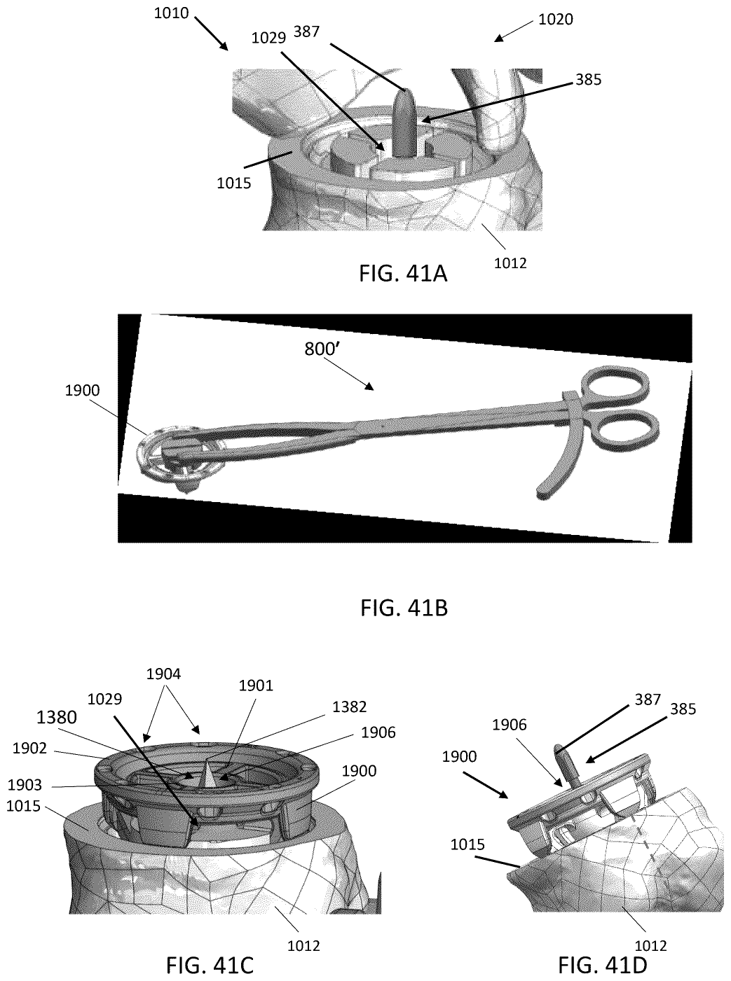

Prior to inserting the reamer attachment 700 to the surgical site, the arm cam be positioned in adduction with slight external rotation. This can aid in assembly of components during the procedure. Use of a bone hook(s), like the hook 1031, can also help increase visualization within the joint space 1010. The reamer attachment 700 can be introduced into the glenohumeral joint space as shown in FIG. 30A, through the rotator interval 1020, such as by passing it above or below the subscapularis. A surgical tool, such as snaps (see, e.g., a tool 800 illustrated in FIG. 30E and a tool 800′ illustrated in FIG. 41B), can be used to hold the reamer attachment 700 and navigate the attachment to this location.

The driver 290 can be separately introduced into the glenohumeral joint space for alignment with the reamer attachment 700. The driver 290 can be designed to follow the axis LB, as well as the longitudinal axis T extending through the transhumeral bone tunnel formed in the humerus 1012, created by the drill 285. In at least some embodiments, the driver 290 can be attached to a quick connect driver handle or paddle handle for ease of manipulation. The driver 290 can be pushed through the transhumeral bone tunnel formed in the humerus 1012, and the tip 294 and distal end 290d of the driver 290 into the glenohumeral joint space by providing a force in the direction J. More particularly, the central longitudinal axis LB extending through the central opening 732 of the reamer attachment 700 can be aligned with the central longitudinal axis LA that extends through the driver 290 and then the driver 290 can be advanced in the direction J, along the longitudinal axis LA, and thus along the longitudinal axes LB and T. The advancement of the driver 290 in the direction J can be done with a slight twisting motion to align the hex-shaped portion 296 of the driver 290 with the hex-shaped portion 734 of the reamer attachment 700, as better illustrated by FIGS. 9C-9E. A further push in the direction J can engage and mate the reamer attachment 700 with the driver 290, which can be felt by the surgeon via tactile feedback when one mates to the other. Further, visual confirmation of the engagement can be seen as the tip 294 protrudes out past the distal-most portion of the reamer attachment 700, i.e., the pilot blades 720, as shown in FIG. 30B. More particularly, as shown, the driver 290 passes into and through the central opening 732 formed through the base 710, the reaming blades 740, the central tunnel 730, and the pilot blades 720, with the tip 294 extending distally beyond the pilot blades 720, and is disposed proximate to the glenoid 1018. Further, in at least some embodiments, a marking 295, as shown a laser marking, can be formed on the shaft of the driver 290 at a location that indicates to a user that the reamer attachment 700 is mated to the shaft 290. As a result, tactile confirmation and at least two visual confirmations of the attachment 700 being secured to the shaft 290 are provided by the illustrated design. During the course of the procedure, it can be helpful to maintain rotation during reaming into and out of the bone to ensure that the reamer attachment 700 and the driver 290 do not disengage, at least because they are assembled with a friction fit.

Subsequently, the rounded distal tip 294 of the driver 290 can be inserted into the pilot hole formed in the surface of the glenoid 1018, moving the driver 290, and thus the reamer attachment 700, in the direction J, towards the glenoid 1018. The action of inserting the rounded distal tip 294 into the pilot hole can be performed without the assistance of a guidewire or other components for guidance which can be beneficial in at least some instances because not using a guidewire provides for one less component, and sometimes fewer steps, to be used and/or engage the surface of the glenoid 1018, among other benefits understood by a person skilled in the art for not using a guidewire. Further, procedures that do not require the use of a guidewire can enhance the usability of the devices and techniques in a confined space, and can also make it casier to attach a reamer to a shaft because it prevents the need of having to go over a guidewire and then onto a shaft guided by that guidewire to properly situate the reamer. If the arm of the patient has moved, the aforementioned arm positioner can be used to align the arm such that the axes LA, LB, and T are colincar, as shown in FIG. 30B. As designed, the pre-drilled pilot hole can guide the trajectory of the driver 290 during reaming.

With reference to FIG. 30C, once the desired orientation between the reamer attachment 700 and the surface of the glenoid 1018 is achieved, the driver 290 can be operated to ream the glenoid 1018. This can be done by hand, with a driver handle for example, and/or it can be done under power, such as using modified trinkle connection like the previously referenced trinkle connection or other such connections known to those skilled in the art. To the extent power is used, generally a “ream” setting should be selected and the reamer attachment 700 typically should be actuated prior to contacting the glenoid surface 1018. The driver 290 and reamer attachment 700 can continue to be operated, moving them in the direction J as shown, until the desired depth is reached and/or until the face of the reamer, defined by the reamer face 710f, (not visible; see FIG. 9B) is flush with a face of the glenoid 1018, as shown in FIG. 30D. If excess force appears to be required to perform the reaming, it may be an indication that the driver 290 is off-axis from the pilot hole. If this occurs, it can be helpful to remove the reamer attachment 700 from bone and realign the driver 290 with the pilot hole.

Turning to FIG. 30E, after completion of glenoid reaming, the reamer attachment 700 and related components can be removed from the surgical site. This can be done, for example, under power, which can minimize a risk of premature disconnection from the driver 290. In some instances, to enhance visualization, a retractor(s) (not shown) can be placed on the humeral resection surface 1015 for protection. The driver 290 can be detached from the reamer attachment 700, for example by pulling back on the driver 290 in a direction K until the reamer attachment 700 contacts an object to disengage the reamer attachment 700 from the driver 290. For example, in the illustrated embodiment, a snaps tool 800 is provided loosely over the driver 290 and as the driver 290 is pulled in the direction K, the reamer attachment 700 can contact the snaps tool 800 to become disengaged from the driver 290. The snaps tool 800 can also protect the humeral resection surface 1015. The snaps tool 800 can enter the surgical site using techniques provided for herein or otherwise known to those skilled in the art, including entering through the rotator interval 1020. In some embodiments, pulling the driver 290 in the direction K can cause the reamer attachment 700 to contact a retractor(s) and/or the humeral resection surface 1015 to cause disengagement. The reamer attachment 700 can be removed from the surgical site, for example through the rotator interval 1020, using a surgical tool like the snaps 800 (see also, e.g., a tool 800′ illustrated in FIG. 41B).

The driver 290 can also be vacated from the surgical site, or alternatively, one or more other tools can be introduced into the glenohumeral joint space in a manner similar to the way the reamer attachment 700 was introduced. The instruments can provide other preparations to the glenoid as desired. Operation of such other tools can occur similar to operation of the reamer attachment 700, though a person skilled in the art will appreciate variations that may be appropriate in view of the purpose and operational parameters of the tool being used. Accordingly, the present disclosure provides for the ability for different tools, including but not limited to variously shaped and configured reamer attachments, as well as other tools for treating a glenoid, to be separately disposed in the glenohumeral joint space, coupled to the driver 290 or other similar shaft, operated, disconnected from the driver 290 or other similar shaft, and then removed from the surgical site. Some non-limiting examples of such tools include a Cobb Elevator surgical tool, which can be used, for example, to remove cartilage before reaming, and/or electrocautery devices, which can be used, for example, to remove soft tissue structures from bone, as well as to mark positions and/or centering location(s) in conjunction with performing a surgical procedure.

Implanting a Glenoid Implant(s) and/or Prosthesis(es)

Prior to inserting a glenoid implant and/or prosthesis, trialing and/or sizing can be performed. This can be done, for example, using the INHANCE™ surgical technique (from Johnson & Johnson of New Brunswick, NJ), which is referenced above and already incorporated by reference herein in its entirety. While performing the trialing, which can involve placing one or more trial or trial implants at the surgical site to determine desired fit and configuration, confirmation should be made that the trial has acceptable backside support, for instance by visually confirming the same via a window(s) of the trial being used. In at least some instances, an impactor tip (see, e.g., the tip 206) can be used to seat the trial(s) during trailing. After the appropriate prosthesis, sometimes referred to as an implant, is determined, steps can be taken to introduce and implant it into the glenoid 1018.

As shown in FIGS. 31A-31B, a glenoid guide 200′ can be introduced into the surgical site to implant a prosthesis, as shown a glenoid implant 204, into the glenoid surface 1018. In at least some embodiments, the glenoid guide 200′ can instead be the glenoid guide 200. The glenoid guide 200′ is similar to the glenoid guide 200, and thus additional description of the same is not needed. As shown, it includes an offset arm 210′ having a proximal end 210p′ with an opening 222′ and a distal portion end 210d′ with an opening 224′, with the proximal end 210p′ having a flat surface 212′ and the distal end 210d′ being configured to receive a component, as shown the implant 204. The distal end 210d′ can include, for example, a post 226′ (not visible) akin to the post 226. The implant 204 can have a concave surface 204c, configured to mimic a surface of the glenoid 1018, the implant 204 being configured to receive a complementary humeral head prosthesis that is implanted at the humeral resection surface 1015. Implants of the nature of the implant 204 are known to those skilled in the art, and thus further disclosure about the glenoid implant are not necessary.

The distal end 210d′ of the glenoid guide 200′, and more particularly the post 226′ (not visible) can be configured to couple to the implant 204. The implant 204 can be securely mated to the distal end 210d′ of the arm 210′ of the guide 200′ using any techniques known to those skilled in the art for coupling two components together, such as a threaded connection created by the threaded post 226′, a snap-fit connection, male-female engagement mechanisms, etc. Further, in at least some embodiments, a glenoid-facing surface 204g of the implant 204 can be treated with cement per the INHANCE™ surgical technique (from Johnson & Johnson of New Brunswick, NJ), details about the technique being available at the link previously provided above, to help better secure the implant with respect to the glenoid 1018.

The implant 204 can be removed from sterile packaging, such as a thermoformed tray and retainer lid. As packaged, the implant can be packaged with an inserter tip. To allow for a touch-free introduction of the implant, the retainer lid can be removed and the post 226′ of the glenoid guide 200′ can be threaded into the inserter tip of the implant 204, as described above. The implant 204 can be placed into the joint space in a manner such that there is axial alignment between a center axis I of the implant 204 and the longitudinal axes LA, LB, and T, as shown in FIG. 31B. As shown in FIG. 31A, the implant 204 can be situated with respect to the glenoid guide 200′ such that the axis I extends centrally through the openings 222′ and 224′. Once the positioning of the implant 204 is at the desired location at the surgical site, the glenoid guide 200′ can be used to provide an impaction force to the implant 204 to secure the implant with respect to the glenoid 1018. More particularly, a force in a direction P can be applied to the flat surface 212′ located on the proximal end 210p′ of the arm 210′, for instance using a hammer or mallet. Application of the force in the direction P can help to drive the implant 204 into the glenoid 1018 for subsequent seating with respect to the glenoid 1018. The implant 204 can be separated from the guide 200′ using known techniques, including unthreading one from the other and/or sliding the post 226′ (not visible) off the implant 204. Other implants that can be inserted in similar manners include the prosthesis 24 of FIG. 1A and the prosthesis 60 of FIG. 1B.

In at least some embodiments, as shown in FIG. 32A, an impactor tool or tip 206 can be coupled to the post 226′ (not visible) of the distal end 210d′ of the arm 210′ of the glenoid guide 200′. In the illustrated embodiment, the impactor tool 206 includes a glenoid-facing surface 206g that is convex and substantially complimentary to the concave surface 204c of the implant 204. In use, as shown in FIG. 32B, the glenoid guide can have a force in the direction P applied to the flat surface 212′, for instance by a hammer or mallet, to help drive the impactor tool 206 into the implant 204 to further seat the implant 204 at the desired location. Use of the impactor tool 206 can help minimize any damage or trauma to the concave surface 204c because the impactor tool 206 can be made of a material that does not typically cause damage (e.g., scratches, dents, etc.) to the concave surface 204c of the implant 204.

A geometry of the implant 204 is typically aligned with the complementary geometry formed in the surface of the glenoid 1018, the complementary geometry formed in the surface of the glenoid 1018 being primarily formed by the reamer attachment 700. In at least some embodiments, as the implant is maneuvered to enter the recesses formed in the surface of the glenoid 1018, it can seat approximately half-way into the surface of the glenoid 1018, though in other instances it can be more or less flush with respect to the surface of the glenoid 1018 than that. In some instances, bone cement can be used within recesses formed in the surface of the glenoid 1018 to further fix the implant within the glenoid 1018.

This resulting configuration of the implant placement can be articulated and assessed to ensure the sizing and configuration is appropriate. The implant can be removed and replaced to obtain desired joint tension. While the illustrated embodiment illustrates the glenoid guide 200′ being used for disposing the implant 204 in the glenoid 1018 without a guidewire, a person skilled in the art, in view of the present disclosures, will understand how the illustrated technique can be adapted for use with a guide wire that passes through the transhumeral tunnel 1022, through the openings 222′ and 224′ of the glenoid guide 200′, and into the pilot hole formed in the surface of the glenoid 1018.

### Using a Humeral Guide(s) to Size a Humeral Resection Surface

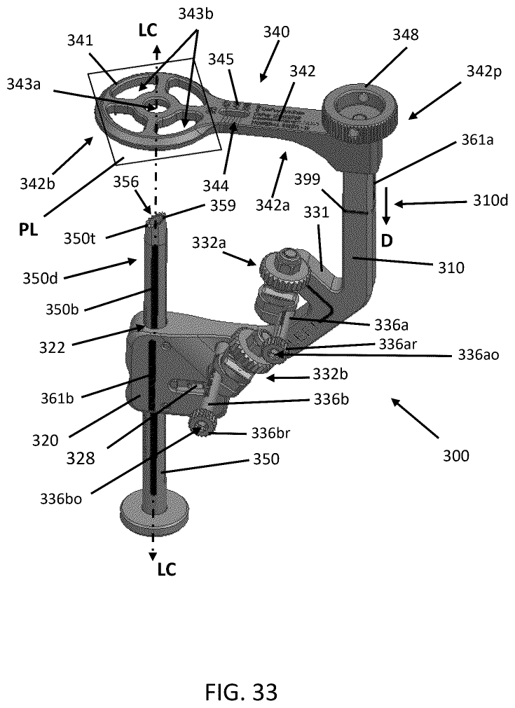

A non-limiting example of preparing a resected surface of the humerus (referred to herein as “humeral resection surface”) after resection of the humeral head to receive a prosthesis during a tissue sparing arthroplasty procedure is illustrated in FIGS. 33-40H. Access to the humeral resection surface 1015 during tissue sparing arthroplasty can be obtained by drilling a transhumeral bone tunnel from the lateral cortex 1023 of the humerus 1012 exiting central and perpendicular to the humeral resection surface 1015. Any force required to prepare the humeral resection surface 1015 to receive a prosthesis can be provided pulling a drive shaft engaged with an attachment placed against the humeral resection surface 1015 through the transhumeral bone tunnel in the lateral direction thereby forcing the attachment against the humeral resection surface 1015.

The guide 300, illustrated in FIG. 33, can be employed during tissue sparing arthroplasty surgery after the humeral head has been resected. It can be used to create a transhumeral axis LC that is substantially perpendicular and centralized on the humeral resection surface 1015, thus providing a precise axis of alignment during humeral preparation. More particularly, the rigid arm 310 operates in conjunction with the hub 320 and the drill cannula 350 to define the location and trajectory along the axis LC at which a bone tunnel is to be drilled through the humerus 1012. FIG. 33 illustrates the humeral sizer attachment 340 coupled to the distal portion 310d of the arm 310 of the guide 300. As described herein, other components can be attached to the arm 310 in lieu of the humeral sizer attachment 340, including but not limited to the handle assembly 1400.

Prior to introducing the humeral guide 300 to the surgical site, one or more retractors can be used to improve access and visualization to the surgical site. These retractors can be the same as were used in previous steps, and/or they can be other retractors. With the retractor(s) in place, the humeral guide 300 can be positioned for use at a surgical site, which in the present embodiment remains the human glenohumeral shoulder joint 1010 at which the glenoid 1018 and the humeral resection surface 1015 of the humerus 1012 are located. In at least some instances it may be advantageous to extend an incision being used to insert guide 300 to the surgical site to accommodate components such as the drill cannula 350 contacting bone.

The support rods 336a, 336b and bone pin clamps 332a, 332b can be attached to the guide 300 prior to delivering the guide 300 to the surgical site. This can be done using a T20 driver or other suitable assembly tool. The bone pin clamps 332a, 332b can be oriented based on the shoulder where the procedure is being performed (i.e., right or left shoulder) and the preferences of the surgeon, as described and illustrated further above, for example with respect to FIGS. 10A-10B. In the illustrated embodiment, the guide 300 is designated for use in a left shoulder procedure and the rods 336a, 336b and bone pin clamps 332a, 332b are appropriately disposed for the same. This configuration can assist in providing desirable anatomical fixation for a humeral drill bit(s) or guide pin(s) (e.g., the drill bit 380).

The humeral sizer attachment 340 can be used to locate the center of the humeral resection surface 1015 and determine the appropriate size for subsequent attachments and/or the prosthesis to be used, such as a stemless implant (e.g., the implant 1900 illustrated in FIGS. 41B-41D). Various sized humeral sizer attachments can be used depending, at least in part, on the size, anatomy, and age of the patient, as well as surgeon preference, among other factors. In the illustrated embodiment of FIG. 34A, the humeral sizer attachment 340 is a medium size, designated by an “M” thereon. The humeral resection surface 1015 can be used to select the sizer attachment that best fits inside a cortical rim of the humeral resection surface 1015. For example, as illustrated in FIG. 34A, the plate portion 342b of the humeral sizer attachment 340 can be aligned with the humeral resection surface 1015 such that the center opening 343a aligns with the centerpoint of the humeral resection surface 1015. The arm portion 342a of the sizer attachment includes a window 344 configured to align with the line 1011 formed on the bicipital groove, the bicipital grove separating a greater tubercle from a lesser tubercle of the humerus 1012. More particularly, the mark placed in the bicipital groove earlier during the procedure can be visible through the window 344. The plate portion 342b can be centered on the humeral resection surface 1015 and the marking 345 on the arm portion 342a that aligns with the mark in the bicipital groove allows the approximate size of the humeral resection surface 1015 to be determined. For example, in the illustrated embodiment, the sizer portion 342b of the attachment 340 is centered with respect to the humeral resection surface and the marking labeled 46 intersects to the cortex 1001 or outermost edge of the humeral resection surface 1015, sometimes referred to as the cortical rim 1001, as well as the line 1011.

As shown in FIG. 34B, the humeral sizer attachment 340 can be coupled to the guide 300, for example by moving it in a direction P, towards the distal end 310d of the arm 310, and rotating the knob 348 in a direction R to tighten it with respect to the arm 310. The distal portion of the humeral sizer attachment 340 or other modular attachment can be inserted through the rotator interval 1020, superior to the subscapularis 1017 and aligned substantially parallel to the humeral resection surface 1015. The hub 320 can be placed proximal to the lateral cortex 1023 of the humerus 1012 to guide the drill cannula 350 until the distal end of the drill cannula 350 meets the lateral cortex 1023 of the humerus 1012, setting the location and trajectory for a transhumeral bone tunnel. The sizer portion 342b can be placed substantially planar with the humeral resection surface 1015 and the appropriate marking 345 determined with respect to the humeral resection surface 1015 can be aligned with the cortical rim 1001 of the bicipital groove to help center the sizer portion 342b on the humeral resection surface 1015 such that the central opening 343a of the sizer portion 342b aligns with the centerpoint of the humeral resection surface 1015. Further, the sizer attachment 340 can be centered anterior to posterior by palpation and/or visual confirmation, and the sizer attachment 340 can be flush or substantially flush with the humeral resection surface 1015. This positioning of the guide 300 and associated humeral sizer attachment 340 ensures that a trajectory for a guide pin or drill passed through the drill cannula 350 is substantially central and substantially perpendicular to the humeral resection surface 1015.

After the guide 300 is placed proximal of the humerus with the plate portion 342b of the sizer attachment 340 substantially centered on the humeral resection surface 1015, the position of the sizer attachment 340 can be held by applying downward pressure in a direction D, towards the humeral resection surface 1015, at an approximate center of the plate portion 342b. Further, the drill cannula 350 can be pushed through the hub opening 322, in a direction B, along the longitudinal axis LC to engage the lateral cortex 1023 of the humerus 1012. The drill cannula 350 can be advanced, for example by applying a force on a bottom of the drill cannula, e.g., the base 358, with a hand, in the direction until the distal tip 350t of the distal portion 350d is pressed against the lateral cortex 1023 of the humerus 1012 marking a proximal end of the intended bone tunnel and defining the entry location for a drilling component. The ratcheting teeth 351 formed on the outer surface of the intermediate portion of the drill cannula 350 can provided for a one-way ratchet mechanism on the inner surface of the hub opening 322 to maintain the position of the drill cannula 350. The ratcheting teeth 351 allow the drill cannula 350 to pass distally through the hub opening 322 but prevents the drill cannula 350 from moving proximally thereby maintaining force on the surface of the bone at the distal end 350d of the drill cannula 350, thus creating a clamping force between humeral attachment 340 and the distal end 350d of the drill cannula 350. The drill cannula 350 passes through the hub opening 322 until the distal tip 350t meets the lateral cortex 1023 of the humerus 1012. The distal tip 350t of the drill cannula 350 and the humeral sizer attachment 340 create a clamping force on the humeral resection surface 1015 to maintain position of the guide 300 with respect to the humerus 1012.

While the drill cannula 350 is advanced in the direction B, a force opposed to the force in the direction B, as shown a force in a direction C, can be applied, for example by hand, to a top surface of the humeral guide 300, as shown at the hub 320. The competing forces in the directions B and C can be created by squeezing a hand, or by squeezing two hands together, providing initial securement to the humerus 1012. The one-way ratchet feature of the drill cannula 350 can maintain final position. As desired, pressure on the humerus 1012 created by the drill cannula 350 can be released by pressing the 328h of the locking screw 328 into the hub 320, allowing the drill cannula 350 to be slid opposite to the direction B, i.e., in the direction C, away from the humerus 1012.

As a result of this placement, the central opening 343a of the humeral sizer attachment 340, the hub opening 322, and the central opening 356 of the drill cannula 350 can all be aligned along the longitudinal axis LC. As shown, the humeral guide 300 is coupled to the humeral resection surface 1015, and thus the humerus 1012, such that a proximal portion 300p of the humeral guide 300, which can include, for example, the hub 320, is disposed below the humeral resection surface 1015, at a location that is opposed to the humeral resection surface 1015, while a distal portion 300d of the humeral guide 300, which can include the distal portion 310d of the arm 310, is disposed proximate to a rotator interval 1020, approximately aligned with the humeral resection surface 1015.

Locking components such as the spring-loaded release button 328 or one-way ratcheting mechanism can maintain the drill cannula 350 against the lateral cortex 1023. In some embodiments, the drill or drill bit 380 can be passed through the opening 356 of the drill cannula 350, and into the lateral cortex 1023 of the humerus 1012, to maintain the drill cannula 350 and the guide 300 in position as the guide 300 is further secured utilizing fixation features 330, such as the bone pin clamps 332a, 332b, disposed on the arm 310 and/or the hub 320 of the guide 300. Operation of the bone pin clamps 332a, 332b is described in detail above with respect to FIGS. 11C-11E. The drill bit 380 can be advanced into the humerus 1012 to assist in providing stability for the humeral guide 300, for instance when placing bone pins (e.g., the bone pins 370a, 370b) into the humerus. For example, the drill bit 380 can be advanced approximately 10 millimeters into the humerus 1012. Visual indication that the drill bit 380 has traveled to a location that provides desired docking of the guide 300 can be detectable by way of the indicator 381. In the illustrated embodiment, when the indicator 381 is disposed at the base 358, that is a visual indication that the drill bit 380 is disposed approximately 10 millimeters into the humerus 1012, which for the illustrated embodiment is a docked position for the guide 300.

After the bone tunnel location and trajectory are reliably set, the guide assembly 300 can be further secured to the humerus 1012 using bone pins 370a, 370b and the bone pin clamps 332a, 332b. The methods disclosed herein for attaching the guide 300 to the humerus 1012 allow for freedom of placement of the bone pins 370a, 370b based, at least in part, on surgeon preference and patient anatomy. Further, the guide 300, and related methods, allow for a more minimally invasive approach than traditional procedures with a smaller deltopectoral incision length and the ability to place the drill cannula 350 and the bone pins 370a, 370b percutaneously. Still further, the provided for design of the guide 300 and related components allow for micro-adjustments to be made prior to drilling a transhumeral pilot hole, which in turn allows fine tune centering of the sizer portion 342b on the humeral resection surface 1015 without requiring removal of the pins 370a, 370b and/or replacement of the pins 370a, 370b in new positions. This, in turn, prevents an excess of holes from being formed in the bone due to repositioning and decreases and/or minimizes the potential for bone fracture.

With the humeral guide 300 placed in position and the distal tip 350t of the drill cannula 350 contacting the lateral cortex 1023, bone pins 370a, 370b can be introduced through the bone pin clamps 332a, 332b to further maintain the location and position of the guide 300 with respect to the humeral resection surface 1015, as shown in FIG. 35B. In at least some instances, the skin incision may need to be extended distally to accommodate the drill cannula 350 contacting bone. Because of the configuration of the bone pins 370a, 370b, the relief section 375 can provide a tactile feel to the user after inserting it through the initial cortical bone, as the bone pins 370a, 370b can quickly advance without pressure to a far cortical wall. Advancing the bone pins 370a, 370b approximately an additional about 3 millimeters to about 5 millimeters can embed the bone pins 370a, 370b in a far cortex while simultaneously securing the threaded section 371 to resist cantilever forces and provide sufficient stability. If the bone pins 370a, 370b can be stopped prior to exiting the far cortex, that can assist in avoiding soft tissue injury.

It can be helpful to place bone pins 370a, 370b in the bone pin clamps 332a, 332b and/or plan placement of the pins 370a, 370b at locations on the humerus 1012 prior to drilling. Such planning can help ensure the pins 370a, 370b do not interfere with bone preparation instruments and final implant placement and impaction. In doing such planning, consideration can be given to soft tissue and neurovascular structures during placement of the pins 370a, 370b. Soft tissue off the bone at the entry point should be cleaned and any tissue wrapping during insertion should typically be avoided. In planning, the superior bone pin 370a should typically enter no higher than an entry point of the drill cannula 350 at the lateral cortex 1023 and can follow a trajectory approximately parallel with a plane that sits entirely, or substantially entirely, with the humeral resection surface 1015. Typically this superior pin 370a should not cross the axis LC of the drill cannula 350. Further, the planned position of the inferior bone pin 370b can be such that there is at least about 2 centimeters of vertical spacing from the superior bone pin 370a and the inferior bone pin 370b, providing added stability. In at least some embodiments, at least the inferior bone pin 370b can be placed percutaneously.

One or both of the rod-receiving portion 1310 and pin-receiving portion 1320 can be in an unlocked position such that the bone pin clamps 332a, 332b can move with respect to the rods 336a, 336b and the bone pins 370a, 370b. The bone pin clamps 332a, 332b can be directed to a preferred position and orientation on the humeral diaphyseal bone, such position being appreciable by a person skilled in the art, in view of the present disclosures. Further, the bone pins 370a, 370b can be of the nature that they provide tactile feedback as they are inserted into the bone.

In at least some instances, the superior bone pin 370a can be placed into bone. The bone pin clamp 332a associated with that pin 370a can be initially finger-tightened, using the locking nut 1340. Alternatively, or additionally, a ratchet wrench can be placed onto the locking nut 1340 and the nut 1340 tightened. The downward pressure in the direction D on the humeral sizer attachment 340 can be maintained while tightening the locking nut to provide counter resistance, helping to ensure no change in the positioning of the humeral guide 300. These same actions can be taken with respect to the inferior bone pin 370b and the bone pin clamp 332b. It is possible that the inferior bone pin 370b can be placed prior to the superior bone pin 370a.

The multi-degree freedom of movement afforded by the bone pin clamps 332a, 332b allows the surgeon to guide the bone pins 370a, 370b into the humerus 1012 at various locations and angles with respect to the humerus 1012 and/or the guide 300, for example in directions U and V as illustrated in FIG. 35B, respectively. In at least some embodiments, the pins 370a, 370b can be threaded, allowing for measured insertion of the pins 370a, 370b into the bone, for example by rotating the pins 370a, 370b relative to the bone. This can also help to provide the tactile feedback. If adequate bone pin purchase is not obtained based on the location of the bone pin clamps 332a, 332b and way the pins 370a, 370b are inserted, a readjustment of the bone pin clamps 332a, 332b location and/or position and/or a readjustment of the humeral guide 300 location and/or position may be appropriate. The adjustability of the bone pin clamps 332a, 332b allows the guide to be micro-adjusted or fine-tuned after the bone pins 370a, 370b are secured within the humerus 1012, for example to fine-tune centering of the humeral sizer attachment 340 on the humeral resection surface 1015, without requiring removal of the pins 370a, 370b and/or replacement of the pins 370a, 370b in new positions. Such micro-adjustments can be achieved by selectively unlocking the locking nut 1340 to make an adjustment(s). It can also be helpful to remove tension from the drill cannula 350, e.g., by pressing the release button 328, and/or re-adjusting a position of the humeral guide 300 and/or the sizer attachment 340 when making the adjustment(s). The micro-adjustments can be performed without removing the bone pins 370a, 370b. The micro-adjustments and/or adjusted tensioning of the drill cannula 350 can be performed to ensure desired placement of the humeral guide 300.

Once the adjustment(s) are complete, the locking nut 1340 can be locked and, if the drill cannula 350 had tension removed, the drill cannula 350 can be re-tensioned to test the new configuration. Any locking or unlocking of the locking nut 1340 can be performed using a wrench. This, in turn, prevents an excess of holes from being formed in the bone due to repositioning and decreases and/or minimizes the potential for bone fracture. A tip of each pin 370a, 370b can enter the near cortex, drop into cancellous bone, and then dock in the far cortex. The threads 371 formed on the pin 370a, 370b can help provide optimal fixation in the near cortex. The pins 370a, 370b can maintain spacing of at least about 5 millimeters apart, have bi-cortical fixation, and be below an entry point of the drill cannula 350 to ensure clearance for humeral bone preparation instrumentation and implants. As pins 370a, 370b are placed, care should typically be given to soft tissue and neurovascular structures to avoid causing the pins 370a, 370b to contact and/or pass through such tissue or structures. A surgeon can plan placement of the pins 370a, 370b onto the bone prior to performing drilling.

When the desired position of the guide 300 is set, the bone pin clamps 332a, 332b can be moved to a locked position by tightening the locking nut 1340 (see FIG. 11E), for example with a wrench, to secure the position of the guide 300 with respect to the humerus 1012. When tightening the clamp nut 1340, it can be helpful to provide counter torque to the respective bone pin 370a, 370b disposed therein and/or the humeral guide 300 to help ensure the central position does not change. The humeral guide 300 can remain at the set location for a remainder of the surgical procedure to assist in guiding other instruments for accurate humeral preparation. The drill bit 380 can be subsequently removed from the distal cannula 350, or it can be used to perform the drilling to form the transhumeral bone tunnel.

As shown in FIG. 35C, the drill bit 380, or a different drill bit or guide pin, can be passed through the drill cannula 350 in a direction W to create the transhumeral bone tunnel following the trajectory set by the guide assembly 300. In the illustrated embodiment, the formed transhumeral bone tunnel is at a different location with respect to the humerus 1012 than the transhumeral bone tunnel described with respect to FIGS. 27A and 28A-28B, with the start of the transhumeral bone tunnel of FIG. 35C being illustrated further down, or more distally down, the lateral cortex 1023. In some embodiments, a primary diameter of the drill bit 380 can be approximately 3.5 mm, which can be a standard size for humeral drills. A proximal end can include a hub similar to the hub 386 of the drive shaft 385 (see FIG. 15), which as shown can include a modified trinkle connection, that can be rotated and/or advanced to provide similar movement at a distal end 380d of the drill bit 380, including at a distal tip 382. A modified trinkle connection can be used to connect the drill 380 to power, and the drill can be driven or otherwise advanced through the drill cannula 350 and into and through the humerus 1012 until a distal tip 382 formed at a distal end 380d of the drill 380 is passed out of the humeral resection surface 1015, through the central opening 343a of the sizer attachment 340. As a result, the drill 380 can exit the humerus 1012 substantially center with respect to and substantially perpendicular to the humeral resection surface 1015, creating a transhumeral tunnel 1029 through which the longitudinal axis LC extends. The drill 380 can be removed from the surgical site once the transhumeral tunnel 1029 has been created, as shown in FIG. 35D.

Once the bone tunnel has been created, guide pins or drivers with various attachment features at their distal tip(s) can be passed through the drill cannula and attached to various modular attachments inserted into the rotator interval. The remaining steps of humeral preparation can be performed through the transhumeral bone tunnel 1029 using the humeral guide 300 to maintain correct position and alignment for each step. As provided for herein, in at least some instances, a handle assembly, like the handle assembly 1400, can be used in conjunction with the guide 300 to perform such steps.

To allow for other instruments or tools to be attached to the guide 300, the humeral sizer attachment 340 can be removed from the guide 300, as shown in FIG. 36A. To remove the sizer attachment 340, the knob 348 can be rotated in a counterclockwise direction CC to allow the humeral sizer attachment 340 to be disconnected from the distal end 310d of the arm 310, as illustrated by arrow K. After the humeral sizer attachment 340 is disconnected, the tension supplied by the drill cannula 350 to the humeral resection surface 1015 may need to be adjusted to ensure rigidity of the humeral guide 300 before performing tasks like humeral reaming.

The adapter 390 can be coupled to the distal end 310d of the arm 310 as shown in FIG. 36B. The alignment line 390m formed on the adapter 390 can be aligned with the alignment line 361a disposed on the distal portion 310d of the arm 310. The adapter 390 can receive the distal end 310d of the arm within the opening 391 such that the adapter 390 can translate along the arm 310 as described herein. In other embodiments, the adapter 390 can be associated with the arm 310 when used with the humeral sizer attachment 340. For example, in some embodiments the adapter 390 can be disposed on the arm 310 throughout an entirety of the procedure because the adapter 390 is not as easily slid on and off the arm 310.

### Using a Humeral Guide(s) to Ream the Humeral Resection Surface

One example of humeral preparation actions that can be performed while using the humeral guide 300 includes reaming the humeral resection surface 1015. As described herein, the reaming can be performed using the reamer attachment 500 in conjunction with the handle assembly 1400.

A size of the reamer attachment 500 and/or the tool 502 associated with the reamer attachment 500 can be selected based on the humeral resection surface 1015 size determined by way of the sizer attachment 340. Prior to insertion into the joint space, the reamer attachment of the appropriate size can be assembled and secured to a handle assembly, like the assembly 1400 of FIGS. 17A-17B.

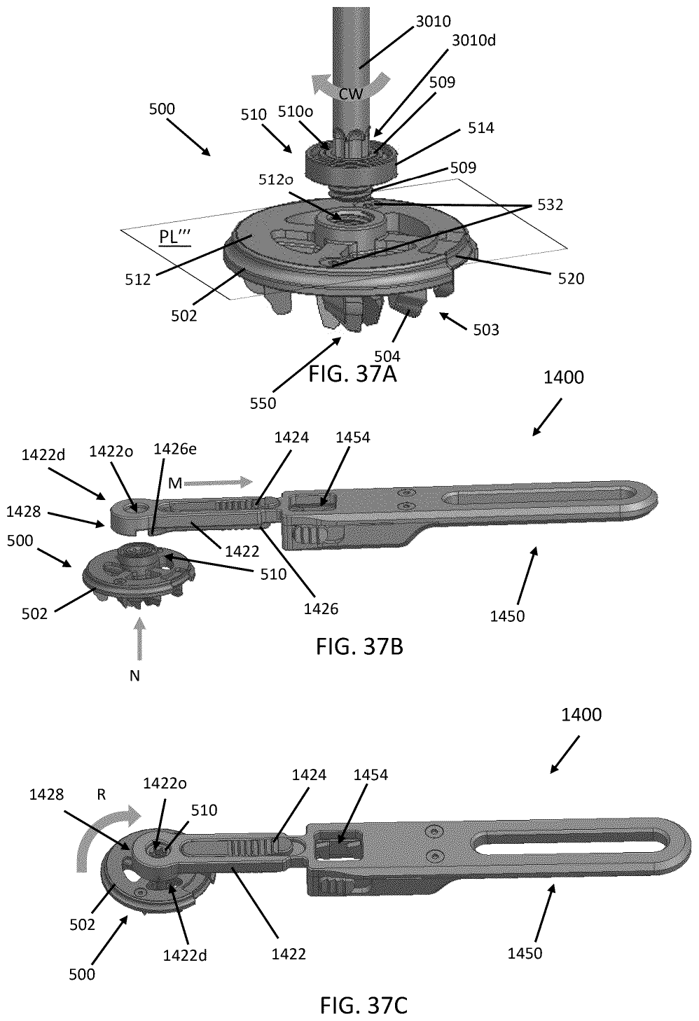

FIG. 37A illustrates an embodiment of how the tool 502 can be associated with the mount 510. As illustrated, the mount 510 can be coupled to the distal end 3010d of the shaft 3010, as shown a T30 driver, by way of a hex-key engagement between that distal end 3010d and walls that define the opening 5100 of the mount to assist in delivering the mount 510 to the tool 502. The mount 510 can be coupled to the tool 502 by rotating the shaft 3010, and thus the mount 510, in a clockwise direction CW, allowing threads on the shaft 509 of the mount 510 to mate with threads formed on walls that define the opening 5120 of the tool 502. After the mount 510 and tool 502 have been attached, the shaft 3010 can be detached from the mount 510.

The assembled reamer attachment 500 can then be coupled to a handle assembly, such as the handle assembly 1400, for use of the attachment 500 to ream the humeral resection surface. As shown in FIG. 37B, the attachment portion 1420 of the handle assembly 1400 can receive the reamer attachment 500 by sliding and/or pulling the latch 1424 proximally, in a direction M towards the gripping portion 1450. The reamer attachment 500 can then be inserted into the chamber 1428 by advancing it in a direction N so it can be coupled to the distal end 1422d of the arm 1422, within the chamber 1428. Once the reamer attachment 500 is properly seated within the chamber 1428, with the mount 510 proximate to the opening 14220, the latch 1424 can be released, causing it to return to its position prior to application of the force in the direction M, allowing the capture plate 1426 to slide back towards the chamber 1428, and in turn capture the reamer attachment 500 to secure its location with respect to the handle assembly 1400 in a locked position or configuration in which the location of the reamer attachment 500 is locked with respect to the arm 1422. More particularly, the arms 1426c, 1426f (only 1426e is visible in FIG. 37B) can engage the mount 510 of the reamer attachment 500 to secure or otherwise lock the reamer attachment 500 to the handle assembly 1400. When the reamer attachment 500 is in the locked position, the reamer tool 502 should be able to spin freely by hand in a direction R as shown in FIG. 37C.

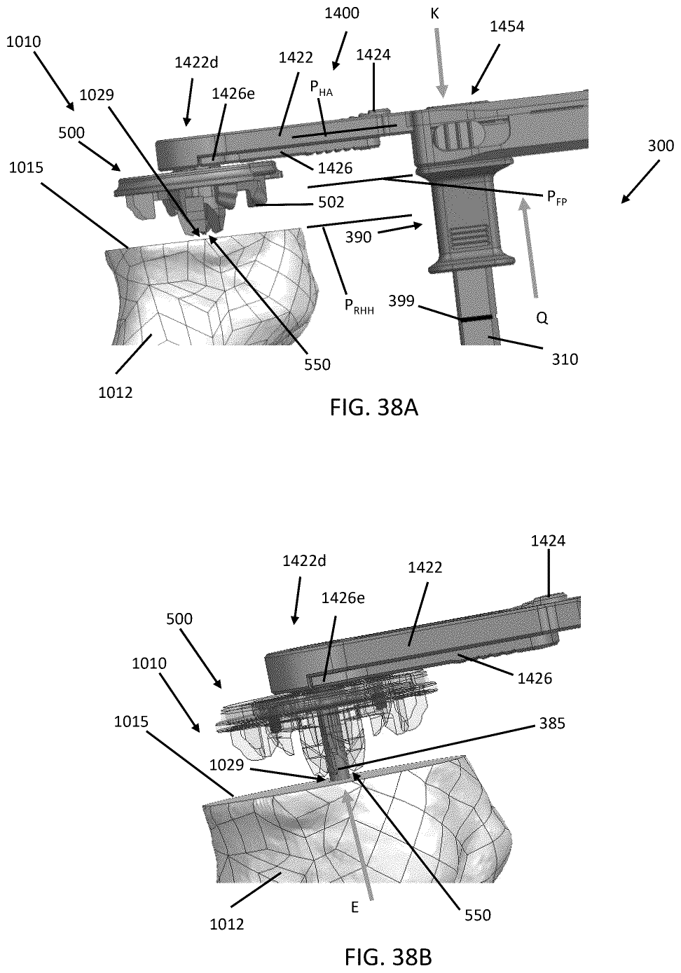

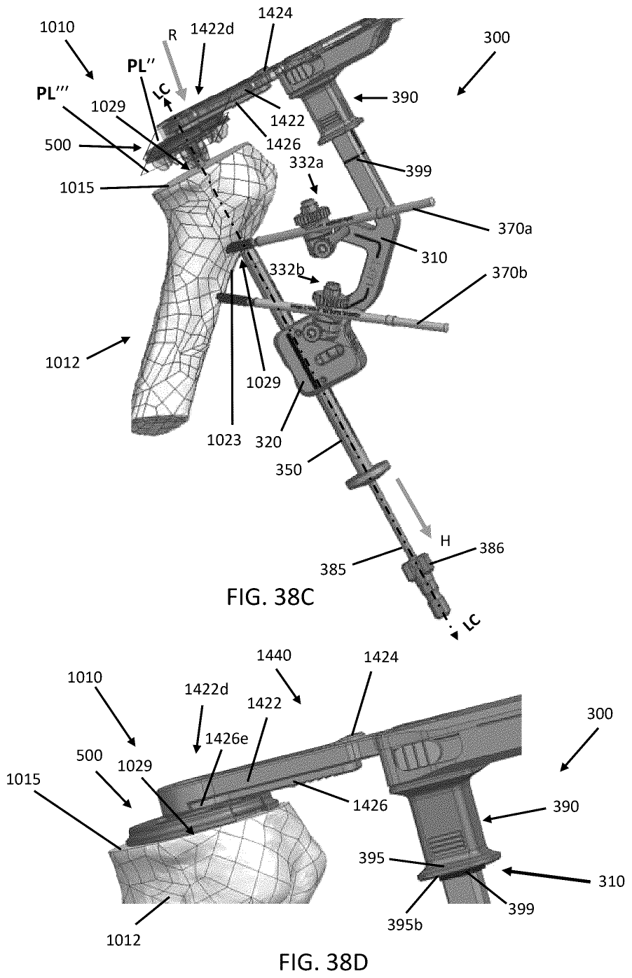

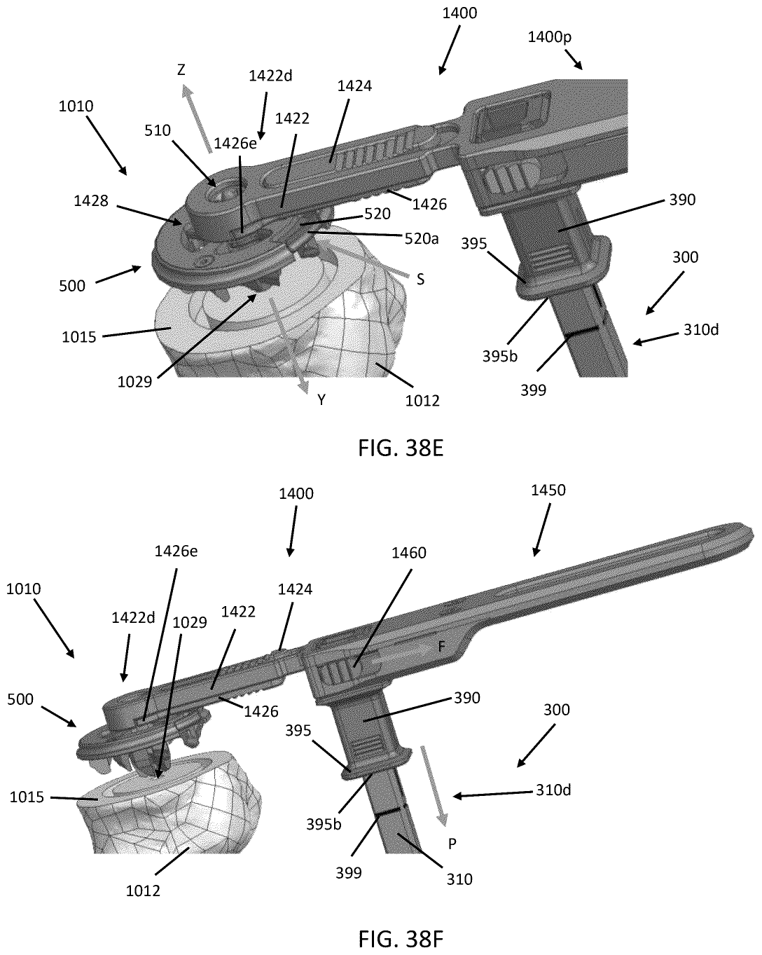

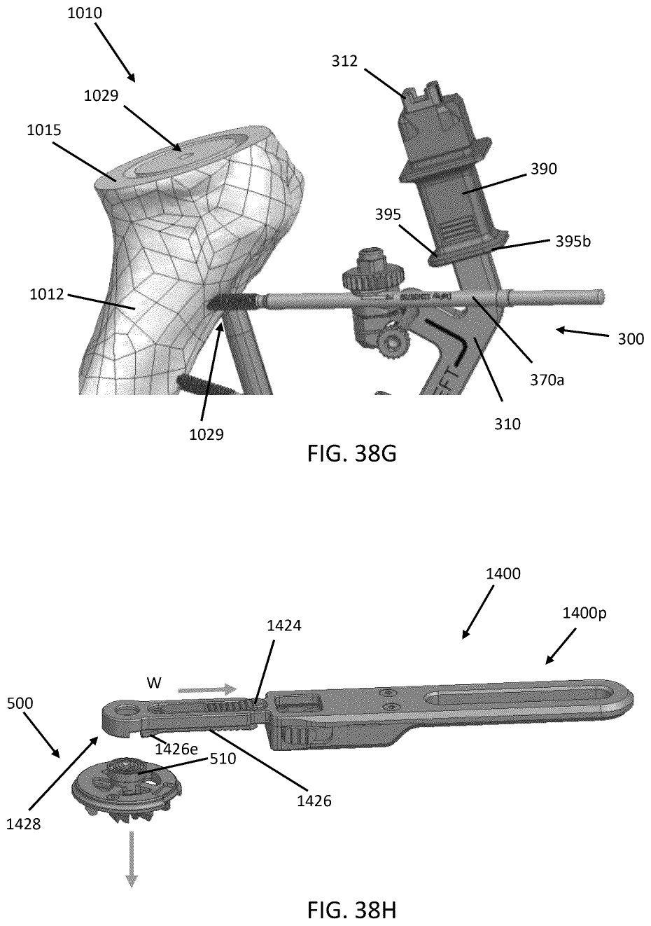

With reference to FIG. 38A, the handle assembly 1400 with the reamer attachment 500 coupled thereto can be navigated into the glenohumeral shoulder joint 1010 through the rotator interval 1020 (illustrated better in other figures) and the reamer attachment 500 can be positioned over the humeral resection surface 1015. In this location, the handle assembly 1400 can be considered to be in a superior position on the humeral resection surface 1015. The handle assembly 1400 can be coupled to the humeral guide 300 by moving the assembly 1400 in the direction K until the attachment feature 312 of the distal portion 310d of the arm 310 is received within the guide-receiving opening 1454 of the assembly 1400 and the guide 300 and handle assembly 1400 are coupled thereto. Further, it at least some embodiments, the adapter 390 can be slid along the arm 310 in the direction Q to engage the handle assembly 1400 through the guide-receiving opening 1454. Securing the location of the handle assembly 1400 to the humeral guide 300 by way of the adapter 390 maintains planar and central axis alignment while the tool, e.g., the reamer 502, associated with the attachment, e.g., the reamer attachment 500, is operated. When the adapter 390 has helped secure the location of the handle assembly 1400 with respect to the humeral guide 300, audible and/or tactile feedback can notify the user of the securement between the two. Visual confirmation of the securement is also present to see, as seen in FIG. 38A, with there being face-to-face contact between the flared portion 392 of the adapter 390 and the handle 1400 with no visible gaps therebetween. When connecting the handle assembly 1400 to the guide 300, proper parallel alignment between a plane PHA extending through the distal portion 1422d of the elongate arm 1422 and a plane PHP defined by the flared portion 392 of the handle should be achieved, thus indicating proper parallel placement between the plane PHA and a plane P_{RHH} extending through the humeral resection surface 1015. By maintaining the handle assembly 1400 at the desired position with respect to the humeral guide 300, planar and central axis alignment can be maintained while treating the humeral resection surface 1015.

The drive shaft 385 can be navigated into the joint space 1010 by moving it in a direction E, as shown in FIG. 38B, to align with the reamer cannulation, i.e., the central opening 550 of the reamer attachment 500. A handle (not shown) can be used to assist in navigating and placing the drive shaft 385 through the drill cannula 350 and delivering the drive shaft 385 to the surgical site. As discussed above, the reamer attachment 500 can have a quick-connect feature that connects to the drill geometry, e.g., a feature the same as or akin to the grove 384′ (see, e.g., FIGS. 19C-19D), once the reamer attachment 500 and drive shaft 385 are properly aligned. In some embodiments, configurations like those described with respect to the reamer attachment 700 and the driver 290 can be used in conjunction with the reamer attachment 500 (or other attachments, e.g., the blazer attachment 600) and the drive shaft 385. An audible and/or tactile click can be noticed once connected. The drive shaft 385 can be passed towards the surgical site by providing a slight side-to-side twisting motion to engage various quick connect features associated with the reamer attachment 500. The drive shaft 385 can be rotated and/or pulled by hand to confirm connection with the reamer attachment 500.

As shown in FIG. 38C, the drive shaft 385 can pass through the drill cannula 350, and thus through the cannula-receiving opening 322 of the hub 320, and into and through the central opening 550 of the reamer attachment 500. The distal end 385d and the distal tip 387 can be captured within the central opening 550 as discussed with respect to various embodiments, such as the descriptions and illustrations of FIG. 18C and FIGS. 19C-19D. Further, the plane PL″″ defined by the reamer attachment 500 (see FIG. 18A), which can be substantially parallel to the plane PL″ defined by the attachment portion 1420 of the handle assembly 1400, can be orthogonal, or substantially orthogonal, to the longitudinal axis LC, as illustrated in FIG. 38C.

With the reamer attachment 500 secured to both the handle assembly 1400 and the drive shaft 385, the reamer attachment 500 can be operated to perform reaming actions on the humeral resection surface 1015. If a handle was used to position the drive shaft 385 at the surgical site and/or couple it to the reamer attachment 500, and thus the handle assembly 1400, the handle can be removed and a power source (not shown), such as a drill or motor, can be coupled to a proximal end of the drive shaft 385. In use, the reamer 502 can be placed in a forward rotation position or configuration, and, in at least some instances, can be started at a low speed prior to having the reamer 502 contact the humeral resection surface 1015. As shown in FIG. 38C, the reamer 502 can be advanced towards the humeral resection surface 1015 by pulling the drive shaft 385, often in a light manner, in a direction H, towards the hub 320 and/or by pushing down in a direction R on the handle assembly 1400 in-line with the mount 510, i.e., at the distal portion 1422d of the arm 1422. The force/forces can be applied to ream the humerus 1012 until the reamer 502 is flush with the humeral resection surface 1015, as illustrated in FIG. 38D. As shown, when the bottom portion 395b of the handle 395 of the adapter 390 reaches the demarcation line 399, that indicates to the surgeon that the reamer 502 is flush, or substantially flush, with the humeral resection surface 1015. For example, the reamer attachment 500 can be advanced towards the humeral resection surface 1015 until the bottom portion 395b of the handle 395 meets the line 399. Reaming the bone to the indicated depth can create a geometry in the humeral resection surface 1015 that corresponds to the geometry of the chosen implant.

After reaming is complete, the reamer attachment 500 can be removed from the humerus 1012. This can be done, for example, under power by pushing up with the drive shaft 385 and/or the handle assembly 1400 in a direction Z to reposition the reamer back up into the joint space 1010, as shown in FIG. 38E. In embodiments in which the reamer attachment 500 includes a release button, like the button 520, the drive shaft 385 can be used to slowly rotate the reamer 502 until the terminal edge 520a of the release button 520 is visible. After the reamer 502 has stopped spinning, the terminal edge 520a can be pushed radially inwards, towards the mount 510, in a direction S, to release the drive shaft 385 from the reamer attachment 500, and thus the handle assembly 1400. The drive shaft 385 can be moved in a direction Y, opposite to the direction Z, to detach the reamer attachment 500 from the drive shaft 385. After the drive shaft 385 is detached from the reamer attachment 500, it can be pulled back such that it is just below the cut plane defined by the reamed humeral resection surface 1015 and is not completely removed because the same drive shaft 385 can be used in subsequent steps, such as aiding in guidance for blazer preparation and/or operation.

As shown in FIG. 38F, the handle assembly 1400 can be subsequently detached from the humeral guide 300 so that the handle assembly 1400 can be removed from the joint space 1010. More particularly, the release latch 1460 can be slid in the direction F, towards the handle 1450 of the handle assembly 1400, and the adapter 390 can be slid in the direction P, along the arm 310, to detach and remove the handle assembly 1400 from the humeral guide 300. The resulting configuration of a reamed humeral resection surface 1015, the handle assembly 1400 disconnected from the guide 300, the guide 300 remaining in place with respect to the humerus 1012, and the adapter 390 sitting proximate to or flush with the attachment portion 312 of the arm 310 is shown in FIG. 38G. The bottom 395b of the handle portion 395 of the adapter 399 can be flush with and/or engage with the demarcation line 399 (not visible as a result).

FIG. 38H illustrates detachment of the reamer attachment 500 from the handle assembly 1400. This typically occurs outside the joint space 1010. As shown, the latch 1424 can be pulled, pushed, or otherwise slid in a direction W, towards the proximal portion 1400p of the handle assembly 1400, to release the mount 510 from the chamber 1428, allowing the reamer attachment 500 to drop away from the handle assembly 1400 as shown. The mount can be disposed of when it is designed for single use.

### Using a Humeral Guide(s) to Broach the Humeral Resection Surface

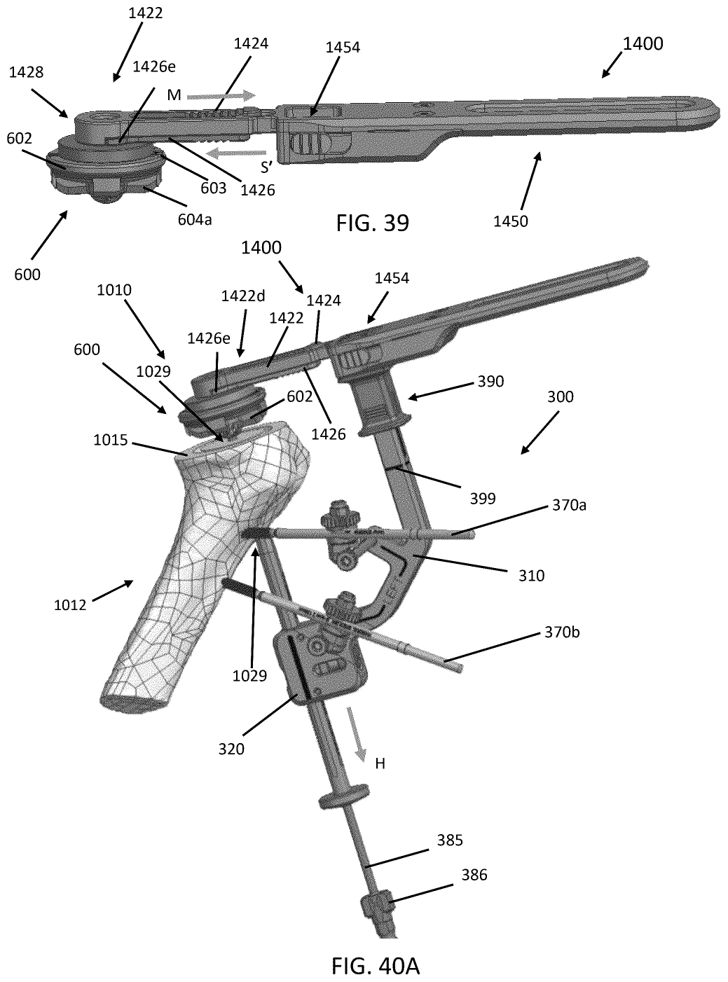

FIG. 39 illustrates the handle assembly 1400 have the blazer attachment 600 coupled thereto. The blazer attachment 600 can be coupled or mounted to the handle assembly 1400 in a manner similar to the reamer attachment 500, with at least some relevant distinctions being described above with respect to FIGS. 20A-20D. Accordingly, the latch 1424, which can be biased towards the chamber 1428 in the direction S′, can be pulled or otherwise slid in the direction M with a sufficient force to overcome the biasing force in the direction S′. The blazer attachment 600 can be brought towards the receiving portion 1452 of the handle assembly 1400 and the mount 610 (not visible in FIG. 39, but see FIGS. 20A and 20C) of the blazer attachment 600 can be disposed within the chamber 1428. The arrow 603 disposed on the blazer 602 can be pointing towards the proximal portion 1400p of the handle assembly 1400, and thus towards the alignment slot 1426s (not visible in FIG. 39, but see FIG. 17B) formed in the capture plate 1426. Once the blazer attachment 600 is disposed in the chamber 1428 as desired, the force applied in the direction M can be removed, allowing the capture plate 1426 to slide back towards the chamber 1428, and in turn capture the blazer attachment 600 to secure its location with respect to the handle assembly 1400. More particularly, the arms 1426c, 1426f (only 1426e is visible) can engage the mount 610 (not visible in FIG. 39, but see FIGS. 20A and 20C) of the blazer attachment 600 to secure or otherwise lock the blazer attachment 600 to the handle assembly 1400. The protrusion 613p (not visible in FIG. 39, but see FIGS. 20A and 20C) can be engaged by walls of the arms 1426c, 1426f that form the alignment slot 1426s of the capture plate 1426 (not visible in FIG. 39, but see FIG. 17B), thus providing known positioning of the blazer fin 604a with respect to the handle assembly 1400. The protrusion 613p can also providing mating capabilities when it engages the walls of the arms 1426e, 1426f that form the alignment slot 1426s. The selected blazer attachment 600 can be based on determinations that were made with the humeral sizer attachment 340. To the extent the mount 610 is not already coupled to the blazer 602, the attachment can be performed, for example using the procedures described above with respect to FIGS. 20A-20C.

With reference to FIG. 40A, the handle assembly 1400 with the blazer attachment 600 coupled thereto can be navigated into the glenohumeral shoulder joint 1010 through the rotator interval 1020 (illustrated better in other figures) and the blazer attachment 600 can be positioned over the reamed humeral resection surface 1015. The handle assembly 1400 can be coupled to the humeral guide 300 in the same manner as described above with respect to FIG. 38A, among other techniques disclosed herein or otherwise known to those skilled in the art in view of the present disclosures. Accordingly, the adapter 390 can be slid along the arm 310 to engage the handle assembly 1400 through the guide-receiving opening 1454. Securing the location of the handle assembly 1400 to the humeral guide 300 by way of the adapter 390 maintains planar and central axis alignment while the tool, e.g., the blazer 602, associated with the attachment, e.g., the blazer attachment 600, is operated.

The drive shaft 385 can be used to operate the blazer attachment 600 as well, the drive shaft 385 being able to confirm and maintain axial alignment during operation of the blazer attachment 600, e.g., during impaction as described herein. The blazer attachment 600 can be coupled to the drive shaft 385 (see FIGS. 40A and 40E) in a manner similar to the way the reamer attachment 500 is coupled to the drive shaft 385 as described at least with respect to FIGS. 38B and 38C. Accordingly, a repetitive description of the same is unnecessary. After the blazer attachment 600 has been secured to the drive shaft 385, the drive shaft 385 can be rotated to confirm connection with the reamer attachment 600.

Subsequently, the blazer attachment 600 can be operated to perform broaching of the reamed humeral resection surface 1015. The drive shaft 385 can be rotated to align the arrow 603 (see FIG. 20C) disposed on the blazer 602 with the most lateral aspect of the humerus and/or as desired for final stemless implant insertion, if such alignment has not already been done and/or maintained. An impaction tool can be introduced into the glenohumeral shoulder joint 1010, for example via the rotator interval 1020, to assist in operating the blazer 602. One non-limiting example of such a tool, an impaction tool 900, also referred to as an impactor handle, is shown in FIG. 40B.

The impaction tool 900 can include a handle portion 902 and a distal receiver 907 configured to receive a removable and replaceable end effector 904, the end effector in the illustrated embodiment being an impactor of an impactor handle adapter, also referred to as a blazer-engagement (or other “blaze” and “broach” terms indicated as being synonymous herein or otherwise known to those skilled in the art in view of the present disclosures) end effector or a flat impactor tip, among other names. The handle portion 902 includes a wide handle 906 disposed at a proximal end 902p of the handle portion 902 and an elongate rod or shaft 908 extending therefrom. In the illustrated embodiment, the wide handle 906 and the elongate rod 908 are of a unitary construction, the elongate rod 908 having a distal end 908d that doubles as a distal end 902d of the handle portion 902. The distal ends 902d, 908d includes the distal receiver 907 that is configured to receive and mate with the end effector 904. The wide handle 906 can include one or more surfaces adapted to be grasped by a user and/or engaged by tools, e.g., a hammer or mallet, to provide a force in a direction A to the tool 900. The applied force can be passed through the elongate rod 908 and to the end effector 904 by way of the elongate rod 908. As shown, an outer surface of the rod 908 can include gripping features 910 formed thereon.

The end effector 904 is illustrated in greater detail in FIG. 40C. It can be securely mated to the distal receiver 907 using any techniques known to those skilled in the art for coupling two components together, such as a threaded connection, a snap-fit connection, male-female engagement mechanisms, etc. In the illustrated embodiment, the end effector 904 includes one or more engagement features, as shown opposed forks or tabs 905 extending from a base 903, configured to engage the handle assembly 1400. Each of the forks 905 can have opposed, flat tips, each fork 905 having a right angle that defines the tip.

As shown in FIGS. 40D-40F, in use, the forks 905 and base 903 can be in contact with and/or disposed around the attachment portion 1420 of the assembly 1400. More particularly, to start, the forks 905 can be positioned in a space formed by the cutouts 1448 (see FIGS. 17A-17C). The configuration of the forks 905 and base 903 allow the end effector 904 to be easily slid along and grasp the attachment portion 1420 of the handle assembly 1400 at a desired location. In the illustrated embodiment, the forks 905 are at least disposed around opposed sides of the body 1422, while the base 903 is in contact with a top surface of the body 1422. As the end effector 904 is being positioned at a desired location, an arm of the patient can be moved in adduction and an external rotation, often slight, can be provided to ensure an axis of impaction, which can be defined by a length of the rod 908, is not obstructed by the anatomy of the patient.

As shown in FIG. 40E, the rod 908 can extend out of the glenohumeral shoulder joint 1010, through the rotator interval (illustrated better in other figures), to allow for a force to be applied to the handle assembly 1400 remotely, via the wide handle 906 (not visible) disposed outside of the body. Accordingly, a force applied to the wide handle 906 can translate through the tool 900, via the rod 908, and to the end effector 904. A person skilled in the art will appreciate the end effector 904 can have other configurations and a variety of other end effectors can be selectively mounted to the attachment end 1420 of the handle assembly 1400, whether for use in broaching actions or other actions performed during surgical procedures that employ the humeral guides and/or the handle assemblies of the present disclosure or otherwise derivable from the present disclosures.

In use, the blazer attachment 600, and thus the blazer 602, can be operated by both operating the drive shaft 385 in a manner similar to operation of the reamer attachment 500, and thus the reamer 502. That is, the blazer attachment 600 can be advanced towards the reamed humeral resection surface 1015 by pulling the drive shaft 385 in the direction H, towards the hub 320, as shown in FIG. 40E. Because operation of the blazer 602 can often require a greater amount force to be applied than is needed to operate the reamer 502, the impaction tool 900 can also impart a force onto the blazer 602. As shown, the impaction tool 900 can be placed such that the end effector 904 contacts the body 1422 of the attachment portion 1420 of the handle assembly 1400. A user can apply a force, such as by impacting or otherwise hitting the wide handle 906, with a hammer or mallet to drive a force through the tool 900, through the attachment portion 1420, and to the blazer attachment 600 in the direction A. Impacting on a center is not able to be achieved due to the anatomy, which is, at least in part, why the present solution(s) is beneficial. In some embodiments, the end effector 904 can include a boss or threaded portion that forms an angle approximately in the range of about 5° to about 10° with respect to a base portion of the end effector 904, which can allow a center to be approached while avoiding tissue interference further proximal of the elongate rod 908. Additionally, or alternatively, a force can be applied to the blazer attachment 600 by sliding the end effector 904 along the body 1422 of the attachment portion 1420, for example towards the location of the blazer attachment 600 in a direction B as illustrated in FIG. 40D. As the force(s) is being applied, it can be helpful to support the elbow of the patient.

The force from the impaction tool 900 and/or the force from the drive shaft 385 can be applied until the blazer 602 sits flush with the reamed humeral resection surface 1015, as shown in FIG. 40F. As shown, when the bottom portion 395b of the handle 395 of the adapter 390 reaches the demarcation line 399, that indicates to the surgeon that the blazer 602 is flush with the reamed humeral resection surface 1015. For example, the blazer attachment 600 can be advanced towards the humeral resection surface 1015 until the bottom portion 395b of the handle 395 meets the line 399.

After broaching is completed, a force in the opposite direction of the direction A, as shown in FIG. 40G a direction A′, can be provided to pull the blazer 602 out of the humerus 1012. After the blazer attachment 600 is free from the prepared bone and is within the glenohumeral shoulder joint 1010, the impaction tool 900 can be disconnected from the attachment end 1420 of the handle assembly 1400 and pulled upwards back through the rotator interval 1020 and out of the body.

Subsequently, the handle assembly 1400 and the blazer attachment 600 coupled thereto, can be disconnected from the drive shaft 385 and removed in a manner similar to the removal of the handle assembly 1400 and the reamer attachment 500. Such actions are supported by FIG. 40H, and other disclosures herein, and such a further explanation of the same is unnecessary. Additionally, the blazer attachment 600 can be disconnected from the handle assembly 1400 using techniques provided for herein, e.g., sliding the latch 1424 to disengage the mount 610 from the capture plate 1426 to allow the blazer attachment 600 to be detached from the handle assembly 1400.

### Using a Humeral Guide(s) to Introduce an Implant(s) to the Humeral Resection Surface

After reaming and broaching the humeral resection surface 1015, an implant, such as an implant 1900 as shown in FIGS. 41B-41D, can be introduced to the glenohumeral shoulder joint 1010. As used herein, the term implant typically refers to a component that is disposed in a surface of a bone and/or tissue, while a prosthesis is referred to a portion that couples to such an implant and extends more outwardly from the implant, bone, and/or tissue (though it can be at least partially disposed in the implant, bone, and/or tissue), although the term implant, in at least some contexts, can refer to a prosthesis as well. A person skilled in the art will appreciate other actions besides reaming and broaching can be performed to the humeral resection surface 1015 prior introducing an implant. For example, one or more holes can be drilled at the surgical site, through the bone, to allow suture to be placed in the hole(s) and subsequently be trapped by the implant 1900 when the implant 1900 is inserted into the humeral resection surface 1015. The suture can then be used to approximate soft tissue, for instance at completion of the procedure.

FIG. 41A illustrates the resulting humeral resection surface 1015 after the reaming described with respect to FIGS. 37A-38H and the broaching with respect to FIGS. 39-40H. The humeral guide 300 (not illustrated) can remain in place. As shown, a humeral drive shaft 385, also referred to as a drive shaft, can remain at the surgical site, with a tip 387 of the humeral guide shaft 385 located proximate to—and in the illustrated embodiment disposed just above—the reamed and broached humeral resection surface 1015. In alternative embodiments, the drive shaft 385 can be replaced by an alternative drive shaft or drill bit 1380 (see, e.g., FIG. 41C). Although FIG. 41C illustrates the drill bit 1380, its inclusion after FIG. 41A and before FIG. 41D is not intended to imply the humeral guide shaft 385 as shown in FIG. 41A is removed and replaced by the drill bit 1380 as shown in FIG. 41C and then returned in place of the drill bit 1380 in FIG. 41D. Rather, the illustration of the drill bit 1380 in FIG. 41C is intended to show another tool that can be used in conjunction with placing the implant 1900 at the surgical site. For example, as discussed with respect to FIGS. 12C and 12D, an embodiment of a drill bit with features to couple to an attachment, such as drill bit 1380, can be used throughout the procedure in lieu of a separate drill bit and drive shaft, such as drill bit 380 and drive shaft 385. Further, FIG. 41C is intended to show additional detail about the implant 1900.

As shown in FIG. 41B, an inserter tool 800′, in the illustrated embodiment a stemless baseplate inserter that operates in a scissors-like manner, also referred to as a grasper, snaps, curved snaps, or a snaps tool, can be used to grasp the implant 1900, as shown a stemless implant, for insertion into the humeral resection surface 1015. Although not illustrated in FIG. 41B, in some embodiments, the tool can grasp one of four central support features 1901 of the implant 1900. A person skilled in the art will appreciate how inserter tools like the tool 800′ can be operated, and thus a further description of the same is unnecessary. Further, the implant 1900 can be similar to those known to those skilled in the art. The implant 1900 generally can be configured in a manner that is complementary to the shape and configuration formed in the humeral resection surface 1015 so that the implant can sit within the humeral resection surface 1015. The implant 1900 can include a top surface 1902 having a plurality of openings or holes 1904 formed therein, the holes 1904 being used, for example, to secure sutures that may be useful in the repair of surrounding soft tissues. For example, suture can be used to repair a subscapularis (if removed; the present disclosure affords the ability to perform procedures without removing the subscapularis, though the present disclosure can be used in conjunction with detaching a subscapularis as well) or other soft tissue that was removed to complete a procedure. The holes 1904 provide a good mounting point to route suture to ensure tissue repair can heal without micromotion.

The stemless implant 1900 can be introduced into the joint space 1010 in a manner similar to the way other instruments have been inserted to the joint space 1010, such as by going through rotator interval 1020, and thus illustration of the same is unnecessary. The implant 1900 can be placed over the tip 387 of the drive shaft 385, which is illustrated with respect to the tip 1382 of the drill bit 1380 in FIG. 41C. A geometry of the implant 1900 should be aligned with the complementary geometry formed in the humeral resection surface 1015, the complementary geometry formed in the humeral resection surface 1015 being primarily formed by the blazer attachment 600. That is, portions of the implant 1900 can be designed to fit in portions broached by the blazer fins 604a, 604b. In at least some embodiments, including as illustrated in FIG. 41D, as the implant 1900 is maneuvered to enter the cuts formed in the humeral resection surface 1015, it can seat such that the implant 1900 is approximately halfway into the humeral resection surface 1015, for instance applying light finger pressure. Further, as shown in FIG. 41D, the tip 387 of the humeral drive shaft 385 can be slid into a central opening or cannulation 1906 formed in the implant 1900 (FIG. 41C shows the same with respect to the tip 1382 of the drill bit 1380). In at least some embodiments, a surface 1903 that defines the central opening 1906 can be tapered to help funnel components into the central opening 1906 as desired. Alternatively, the humeral guide shaft 385 can be advanced into the central opening 1906 after the implant 1900 has been placed on the prepared humeral cut plane, i.e., the humeral resection surface 1015.

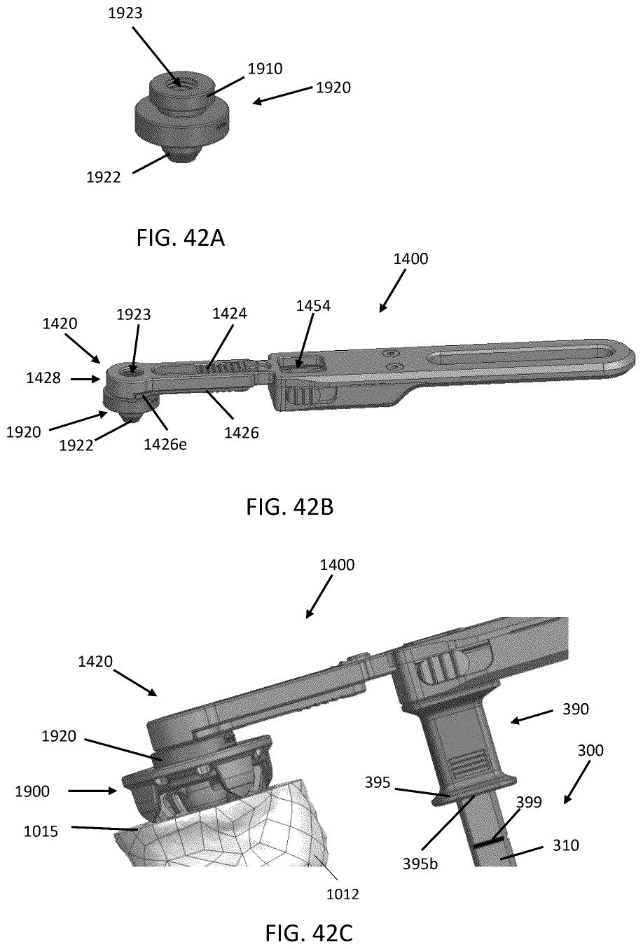

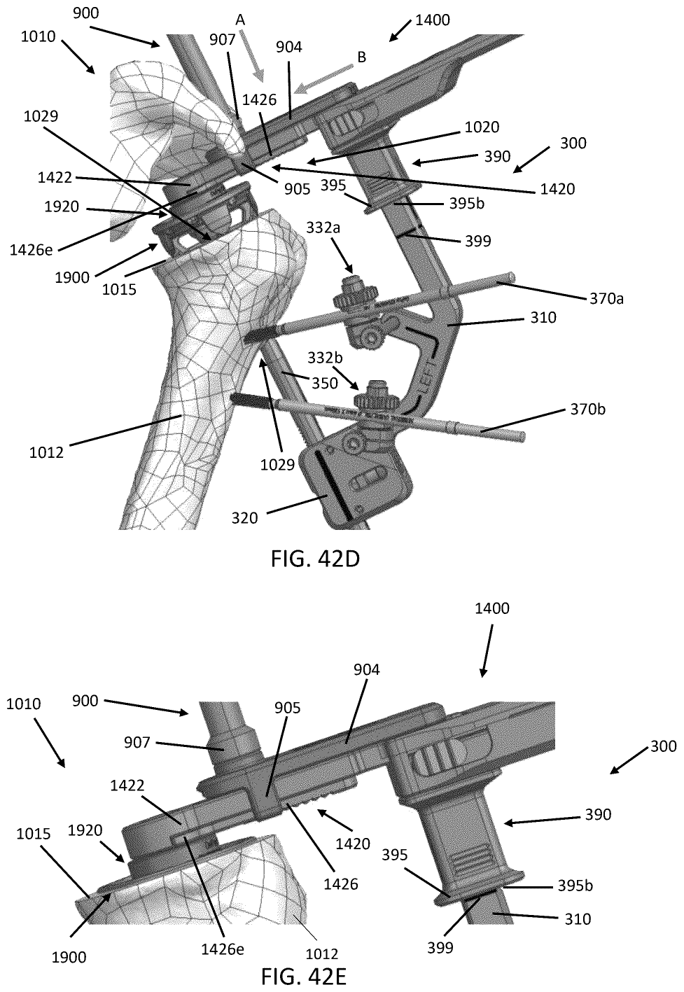

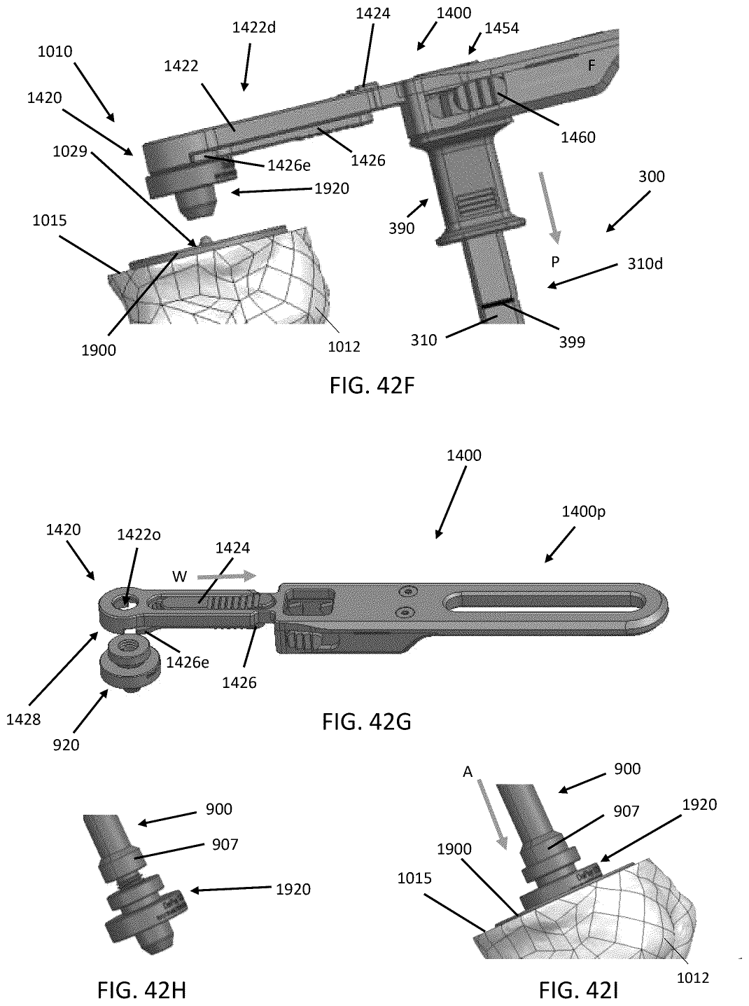

The handle assembly 1400 can again be prepared for use, this time by coupling an implant adapter 1920, illustrated in FIG. 42A, to the attachment portion 1420 of the handle assembly 1400, as shown in FIG. 42B. This attachment of the adapter 1920 to the handle assembly 1400 can be made using similar techniques as described herein, such as sliding the latch 1424 away and then towards the receiving chamber 1428 to capture a mount 1910 of the adapter 1920 with the capture plate 1426, and thus further discussion of the same is unnecessary. As shown, the implant adapter 1920 can include a central boss 922 extending distally from a distal surface of the adapter 1920. The boss 1922 can have a central opening 1923 formed therein for receiving the drive shaft 385 and/or for receiving a shaft to assist in placing the adapter 1920 in the chamber 1428 of the attachment portion 1420 of the handle assembly 1400.

After introducing the handle assembly 1400, and thus the adapter 1920, to the joint space 1010 using techniques already provided for when inserting the handle assembly 1400 to the surgical site, the central boss 1922 can be positioned into the central opening 906 of the implant 1900, as shown in FIG. 42C. The taper 1903 can help position the boss 1922 in the central opening 1906. As also shown in FIG. 42C, the guide 300—as pictured the arm 310 and the adapter 390—is used in conjunction with the handle assembly 1400. As with other aspects of the disclosed techniques, attaching the handle assembly 1400 to the humeral guide 300 can maintain the planar and central axis alignment during implant insertion and impaction.

Similar to the operation of the blazer attachment 600, the impaction tool 900 can be used to operate the adapter 1920. Prior to impaction, the arm of the patient can be moved in adduction and an external rotation, often slight, and extension can be used to ensure the axis of impaction is not obstructed by patient anatomy, thus providing unobstructed implant impaction. One or more retractors can be used to manipulate the tissue to ensure it is clear of the implant before impaction. The tool 900 can be introduced and placed with respect to the handle assembly 1400 in a similar manner as described above when used with the blazer attachment 600. The tool 900 can be slid along the arm 1422 in the direction B, towards the implant 1900, to advance the implant, as shown in FIG. 42D. The surgeon can apply a force, such as by impacting or otherwise hitting the wide handle 906 (see FIG. 40B), with a hammer or mallet to drive a force through the tool 900, through the attachment portion 1420, to the implant impactor 1920, and to the implant 1900 in the direction A, as also shown in FIG. 42D. The implant impactor 920 is used as the end effector of the tool 900, although in other embodiments a different end effector may be used. The humeral drive shaft 385 can also be operated to help move the implant 1900 into the prepared humeral resection surface 1015. Similar details as to use of the tool 900 for the blazer attachment 600 can also be applicable to use of the tool 900 with respect to the implant adapter 1920 and the implant 1900, and thus are not described further with respect to these figures. Likewise, the drill bit 1380 can be used similar to the drive shaft 385 for the blazer attachment 600, and thus details related to the same are not described further with respect to these figures. While the implant 1900 can be fully implanted into the humeral resection surface 1015, it can be advantageous to leave the implant 1900 slightly proud, as shown in FIGS. 42E-42F, with the surface 1902 disposed above the humeral resection surface 1015, to help facilitate humeral head prosthesis assembly. As shown, when the bottom portion 395b of the handle 395 of the adapter 390 reaches the demarcation line 399, that indicates to the surgeon that the implant 1900 is at the desired location with respect to the reamed and broached humeral resection surface 1015. For example, the implant 1900 can be advanced towards the humeral resection surface 1015 until the bottom portion 395b of the handle 395 meets the line 399.

As the procedure is being performed, it can be helpful to make sure that the implant 1900 is positioned in line with the broached geometry. One way to help this can be by moving the arm of the patient in external rotation and extension to ensure the axis of impaction is not obstructed by the anatomy of the patient. Obstruction can be obviated, for example, by using a displacement wrap or subscapularis reducer, additional details of which are disclosed in the previously mentioned U.S. Patent Application Publication No. 2024/0108433. Further, while placing the implant 1900, it can be helpful to ensure there is no soft tissue trapped between the implant 1900 and the humeral resection surface 1015 while placing and advancing the implant 1900 to its final position.

The impaction tool 900 and the handle assembly 1400 can be subsequently removed from the surgical site using techniques already described herein. This can thus include, but is not limited to, actions like disconnecting the handle assembly 1400 from the humeral guide 300 by way of the adapter 390 and/or the slider 1460, as illustrated in FIG. 42F, and detaching the implant adapter 1920 using techniques described herein for detaching the reamer attachment 500 and/or the blazer attachment 600, among other attachments, as illustrated in FIG. 42G. A full explanation of the operations occurring in FIGS. 42F and 42G is unnecessary in view of previous descriptions related to similar figures (see, e.g., FIGS. 38F, 38H, and 40H).

Optionally, a second impaction can be performed. As shown in FIG. 42H, the distal receiver 907 of the impaction tool 900 can have the implant adapter 1920 coupled thereto, for example by threading one onto the other. The tool 900 and implant adapter 1920 can be introduced to the surgical site, such as through the rotator interval, and additional impaction can be supplied to the implant 1900 by the adapter 1920, such as by applying a force in a direction A to the tool 900 as shown.

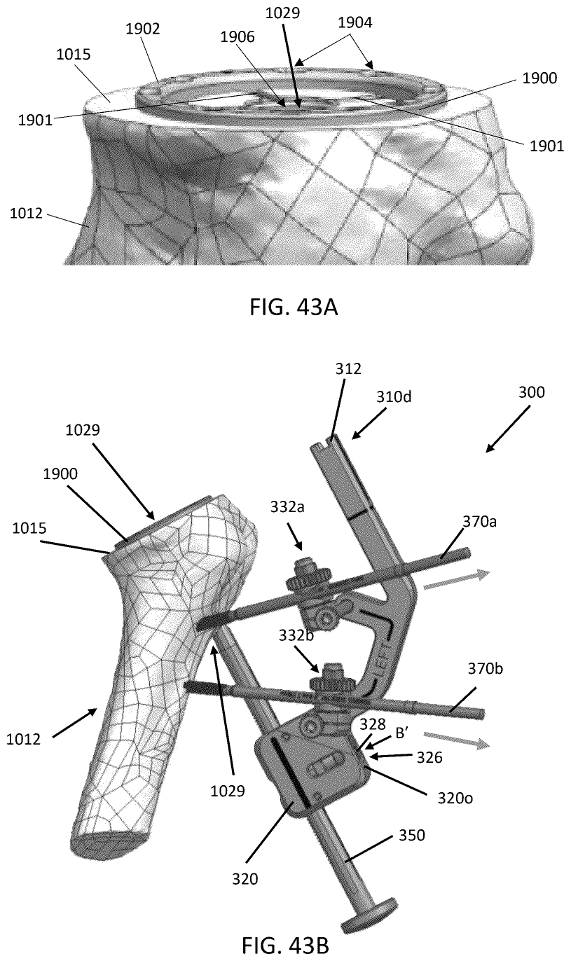

The resulting implant being disposed in the humeral resection surface 1015 is illustrated in FIG. 43A. As shown, the top surface 1902 of the implant 1900 can remain proud of the humeral resection surface 1015, ready to receive a prosthesis. The holes 1904 and/or central opening 1906 can be accessible for use in mating the prosthesis to the implant 1900. FIG. 43B illustrates the humeral guide 300 still attached to the humerus 1012 after the implant 1900 has been disposed in the humeral resection surface 1015, but in the process of being detached therefrom. As shown, the pins 370a, 370b can be removed by unlocking the locking nuts 1340 of the bone pin clamps 332a 332b, for instance by using a wrench, and applying a force (e.g., pulling, rotating, etc.) in a direction C to remove the pins 370a, 370b from the surgical site. In some instances, a power tool in reverse can be used to remove the pins 370a, 370b from the humerus 1012, and thus the surgical site. The release button 328 can be depressed in the direction B′ and the drill cannula 350 can be slid in a direction D, away from the lateral cortex 1023 of the humerus 1012, to remove it from the surgical site. The removal of contact between the humerus and each of the pins 370a, 370b and the drill cannula 350 allows the humeral guide 300, and any components associated therewith (e.g., the arm 310, the hub 320, the bone pin clamps 332a, 332b, the drill cannula 350, the pins 370a, 370b, etc.) to be removed from the surgical site.

In alternative embodiments, any handle assembly provided for herein or otherwise known to those skilled in the art can have a tool mated to an attachment portion like the attachment portion 1420 for use in installing the prosthesis. For example, the tool can be an implant adapter that is operated in a manner similar to other tools, such as by using a guide pin or drill bit (e.g., the drill bits 380, 380′, 1380) or humeral drive shafts (e.g., the humeral drive shafts 385, 385′, 385″) to impact the implant adapter into the implant 1900 to drive the implant 1900 into the humeral resection surface 1015.

### Inserting a Prosthesis(es) to the Implant(s)

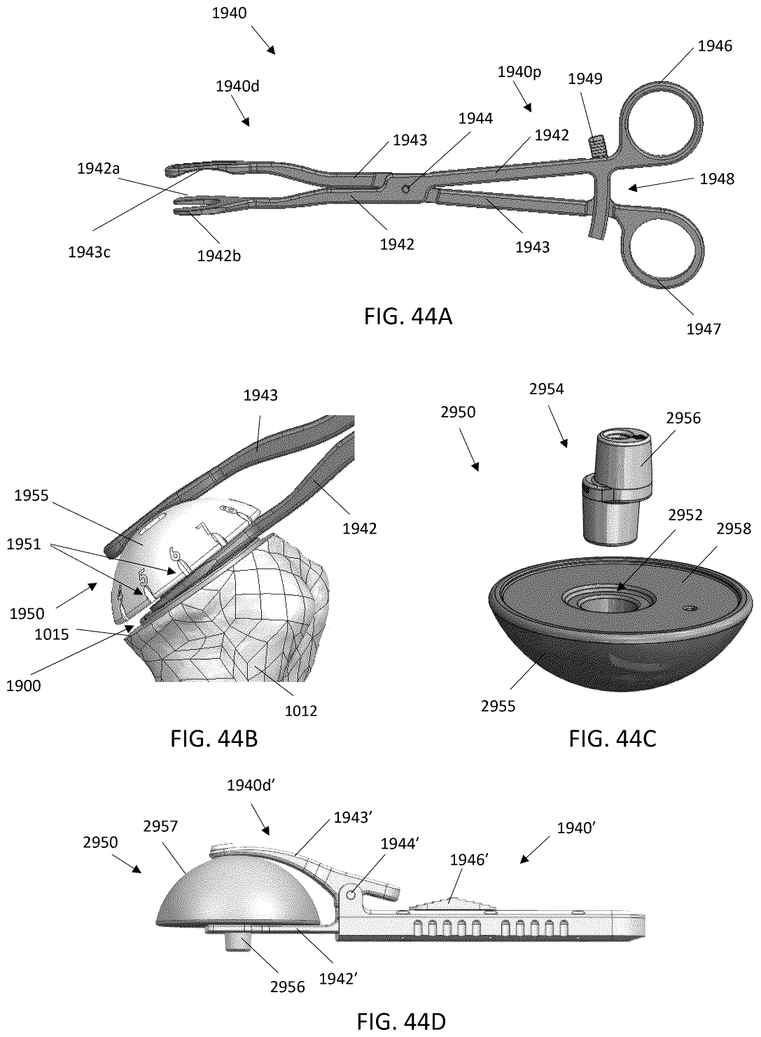

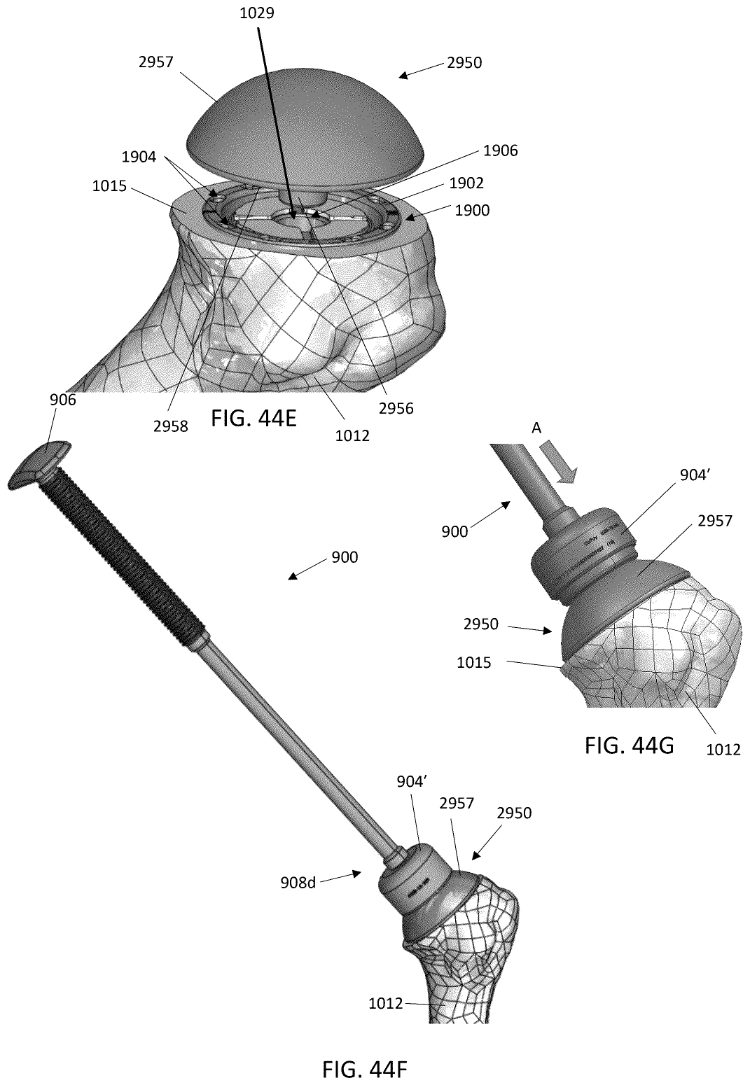

FIGS. 44A-44G illustrate non-limiting embodiments of disposing and/or implanting a humeral head trial and a humeral head prosthesis at the surgical site, along with non-limiting embodiments of tools that can be used in conjunction with the same.

FIG. 44A illustrates a humeral head prosthesis inserter 1940, also referred to as a forked humeral head prosthesis insertion tool, that can be used to insert at least one of a humeral head trial and/or a humeral head prosthesis to the surgical site. The insertion tool 1940 can include grasping arms 1942, 1943 coupled at a pivot point 1944 in a scissors-like manner such that at least one of the arms pivots with respect to the other to allow an object to be grasped therebetween. The arms 1942, 1943 located at a distal portion 1940d of the tool 1940 can be configured to grasp and maintain a trial and/or a prosthesis, such as a humeral head trial 1950 (see FIG. 44B), therebetween. As shown, the first arm 1942, which at the distal portion 1940d is disposed at a bottom as compared to the second arm 1943, can be forked, having tang 1942a, 1942b, to allow a male taper post (not shown, but akin to a post 2956 illustrated as part of a prosthesis 2950, as illustrated in FIGS. 44C and 44D) of the humeral head trial 1950 to sit between the two tangs 1942a, 1942b. The second arm 1943, which at the distal portion 1940d is disposed at a top as compared to the first arm 1942, can include a concave portion 1943c that can be contoured to mimic, or substantially mimic, a curvature of a top surface 1955 of the humeral head trial 1950, providing better securement of the humeral head trial 1950 relative to the tool 1940. Scissors-like or snap-like handles 1946, 1947 can be disposed on the arms 1942, 1943, respectively, at a proximal portion 1940p of the tool 1940, enabling pivoting motion between the two arms 1942, 1943.

As shown, at the proximal portion 1940p, the first arm 1942 is at the top and the second arm 1943 is at the bottom relative to each other. In at least some embodiments, a ratcheting mechanism 1948 can be disposed between the two arms 1942, 1943 to allow for more controlled, incremental pivoting movement of the arms 1942, 1943. For example, a pawl-and-ratchet and/or a teeth-and-engagement post configuration can be utilized on the ratcheting mechanism 1948 to enable such incremental movement. In the illustrated embodiment, teeth 1949 can be seen on one surface of the ratcheting mechanism 1948, and a person skilled in the art will appreciate an engagement post (not visible) that can be disposed on an opposed surface of the ratcheting mechanism 1948 to enable the incremental movement. A person skilled in the art will likewise appreciate a variety of other configurations that can be used to enable pivoting movement and/or controlled, incremental pivoting movement. Allowing for more controlled, incremental pivoting can allow for a better grasping of the humeral head trial and/or prosthesis without possibly squeezing it tighter than desired, thus avoiding possible damage to the humeral head trial and/or prosthesis due to squeezing it too tightly.

FIG. 44B illustrates one example of a humeral head trial 1950 for use in at least one of assessing range of motion and/or final implant head size. The illustrated trial 1950 is an INHANCE™ humeral head trial, and thus use of the same is understood by a person skilled in the art, in view of the present disclosures and the previous surgical techniques incorporated herein by reference. The trial 1950 has a convex shape or top surface 1955, similar to a humeral head, and intended to mimic a shape of a prosthesis to be implanted and mated to the implant 1900. As shown, a plurality of sequential numbers can be disposed thereon (e.g. 4, 5, 6, 7, and 8 are visible). These numbers identify markings 1951 that help the user know a desired position and/or size of the eventual prosthesis that will be used in conjunction with the implant 1900, which can be informed by a range of motion of the patient.

As shown in FIG. 44B, the arms 1942, 1943 can deliver the humeral head trial 1950 to the implant 1900 disposed in the prepared humeral resection surface 1015. The delivery can be done in a manner similar to the delivery of other components during the surgical procedure in which an instrument or tool (e.g., handle assembly 1400) delivers a humeral bone preparation attachment or tool (e.g., attachments 500, 500′, 600, 600′, 1920) to the humeral resection surface 1015. Accordingly, the insertion tool 1940 can be used to pass the humeral head trial 1950 into the glenohumeral shoulder joint 1010, through the rotator interval 1020, and to a location that is proximate to the implant 1900. During insertion, and akin with the other insertion procedures provided for herein, one or more retractors can be used to manipulate tissue to improve visualization and/or prevent damage to tissue.

An initial size of the trial 1950 can be estimated using the humeral resection surface size and/or other dimensions and/or determinations made during the course of the surgical procedure. Multiple trials 1950 may need to be inserted to make final determinations about the desired size and configuration of the prosthesis being used for final insertion and implantation. An explanation of additional actions related to using the trial(s) 1950 to determine a final size and/or configuration of the humeral head prosthesis are unnecessary in view of such procedures being related to the INHANCE™ surgical procedure.

Upon determination of the final size and/or configuration of the humeral head prosthesis that is going to be mated to the implant 1900 at the surgical site, the humeral head prosthesis can be assembled. FIG. 44C illustrates one embodiment of such a prosthesis 2950, and ways by which it can be assembled in view of the exploded view provided. As shown in FIG. 44C, the prosthesis 2950 can include a central opening 2952 therein for receiving, by way of example, an offset taper adapter 2954 having a male taper post 2956 configured to extend proud of a substantially flat facial surface 2958 of the prosthesis 2950, also referred to as a distal bearing surface. This assembly can occur outside of the joint space 1010, outside of the body, for example at a back table in the surgery room. The prosthesis 2950 can also include an outer concave surface 2955 that can be formed to mimic the performance of a humeral head.

The prosthesis 2950 having the adapter 2954 disposed therein can be grasped by an insertion tool, such as the insertion tool 1940. FIG. 44D illustrates an alternative insertion tool 1940′, which can also be a forked humeral head prosthesis insertion tool. The insertion tool 1940′ includes grasping arms 1942′, 1943′ disposed at a distal end 1940d′ of the tool 1940′. As shown, the top arm 1943′ can be configured to rotate about a pivot point 1944′, relative to the bottom arm 1942′, to grasp objects, as shown the prosthesis 2950, disposed between the arms 1942′, 1943′. In the illustrated embodiment, prior to having the top arm 1943′ engage the outer concave surface 2955, a single use protector sleeve 2957, such as a plastic sleeve, can be disposed over the prosthesis 2950 to protect the prosthesis from damage during insertion. The bottom arm 1942′ can be forked (not illustrated) to allow the male taper post 2956 to sit between the two forks. A slider tab 1946′ disposed proximal of the pivot point 1944′ can be used to selectively open and close the grasping arms 1942′ of the tool 1940′. For example, sliding the slider tab 1946′ towards the arms 1942′, 1943′ as illustrated can cause the arm 1943′ to rotate towards the arm 1942′ to grasp objects therebetween, while sliding the slider tab 1946′ away from the arms 1942′, 1943′ can cause the arm 1943′ to rotate away from the arm 1942′ to release objects previously grasped therebetween. A person skilled in the art will appreciate that other tools can be used in lieu of the insertion tools 1940, 1940′ to deliver a prosthesis or other implant, such as instruments having more of a forked bottom portion and/or a curved top portion that can secure the prosthesis 2950 using a more forceps-based approach.

The prosthesis 2950 can be delivered to the implant 1900 disposed in the prepared humeral resection surface 1015 in a manner akin to delivery of the humeral head trial 1950. During insertion, and akin with the other insertion procedures provided for herein, one or more retractors can be used to manipulate tissue to improve visualization and/or prevent damage to tissue. Such retractors can be used throughout the remainder of the procedure to manipulate tissues to ensure it is clear of the implant and prosthesis.

FIG. 44E illustrates the prosthesis 2950 disposed proximate to the implant 1900 at the prepared humeral resection surface 1015. The insertion tool 1940′ can position and hold the prosthesis 2950 at this location, though the tool 1940′ has been removed from the illustration to focus on the prosthesis 2950 and the implant 1900. As shown, the male taper post 2956 can be aligned with the central opening 1906, in this instance serving as a female taper bore, on the stemless implant 1900. In conjunction with aligning the prosthesis 2950 with the implant 1900, and eventually mating the two together, tissue can be manipulated to ensure a clear view of the implant. This can include, for example using and/or maintaining the use of one or more displacement wraps or subscapularis reducers as provided for elsewhere in the present disclosure and/or using one or more retractor(s) to manipulate tissue to ensure the tissue is clear of the implant.

After initially placing the prosthesis 2950 in contact with the implant 1900, such as by inserting the male taper post 2956 into the female taper bore 1906, the insertion tool 1940′ can be removed from the surgical site, for example via the rotator interval 1020, and the impaction tool 1900 can be reintroduced to the surgical site, also via the rotator interval 1020 for example. As shown in FIGS. 44F and 44G, the impaction tool can include a concave impactor tip or end effector 904′ securely coupled to the distal end 908d of the elongate rod 908 in lieu of the end effector 904. The concave impactor tip 904′ can be configured to have a complementary surface to the outer surface of the prosthesis 2950, and then an impaction force in the direction A, such as by way of a hammer or mallet, can be applied to the wide handle 906 in a similar manner as described above. The impaction force in the direction A can impact the prosthesis 2950 and advance the prosthesis 2950 into further engagement with the implant 1900 while also advancing the implant 1900 further into the bone until the top surface 1902 sits flush against the humeral resection surface 1015. Thus, the impaction tool 900 can be used to finalize the placement of the prosthesis 2950 with respect to the implant 1900, and thus the humeral resection surface 1015. The impaction tool 900 can then be removed from the surgical site.

In alternative embodiments, a handle assembly like the handle assembly 1400, or other handle assemblies provided for herein or otherwise derivable from the present disclosures can have a tool mated to its attachment portion (e.g., the attachment portion 1420) for use in installing the prosthesis. For example, the tool can be a disc that engages the prosthesis 2950 and then an impacting force, such as one provided for by the impaction tool 900, can be supplied to impact the disc into the prosthesis 2950 so that the prosthesis 2950 mates to the implant 1900.

The final combination of the prosthesis 2950 and implant 1900, also referred to as a final construct, can be such that the top surface 1902 of the implant 1900 sits flush with the humeral resection surface 1015 and the facial surface 2958 of the prosthesis 2950 sits flush with both the top surface 1902 of the implant and the humeral resection surface 1015. This resulting configuration of the final construct can be articulated and assessed to ensure the sizing and configuration is appropriate. The prosthesis 2950 can be removed and replaced to obtain desired joint tension. After delivery of all implants and prostheses, and after the final construct has been properly assessed and tested, all instrumentation and the like can be removed from the surgical site and the surgical site can be closed using techniques known to those skilled in the art. Further, any displacement wraps or subscapularis reducers can be released to free the tissues, as well as any other instruments used to provide visualization can be removed. Standard techniques for closing access to the glenohumeral shoulder joint 1010 can be performed to finish the procedure.

### Digital Navigation Techniques and Kits

Any and all of the aspects of the present disclosure can be incorporated into various smart techniques that allow for digital navigation of any and all of the various components disclosed. Accordingly, the humeral resection guides, the glenoid guides, the humeral guides, and all associated components described herein, can be used with digital navigation techniques and programming. Further, in view of the ability for these various components to be used in smart techniques, some of the instrumentation, and/or features thereof, may not be required and/or can be used in conjunction with some or all of the various aspects of the disclosed procedures.

The present disclosure also contemplates the possibility that the various systems, instruments, devices, tools, components, etc. can be provided together as a surgical repair kit. This can include, by way of non-limiting example different sizes and configurations of: humeral resection guides (e.g., resection guide 100 and related components); glenoid guides (e.g., glenoid guide 200 and related components); humeral guides (e.g., humeral guide 300, 300′); drill cannulas (e.g., drill cannula 350); bone pins (e.g., bone pins 370a, 370b); sizer handles (e.g., sizer handle 280); drivers (e.g., driver 290); drill bits or guide pins (e.g., drill bits 380, 380′, 1380, and/or humeral drive shafts (e.g., drive shafts 385, 385′, 385″); adapters (e.g., adapter 390); handle assemblies (e.g., handle assembly 1400); attachments (e.g., humeral sizer attachment 340, 1140, reamer attachment 500, 500′, blazer attachment 600, 600′, reamer attachment 700, implant adapter 1920); retractors (e.g., retractors 101a, 101b, 101c); delivery and/or extraction tools (e.g., tool 800, 800′); impaction tools (e.g., tool 900) and related end effectors (e.g., end effector 904, 904′); implant delivery tools (e.g., insertion tool 1940, 1940′); sizer plates (e.g., sizer plates 1172, 1172′, INHANCE™ humeral head trials); and implants and end effectors (e.g., glenoid implant 204, impactor tool 206, implant 1900, prosthesis 1950, 1950′), among other devices, tools, components, etc.

### Other Considerations

The devices, tools, components, and the like described herein can be processed before use in a surgical procedure. First, a new or used instrument can be obtained and, if necessary, cleaned. The instrument can then be sterilized. In one sterilization technique, the instrument can be placed in a closed and sealed container, such as a plastic or TYVEK bag. The container and its contents can then be placed in a field of radiation that can penetrate the container, such as gamma radiation, x-rays, or high-energy electrons. The radiation can kill bacteria on the instrument and in the container. The sterilized instrument can then be stored in the sterile container. The sealed container can keep the instrument sterile until it is opened in the medical facility. Other forms of sterilization known in the art are also possible. This can include beta or other forms of radiation, ethylene oxide, steam, or a liquid bath (e.g., cold soak). Certain forms of sterilization may be better suited to use with different portions of the devices, tools, components, and the like due to the materials utilized, the presence of electrical components, etc.

Examples of the above-described embodiments can include the following:

1. A surgical method, comprising:
exposing a glenohumeral joint and glenohumeral joint space;
coupling a resection guide to at least one of a humeral head or a humerus of the glenohumeral joint;
introducing a cutting instrument to the glenohumeral joint space;
resecting at least a portion of the humeral head located at the glenohumeral joint using the resection guide to guide the cutting instrument and creating a humeral resection surface;
removing the resection guide and the cutting instrument from the glenohumeral joint space;
coupling a humeral guide to at least one of the humeral resection surface or the humerus such that a proximal portion of the humeral guide is disposed below the humeral resection surface, at a location that is opposed to the humeral resection surface, and a distal portion of the humeral guide is disposed proximate to a rotator interval, approximately aligned with the humeral resection surface;
coupling a humeral sizer attachment to the distal end of the humeral guide;
identifying an approximate center point of the humeral resection surface with a distal portion of the humeral sizer attachment;
forming a transhumeral bone tunnel in the humerus using the humeral guide and the humeral sizer attachment to guide a drill bit through the humerus and the humeral resection surface;
de-coupling the humeral sizer attachment from the distal end of the humeral guide;
introducing a distal end of a handle assembly to the glenohumeral joint space, the distal end of the handle assembly having coupled thereto a humeral bone preparation instrument;
coupling the handle assembly to the distal end of the humeral guide;
passing a guide pin through the transhumeral bone tunnel;
coupling the guide pin to the humeral bone preparation instrument;
operating the humeral bone preparation instrument to treat the humeral resection surface;
de-coupling the guide pin from the humeral bone preparation instrument; and
removing the distal end of the handle assembly and the humeral bone preparation instrument from the glenohumeral joint space.

2. The method of example 1, further comprising performing an inferior release of a subscapularis tendon of the glenohumeral joint.

3. The method of example 1 or example 2, further comprising performing a superior release of a subscapularis tendon.

4. The method of any of examples 1 to 3, further comprising marking one or more anatomical landmarks within the glenohumeral joint space.

5. The method of example 4, wherein marking one or more anatomical landmarks within the glenohumeral joint space further comprises marking a bicipital groove of the humerus.

6. The method of any of examples 1 to 5, further comprising moving a subscapularis tendon one of inferiorly or superiorly and holding it in place with a displacement wrap.

7. The method of any of examples 1 to 6, wherein resecting at least a portion of a humeral head located at the glenohumeral joint further comprises:
engaging the humeral head with a superior arm of a resection guide;
passing at least one bone pin through a portion of the resection guide and into the humeral head; and
passing the cutting instrument through a guide slot formed on the resection guide.

8. The method of example 7, wherein engaging the humeral head with a superior arm of a resection guide further comprises engaging the humeral head at a location that is at least one of at or proximate to a supraspinatus attachment point on the humeral head.

9. The method of example 7 or example 8, further comprising aligning the guide slot to at least one of a bicipital groove of the humerus or a rotator interval.

10. The method of any of examples 7 to 9, further comprising aligning a vertical alignment plate of the resection guide with an elongate shaft of the humerus to set a location of the superior arm.

11. The method of example 10, wherein a vertical guide rod is coupled to at least one of the vertical alignment plate or a handle coupled to the vertical alignment plate, the vertical guide rod extending along the elongate shaft of the humerus in conjunction with aligning the vertical alignment plate of the resection guide with the elongate shaft of the humerus.

12. The method of example 10 or example 11, further comprising moving the vertical alignment plate to change an angle of inclination of a cutting plane defined by the resection guide.

13. The method of any of examples 7 to 12, wherein passing at least one bone pin through a portion of the resection guide and into the humeral head occurs such that the at least one bone pin does not pass through soft tissue in the glenohumeral joint space.

14. The method of any of examples 7 to 13, further comprising:
mating a handle to the resection guide; and
checking angular alignment with a forearm using the handle.

15. The method of example 14, further comprising manipulating the handle to adjust a location of the superior arm of the resection guide.

16. The method of any of examples 7 to 15, further comprising mating an extender to the resection guide, the extender being configured to extend a cutting plane defined by the superior arm of the resection guide inferior to the subscapularis tendon.

17. The method of example 16, further comprising passing at least one inferior bone pin through a portion of the extender and into the humeral head such that the at least one inferior bone pin does not pass through soft tissue in the glenohumeral joint space.

18. The method of any of examples 1 to 17, further comprising:
introducing a reamer to the glenohumeral joint space;
reaming a glenoid surface of a glenoid of the glenohumeral joint using the reamer; and
removing the reamer from the glenohumeral joint space.

19. The method of example 18, further comprising:
introducing a glenoid sizer plate to the glenohumeral joint;
using the glenoid sizer plate to at least one of determine a size of the glenoid surface or determine a preferred location on the glenoid surface; and
removing the glenoid sizer plate from the glenohumeral joint space.

20. The method of example 19, wherein introducing a glenoid sizer plate to the glenohumeral joint space comprises using a sizer handle to pass the glenoid sizer through the rotator interval, the glenoid sizer being positioned substantially flush with the glenoid surface.

21. The method of example 19 or example 20, further comprising passing a drill bit through the glenoid sizer to form a pilot hole in the glenoid surface.

22. The method of any of examples 18 to 21, further comprising:
coupling a glenoid guide to at least one of the humeral resection surface or the humerus such that a distal portion of the glenoid guide is disposed between the humeral resection surface and a surface of a glenoid of the glenohumeral joint; and
forming a second transhumeral bone tunnel using the glenoid guide to guide a drill bit through the humerus.

23. The method of example 22, wherein the drill bit passes through the second transhumeral bone tunnel to then pass through the glenoid sizer to form a pilot hole in the glenoid surface.

24. The method of example 23, wherein the drill bit is a stepped drill bit with a smaller diameter at a distal tip such that the pilot hole is smaller than the second transhumeral bone tunnel.

25. The method of any of examples 22 to 24, wherein coupling a glenoid guide to at least one of the humeral resection surface and the humerus further comprises:
passing a glenoid guide drill cannula through a proximal end of the glenoid guide; and
coupling a distal end of the glenoid guide drill cannula to a lateral surface of the humerus.

26. The method of example 25, wherein coupling a distal end of the drill cannula to a lateral surface of the humerus sets a trajectory for the drill bit forming the second transhumeral bone tunnel that is substantially central and substantially perpendicular to the surface of the glenoid.

27. The method of any of examples 22 to 26, further comprising passing the drill bit through the glenoid guide drill cannula and through the humerus to form the second transhumeral bone tunnel.

28. The method of any of examples 22 to 27, further comprising removing the glenoid guide from the glenohumeral joint space prior to the actions of introducing a reamer to the glenohumeral joint space and reaming the glenoid surface using the reamer occur.

29. The method of any of examples 22 to 28, wherein reaming the glenoid surface using the reamer further comprises:
passing a driver through the second transhumeral bone tunnel;
coupling the reamer to the driver; and
operating the driver to ream the surface of the glenoid.

30. The method of example 29, further comprising:
de-coupling the reamer from the driver; and
removing at least the reamer from the glenohumeral joint space.

31. The method of any of examples 18 to 30, further comprising:
introducing a glenoid implant into the glenohumeral joint space; and
implanting the glenoid implant into the glenoid surface.

32. The method of example 31, wherein introducing a glenoid implant into the glenohumeral joint space further comprises:
passing the glenoid implant through the rotator interval; and
implanting the glenoid implant into the glenoid surface,
wherein the glenoid implant is removably coupled to a glenoid guide such that the glenoid guide is used to pass the glenoid implant through the rotator interval for introduction into the glenohumeral joint space, and
wherein the glenoid guide can be the same glenoid guide of example 22 or it can be a different glenoid guide.

33. The method of example 32, further comprising:
detaching the glenoid implant from the glenoid guide;
introducing an impactor tool into the glenohumeral space by passing the impactor tool through the rotator interval; and
driving the impactor tool into the glenoid implant to further seat the glenoid implant into the glenoid surface,
wherein the impactor tool is coupled to a glenoid guide such that the glenoid guide is used to pass the glenoid implant through the rotator interval for introduction into the glenohumeral joint space, and
wherein the glenoid guide can be the same glenoid guide of example 22 or example 32, or it can be a different glenoid guide.

34. The method of any of examples 1 to 33, wherein identifying an approximate center point of the humeral resection surface with a distal portion of the humeral sizer attachment, further comprises:
positioning the distal portion of the humeral sizer attachment such that is aligned to be substantially parallel with the humeral resection surface; and
aligning an anatomical landmark of the humerus with an indicator of the humeral sizer attachment.

35. The method of any of examples 1 to 34, wherein coupling a humeral guide to at least one of the humeral resection surface or the humerus further comprises:
passing a drill cannula through a hub of the humeral guide; and
coupling a distal end of the drill cannula to a lateral cortex of the humerus.

36. The method of any of examples 1 to 35, wherein forming a transhumeral bone tunnel in the humerus further comprises passing a drill bit through the drill cannula and through the humerus to form the transhumeral bone tunnel.

37. The method of any of examples 1 to 36, further comprising:
coupling at least one bone pin to the humeral guide; and
passing the at least one bone pin into the humerus.

38. The method of example 37, wherein coupling at least one bone pin to the humeral guide further comprises:
passing the bone pin through a bone pin-receiving fixation feature coupled to the humeral guide; and
operating the bone pin-receiving fixation feature to secure a position of the bone pin with respect to the bone pin-receiving fixation feature.

39. The method of example 38, further comprising operating the bone pin-receiving fixation feature to adjust at least one of a location of the bone pin with respect to the humerus or an angle of entry of the bone pin with respect to the humerus.

40. The method of example 38 or example 39, further comprising operating the bone pin-receiving fixation feature to adjust a location of the bone pin-receiving fixation feature with respect to a support rod coupled to the humeral guide and on which the bone pin-receiving fixation feature is disposed.

41. The method of any of examples 38 to 40, further comprising:
coupling at least one support rod to the humeral guide; and
coupling the at least one bone pin-receiving fixation feature to the at least one support rod.

42. The method of any of examples 38 to 41, wherein the bone pin-receiving fixation feature comprises a bone pin clamp having at least one bone-pin receiving opening for receiving the bone pin, the bone pin clamp being configured to be able to adjust at least one of a location of the bone pin with respect to the humerus or an angle of entry of the bone pin with respect to the humerus.

43. The method of example 42, wherein the bone pin clamp has at least one support-rod receiving opening for receiving at least one support rod of the humeral guide, the bone pin clamp being configured to be able to adjust a location of the clamp with respect to the at least one support rod on which the clamp is disposed.

44. The method of example 42 or example 43, wherein the bone pin clamp is configured to provide multiple degrees of freedom for at least one of the bone pin and the support rod received therein.

45. The method of example 44, wherein the multiple degrees of freedom comprise at least two of sliding, rotation, and orientation.

46. The method of example 45, wherein the multiple degrees of freedom comprise each of sliding, rotation, and orientation.

47. The method of any of examples 1 to 46, further comprising:
after removing the humeral bone preparation instrument from the glenohumeral joint space, introducing the distal end of the handle assembly to the glenohumeral joint space, the distal end of the handle assembly having coupled thereto a second humeral bone preparation instrument;
coupling the handle assembly to the distal end of the humeral guide;
passing a guide pin through the transhumeral bone tunnel;
coupling the guide pin to the second humeral bone preparation instrument;
operating the second humeral bone preparation instrument to treat the humeral resection surface;
de-coupling the guide pin from the second humeral bone preparation instrument; and
removing the second humeral bone preparation instrument from the glenohumeral joint space.

48. The method of any of examples 1 to 47, wherein coupling the handle assembly to the distal end of the humeral guide, in one or both instances with respect to example 47, further comprises:
positioning a receiving opening formed in the handle assembly over the distal end of the humeral guide;
passing the distal end of the humeral guide into the receiving opening formed in the handle assembly; and
securing the handle assembly to the distal end of the humeral guide using a slidable adapter disposed on the distal end of the humeral guide.

49. The method of example 48, further comprising disposing the slidable adapter on the distal end of the humeral guide.

50. The method of example 49, wherein disposing the slidable adapter on the distal end of the humeral guide occurs after de-coupling the humeral sizer attachment from the distal end of the humeral guide.

51. The method of any of examples 48 to 50, wherein operating the humeral bone preparation instrument to treat the humeral resection surface, in one or both instances with respect to any of examples 48 to 50, further comprises continuing to operate the humeral bone preparation instrument, or the respective second humeral bone preparation instrument where applicable, until a bottom portion of the slidable adapter reaches a demarcation line formed on an arm of the humeral guide, which signals that the humeral bone preparation instrument, or the respective second humeral bone preparation instrument where applicable, disposed on a distal end of the handle assembly is at least one of flush or substantially flush with the humeral resection surface.

52. The method of any of examples 1 to 51, wherein operating the humeral bone preparation instrument to treat the humeral resection surface, in one or both instances with respect to any of examples 47 to 51, further comprises using the guide pin to move the humeral bone preparation instrument, or the respective second humeral bone preparation instrument where applicable, towards the humeral resection surface to provide treatment to the humeral resection surface.

53. The method of any of examples 1 to 52, wherein operating the humeral bone preparation instrument to treat the humeral resection surface, in one or both instances with respect to any of examples 47 to 52, further comprises applying a force to the handle assembly to move the humeral bone preparation instrument, or the respective second humeral bone preparation instrument where applicable, towards the humeral resection surface to provide treatment to the humeral resection surface.

54. The method of example 53, wherein applying a force to the handle assembly further comprises:
engaging the handle assembly with an impaction tool; and
applying a force to the impaction tool to operate the humeral bone preparation instrument to treat the humeral resection surface.

55. The method of any of examples 47 to 54, wherein the humeral bone preparation instrument comprises a reamer.

56. The method of any of examples 47 to 54, wherein the second humeral bone preparation instrument comprises a blazer.

57. The method of any of examples 1 to 56, further comprising coupling a humeral bone preparation instrument to the handle assembly.

58. The method of example 57, wherein coupling a humeral bone preparation instrument to the handle assembly further comprises:
positioning the humeral bone preparation instrument such that a portion thereof is disposed within a chamber of the distal end of the handle assembly; and
causing a capture plate of the handle assembly to slide towards the distal terminal end of the handle assembly to engage a portion of the humeral bone preparation instrument disposed within the chamber of the distal end of the handle assembly such that the humeral bone preparation instrument is secured with respect to the handle assembly.

59. The method of any of examples 1 to 58, further comprising:
introducing a humeral implant to the glenohumeral joint space;
implanting the humeral implant into the humeral resection surface;
introducing a humeral head prosthesis to the glenohumeral joint space; and
coupling the humeral head prosthesis to the humeral implant,
wherein for methods in which the glenoid implant is implanted into the glenoid surface, the glenoid implant is configured to operate like an anatomic glenoid surface.

60. The method of example 59, wherein implanting the humeral implant into the humeral resection surface further comprises:
introducing the distal end of the handle assembly to the glenohumeral joint space, the distal end of the handle assembly having coupled thereto an implant adapter; and
operating the implant adapter to implant the humeral implant into the humeral resection surface.

61. The method of example 60, wherein operating the implant adapter to implant the humeral implant into the humeral resection surface further comprises applying a force to the handle assembly to move the implant adapter towards the implant.

62. The method of example 61, wherein applying a force to the handle assembly further comprises:
engaging the handle assembly with an impaction tool; and
applying a force to the impaction tool to operate the implant adapter to implant the humeral implant into the humeral resection surface.

63. The method of any of examples 59 to 62, further comprising:
prior to introducing the humeral head prosthesis to the glenohumeral joint space, de-coupling the humeral guide from the respective at least one of the humeral resection surface or the humerus; and
removing the humeral guide from a surgical site that includes the glenohumeral joint space at which the method is being performed.

64. The method of any of examples 59 to 63, wherein coupling the humeral head prosthesis to the humeral implant further comprises:
placing the humeral head prosthesis in contact with the humeral implant; and
impacting the humeral head prosthesis with an impaction tool to drive the humeral head prosthesis into a secure engagement with the humeral implant.

65. The method of any of examples 1 to 58 in which the glenoid implant has been introduced, the glenoid implant being configured to receive a glenoid prosthesis that is configured to operate like an anatomic humeral head, the method further comprising:
introducing a humeral implant to the glenohumeral joint space, the humeral implant being configured to provide a surface for receiving a glenoid prosthesis that is configured to operate like an anatomic humeral head;
implanting the humeral implant into the humeral resection surface;
introducing a glenoid prosthesis that is configured to operate like an anatomic humeral head to the glenohumeral joint space; and
coupling the glenoid prosthesis to the glenoid implant.

66. The method of example 65, wherein implanting the humeral implant into the humeral resection surface further comprises:
introducing the distal end of the handle assembly to the glenohumeral joint space, the distal end of the handle assembly having coupled thereto an implant adapter; and
operating the implant adapter to implant the humeral implant into the humeral resection surface.

67. The method of example 66, wherein operating the implant adapter to implant the humeral implant into the humeral resection surface further comprises applying a force to the handle assembly to move the implant adapter towards the implant.

68. The method of example 67, wherein applying a force to the handle assembly further comprises:
engaging the handle assembly with an impaction tool; and
applying a force to the impaction tool to operate the implant adapter to implant the humeral implant into the humeral resection surface.

69. The method of any of examples 1 to 68, wherein the method is performed with a transhumeral approach.

70. The method of example 69, wherein the introducing actions performed during the method are performed such that whatever is being introduced into the glenohumeral joint space is introduced from a location that is lateral to the humeral resection surface.

71. The method of example 69 or example 70, wherein the introducing actions performed during the method are performed such that the introduction occurs substantially orthogonal to a humeral cut plane.

72. The method of any of examples 69 to 71, wherein the introducing actions occur through the rotator interval.

73. The method of any of examples 1 to 72, wherein the method is performed while keeping the subscapularis tendon intact.

74. The method of any of examples 1 to 73, wherein the method is performed without distracting the humeral head from its joint.

75. A surgical method, comprising:
coupling a resection guide to at least one of a humeral head or a humerus of a glenohumeral joint;
resecting at least a portion of the humeral head located at the glenohumeral joint using the resection guide to guide a cutting instrument and creating a humeral resection surface;
removing the resection guide from the glenohumeral joint space;
coupling a humeral guide to at least one of the humeral resection surface or the humerus such that a proximal portion of the humeral guide is disposed below the humeral resection surface, at a location that is opposed to the humeral resection surface, and a distal portion of the humeral guide is disposed proximate to a rotator interval, approximately aligned with the humeral resection surface;
forming a transhumeral bone tunnel in the humerus using the humeral guide to guide a drill bit through the humerus and the humeral resection surface;
coupling a handle assembly to the distal end of the humeral guide, the handle assembly having a humeral bone preparation instrument coupled to a distal end thereof; and
operating the humeral bone preparation instrument by way of a guide pin disposed in the transhumeral bone tunnel to treat the humeral resection surface, wherein a subscapularis tendon remains intact throughout the method.

76. The method of example 75, further comprising:
exposing a glenohumeral joint and glenohumeral joint space;
introducing the cutting instrument to the glenohumeral joint space;
after removing the resection guide from the glenohumeral joint space and coupling the humeral guide to at least one of the humeral resection surface or the humerus, coupling a humeral sizer attachment to the distal end of the humeral guide;
identifying an approximate center point of the humeral resection surface with a distal portion of the humeral sizer attachment;
prior to coupling a handle assembly to the distal end of the humeral guide, de-coupling the humeral sizer attachment from the distal end of the humeral guide;
introducing the distal end of the handle assembly to the glenohumeral joint space;
prior to operating the humeral bone preparation instrument by way of a guide pin disposed in the transhumeral bone tunnel to treat the humeral resection surface, passing a guide pin through the transhumeral bone tunnel; and
coupling the guide pin to the humeral bone preparation instrument,
wherein forming a transhumeral bone tunnel in the humerus using the humeral guide to guide a drill bit through the humerus and the humeral resection surface further comprises forming the transhumeral bone tunnel in the humerus using the humeral guide and the humeral sizer attachment to guide the drill bit through the humerus and the humeral resection surface.

77. The method of example 76, further comprising:
de-coupling the guide pin from the humeral bone preparation instrument; and
removing the distal end of the handle assembly and the humeral bone preparation instrument from the glenohumeral joint space.

78. The method of any of examples 75 to 77, further comprising any of the methods of examples 2 to 74.

79. A surgical kit, comprising:
a resection guide for resecting a humeral head, the resection guide including:
   a superior radial arm having a proximal portion with at least one bone pin receiving opening formed therein and a distal portion configured to engage a humeral head of a humerus, a superior surface of the proximal portion of the superior radial arm defining a resecting plane of the resection guide;
   a guide slot formed on the superior radial arm, the guide slot being defined by a ledge of the distal portion of the superior radial arm extending over the superior surface of the proximal portion of the superior radial arm, and the guide slot being configured to receive a cutting instrument therethrough and guide the cutting instrument along the resecting plane while cutting the humeral head, keeping the cutting instrument one of parallel or substantially parallel to the resecting plane; and
a humeral guide configured to couple to at least one of a humeral resection surface or a humerus, the humeral guide including:
   a rigid arm having a proximal portion and a distal portion, the distal portion being configured to have an attachment coupled thereto, and the proximal portion having a cannula-receiving opening formed therein such that a plane defined by a primary surface of a distal end of an attachment coupled to the distal portion of the rigid arm is substantially orthogonal with a longitudinal axis extending through the cannula-receiving opening that defines a path of travel for a drill cannula;
   at least one support rod configured to be coupled to the rigid arm; and
   at least one bone pin clamp coupled to the at least one support rod, the at least one bone pin clamp being configured to provide multiple degrees of freedom such that a bone pin coupled to the at least one bone pin clamp can be manipulated across multiple degrees of freedom.

80. The surgical kit of example 79, wherein the resection guide further comprises: a vertical alignment plate extending distally from the superior radial arm, a length of the vertical alignment plate and the superior surface of the proximal portion of the superior radial arm forming an angle therebetween, the angle defining a resecting angle of the resection guide, and thus an angle of the resecting plane of the resection guide.

81. The surgical kit of example 79 or example 80, further comprising at least one handle-receiving opening formed in the vertical alignment plate, the opening being configured to receive a version handle for at least one of manipulating a location of the superior radial arm with respect to the humerus or checking for version alignment between the resection guide and the humerus.

82. The surgical kit of example 81, further comprising a version handle configured to be coupled to the vertical alignment plate and configured to at least one of manipulate a location of the superior radial arm with respect to the humerus or check for version alignment between the resection guide and the humerus.

83. The surgical kit of any of examples 79 to 82, further comprising a removable extender coupled to the proximal portion of the superior radial arm and configured to extend the resecting plane inferiorly, the removable extender including at least one inferior bone pin receiving opening.

84. The surgical kit of any of examples 79 to 83, further comprising a drill cannula, the drill cannula being configured to pass into and through the cannula-receiving opening to engage an opposed surface of a bone of the one or more bones at which the distal end of the attachment coupled to the distal end of the rigid arm is located.

85. The surgical kit of any of examples 79 to 84, wherein the at least one bone pin clamp comprises:
a guide-coupling portion configured to couple to the at least one support rod; and
a pin-engaging portion configured to selectively unlock and lock the bone pin such that each of a location of entry of the bone pin into bone and an angle of entry of the bone pin into bone can be adjusted.

86. The surgical kit of any of examples 79 to 85, further comprising:
an attachment configured to be coupled to the distal end of the rigid arm, the attachment having a distal end with a primary surface that defines a plane that is substantially orthogonal to the longitudinal axis extending through the cannula-receiving opening that defines the path of travel for a drill cannula.

87. The surgical kit of example 86, wherein the attachment comprises a sizer attachment configured to at least one of define a central location of a receiving surface of a bone of the one or more bones at the surgical site or be used in determining a size of the receiving surface.

88. The surgical kit of example 86 or example 87, wherein the attachment comprises a handle assembly having a humeral bone preparation instrument coupled to a distal end thereof and configured to position the humeral bone preparation instrument proximate to a bone of the one or more bones at the surgical site such that a plane defined by a primary surface of the humeral bone preparation instrument is substantially orthogonal to the longitudinal axis extending through the cannula-receiving opening that defines the path of travel for a drill cannula.

89. The surgical kit of example 88, wherein the handle assembly further comprises a guide-receiving opening configured to be coupled to the distal end of the receiving arm to fixedly couple the handle assembly to the guide assembly for operating of the humeral bone preparation instrument.

90. The surgical kit of example 88 or example 89, further comprising a tool attachment, the tool attachment comprising:
the bone preparation instrument;
a mount having a proximal end configured to be grasped by the handle assembly and a distal end coupled to the bone preparation instrument;
a slidable capture plate having an opening formed therein, the capture plate disposed between the bone preparation instrument and the mount, and the capture plate being moveable between a locked position in which it can be configured to engage a guide pin disposed through the opening of the slidable capture plate to couple the guide pin to the handle assembly, and an unlocked position in which the guide pin is able to be decoupled from the capture plate, and thus the handle assembly.

91. The surgical kit of any of examples 88 to 90, wherein the handle assembly comprises:
an arm having a proximal portion and a distal portion;
an attachment portion disposed at the distal portion of the arm, the attachment portion being configured to receive a bone preparation instrument for use at the location where the bone is to be treated; and
a receiving portion disposed on the proximal portion of the arm, the receiving portion being configured to allow the handle assembly to be selectively coupled to a guide that provides proper positioning for the handle assembly to position a distal end of the attachment portion proximate to the location where the bone is to be treated.

92. The surgical kit of any of examples 79 to 91, further comprising:
a glenoid guide configured to couple to at least one of the humeral resection surface of the humerus.

93. The surgical kit of example 92, wherein the glenoid guide comprises:
a proximal portion having a proximal opening formed therein;
a distal portion having a distal opening formed therein, the distal opening being colinear with the proximal opening; and
an offset arm extending from the proximal portion to the distal portion, the offset arm being offset with respect to a longitudinal axis extending through the distal and proximal openings.

Although the procedures provided for herein are described in conjunction with performing an anatomical shoulder procedure in which the resected humeral head is replaced with a prosthesis that mimics a humeral head, the instruments and procedures provided for herein can also be used and applied in a reverse shoulder procedure in which the humeral head prosthesis is disposed on the glenoid and the implant placed on the humerus is a humeral head prosthesis receiving surface. A person skilled in the art, in view of the present disclosures, will understand how reverse shoulder techniques can be implemented in view of the present disclosures.

One skilled in the art will appreciate further features and advantages of the disclosure based on the above-described embodiments and techniques. Accordingly, the disclosure is not to be limited by what has been particularly shown and described, except as indicated by the appended claims. By way of example, while the present disclosure primarily focuses on shoulder arthroplasty procedures, and the use of humeral resection guides, glenoid guides, humeral guides, handle assemblies, and other components, instruments, tools, implants, etc. in conjunction with the same, the disclosed techniques, devices, and the like can be used and/or adapted for use with other shoulder procedures and/or for use in surgical procedures in other locations in the body. Accordingly, references to shoulder anatomy and/or guides herein being “humeral” or for the “glenoid” are not limiting to such use, and the disclosures herein can be used in procedures and guides for other anatomies (e.g., boney anatomies), whether human or other animals. A person skilled in the art, in view of the present disclosures, will be able to adapt some or all of the various systems, instruments, tools, and techniques disclosed herein for use in surgical procedures in other locations and/or for use with non-humans.

Further, a person skilled in the art will appreciate that various features or other disclosures associated with one embodiment of a device, system, component, and/or surgical technique can be used in other devices, systems, components, and/or surgical techniques disclosed herein or otherwise derivable therefrom. It is within the skill of a person skilled in the art to be able to apply teachings, or part of teachings, from one such device, system, component, and/or surgical technique to one or more other devices, systems, components, and/or surgical techniques. While various descriptions and claims describe portions of a process, and up to an entire process, for performing a surgical procedure (e.g., an entire process possibly beginning at exposing a glenohumeral joint and glenohumeral joint space and concluding with disposing any implant(s) and/or prosthesis(es) to the surgical site and closing access to the surgical site, the various bone preparation actions provided for herein occurring therebetween), a person skilled in the art will appreciate that smaller aspects or portions of the procedures can also be described and claimed without requiring all of the actions. Likewise, a person skilled in the art will appreciate certain aspects of the described procedures that can be mixed and matched with each other and/or with other known techniques, as well as portions of a procedure that may not be necessary in at least some instances. The present disclosure is by no means intended to require all described actions to be performed in a single procedure, though they can be, nor is the present disclosure intended to require or not require certain disclosed actions unless otherwise indicated.

To the extent the present disclosure does not describe materials that can be used to manufacture the various devices and the like disclosed herein (e.g., humeral resection guides, glenoid guides, humeral guides, handle assemblies, implants, end effectors, and other components, instruments, tools, etc.) and/or does not identify particular dimensions and the like for such devices and the like, a person skilled in the art will appreciate typical materials and dimensions that are appropriate. All publications and references cited herein are expressly incorporated herein by reference in their entirety.

1. A surgical method, comprising:
exposing a glenohumeral joint and glenohumeral joint space;
coupling a resection guide to at least one of a humeral head or a humerus of the glenohumeral joint;
introducing a cutting instrument to the glenohumeral joint space;
resecting at least a portion of the humeral head located at the glenohumeral joint using the resection guide to guide the cutting instrument and creating a humeral resection surface;
removing the resection guide and the cutting instrument from the glenohumeral joint space;
coupling a humeral guide to at least one of the humeral resection surface or the humerus such that a proximal portion of the humeral guide is disposed below the humeral resection surface, at a location that is opposed to the humeral resection surface, and a distal portion of the humeral guide is disposed proximate to a rotator interval, approximately aligned with the humeral resection surface;
coupling a humeral sizer attachment to the distal end of the humeral guide;
identifying an approximate center point of the humeral resection surface with a distal portion of the humeral sizer attachment;
forming a transhumeral bone tunnel in the humerus using the humeral guide and the humeral sizer attachment to guide a drill bit through the humerus and the humeral resection surface;
de-coupling the humeral sizer attachment from the distal end of the humeral guide;
introducing a distal end of a handle assembly to the glenohumeral joint space, the distal end of the handle assembly having coupled thereto a humeral bone preparation instrument;
coupling the handle assembly to the distal end of the humeral guide;
passing a guide pin through the transhumeral bone tunnel;
coupling the guide pin to the humeral bone preparation instrument;
operating the humeral bone preparation instrument to treat the humeral resection surface;
de-coupling the guide pin from the humeral bone preparation instrument; and
removing the distal end of the handle assembly and the humeral bone preparation instrument from the glenohumeral joint space.

2-6. (canceled)

7. The method of claim 1, wherein resecting at least a portion of a humeral head located at the glenohumeral joint further comprises:
engaging the humeral head with a superior arm of a resection guide;
passing at least one bone pin through a portion of the resection guide and into the humeral head; and
passing the cutting instrument through a guide slot formed on the resection guide.

8-15. (canceled)

16. The method of claim 7, further comprising mating an extender to the resection guide, the extender being configured to extend a cutting plane defined by the superior arm of the resection guide inferior to the subscapularis tendon.

17. (canceled)

18. The method of claim 1, further comprising:
introducing a reamer to the glenohumeral joint space;
reaming a glenoid surface of a glenoid of the glenohumeral joint using the reamer; and
removing the reamer from the glenohumeral joint space.

19-21. (canceled)

22. The method of claim 18, further comprising:
coupling a glenoid guide to at least one of the humeral resection surface or the humerus such that a distal portion of the glenoid guide is disposed between the humeral resection surface and a surface of a glenoid of the glenohumeral joint; and
forming a second transhumeral bone tunnel using the glenoid guide to guide a drill bit through the humerus.

23-33. (canceled)

34. The method of claim 1, wherein identifying an approximate center point of the humeral resection surface with a distal portion of the humeral sizer attachment, further comprises:
positioning the distal portion of the humeral sizer attachment such that is aligned to be substantially parallel with the humeral resection surface; and
aligning an anatomical landmark of the humerus with an indicator of the humeral sizer attachment.

35. The method of claim 1, wherein coupling a humeral guide to at least one of the humeral resection surface or the humerus further comprises:
passing a drill cannula through a hub of the humeral guide; and
coupling a distal end of the drill cannula to a lateral cortex of the humerus.

36-46. (canceled)

47. The method of claim 1, further comprising:
after removing the humeral bone preparation instrument from the glenohumeral joint space, introducing the distal end of the handle assembly to the glenohumeral joint space, the distal end of the handle assembly having coupled thereto a second humeral bone preparation instrument;
coupling the handle assembly to the distal end of the humeral guide;
passing a guide pin through the transhumeral bone tunnel;
coupling the guide pin to the second humeral bone preparation instrument;
operating the second humeral bone preparation instrument to treat the humeral resection surface;
de-coupling the guide pin from the second humeral bone preparation instrument; and
removing the second humeral bone preparation instrument from the glenohumeral joint space.

48. The method of claim 1, wherein coupling the handle assembly to the distal end of the humeral guide, in one or both instances with respect to claim 47, further comprises:
positioning a receiving opening formed in the handle assembly over the distal end of the humeral guide;
passing the distal end of the humeral guide into the receiving opening formed in the handle assembly; and
securing the handle assembly to the distal end of the humeral guide using a slidable adapter disposed on the distal end of the humeral guide.

49-56. (canceled)

57. The method of claim 1, further comprising coupling a humeral bone preparation instrument to the handle assembly.

58. (canceled)

59. The method of claim 1, further comprising:
introducing a humeral implant to the glenohumeral joint space;
implanting the humeral implant into the humeral resection surface;
introducing a humeral head prosthesis to the glenohumeral joint space; and
coupling the humeral head prosthesis to the humeral implant,
wherein for methods in which the glenoid implant is implanted into the glenoid surface, the glenoid implant is configured to operate like an anatomic glenoid surface.

60-64. (canceled)

65. The method of claim 1 in which the glenoid implant has been introduced, the glenoid implant being configured to receive a glenoid prosthesis that is configured to operate like an anatomic humeral head, the method further comprising:
introducing a humeral implant to the glenohumeral joint space, the humeral implant being configured to provide a surface for receiving a glenoid prosthesis that is configured to operate like an anatomic humeral head;
implanting the humeral implant into the humeral resection surface;
introducing a glenoid prosthesis that is configured to operate like an anatomic humeral head to the glenohumeral joint space; and
coupling the glenoid prosthesis to the glenoid implant.

66-68. (canceled)

69. The method of claim 1, wherein the method is performed with a transhumeral approach.

70. The method of claim 69, wherein the introducing actions performed during the method are performed such that whatever is being introduced into the glenohumeral joint space is introduced from a location that is lateral to the humeral resection surface.

71. The method of claim 69, wherein the introducing actions performed during the method are performed such that the introduction occurs substantially orthogonal to a humeral cut plane.

72. The method of claim 69, wherein the introducing actions occur through the rotator interval.

73. The method of claim 1, wherein the method is performed while keeping the subscapularis tendon intact.

74. The method of claim 1, wherein the method is performed without distracting the humeral head from its joint.

75. A surgical method, comprising:
coupling a resection guide to at least one of a humeral head or a humerus of a glenohumeral joint;
resecting at least a portion of the humeral head located at the glenohumeral joint using the resection guide to guide a cutting instrument and creating a humeral resection surface;
removing the resection guide from the glenohumeral joint space;
coupling a humeral guide to at least one of the humeral resection surface or the humerus such that a proximal portion of the humeral guide is disposed below the humeral resection surface, at a location that is opposed to the humeral resection surface, and a distal portion of the humeral guide is disposed proximate to a rotator interval, approximately aligned with the humeral resection surface;
forming a transhumeral bone tunnel in the humerus using the humeral guide to guide a drill bit through the humerus and the humeral resection surface;
coupling a handle assembly to the distal end of the humeral guide, the handle assembly having a humeral bone preparation instrument coupled to a distal end thereof; and
operating the humeral bone preparation instrument by way of a guide pin disposed in the transhumeral bone tunnel to treat the humeral resection surface,
wherein a subscapularis tendon remains intact throughout the method.

76. The method of claim 75, further comprising:
exposing a glenohumeral joint and glenohumeral joint space;
introducing the cutting instrument to the glenohumeral joint space;
after removing the resection guide from the glenohumeral joint space and coupling the humeral guide to at least one of the humeral resection surface or the humerus, coupling a humeral sizer attachment to the distal end of the humeral guide;
identifying an approximate center point of the humeral resection surface with a distal portion of the humeral sizer attachment;
prior to coupling a handle assembly to the distal end of the humeral guide, de-coupling the humeral sizer attachment from the distal end of the humeral guide;
introducing the distal end of the handle assembly to the glenohumeral joint space;
prior to operating the humeral bone preparation instrument by way of a guide pin disposed in the transhumeral bone tunnel to treat the humeral resection surface, passing a guide pin through the transhumeral bone tunnel; and
coupling the guide pin to the humeral bone preparation instrument,
wherein forming a transhumeral bone tunnel in the humerus using the humeral guide to guide a drill bit through the humerus and the humeral resection surface further comprises forming the transhumeral bone tunnel in the humerus using the humeral guide and the humeral sizer attachment to guide the drill bit through the humerus and the humeral resection surface.

77-93. (canceled)
